(19) United States
(12) Patent Application Publication
(10) Pub. No.: US 2025/0009873 A1
(43) Pub. Date: Jan. 9, 2025

(54) METHODS OF PREVENTING, TREATING, OR REDUCING THE SEVERITY OF COVID-19 IN IMMUNOCOMPROMISED BLOOD CANCER PATIENTS

(71) Applicant: CITY OF HOPE, Duarte, CA (US)

(72) Inventors: Don J. DIAMOND, Duarte, CA (US); Flavia CHIUPPESI, Duarte, CA (US)

(73) Assignee: CITY OF HOPE, Duarte, CA (US)

(21) Appl. No.: 18/711,088

(22) PCT Filed: Nov. 17, 2022
(86) PCT No.: PCT/US2022/080073

§ 371 Date: May 16, 2024

Related U.S. Application Data
(60) Provisional application No. 63/280,557, filed on Nov. 17, 2021.
(60) Provisional application No. 63/280,561, filed on Nov. 17, 2021.

(51) Int. Cl. A61K 39/215; A61K 39/00; A61P 37/04; C07K 14/005

(52) U.S. Cl. CPC A61K 39/215; A61P 37/04; C07K 14/005; A61K 2039/53; C12N 2770/20022; C12N 2770/20034; C12N 2770/20071

(57) ABSTRACT

Disclosed are methods of preventing or treating a coronavirus infection in a blood cancer patient having received a cellular therapy by administration of a synthetic MVA-based vaccine.

### CROSS-REFERENCE TO RELATED APPLICATIONS

This application claims the benefit of U.S. Provisional Patent Application No. 63/280,557, filed on Nov. 17, 2021, and U.S. Provisional Patent Application No. 63/280,561, filed on Nov. 17, 2021, the entire contents of each of which are incorporated by reference.

### SEQUENCE LISTING

This application contains a Sequence Listing, which was submitted in XML format via Patent Center, and is hereby incorporated by reference in its entirety. The XML copy, created on Nov. 16, 2022, is named 0544358221WO00.xml and is 590 KB in size.

### BACKGROUND

On Feb. 4, 2020, the Secretary of Health and Human Services (HHS) determined that there was a public health emergency concerning the spread of a novel coronavirus, later named “severe acute respiratory syndrome coronavirus 2” (SARS-CoV-2), and the disease it causes has been named “Coronavirus Disease 2019” (COVID-19). Since then, SARS-CoV-2 has caused a global pandemic with almost 250M cases and 5M fatalities (as of Nov. 1, 2021). Preventing the incidence of COVID-2019-associated morbidity and mortality while allowing a return to normal activities may best be accomplished by prophylactic vaccination against SARS-CoV-2. Spike (S)-based vaccines appear to protect from hospitalization and severe disease, yet, as virus variants arise with mutations primarily within the virus S-protein, there is concern that vaccine-induced immunity might be insufficient to control disease. To hasten the end of the pandemic and protect against the spread of variants, a preventative SARS-CoV-2 vaccine, COH04S1, which targets both S- and the less variant prone nucleocapsid (N) protein, was developed.

Following the emergence of Alpha (B.1.1.7), Beta (B.1.351), Gamma (P.1), and Delta (B.1.617), Omicron subvariants have emerged as predominant SARS-CoV-2 VOC, which includes BA.1, BA.2, BA.2 sub-lineages such as BA.2.12.1, BA.4, BA.5, BA.2.75, and more recent subvariants such as BQ.1, BQ.1.1, AND XBB. Omicron subvariants have exceptional capacity to evade neutralizing antibodies (NAb) due to numerous mutations in the S protein that dramatically exceed S mutations of other earlier occurring SARS-CoV-2 VOC, thereby posing a unique challenge for COVID-19 vaccination. Several studies reported reduced clinical effectiveness against Omicron variants by approved COVID-19 vaccines, which were designed to elicit protective immunity solely based on the S protein of the Wuhan-Hu-1 reference strain. While waning vaccine efficacy against VOC can be counteracted by repeated booster vaccination, COVID-19 booster vaccines with altered or variant-matched antigen design have been developed to specifically enhance the stimulation of cross-protective immunity against emerging SARS-CoV-2 VOC.

The Centers for Disease Control and Prevention (CDC) lists immunocompromised patients such as hematology patients who received therapeutic procedures for hematologic malignancy as high risk for COVID-19. SARS-CoV-2 infection is expected to be very serious in the vulnerable population of hematology patients, including recipients of autologous (auto) and/or allogeneic (allo) hematopoietic cell transplant (HCT), and recipients of chimeric antigen receptor (CAR)-T cell therapy. Due to their immunocompromised status, hematology patients are at increased risk for severe COVID-19 disease, including respiratory complications and exacerbated lethality of the infection. There is very limited data and multiple critical gaps in our knowledge of the epidemiology and clinical manifestations of COVID-19 in hematology patients. Given the serious impact of other respiratory viruses in this vulnerable patient population, it is anticipated that hematology recipients of cell therapy may develop severe clinical disease, profoundly impacting the therapy outcomes, such as morbidity and survival.

As of Jan. 25, 2021, the Center for International Blood and Marrow Transplant Research (CIBMTR; www.cibmtr.org/Covid19/Pages/default.aspx) reported 1362 COVID-19 infections in HCT recipients, with a ˜15% mortality rate (195 deaths), as communicated by 200 CIBMTR participating centers (162 US, 38 non-US). Currently, there are several drugs and investigational agents being evaluated in clinical trials in many nations, and other agents may be available through compassionate use programs. Nonetheless, no treatment has been proven effective against newer variants. Moreover, most trials require patients to be off immunosuppression for a certain period of time to be eligible. This may not be feasible in patients who are receiving therapy for hematologic malignancy. It is unclear how the different SARS-CoV-2 vaccine candidates and emergency use authorization (EUA) vaccines will specifically affect different forms of immune abnormalities. Given the diversity of various immunocompromised patient populations, it is possible that candidate SARS-CoV-2 vaccines may differ in their efficacy and safety for these patients.

Despite a high vaccination rate, patients with malignant disease may be at high risk for lethal COVID-19 infection due to poor immune response to COVID-19 infections or vaccination. This disclosure provides vaccines using a synthetic MVA platform for preventing COVID-19 in these patients, as well as for preventing or lessening the severity of COVID-19 in immunocompromised blood cancer patients.

### SUMMARY

The present technologies provide methods of vaccinating or protecting against a coronavirus infection, or preventing or treating COVID-19, in an immunocompromised subject, for example, a blood cancer patient who has received a cellular therapy, by administration of a synthetic MVA-based vaccine.

In some aspects, provided are methods of vaccinating or protecting a subject against coronavirus disease 2019 (COVID-19) caused by a coronavirus infection, comprising administering to the subject a composition comprising a synthetic MVA (sMVA) vector comprising or capable of expressing one or more DNA sequences encoding a spike (S) protein and a nucleocapsid (N) protein or variants or mutants thereof, wherein the subject is a blood cancer patient that has been treated for a hematological malignancy with a cellular therapy.

In some aspects, provided are methods of preventing a coronavirus infection in a subject, comprising administering to the subject a composition comprising a synthetic MVA (sMVA) vector comprising or capable of expressing one or more DNA sequences encoding a spike (S) protein and a nucleocapsid (N) protein or variants or mutants thereof, wherein the subject is a blood cancer patient that has been treated for a hematological malignancy with a cellular therapy.

In some aspects, provided are methods of preventing, treating, or reducing the severity of COVID-19 caused by a coronavirus infection in a subject, comprising administering to the subject a composition comprising a synthetic MVA (sMVA) vector comprising or capable of expressing one or more DNA sequences encoding a spike (S) protein and a nucleocapsid (N) protein or variants or mutants thereof, wherein the subject is a blood cancer patient that has been treated for a hematological malignancy with a cellular therapy.

In some aspects, provided are methods of treating COVID-19 caused by a coronavirus infection in a subject, comprising administering to the subject a composition comprising a synthetic MVA (sMVA) vector comprising or capable of expressing one or more DNA sequences encoding a spike (S) protein and a nucleocapsid (N) protein or variants or mutants thereof, wherein the subject is a blood cancer patient that has been treated for a hematological malignancy with a cellular therapy.

In some aspects, provided are methods of vaccinating or protecting a subject against coronavirus disease 2019 (COVID-19) caused by a coronavirus infection, comprising administering to the subject a composition comprising a synthetic MVA (sMVA) vector comprising or capable of expressing one or more DNA sequences encoding a spike (S) protein and a nucleocapsid (N) protein or variants or mutants thereof, wherein the subject is a blood cancer patient who has had or is likely to have a poor immune response to a different COVID-19 vaccination.

In some aspects, provided are methods of preventing a coronavirus infection in a subject, comprising administering to the subject a composition comprising a synthetic MVA (sMVA) vector comprising or capable of expressing one or more DNA sequences encoding a spike (S) protein and a nucleocapsid (N) protein or variants or mutants thereof, wherein the subject is a blood cancer patient who has had or is likely to have a poor immune response to a different COVID-19 vaccination.

In some aspects, provided are methods of preventing, treating, or reducing the severity of COVID-19 caused by a coronavirus infection in a subject, comprising administering to the subject a composition comprising a synthetic MVA (sMVA) vector comprising or capable of expressing one or more DNA sequences encoding a spike (S) protein and a nucleocapsid (N) protein or variants or mutants thereof, wherein the subject is a blood cancer patient who has had or is likely to have a poor immune response to a different COVID-19 vaccination.

In some aspects, provided are methods of treating COVID-19 caused by a coronavirus infection in a subject, comprising administering to the subject a composition comprising a synthetic MVA (sMVA) vector comprising or capable of expressing one or more DNA sequences encoding a spike (S) protein and a nucleocapsid (N) protein or variants or mutants thereof, wherein the subject is a blood cancer patient who has had or is likely to have a poor immune response to a different COVID-19 vaccination.

In some aspects, provided are compositions for use in a method of vaccinating or protecting a subject against coronavirus disease 2019 (COVID-19) caused by a coronavirus infection, wherein the composition comprises a synthetic MVA (sMVA) vector comprising or capable of expressing one or more DNA sequences encoding a spike (S) protein and a nucleocapsid (N) protein or variants or mutants thereof, and wherein the subject is a blood cancer patient that has been treated for a hematological malignancy with a cellular therapy.

In some aspects, provided are compositions for use in a method of preventing a coronavirus infection in a subject, wherein the composition comprises a synthetic MVA (sMVA) vector comprising or capable of expressing one or more DNA sequences encoding a spike (S) protein and a nucleocapsid (N) protein or variants or mutants thereof, and wherein the subject is a blood cancer patient that has been treated for a hematological malignancy with a cellular therapy.

In some aspects, provided are compositions for use in a method of preventing, treating, or reducing the severity of COVID-19 caused by a coronavirus infection in a subject, wherein the composition comprises a synthetic MVA (sMVA) vector comprising or capable of expressing one or more DNA sequences encoding a spike (S) protein and a nucleocapsid (N) protein or variants or mutants thereof, and wherein the subject is a blood cancer patient that has been treated for a hematological malignancy with a cellular therapy.

In some aspects, provided are compositions for use in a method of treating COVID-19 caused by a coronavirus infection in a subject, wherein the composition comprises a synthetic MVA (sMVA) vector comprising or capable of expressing one or more DNA sequences encoding a spike (S) protein and a nucleocapsid (N) protein or variants or mutants thereof, and wherein the subject is a blood cancer patient that has been treated for a hematological malignancy with a cellular therapy.

In some aspects, provided are compositions for use in a method of vaccinating or protecting a subject against coronavirus disease 2019 (COVID-19) caused by a coronavirus infection, wherein the composition comprises a synthetic MVA (sMVA) vector comprising or capable of expressing one or more DNA sequences encoding a spike (S) protein and a nucleocapsid (N) protein or variants or mutants thereof, and wherein the subject is a blood cancer patient who has had or is likely to have a poor immune response to a different COVID-19 vaccination.

In some aspects, provided are compositions for use in a method of preventing a coronavirus infection in a subject, wherein the composition comprises a synthetic MVA (sMVA) vector comprising or capable of expressing one or more DNA sequences encoding a spike (S) protein and a nucleocapsid (N) protein or variants or mutants thereof, and wherein the subject is a blood cancer patient who has had or is likely to have a poor immune response to a different COVID-19 vaccination.

In some aspects, provided are compositions for use in a method of preventing, treating, or reducing the severity of COVID-19 caused by a coronavirus infection in a subject, wherein the composition comprises a synthetic MVA (sMVA) vector comprising or capable of expressing one or more DNA sequences encoding a spike (S) protein and a nucleocapsid (N) protein or variants or mutants thereof, and wherein the subject is a blood cancer patient who has had or is likely to have a poor immune response to a different COVID-19 vaccination.

In some aspects, provided are compositions for use in a method of treating COVID-19 caused by a coronavirus infection in a subject, wherein the composition comprises a synthetic MVA (sMVA) vector comprising or capable of expressing one or more DNA sequences encoding a spike (S) protein and a nucleocapsid (N) protein or variants or mutants thereof, and wherein the subject is a blood cancer patient who has had or is likely to have a poor immune response to a different COVID-19 vaccination.

In some embodiments, the cellular therapy is selected from the group consisting of an autologous hematopoietic cell transplant, an allogeneic hematopoietic cell transplant, a chimeric antigen receptor (CAR)-T cell therapy, and a combination thereof.

In some embodiments, the subject received the cellular therapy at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, or at least 12 months prior to administration of the composition.

In some embodiments, the coronavirus infection is caused by the Wuhan-Hu-1 reference strain or a variant of concern (VOC) selected from the group consisting of B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments, the composition is administered by intramuscular injection, intranasal instillation, intradermal injection, and/or scarification.

In some embodiments, the composition is administered to the subject in a single dose, two doses, three doses, four doses, or more than four doses.

In some embodiments, the composition is administered to the subject in a prime dose followed by one or more booster doses.

In some embodiments, an interval between the prime dose and the first booster dose is about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, or about 16 weeks.

In some embodiments, the prime dose is between 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose. In some embodiments, the prime dose is about 1.0×10⁷ PFU/dose. In some embodiments, the prime dose is about 1.5×10⁷ PFU/dose. In some embodiments, the prime dose is about 2.0×10⁷ PFU/dose. In some embodiments, the prime dose is about 2.5×10⁷ PFU/dose. In some embodiments, the prime dose is about 3.0×10⁷ PFU/dose. In some embodiments, the prime dose is about 3.5×10⁷ PFU/dose. In some embodiments, the prime dose is about 4.0×10⁷ PFU/dose. In some embodiments, the prime dose is about 4.5×10⁷ PFU/dose. In some embodiments, the prime dose is about 5×10⁷ PFU/dose. In some embodiments, the prime dose is about 5.5×10⁷ PFU/dose. In some embodiments, the prime dose is about 6×10⁷ PFU/dose. In some embodiments, the prime dose is about 6.5×10⁷ PFU/dose. In some embodiments, the prime dose is about 7×10⁷ PFU/dose. In some embodiments, the prime dose is about 7.5×10⁷ PFU/dose. In some embodiments, the prime dose is about 8×10⁷ PFU/dose. In some embodiments, the prime dose is about 8.5×10⁷ PFU/dose. In some embodiments, the prime dose is about 9×10⁷ PFU/dose. In some embodiments, the prime dose is about 9.5×10⁷ PFU/dose. In some embodiments, the prime dose is about 1×10⁸ PFU/dose. In some embodiments, the prime dose is about 1.5×10⁸ PFU/dose. In some embodiments, the prime dose is about 2×10⁸ PFU/dose. In some embodiments, the prime dose is about 2.5×10⁸ PFU/dose. In some embodiments, the prime dose is about 3×10⁸ PFU/dose. In some embodiments, the prime dose is about 3.5×10⁸ PFU/dose. In some embodiments, the prime dose is about 4×10⁸ PFU/dose. In some embodiments, the prime dose is about 4.5×10⁸ PFU/dose. In some embodiments, the prime dose is about 5×10⁸ PFU/dose.

In some embodiments, the booster dose is between 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose. In some embodiments, the booster dose is about 1.0×10⁷ PFU/dose. In some embodiments, the booster dose is about 1.5×10⁷ PFU/dose. In some embodiments, the booster dose is about 2.0×10⁷ PFU/dose. In some embodiments, the booster dose is about 2.5×10⁷ PFU/dose. In some embodiments, the booster dose is about 3.0×10⁷ PFU/dose. In some embodiments, the booster dose is about 3.5×10⁷ PFU/dose. In some embodiments, the booster dose is about 4.0×10⁷ PFU/dose. In some embodiments, the booster dose is about 4.5×10⁷ PFU/dose. In some embodiments, the booster dose is about 5×10⁷ PFU/dose. In some embodiments, the booster dose is about 5.5×10⁷ PFU/dose. In some embodiments, the booster dose is about 6×10⁷ PFU/dose. In some embodiments, the booster dose is about 6.5×10⁷ PFU/dose. In some embodiments, the booster dose is about 7×10⁷ PFU/dose. In some embodiments, the booster dose is about 7.5×10⁷ PFU/dose. In some embodiments, the booster dose is about 8×10⁷ PFU/dose. In some embodiments, the booster dose is about 8.5×10⁷ PFU/dose. In some embodiments, the booster dose is about 9×10⁷ PFU/dose. In some embodiments, the booster dose is about 9.5×10⁷ PFU/dose. In some embodiments, the booster dose is about 1×10⁸ PFU/dose. In some embodiments, the booster dose is about 1.5×10⁸ PFU/dose. In some embodiments, the booster dose is about 2×10⁸ PFU/dose. In some embodiments, the booster dose is about 2.5×10⁸ PFU/dose. In some embodiments, the booster dose is about 3×10⁸ PFU/dose. In some embodiments, the booster dose is about 3.5×10⁸ PFU/dose. In some embodiments, the booster dose is about 4×10⁸ PFU/dose. In some embodiments, the booster dose is about 4.5×10⁸ PFU/dose. In some embodiments, the booster dose is about 5×10⁸ PFU/dose.

In some embodiments, the booster dose is administered in a dosage the same as the prime dose. In some embodiments, the booster dose is administered in a dosage lower than the prime dose.

In some embodiments, the subject suffers from or previously suffered from a hematological malignancy. In some embodiments, the hematological malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

In some embodiments, the subject has previously received one or more COVID-19 vaccines. In some embodiments, the previously received COVID-19 vaccine is an mRNA-, adenovirus-, or protein-based vaccine. In some embodiments, the previously received COVID-19 vaccine is Pfizer-BioNTech COVID-19 vaccine (BNT162b2), Moderna COVID-19 vaccine (mRNA-1273), Janssen COVID-19 vaccine (Ad26.COV2.S), Novavax COVID-19 vaccine adjuvanted, or Oxford-AstraZeneca ChAdOx1 nCoV-19 vaccine (AZD1222). In some embodiments, the previously received COVID vaccine comprises an S antigen or a coding sequence for an S antigen only. In some embodiments, the subject has received the previous vaccination at least 1 month, at least 2 months, at least 3 months, at least 4 months, at least 4 months, at least 5 months, at least 6 months, at least 7 months, at least 8 months, at least 9 months, at least 10 months, at least 11 months, or at least 12 months prior to administration of the composition.

### BRIEF DESCRIPTION OF THE DRAWINGS

FIG. 1 shows a study design schema.

FIGS. 2A-2B show treatment schemas. FIG. 2A: treatment schema with two doses of COH04S1 or Pfizer vaccine. FIG. 2B: amended treatment schema with four doses. This schema only applies to patients COH208, COH210, COH211, COH212, COH213. COH04S1, investigational COVID-19 vaccine; Pfizer, Comirnaty or similar SOC vaccine; AE, adverse events; EOT, end of treatment; PIA, primary immune assessment.

FIG. 3 shows a study design schema of COH04S1 booster in patients with poor COVID-19 immunity.

FIG. 4 shows a treatment schema. AE, adverse events; EOT, end of treatment; PIA, primary immune assessment. * represents the visits that may be tele-health visits with home phlebotomy.

FIG. 5 shows clinical trial enrollment status and patients' therapy group. Top table shows enrollment in the lead-in safety portion (6 patients/therapy type). Bottom table shows enrollment in the randomized portion. *=patients following the amended study scheme (FIG. 2B).

FIG. 6 shows spike (S), receptor binding domain (RBD), and nucleocapsid (N) binding antibody titers (binding antibody units (BAU)/ml) in COH04S1 vaccinated patients at different time points post-vaccination. d=day. *=value above threshold, needs re-testing.

FIG. 7 shows a statistical evaluation of spike (S), receptor binding domain (RBD), and nucleocapsid (N) binding antibody titers (binding antibody units (BAU)/ml) in COH04S1 vaccinated patients at different time points post-vaccination. Wilcoxon matched pairs signed rank test was used. P values<0.05 are indicated above the bars. Red and blue dotted lines indicate the IgG BAU/ml titers measured in BNT162b2-vaccinated healthcare workers (red, n=30), and COH04S1-vaccinated healthy volunteers (blue, N=30) at six months post-vaccination.

FIG. 8 shows neutralizing antibodies against SARS-CoV-2 ancestral strain (D614G), Beta, Delta, and Omicron BA.1 variants in serum of individual patients at baseline and at different timepoints post-vaccination. Day 56 D614G NT50 fold-increase compared to baseline is shown in each panel.

FIG. 9 shows a statistical evaluation of neutralizing antibodies against SARS-CoV-2 ancestral strain (D614G), Beta, Delta, and Omicron BA.1 variants in serum of COH04S1 vaccinated patients at baseline and at different timepoints post-vaccination. Black dotted line represents lower limit of detection. Red and blue dotted lines indicate the D614G-specific NT50 titers measured in BNT162b2-vaccinated healthcare workers (red, n=30), and COH04S1-vaccinated healthy volunteers (blue, N=30) at day 56 post-vaccination.

FIG. 10 shows spike (S)-, nucleocapsid (N)-, and membrane (M)-specific T cells evaluation using IFNγ ELISPOT on PBMC samples from individual COH04S1 vaccinated patients at different timepoints post-vaccination. Day 56 fold-increase in S- and N-IFNγ T cells compared to baseline is shown in each panel. Black dotted line represents the arbitrary threshold of positivity (50 spots/10⁶ cells).

FIG. 11 shows a statistical evaluation of Spike (S)-, Nucleocapsid (N)-, and Membrane (M)-specific T cells secreting IFNγ in PBMC samples of COH04S1-vaccinated patients at baseline and at different timepoints post-vaccination. Black dotted line represents the arbitrary threshold of positivity (50 spots/10⁶ cells). Red and blue dotted lines indicate the D614G-specific NT50 titers measured in BNT162b2-vaccinated healthcare workers (red, n=30), and COH04S1-vaccinated healthy volunteers (blue, N=30) at day 56 post-vaccination.

FIG. 12 shows a statistical evaluation of spike (S)-, nucleocapsid (N)-, and membrane (M)-specific T cells secreting IL-4 in PBMC samples of COH04S1-vaccinated patients at baseline and at different timepoints post-vaccination. Black dotted line represents the arbitrary threshold of positivity (50 spots/10⁶ cells).

FIG. 13 shows a statistical evaluation of spike (S)-, and nucleocapsid (N)-specific activation-induced marker (AIM)+ T cells in PBMC samples of COH04S1-vaccinated patients at baseline and at different timepoints post-vaccination.

FIGS. 14A-14C show sMVA construction. FIG. 14A is a schematic of an MVA genome. The MVA genome is about 178 kbp in length and contains about 9.6 kbp inverted terminal repeat (ITR) sequences. FIG. 14B shows the three sMVA fragments, F1, F2, and F3. The three subgenomic sMVA fragments (F1-F3) comprise about 60 kbp of the left, central, and right parts of the MVA genome as indicated. sMVA F1/F2 and F2/F3 share about 3 kbp overlapping homologous sequences for recombination (dotted crossed lines). Approximate genome positions of commonly used MVA insertion (Del2, IGR69/70, Del3) are indicated. FIG. 14C shows terminal concatemer resolution-hairpin loop-concatemer resolution (CR/HL/CR) sequences. Each of the sMVA fragments contains at both ends a sequence composition comprising a duplex copy of the MVA terminal HL flanked by CR sequences. BAC=bacterial artificial chromosome vector.

FIG. 15 shows the DNA sequence of an sMVA backbone with possible locations of insertion sites Del2, IGR69/70, and Del3, according to some embodiments.

FIG. 16 shows the DNA sequence of an F1 nucleotide fragment that includes a portion of the full MVA backbone sequence flanked by an MVA CR/HL/CR sequence with insertion sites, according to some embodiments.

FIG. 17 shows the DNA sequence of an F2 nucleotide fragment that includes a portion of the full MVA backbone sequence flanked by an MVA CR/HL/CR sequence with insertion sites, according to some embodiments.

FIG. 18 shows the DNA sequence of an F3 nucleotide fragment that includes a portion of the full MVA backbone sequence flanked by an MVA CR/HL/CR sequence with insertion sites, according to some embodiments.

### DETAILED DESCRIPTION

Disclosed herein are methods of vaccinating or protecting against a coronavirus infection, or preventing or treating COVID-19, in an immunocompromised subject (e.g., someone who has a weakened immune system). People can be immunocompromised either due to a medical condition or from receipt of immunosuppressive medications or treatments. In some embodiments, an immunocompromised subject may be a subject who has had or is having a hematological malignancy (blood cancer), and/or who has received or is receiving treatment (e.g., cellular therapy) for the hematological malignancy, including, for example, an autologous hematopoietic cell transplant, an allogeneic hematopoietic cell transplant, a chimeric antigen receptor (CAR)-T cell therapy, and/or a combination thereof.

According to some embodiments, the methods provide for an optimal timing after the cellular therapy to improve the chances that the subject's immune system response will result in preventing, treating, or reducing the severity of COVID-19 caused by a coronavirus infection, or preventing infection by the coronavirus, in the immunocompromised subject.

According to some embodiments, the methods disclosed herein use a recombinant synthetic MVA vector that is designed to express more than one SARS-CoV-2 proteins, mutant proteins, variant proteins, or immunogenic portions thereof. The rsMVA vector may improve the subject's immune system response in preventing, treating, or reducing the severity of COVID-19 caused by a coronavirus infection, or preventing infection by the coronavirus, in a blood cancer patient who has had a poor immune response to a different COVID-19 vaccination previously received or is likely to have a poor immune response to a different COVID-19 vaccination. According to some embodiments, the different COVID-19 vaccination is an mRNA vaccine and/or a vaccine that expresses or is capable of expressing only a single SARS-CoV-2 protein, mutant protein, variant protein, (or immunogenic portion thereof), e.g., the SARS-CoV-2 Spike protein (or a mutant, variant and/or immunogenic portion thereof).

The coronavirus infection that causes COVID-19 may be SARS-CoV-2 or any variants thereof according to the embodiments disclosed herein. In some embodiments, the coronavirus infection treated by the methods and compositions disclosed herein is caused by a variant of SARS-CoV-2. For example, a variant of SARS-CoV-2 may be a variant of concern including but not limited to B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

The methods disclosed herein include a step of administering to the subject a composition that includes a recombinant synthetic MVA (rsMVA) vector or reconstituted virus that expresses or is capable of expressing one or more heterologous DNA sequences encoding a SARS-CoV-2 Spike (S) protein and a SARS-CoV-2 Nucleocapsid (N) protein; or variants or mutants of the S protein and N protein. In some embodiments, the composition is a vaccine composition or any immunogenic composition capable of providing full or partial protection against infection by a coronavirus that causes COVID-19. In other embodiments, the composition is a vaccine composition or any immunogenic composition capable of providing full or partial protection from developing moderate or severe COVID-19 symptoms. In other embodiments, the composition is a vaccine composition or any immunogenic composition capable of providing protection from hospitalization or death as a result of developing COVID-19.

In some embodiments, the blood cancer patient suffers from or previously suffered from a hematological malignancy such as a B-cell hematological malignancy (i.e., the subject is a “blood cancer patient”). Hematologic malignancies include cancers affecting blood cells and bone marrow including, but not limited to, leukemias (e.g., acute lymphocytic leukemia (ALL), chronic lymphocytic leukemia (CLL), acute myeloid leukemia (AML), chronic myeloid leukemia (CML)), myelomas, and lymphomas (e.g., Hodgkin's and non-Hodgkin's (NHL)). For example, in some embodiments, the subject suffered from a hematological malignancy within the last two years. In some embodiments, the subject suffers from or previously suffered from a B-cell lymphoid malignancy such as CLL, B-NHL, Hodgkin lymphoma, B-cell ALL, or multiple myeloma.

In some embodiments, the subject (i.e., blood cancer patient) is an immunocompromised blood cancer patient that has previously received one or more cellular therapies including an autologous hematopoietic cell transplant, an allogeneic hematopoietic cell transplant, and a chimeric antigen receptor (CAR) T cell therapy. In some embodiments, the subject (i.e., blood cancer patient) received the cellular therapy within 1 week to 6 months prior to administration of a composition disclosed herein. In some embodiments, the subject (i.e., blood cancer patient) received the cellular therapy within 6 to 12 months prior to administration of a composition disclosed herein. In some embodiments, the subject (i.e., blood cancer patient) received the cellular therapy greater than or equal to 12 months prior to administration of a composition disclosed herein. In some embodiments, the subject (i.e., blood cancer patient) received the cellular therapy at least 1 week, at least 2 weeks, at least 3 weeks, at least 4 weeks, at least 1 month, at least 2 months, at least 3 months, at least 4 months, at least 4 months, at least 5 months, at least 6 months, at least 7 months, at least 8 months, at least 9 months, at least 10 months, at least 11 months, or at least 12 months prior to administration of a composition disclosed herein. In some embodiments, the subject received the cellular therapy between 3 months and 6 months, between 6 months and 12 months, or more than 12 months prior to administration of a composition disclosed herein.

In some embodiments, the subject has previously received one or more COVID-19 vaccines such as an mRNA vaccine or a vaccine targeting the S antigen only but developed poor immune response to the previous vaccination. In some embodiments, the subject received the previous vaccination at least 1 month, at least 2 months, at least 3 months, at least 4 months, at least 4 months, at least 5 months, at least 6 months, at least 7 months, at least 8 months, at least 9 months, at least 10 months, at least 11 months, or at least 12 months prior to administration of an rsMVA composition disclosed herein.

The composition may be administered to the subject in any suitable manner. In some embodiments, the composition is administered to the subject parenterally, e.g., by intramuscular injection. In some embodiments, the composition is administered to the subject by intranasal instillation. In some embodiments, the composition is administered to the subject by intradermal injection. In some embodiments, the composition is administered to the subject by scarification.

The compositions disclosed herein may be given to a subject as a single, stand-alone dose. Thus, in some embodiments, the composition is administered to the subject as a single dose. In other embodiments, the compositions may be given as a multiple-dose regimen. For example, in some embodiments, the composition is administered to the subject as a prime dose followed by a booster dose. In some embodiments, the composition is administered to the subject as a prime dose, followed by a first booster dose and a second booster dose. In some embodiments, one or more additional doses are administered to the subject after administration of the prime and booster doses. In some embodiments, the composition is administered to the subject as a single dose as a booster dose to the previous vaccination. In some embodiments, one or more additional doses are administered to the subject as booster doses to the previous vaccination.

According to the embodiments disclosed herein, the compositions for preventing, treating, or reducing the severity of COVID-19 caused by SARS-CoV-2 (or variants thereof) disclosed herein may be interchangeable with other commercially available COVID-19 vaccine compositions, such that the prime dose is different than the booster dose or doses, or such that the booster dose or doses are different from each other or the prime dose. In other words, each dose may be a different vaccine composition. In some embodiments, the subject has previously received a different SARS-CoV-2 vaccine before administration of the vaccine compositions disclosed herein. For example, the previously received SARS-CoV-2 vaccine is an mRNA vaccine or a vaccine composition comprising the S antigen only. In other embodiments, the subject receives a different SARS-CoV-2 vaccine after a prime dose of the compositions is given. The compositions disclosed herein may be given as any one or more of the doses administered to a subject.

In a multiple-dose regimen the first (or only) booster dose may be administered such that the interval between the prime dose and the booster is about 2 weeks, about 3 weeks, about 4 weeks, or about 30 days. Alternatively, in some embodiments, the booster administration may be delayed, such that the interval between the prime dose and the booster is greater than 30 days, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, or longer than 16 weeks. In some embodiments, the interval between the prime dose and the booster is about 90 days or longer than 90 days. In certain embodiments, the interval between the prime dose and the booster is about 8 weeks.

In some embodiments, the multiple-dose regimen includes one or more additional booster doses (e.g., a second booster dose, a third booster dose, and so on). Said booster doses may be administered such that the interval between each booster dose is delayed as compared to the interval between the prime dose and the first booster. In certain embodiments, the interval between each booster dose is about 8 weeks, about 9 weeks, about 10 weeks, about 11 weeks, about 12 weeks, about 13 weeks, about 14 weeks, about 15 weeks, about 16 weeks, or longer than 16 weeks. In some embodiments, the interval between each booster dose is about 90 days or longer than 90 days. In some embodiments, the interval between each booster dose is about 90 days or longer than 90 days. In certain embodiments, the interval between each booster dose is between about 6 months to about 1 year. The interval between each booster may be on an annual or semi-annual schedule to account for additional variants that may arise each year.

In some embodiments, the prime dose is between 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose.

In some embodiments, the booster dose is in the same dosage as the prime dose. In some embodiments, the booster dose is a lower dosage than the prime dose. In some embodiments, the booster dose (e.g., the first booster dose or the second booster dose) is between 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose. The booster dose may be in a dosage the same as the prime dose or lower than the prime dose.

The rsMVA vectors used in accordance with the embodiments disclosed herein include a synthetic MVA genome that serves as a viral backbone (referred to herein as the sMVA backbone or sMVA genome backbone) and one or more heterologous nucleotide sequences that encode (i) a SARS-CoV-2 S protein (or variant, mutant, and/or immunogenic fragment thereof) and (ii) a SARS-CoV-2 N protein (or variant, mutant, and/or immunogenic fragment thereof).

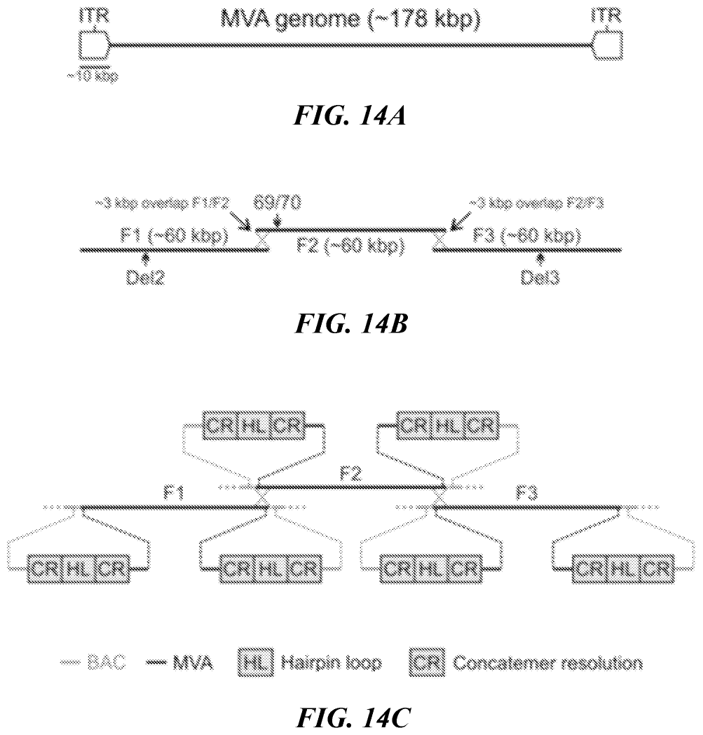

In some embodiments, the sMVA backbone includes an internal unique region (UR) of a parental MVA genome flanked on each side by at least a portion of an inverted terminal repeat (ITR) region of the parental MVA genome (FIG. 14A). The parental genome used to generate the sMVA backbone may be any known MVA strain or variant thereof including, but not limited to, MVA strain Acambis (Accession #AY603355) or MVA strain Antoine (NCBI Accession #U94848). In some embodiments, the sMVA backbone is identical to the corresponding full-length parental MVA genome sequence. In other embodiments, the sMVA backbone is identical or substantially identical to the corresponding full-length parental MVA genome sequence, but includes one or more unintentional mutations originating from chemical synthesis, cloning, propagation, or other errors resulting from the production of the synthetically made virus. For example, in some embodiments, the sMVA backbone has a single T to A nucleotide alteration located in the IGR at three base pairs downstream of open reading frame (ORF) 021 L. In some embodiments, the sMVA backbone has at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to the corresponding full-length parental MVA genome sequence.

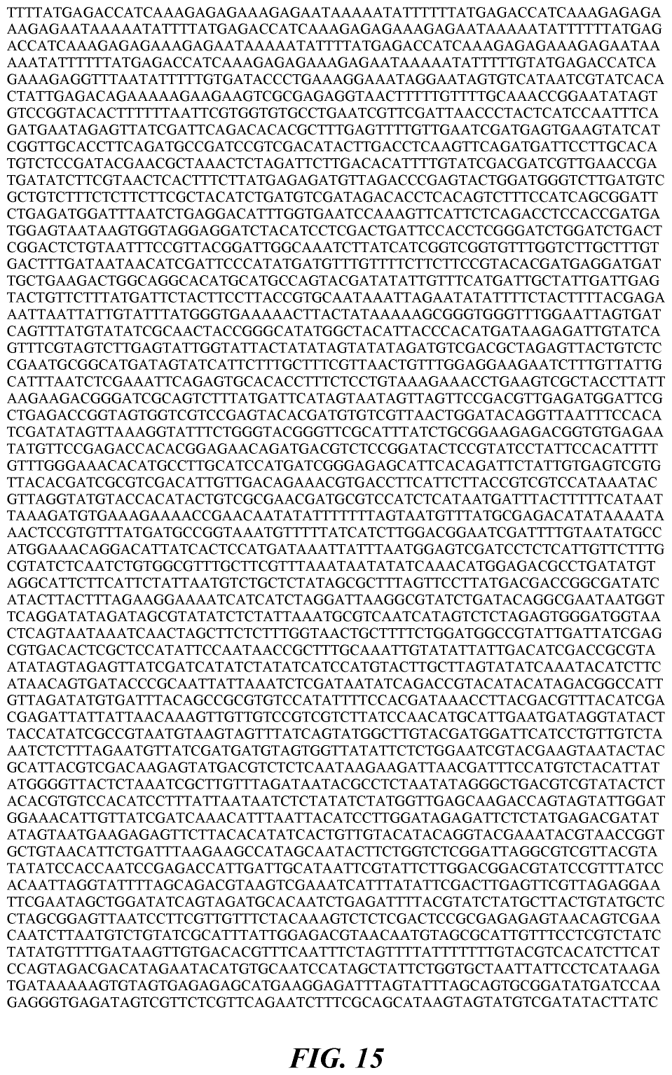

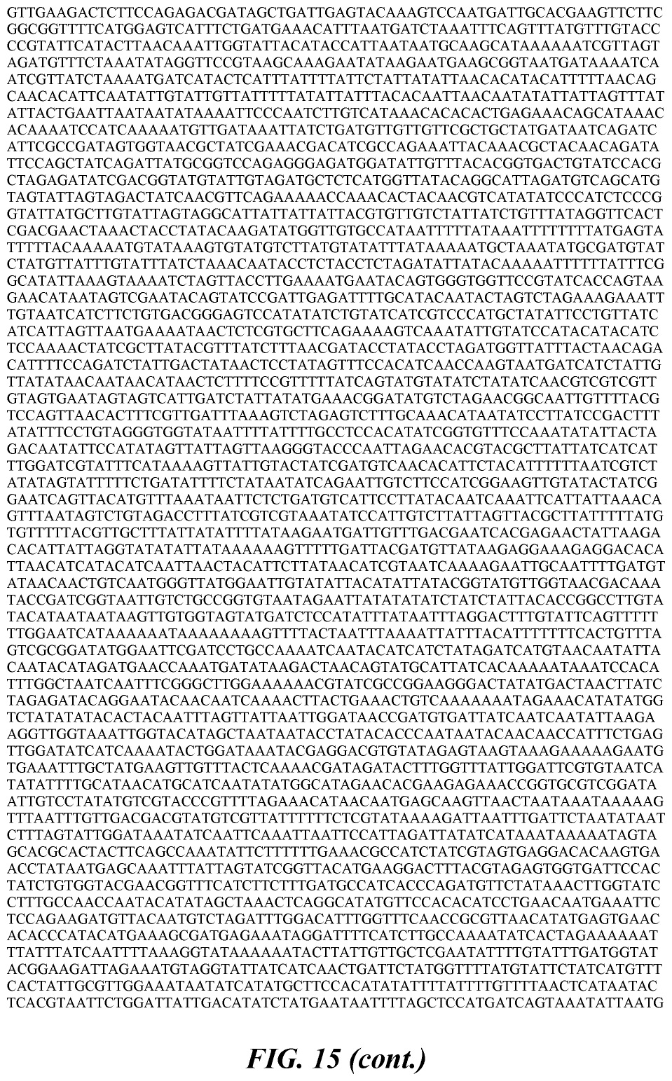

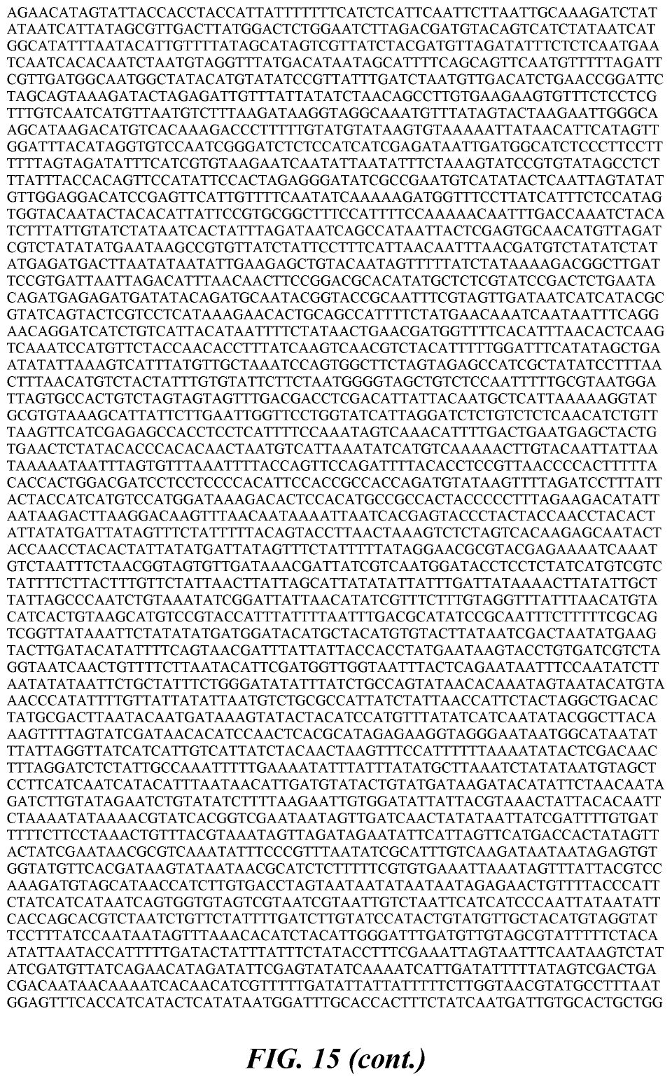

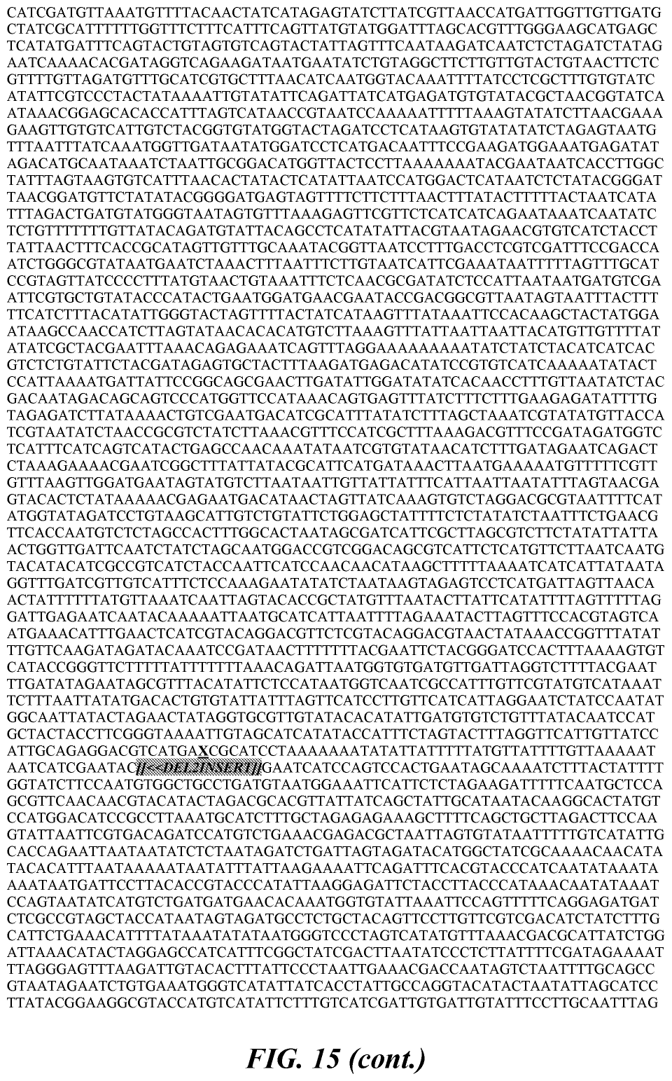

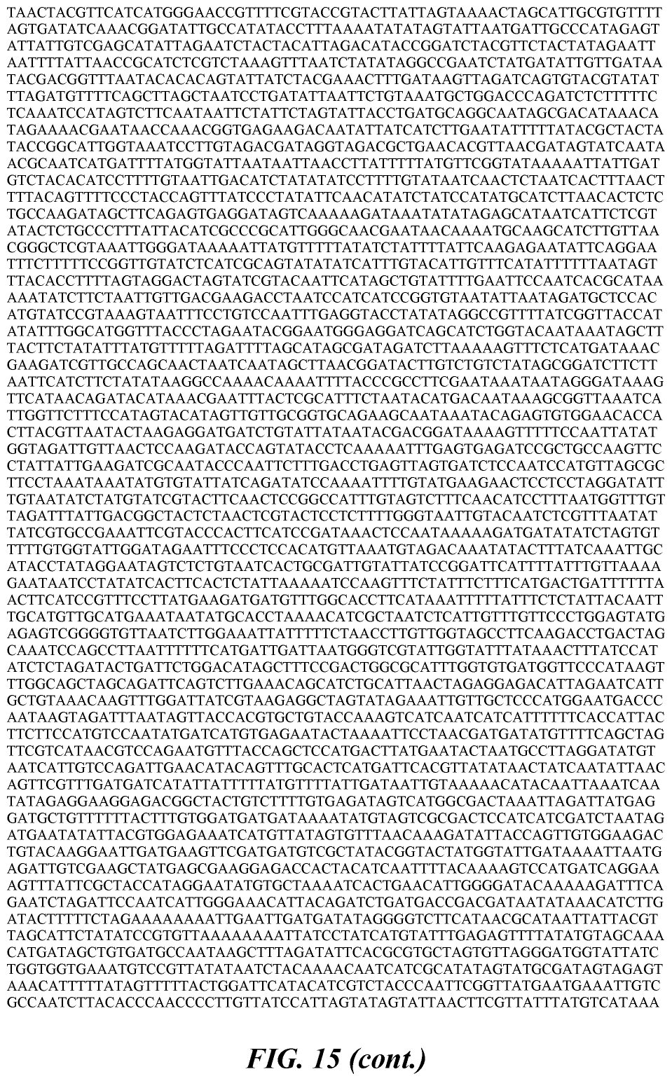

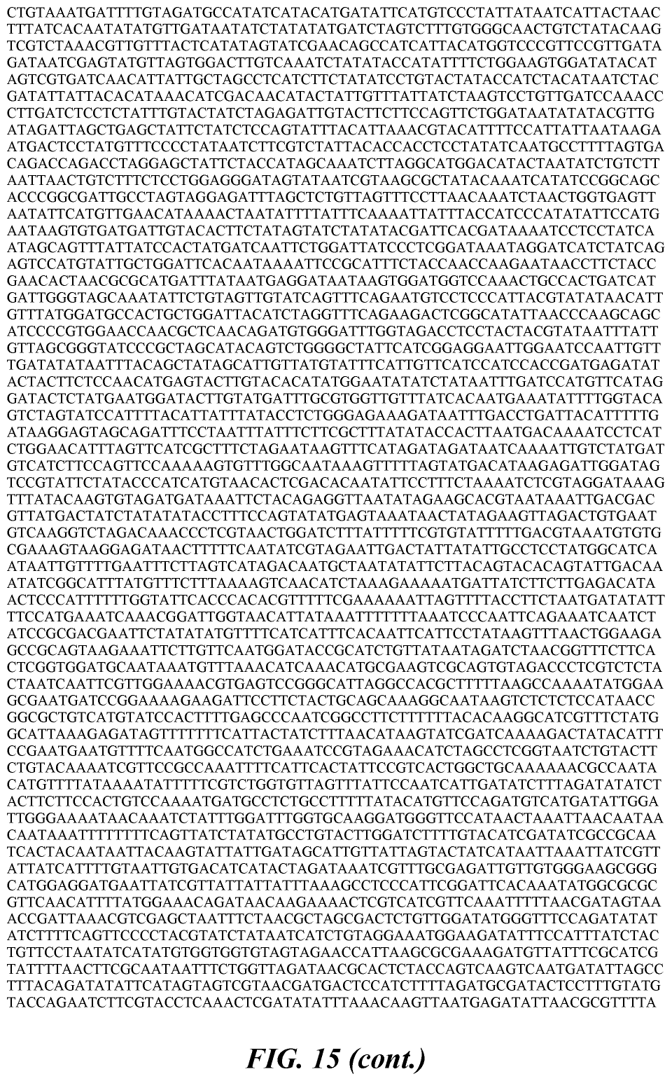

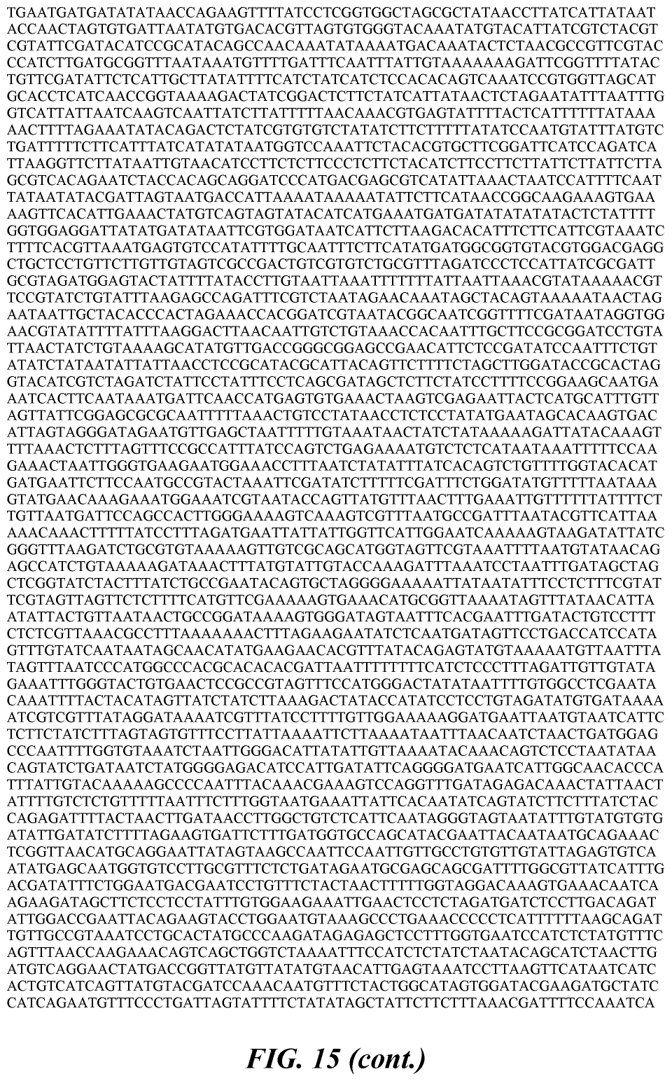

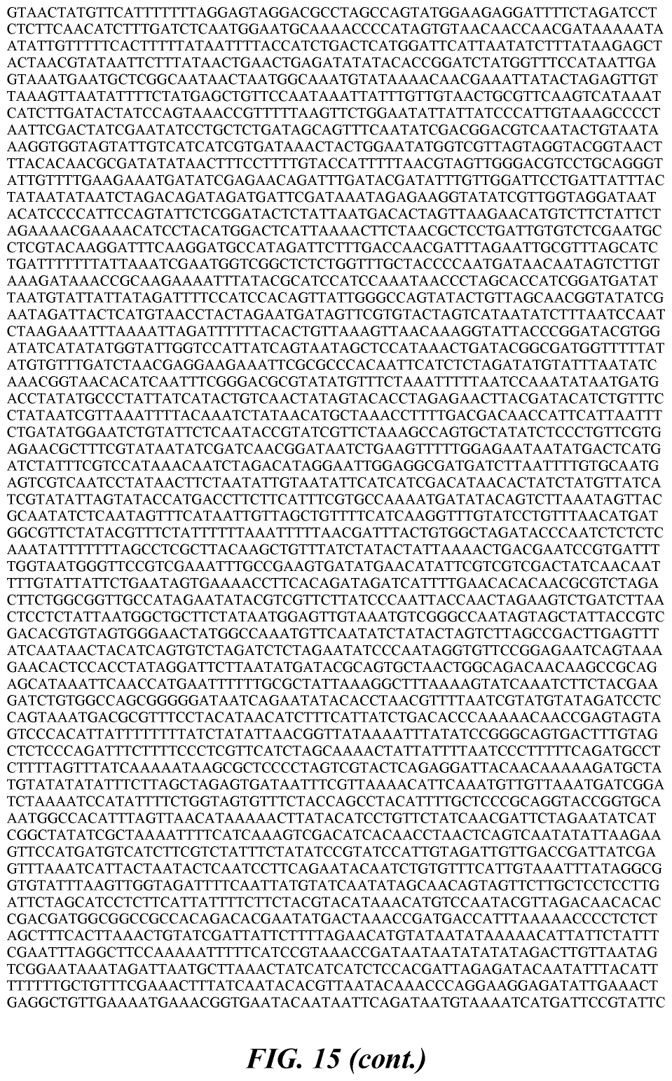

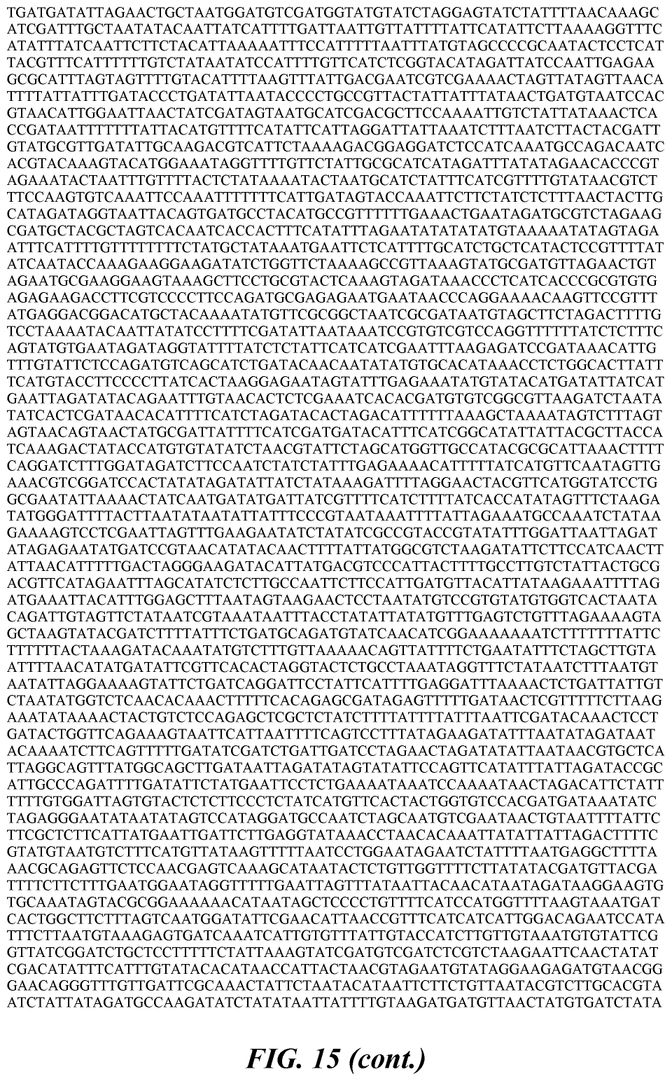

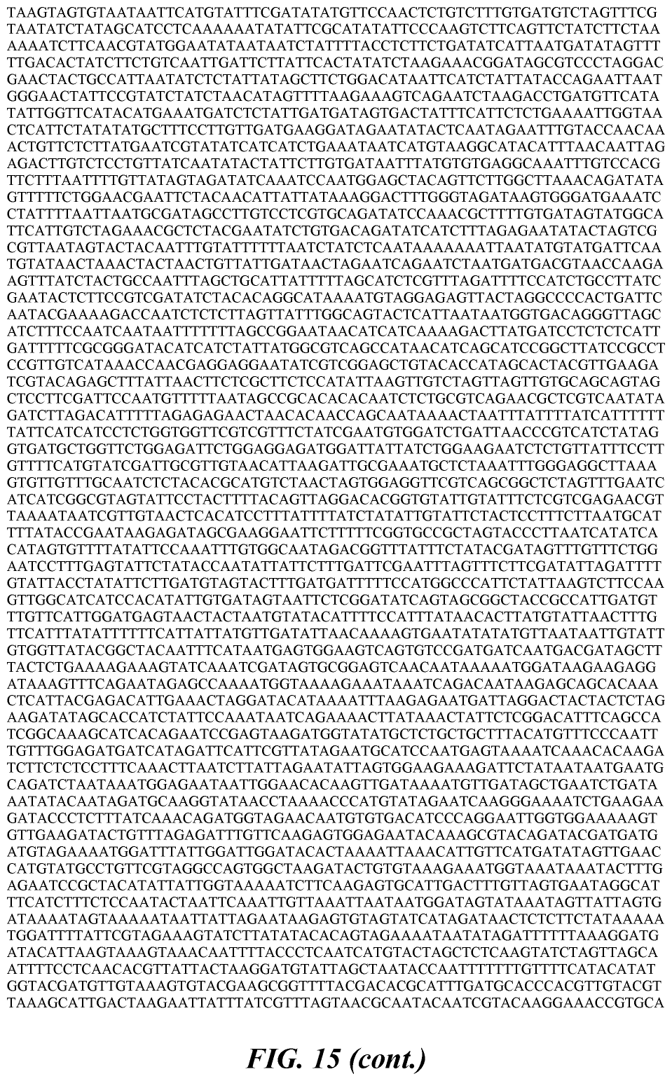

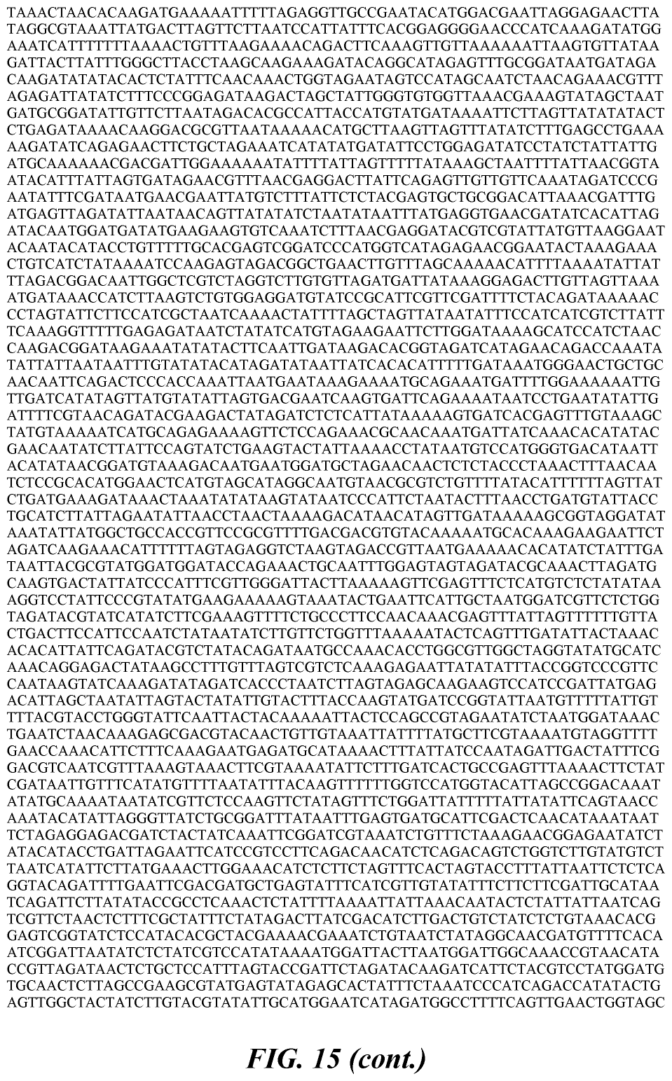

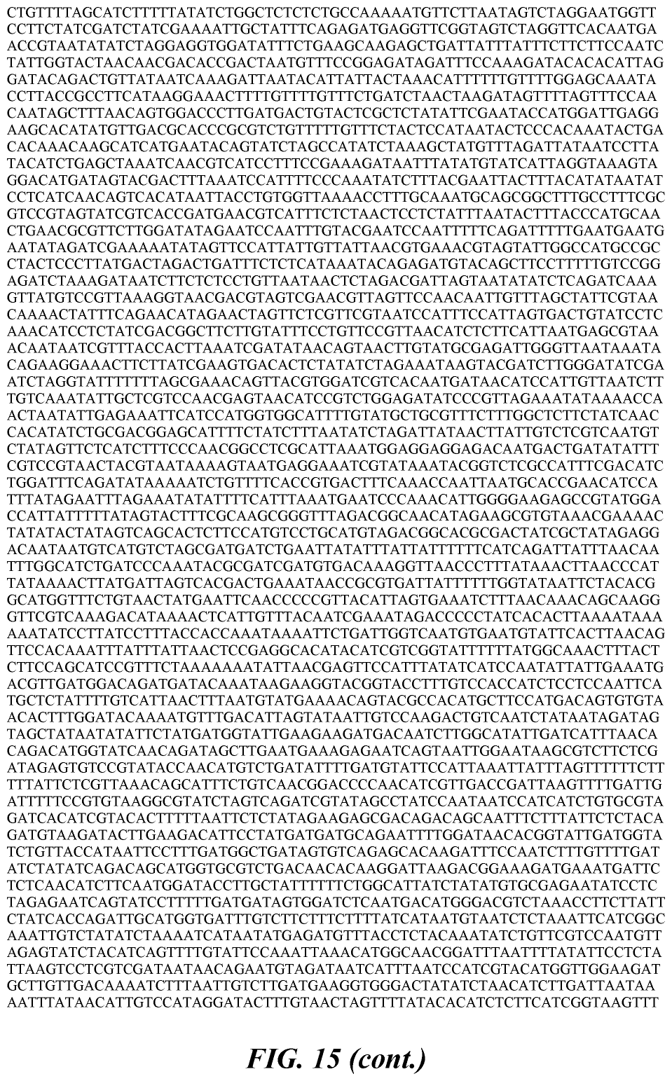

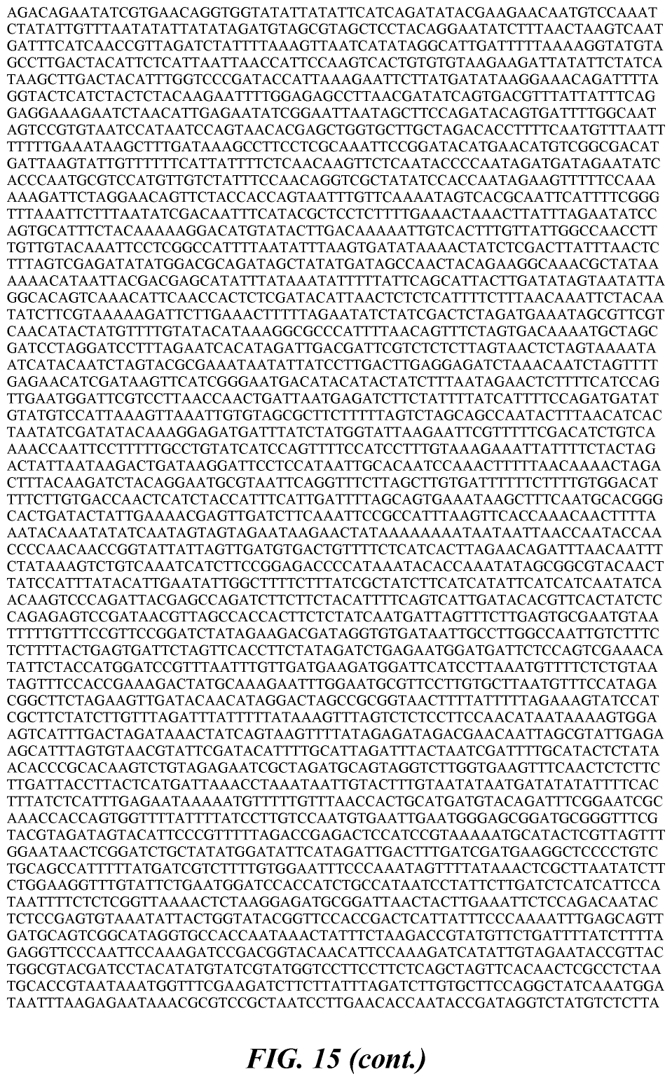

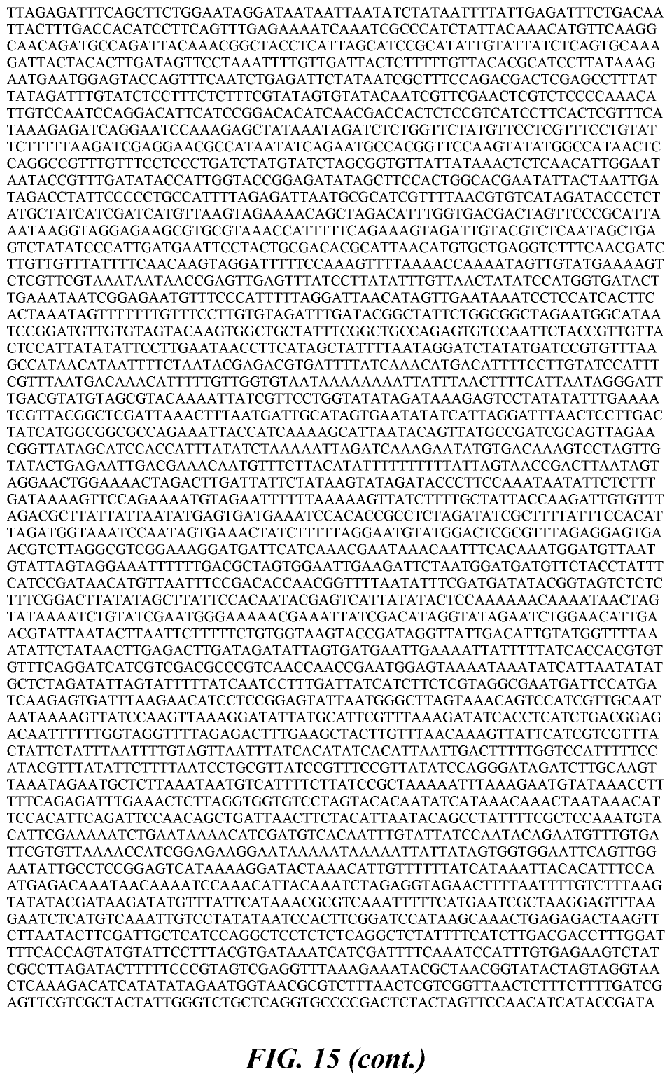

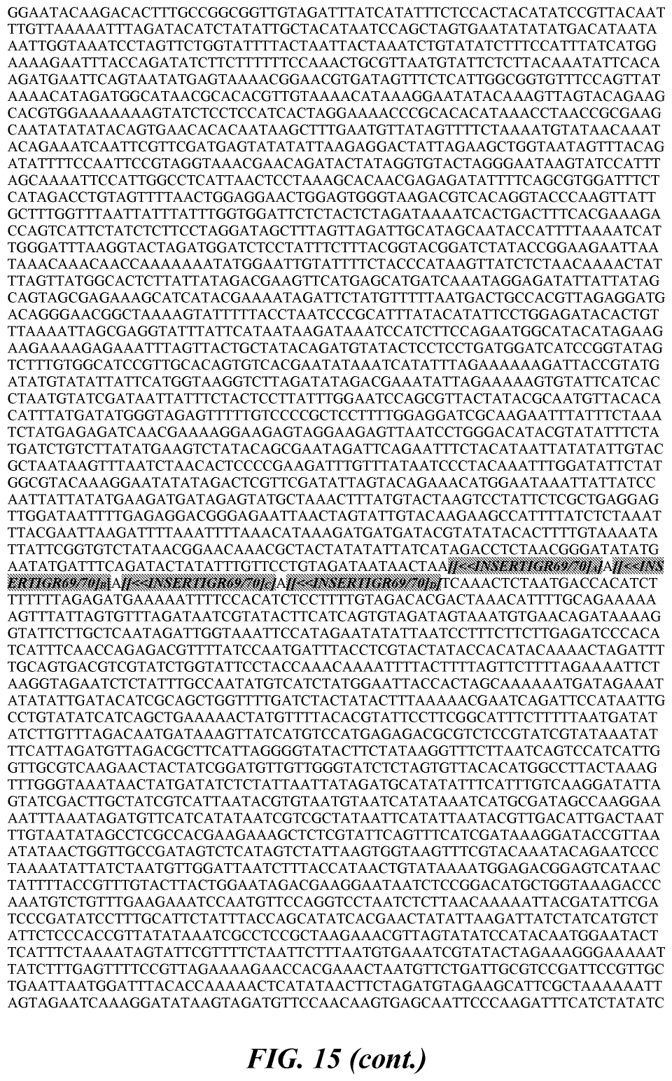

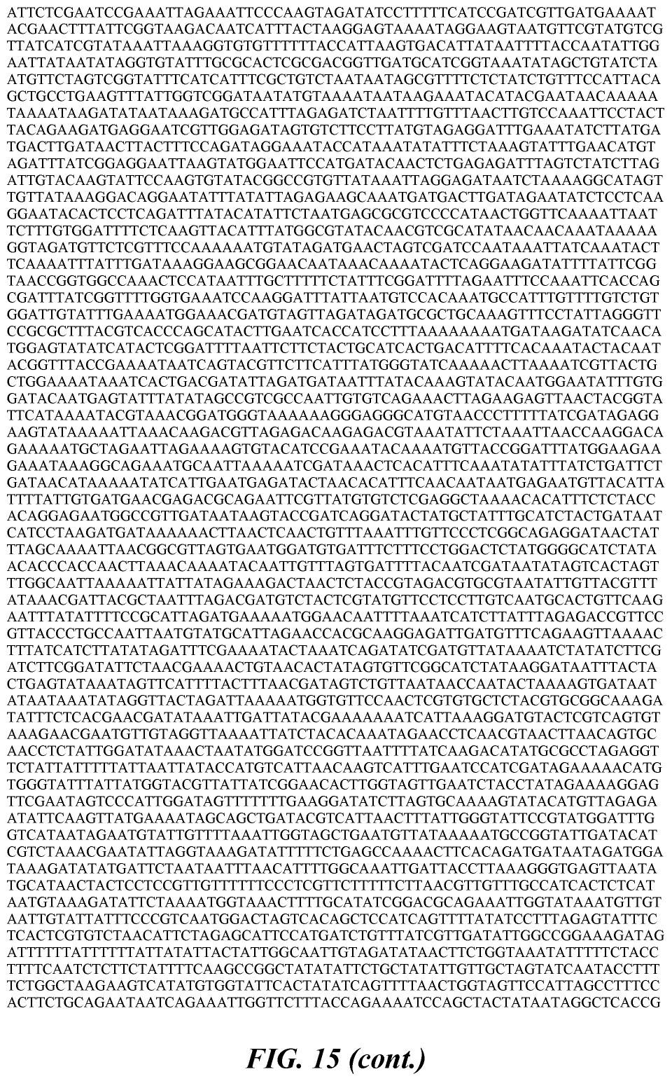

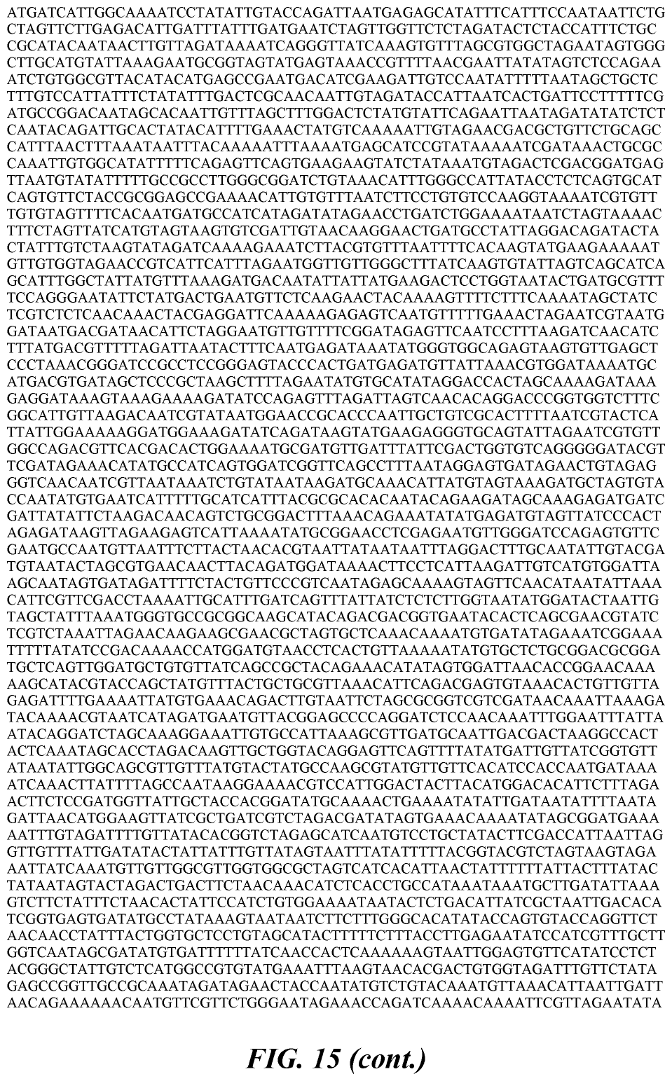

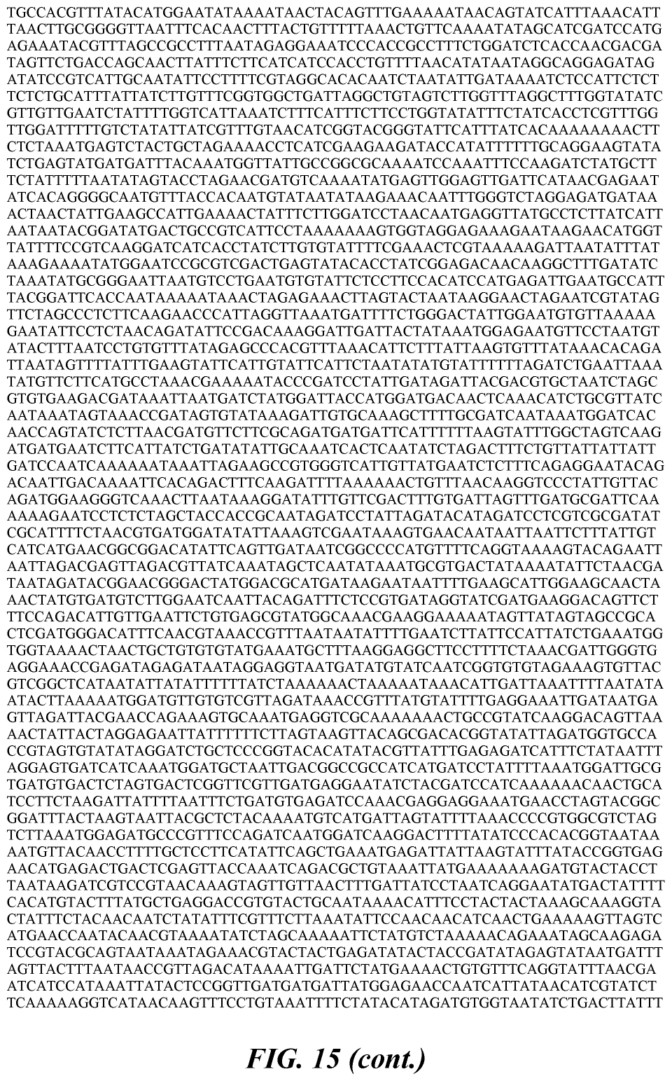

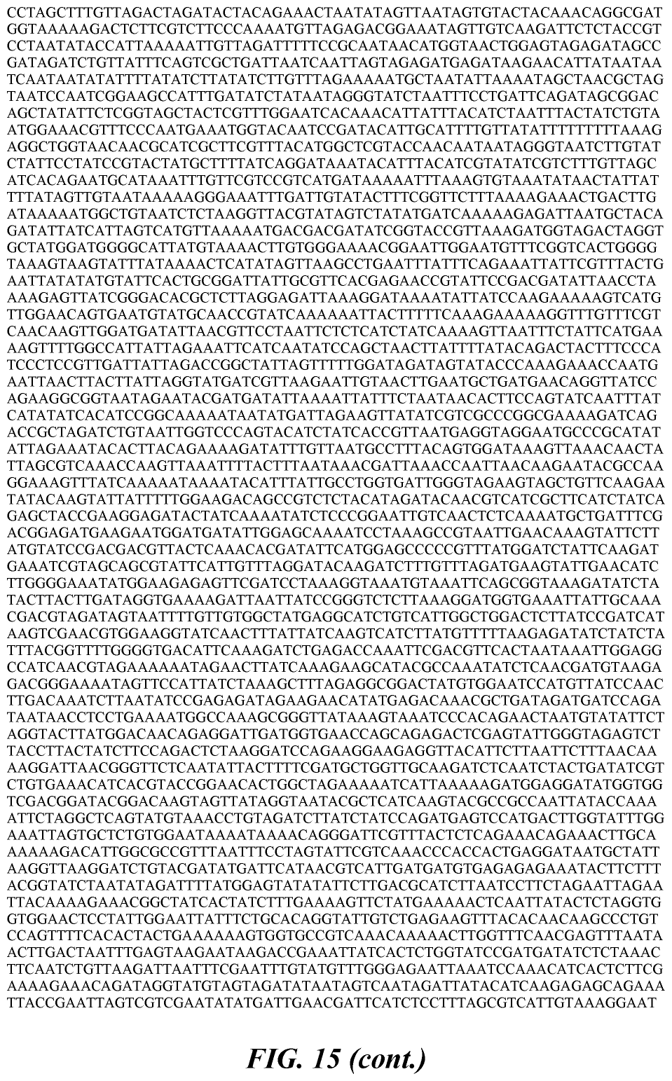

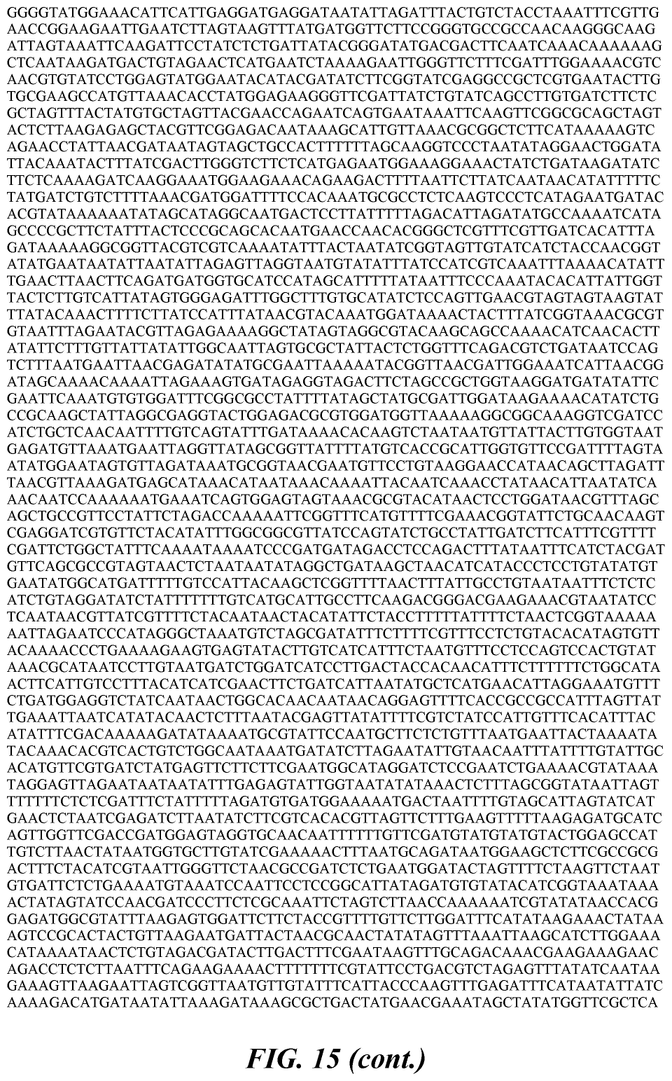

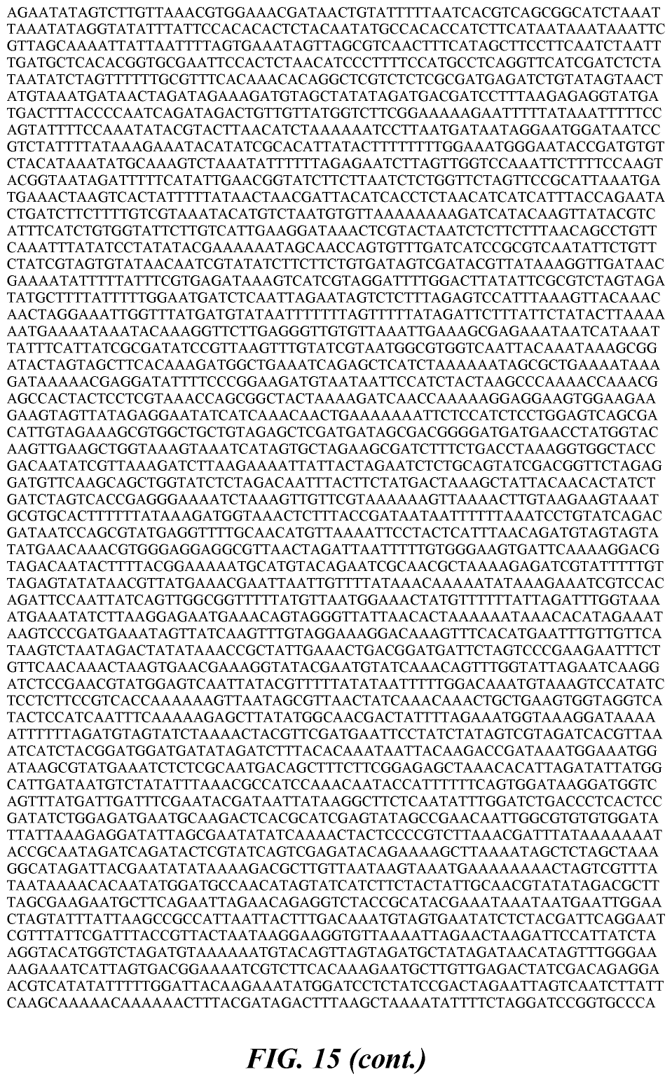

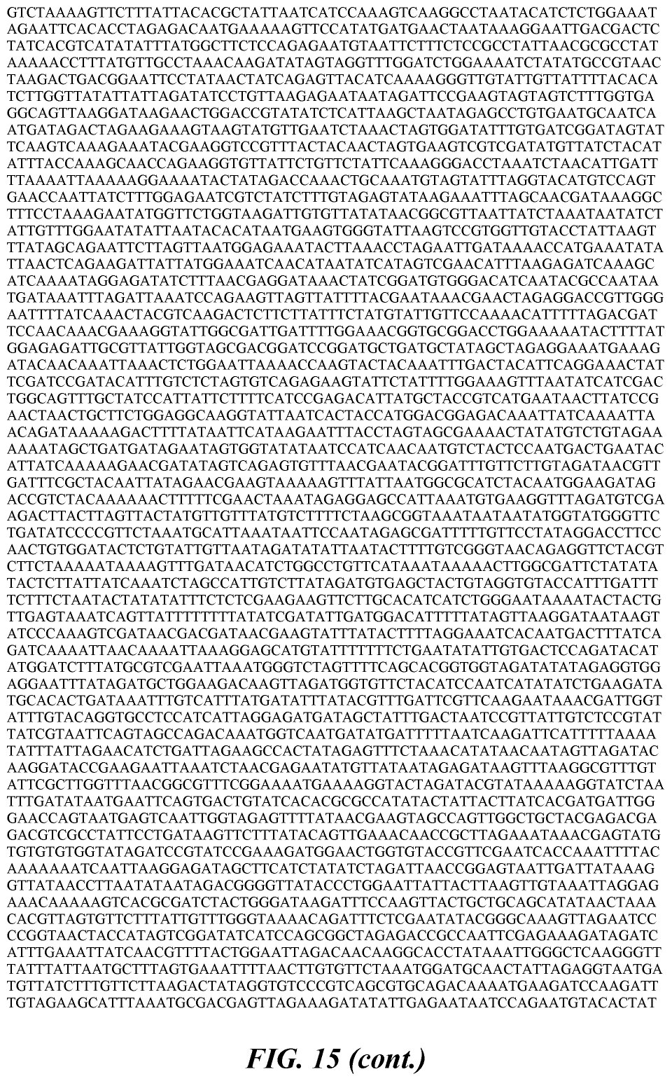

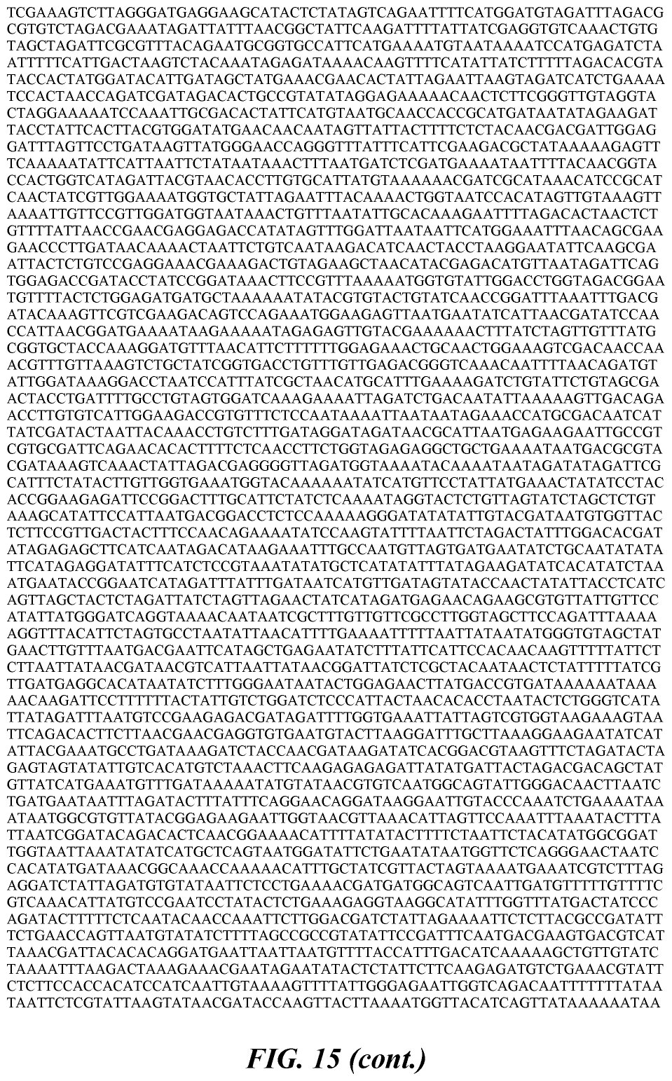

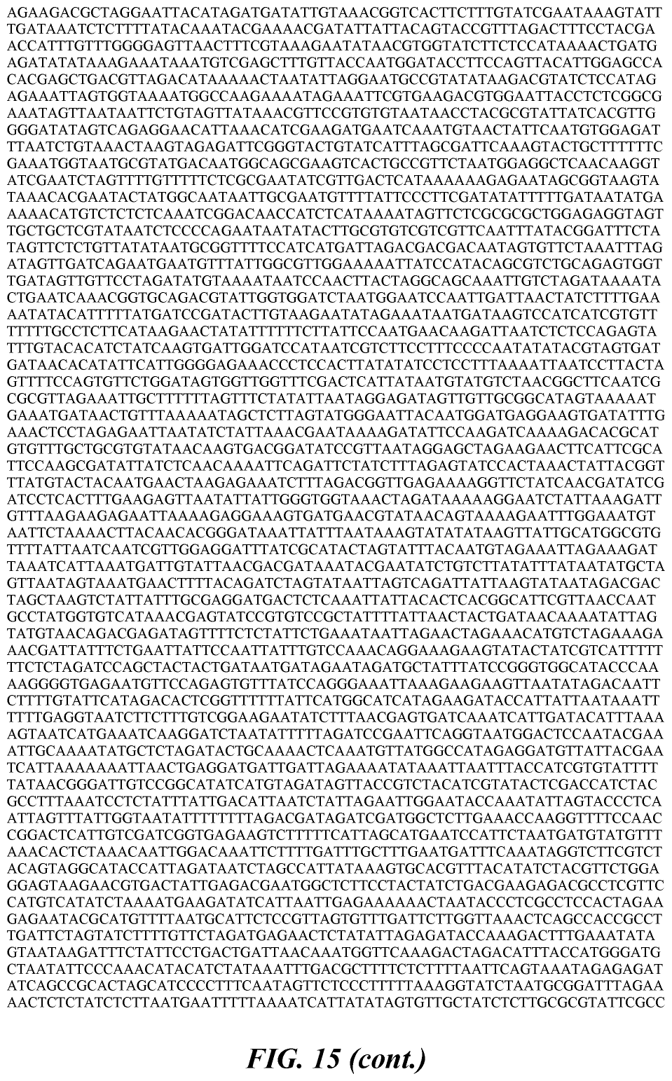

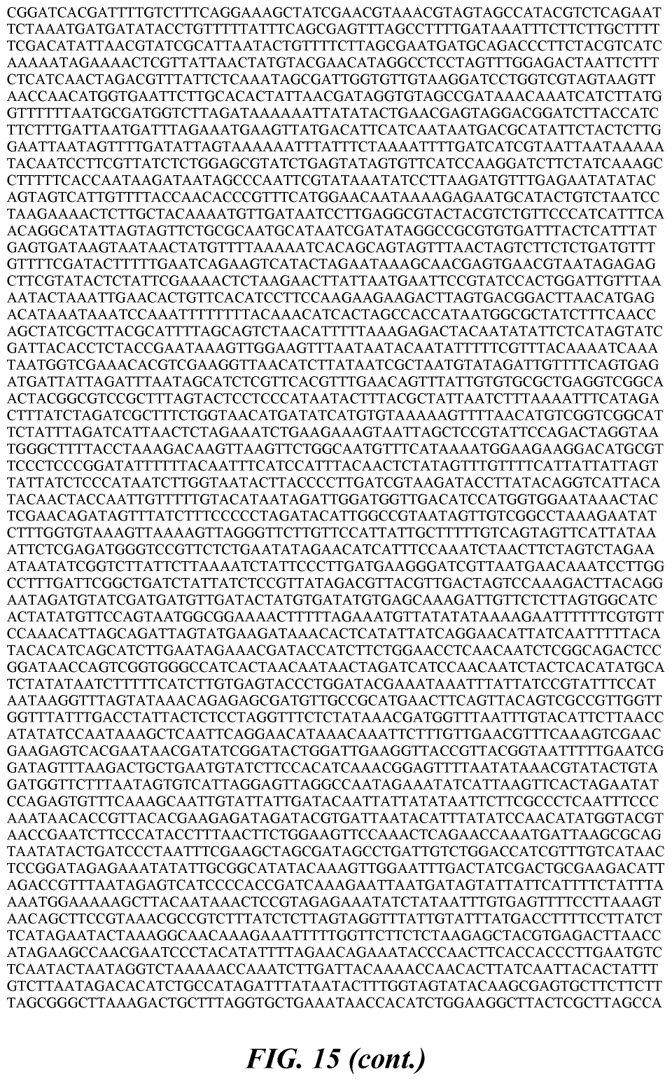

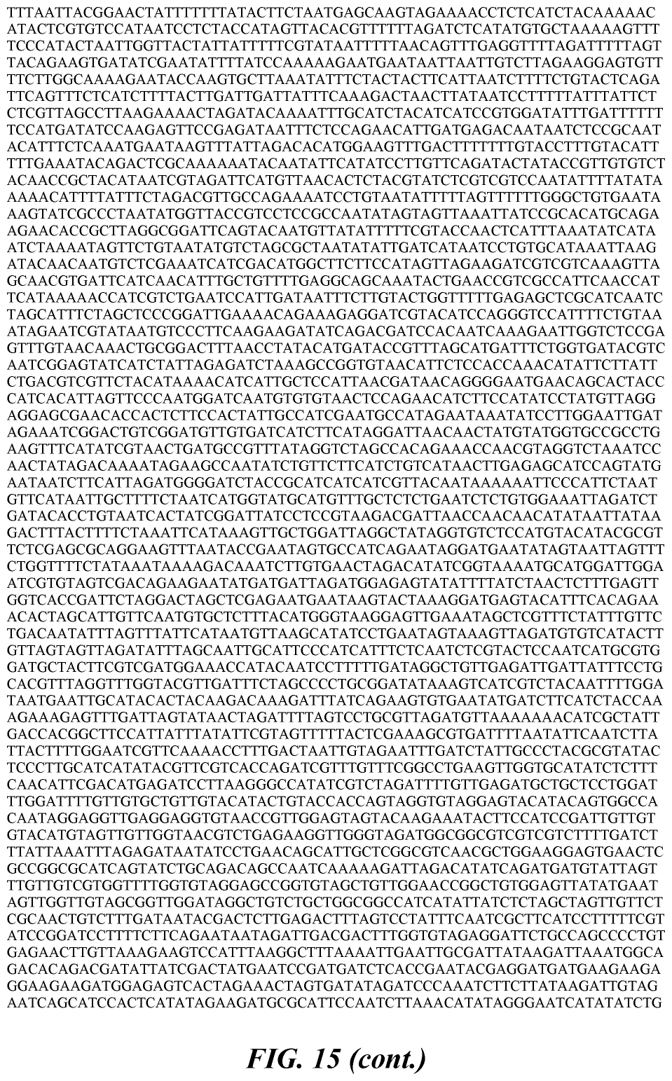

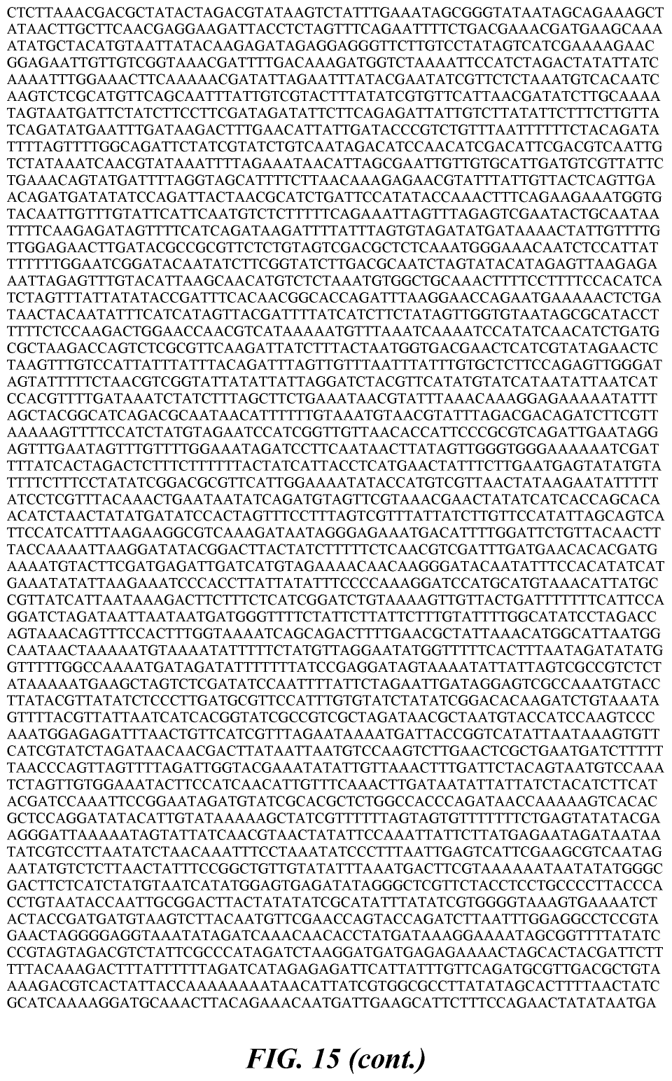

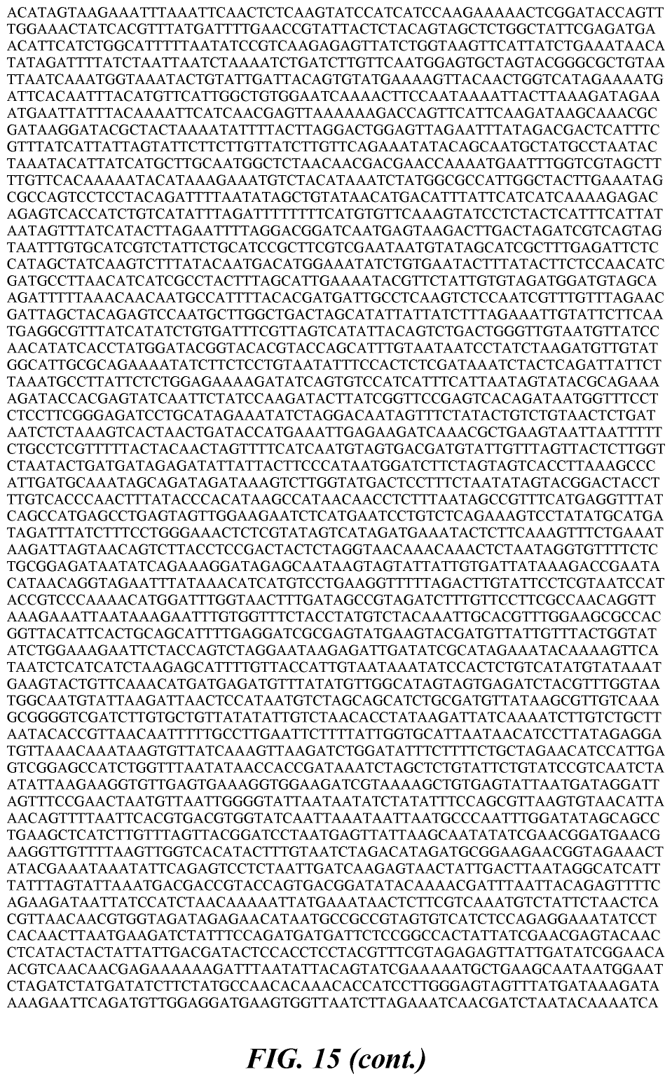

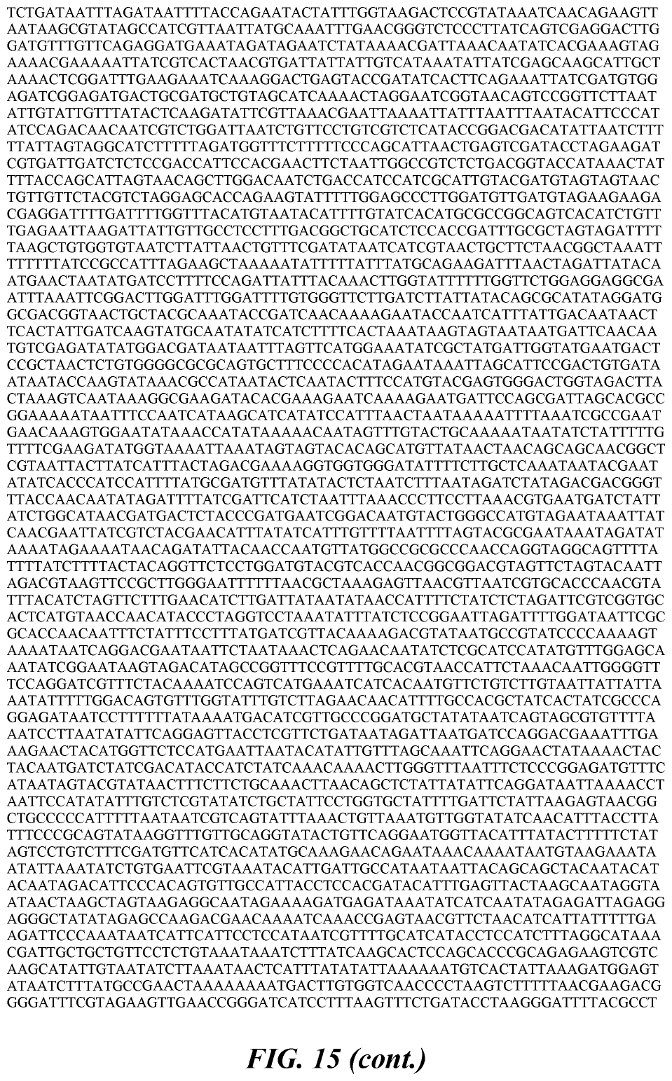

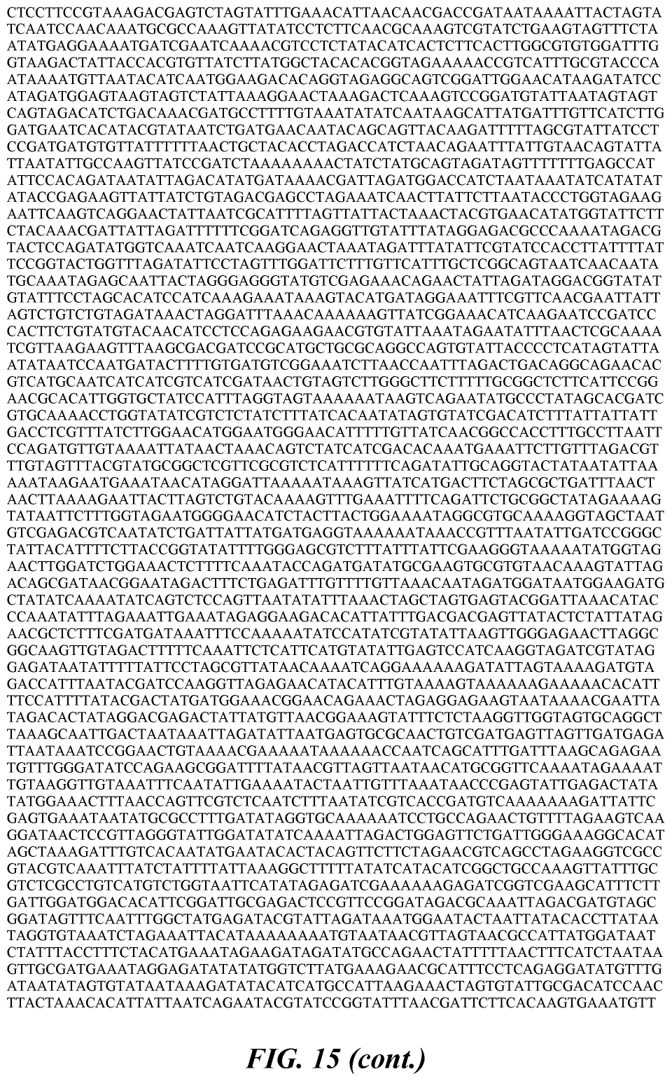

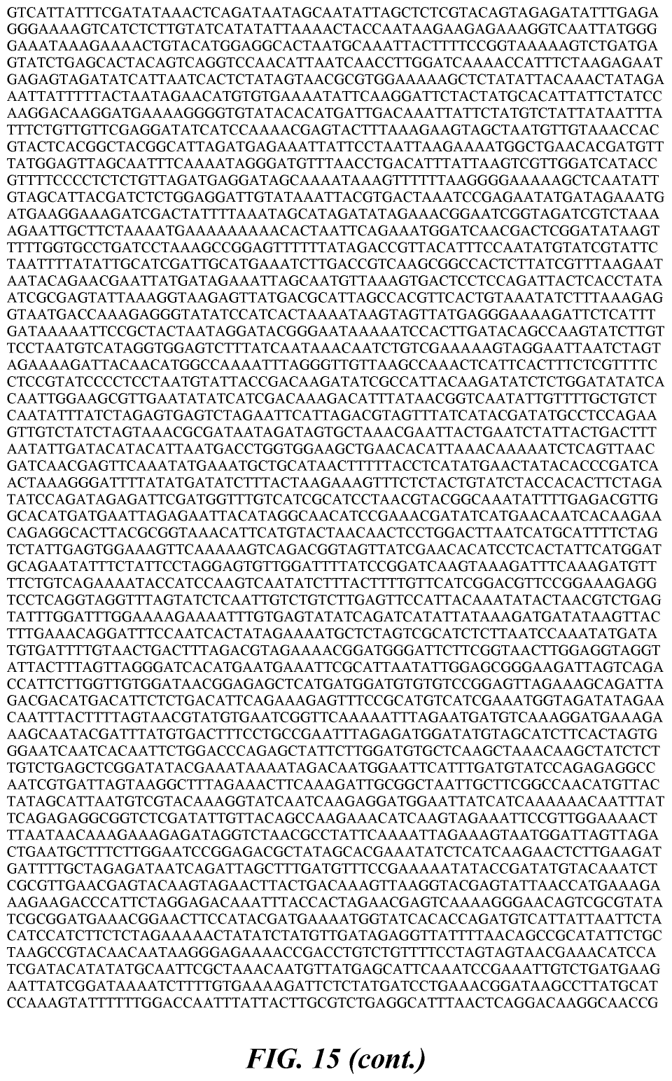

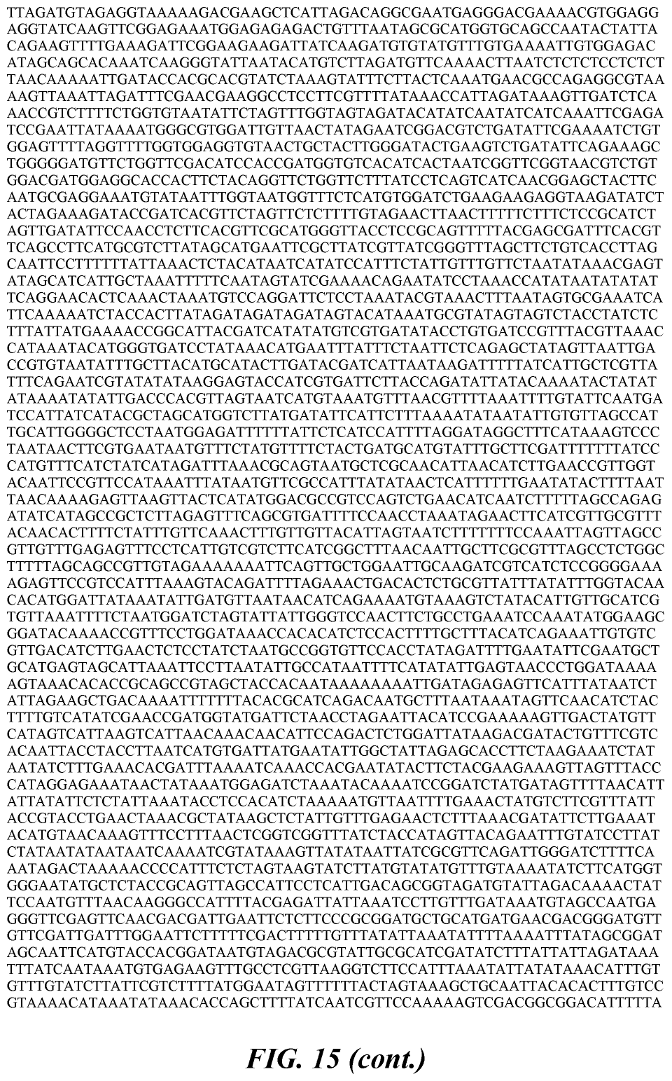

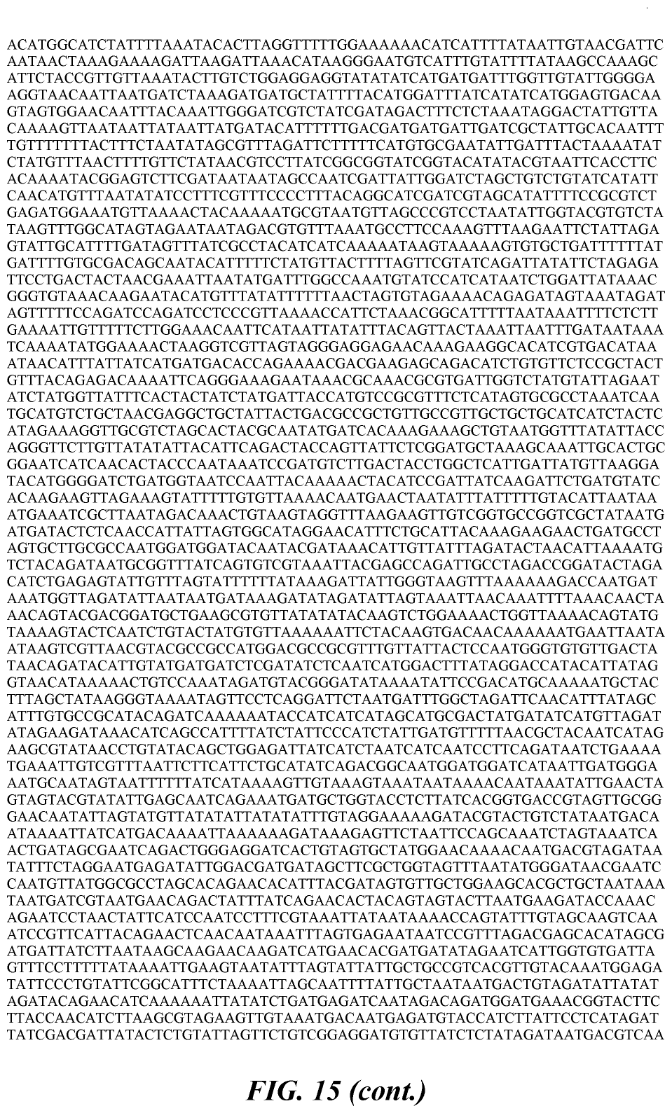

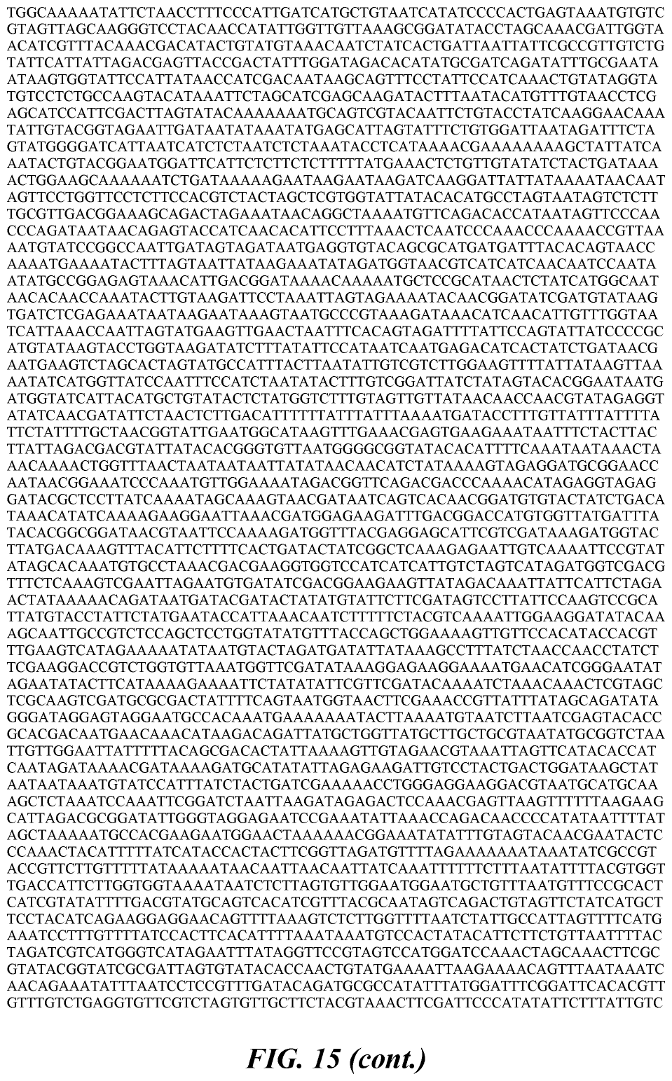

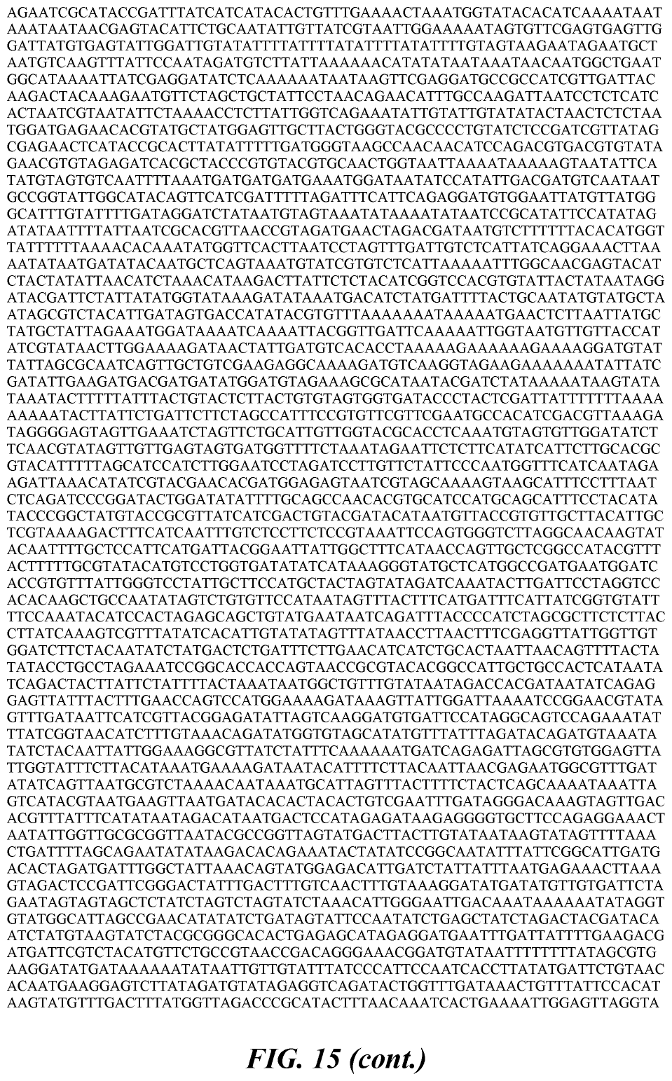

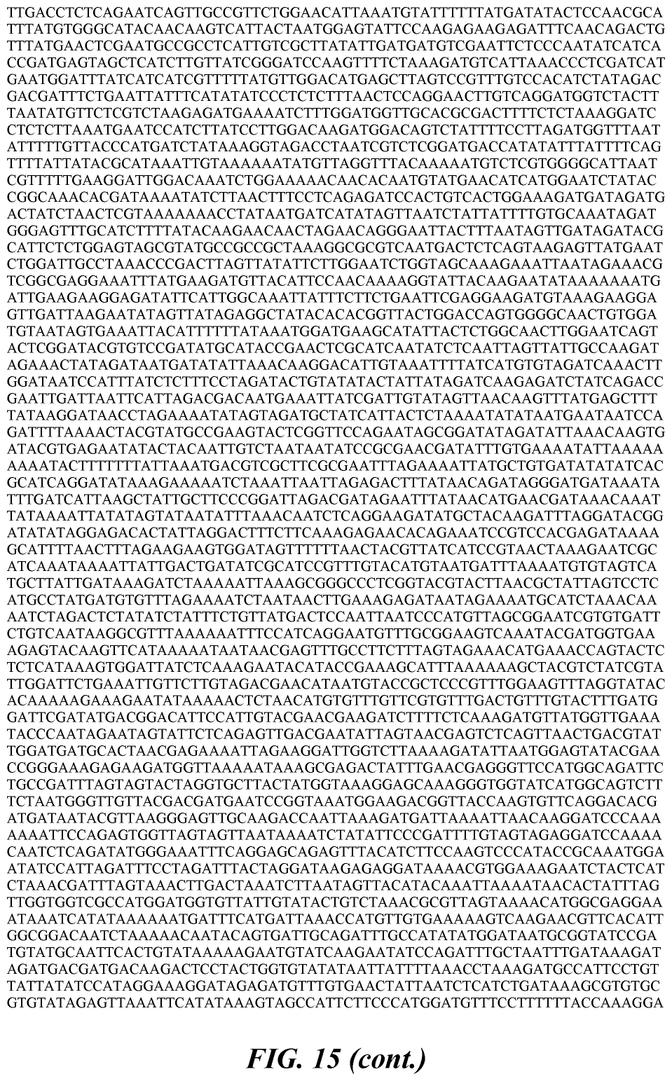

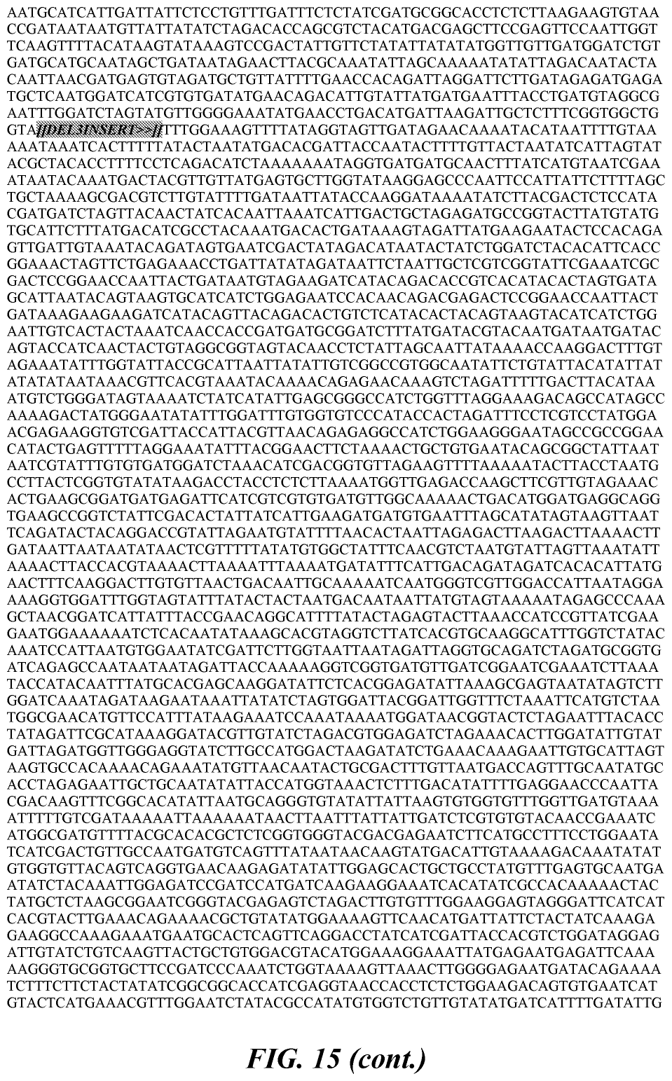

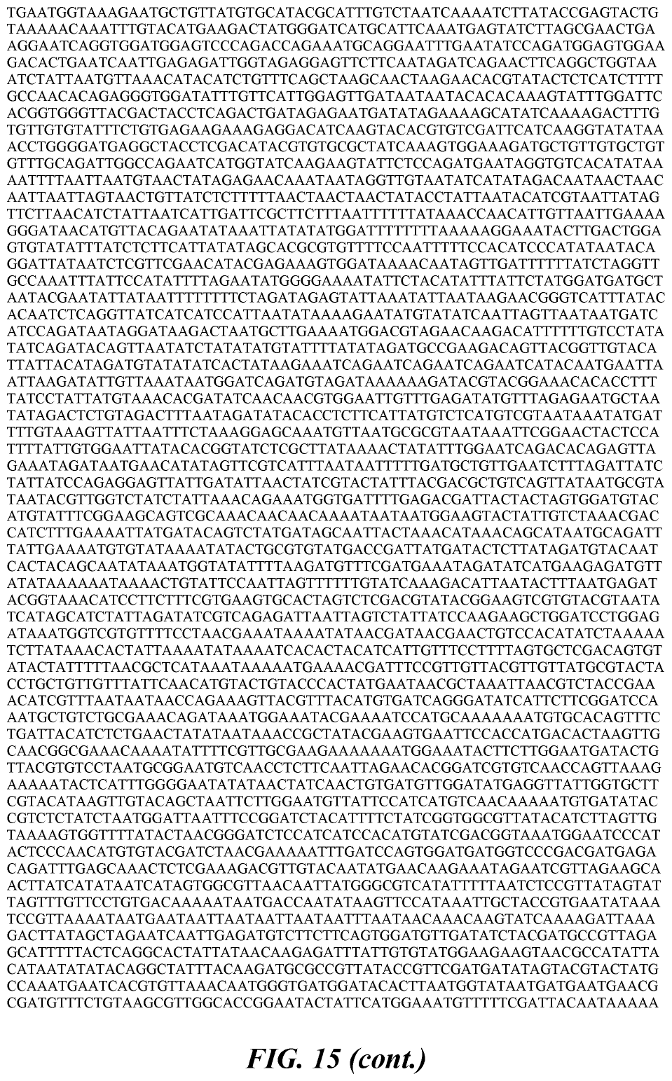

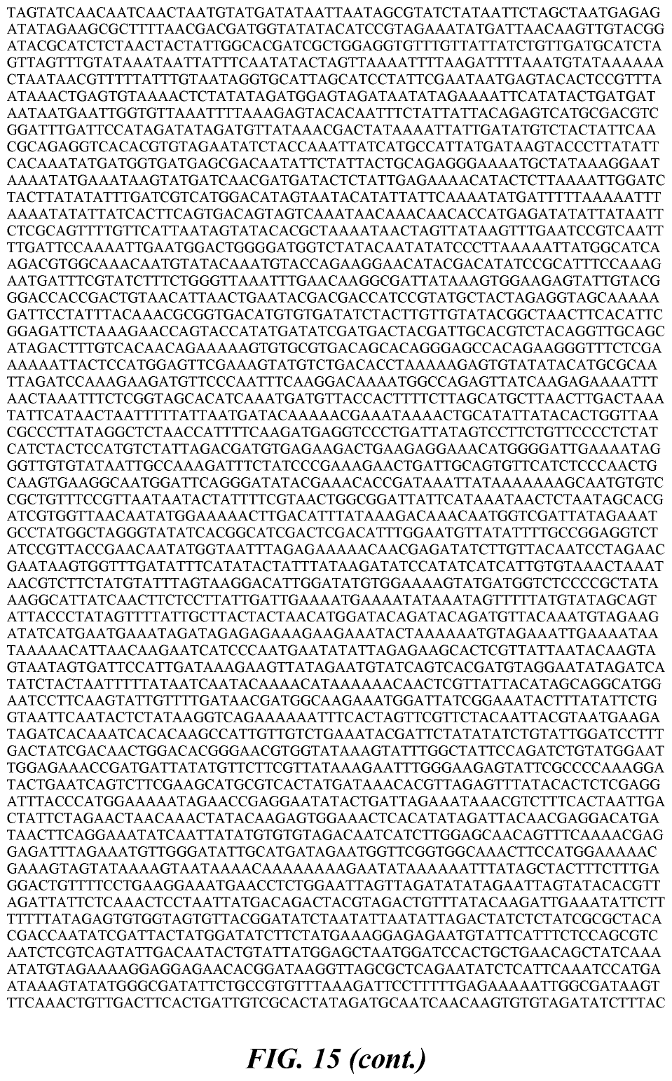

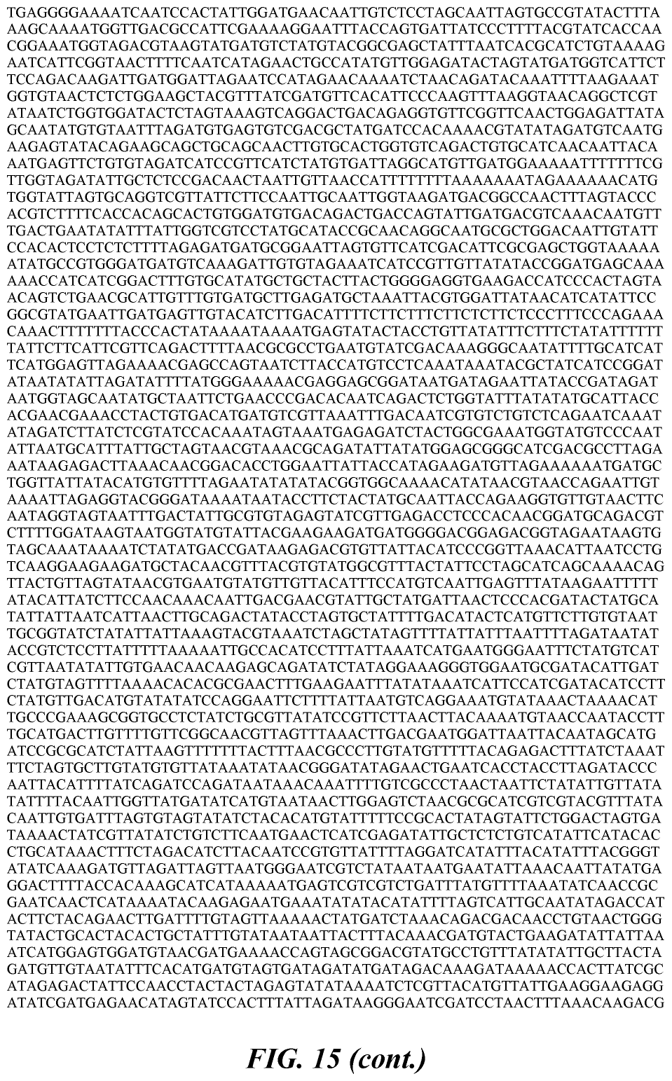

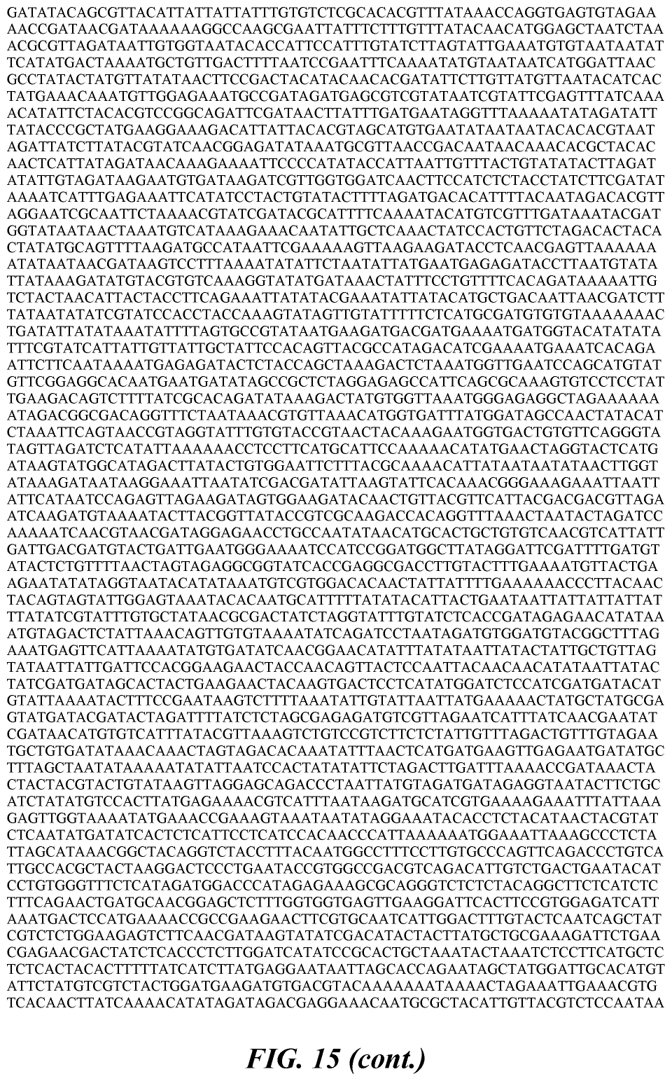

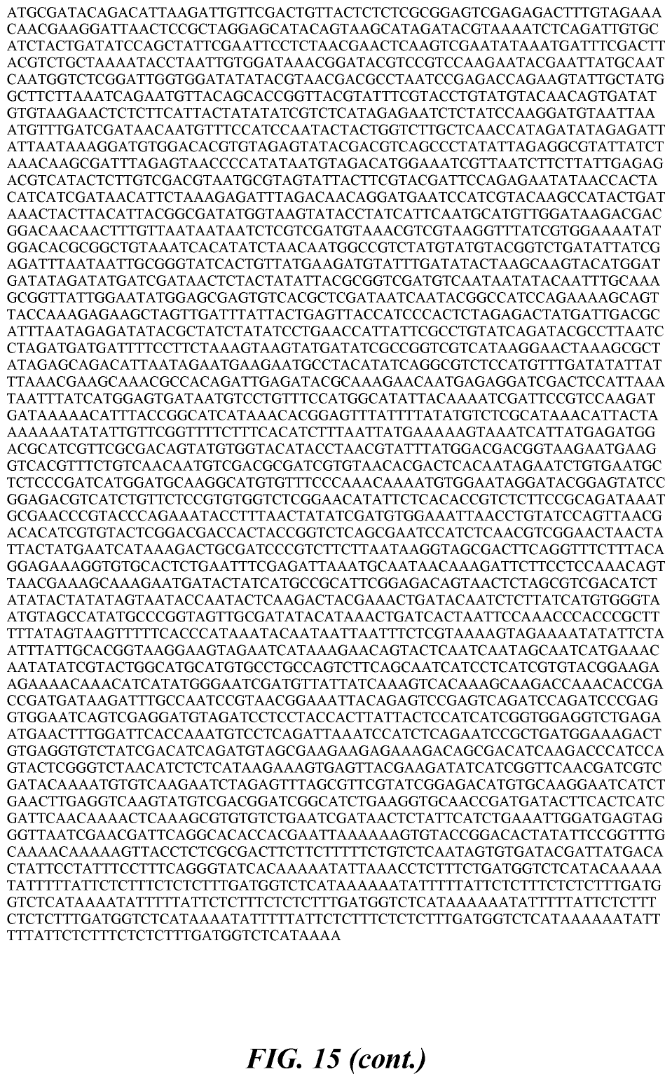

In some embodiments heterologous nucleotide sequences that encode one or more SARS-CoV-2 proteins (or variants, mutants, and/or immunogenic fragments thereof) are inserted into one or more MVA insertion sites. Non-limiting examples of insertion sites that may be used to insert the heterologous nucleotide sequences include, but are not limited to, Del2, IGR69-70, and Del3. FIG. 15 shows the sequence of an sMVA backbone (SEQ ID NO: 1) and the possible locations of insertion sites Del2 ([[<<DEL2INSERT]], where “<<” indicates the heterologous nucleotide insert at Del2 is the reverse complement of the SARS-CoV-2 protein sequence (or variants, mutants, and/or immunogenic fragments thereof) sequence), IGR69-70 ([[<<INSERTIGR69/70]A], [[<<INSERTIGR69/70]B], [[<<INSERTIGR69/70]c], or [[<<INSERTIGR69/70]D], where “<<” indicates the heterologous nucleotide insert at Del2 is the reverse complement of the SARS-CoV-2 protein sequence (or variants, mutants, and/or immunogenic fragments thereof), and each insertion site is representative of four alternative insertion sites for IGR69/70: A, B, C, or D), and Del3 ([[DEL3INSERT>>]], where “>>” indicates the heterologous nucleotide insert at Del3 is the forward SARS-CoV-2 protein sequence (or variant, mutant, and/or immunogenic fragment thereof) according to some embodiments. FIG. 15 also includes an “X” (in bold underline) that may be a T or an A according to some embodiments.

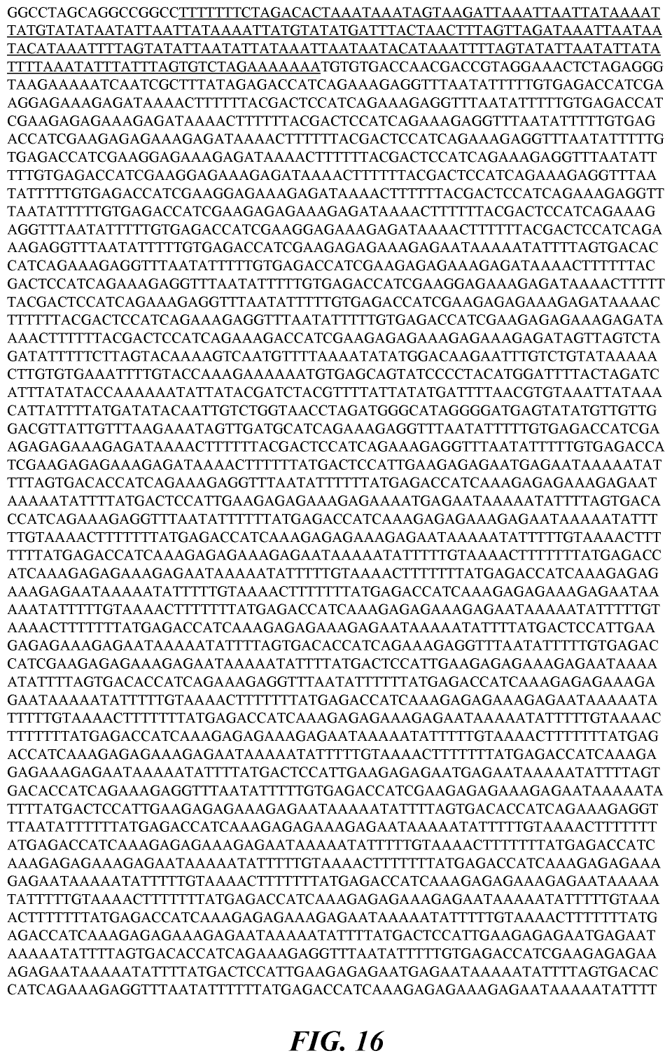

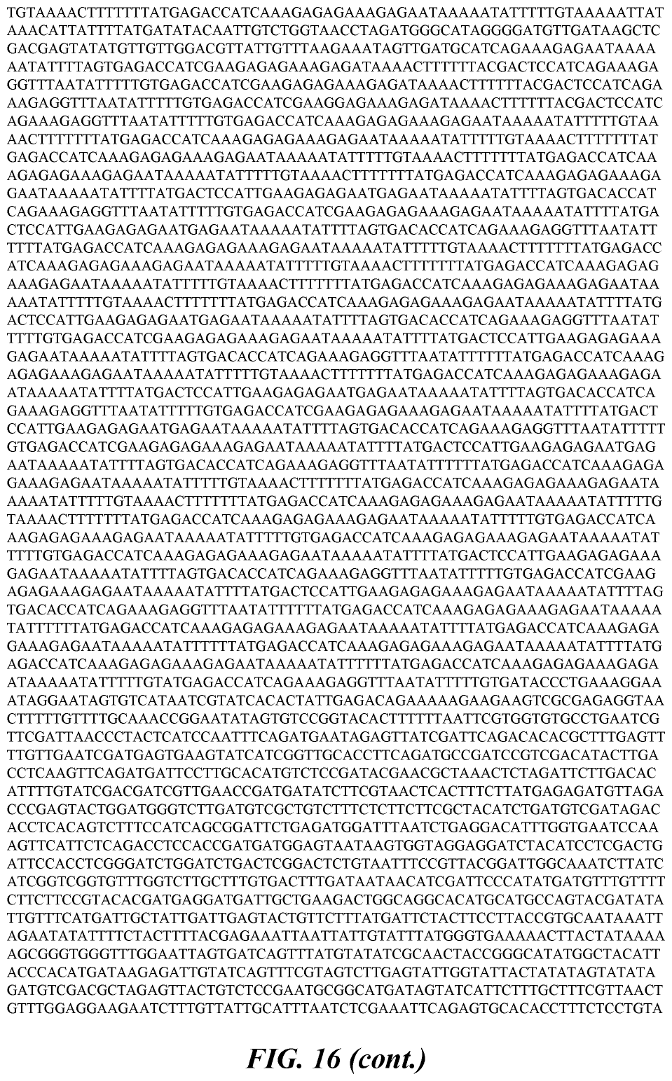

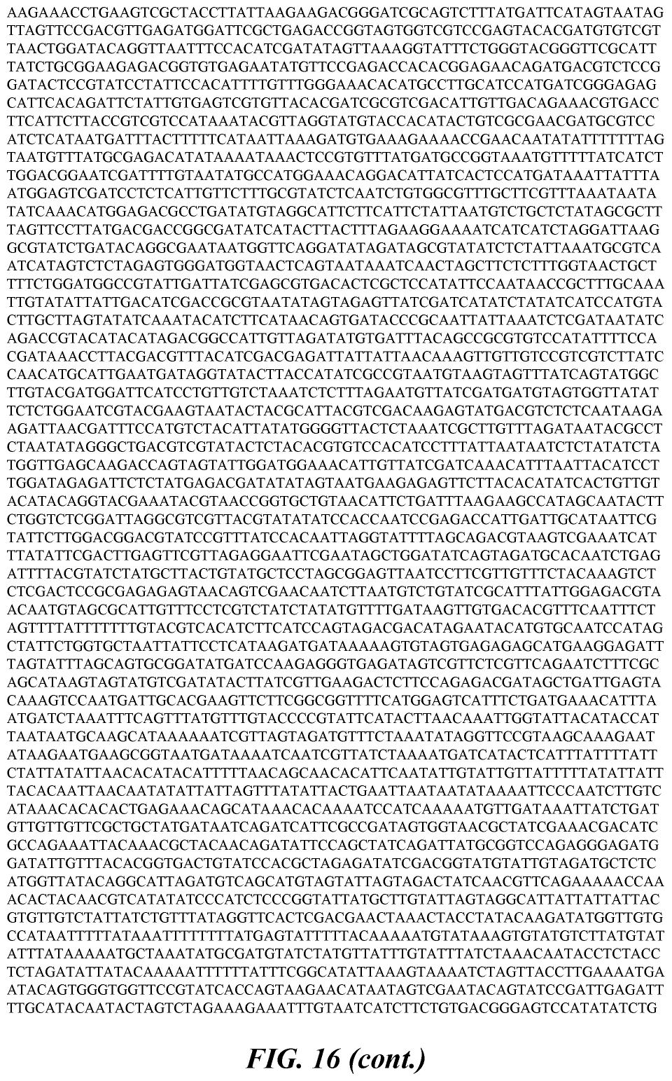

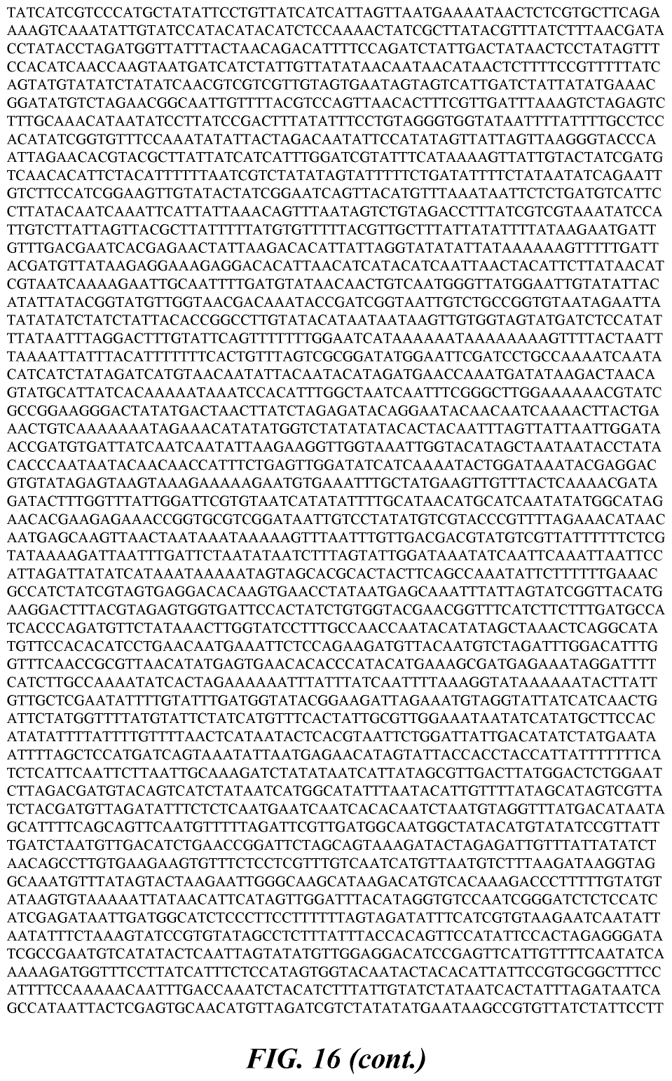

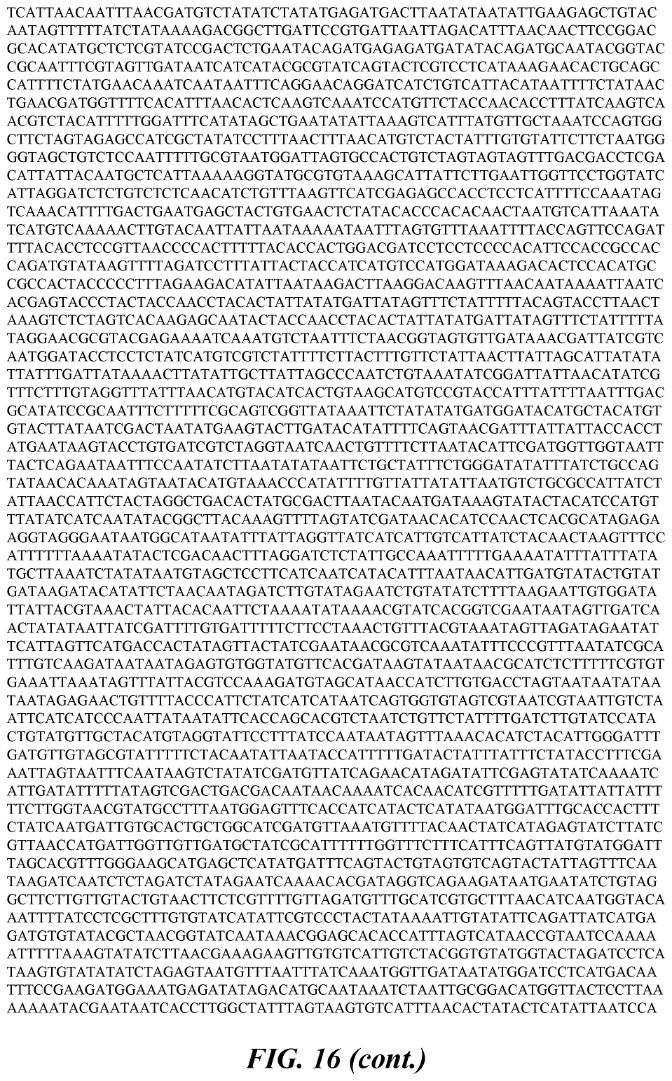

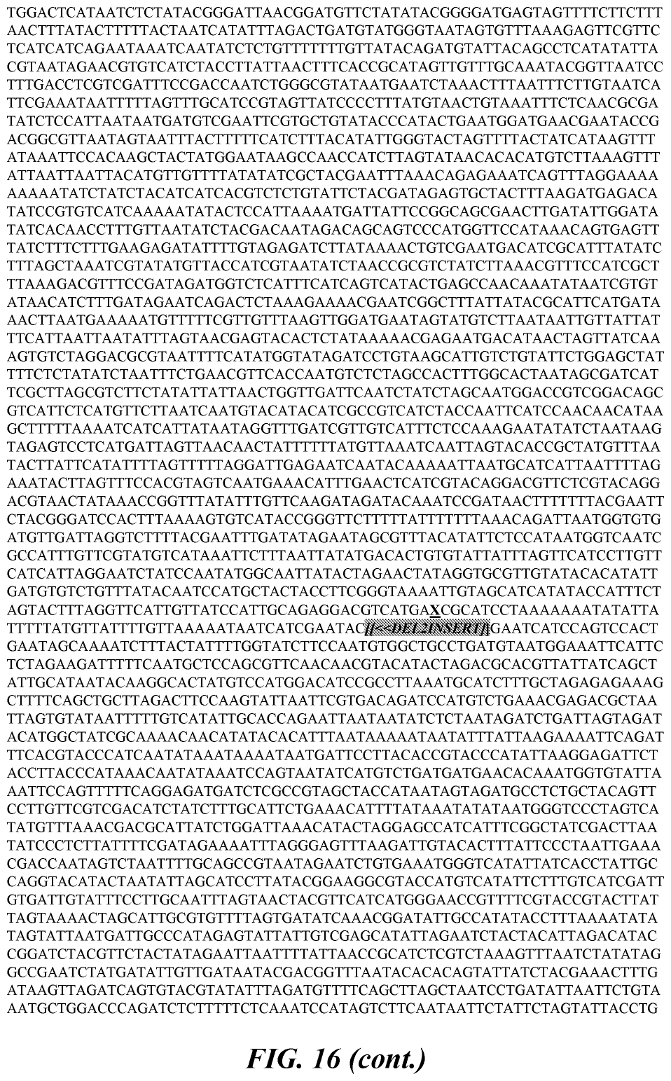

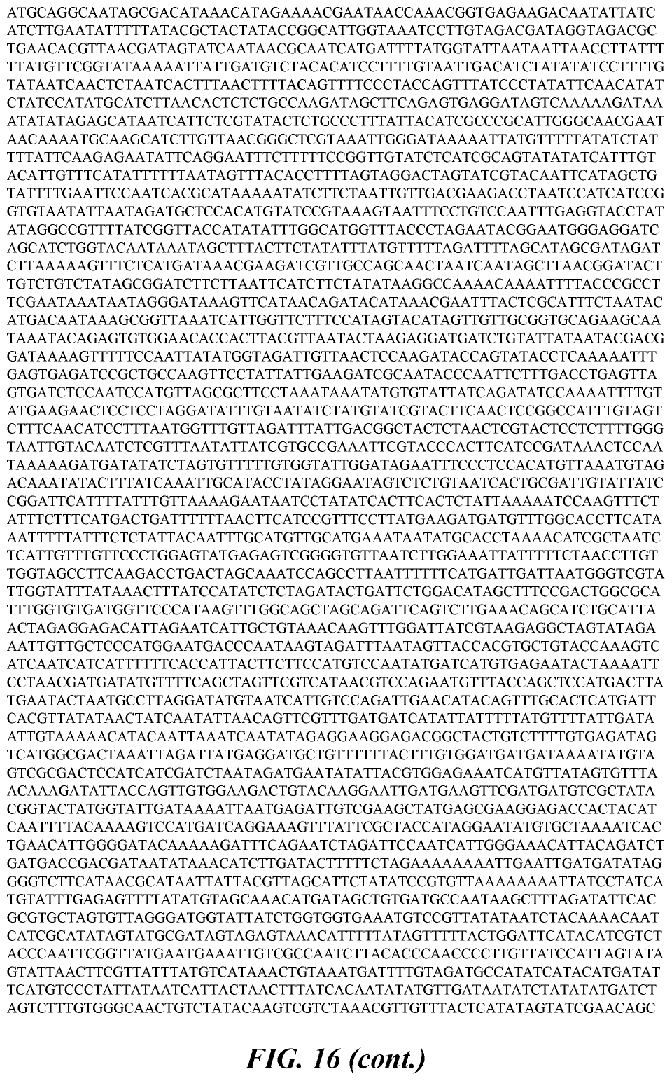

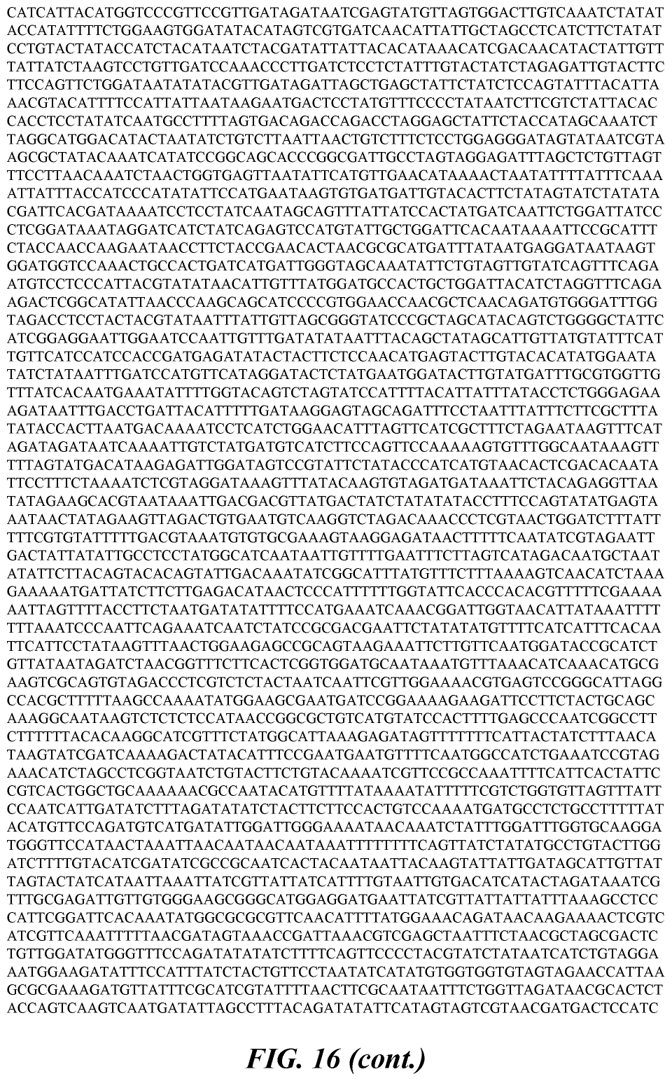

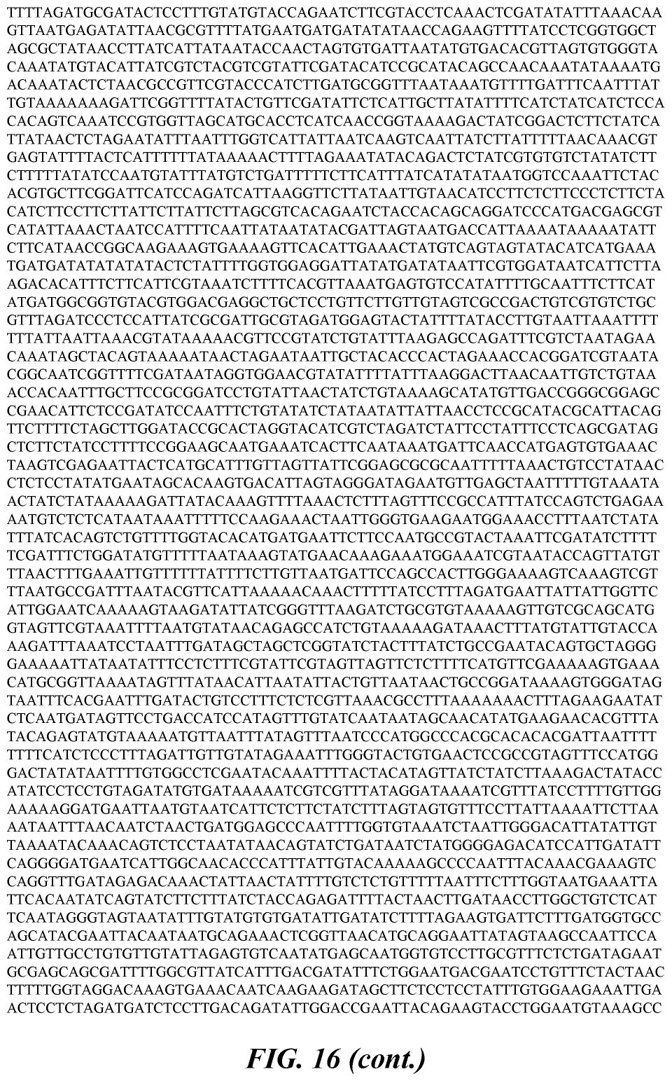

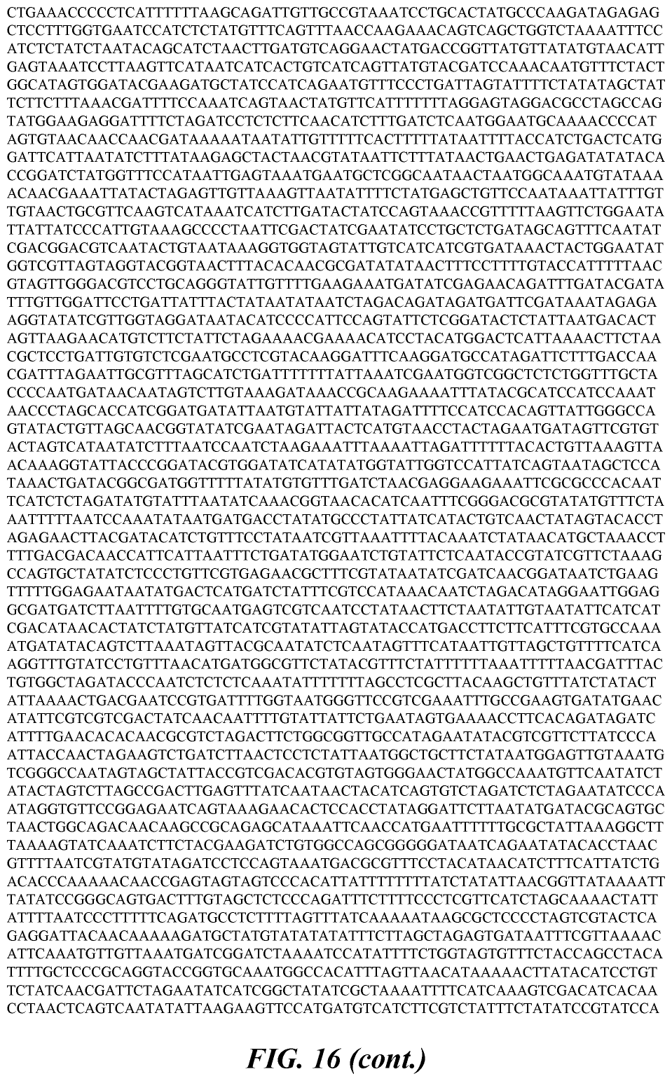

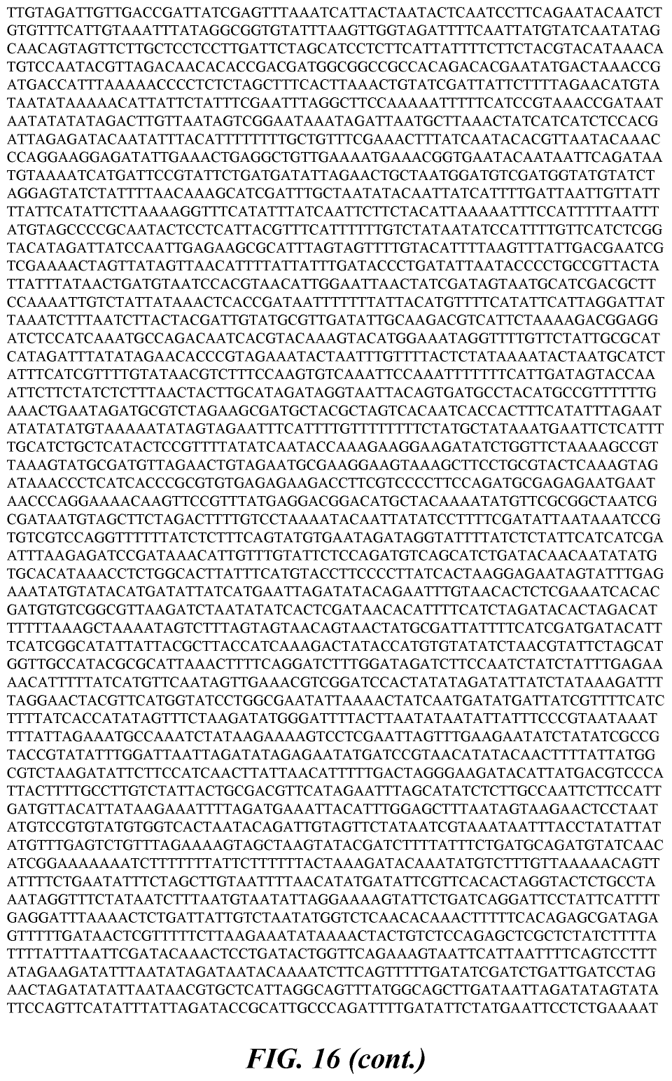

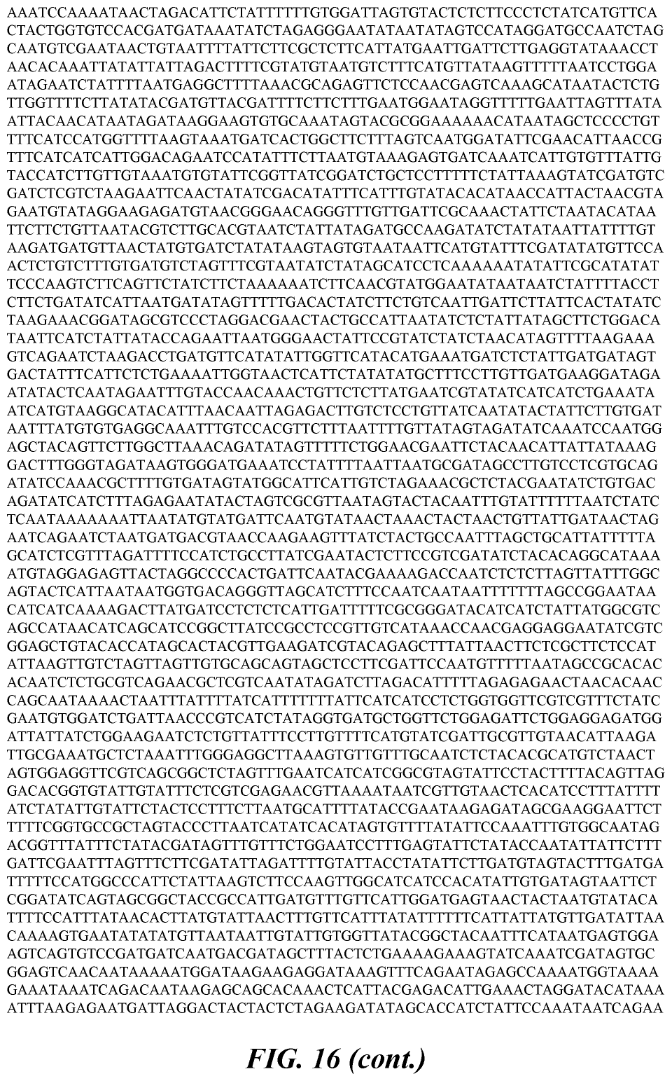

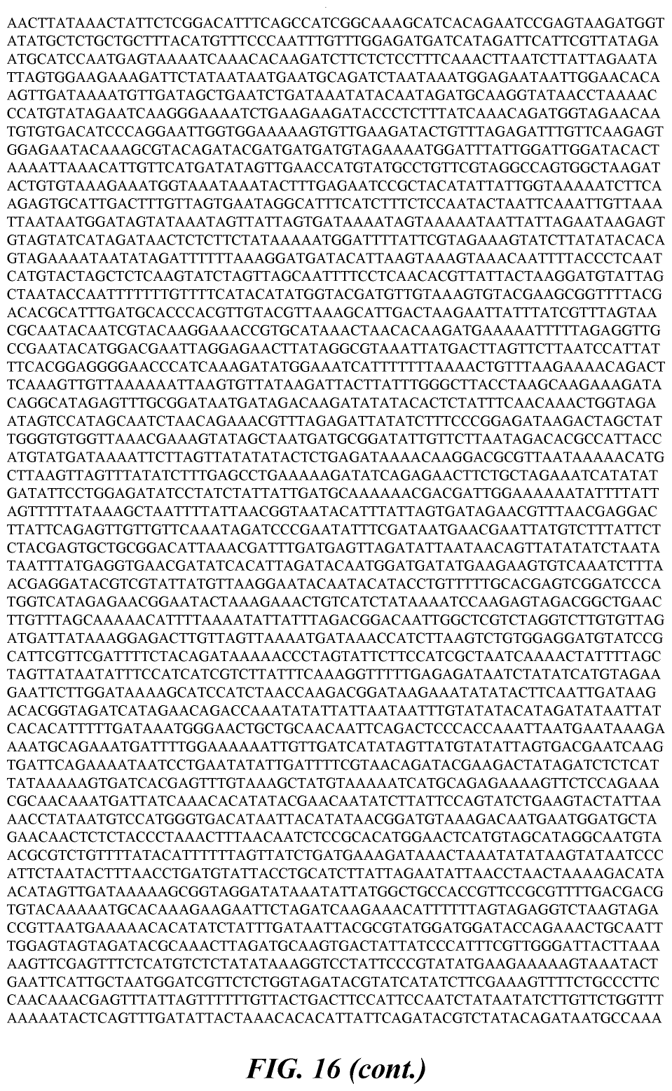

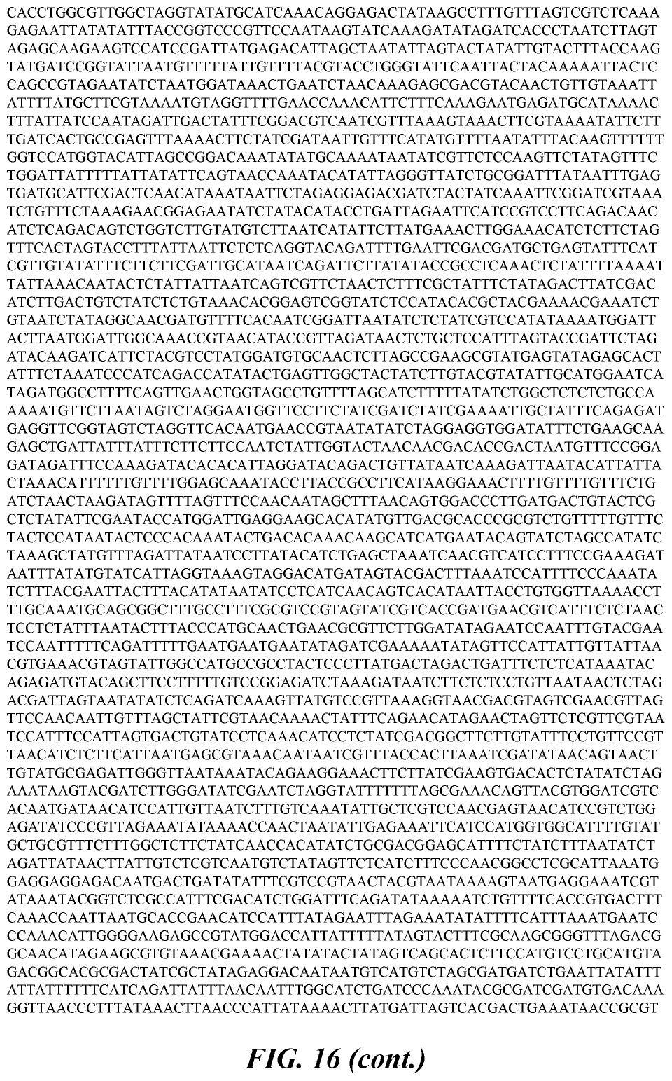

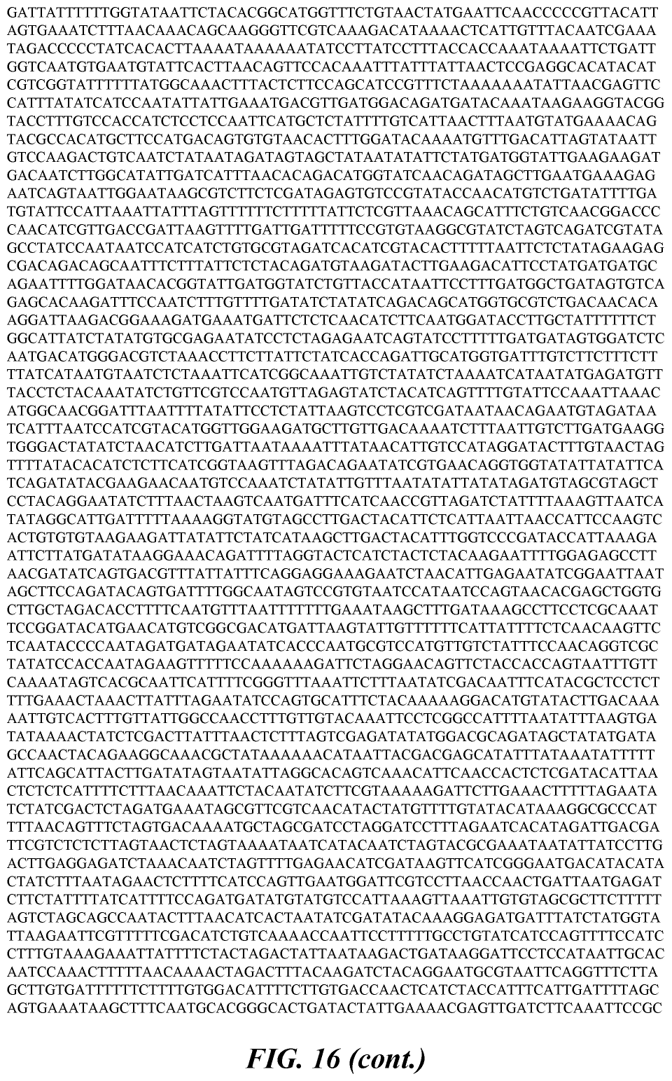

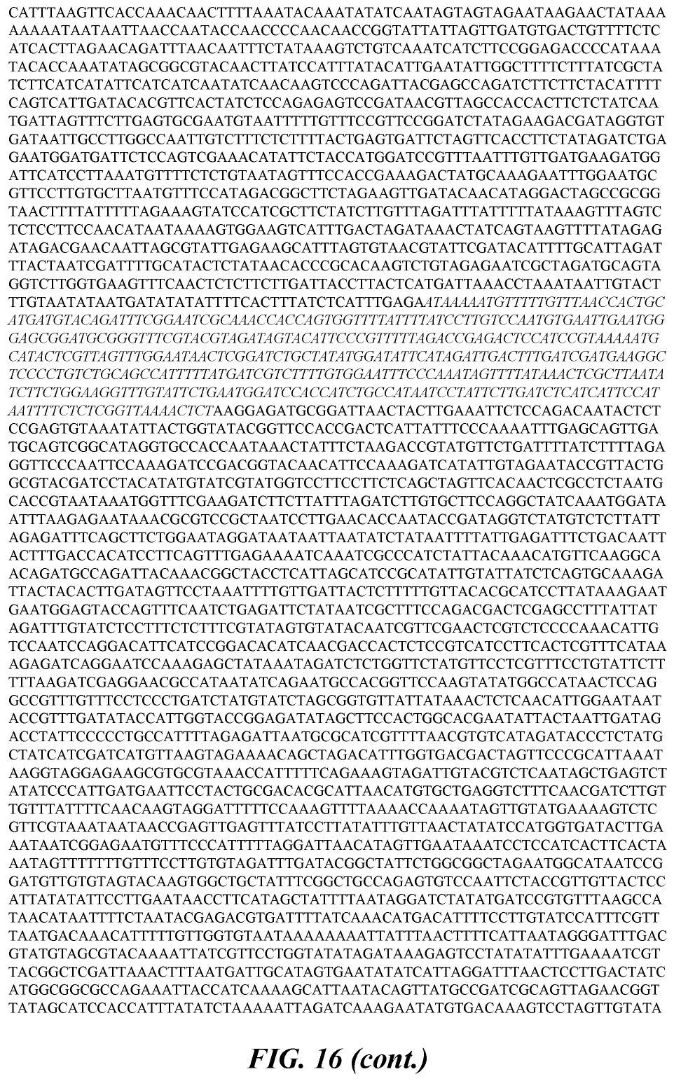

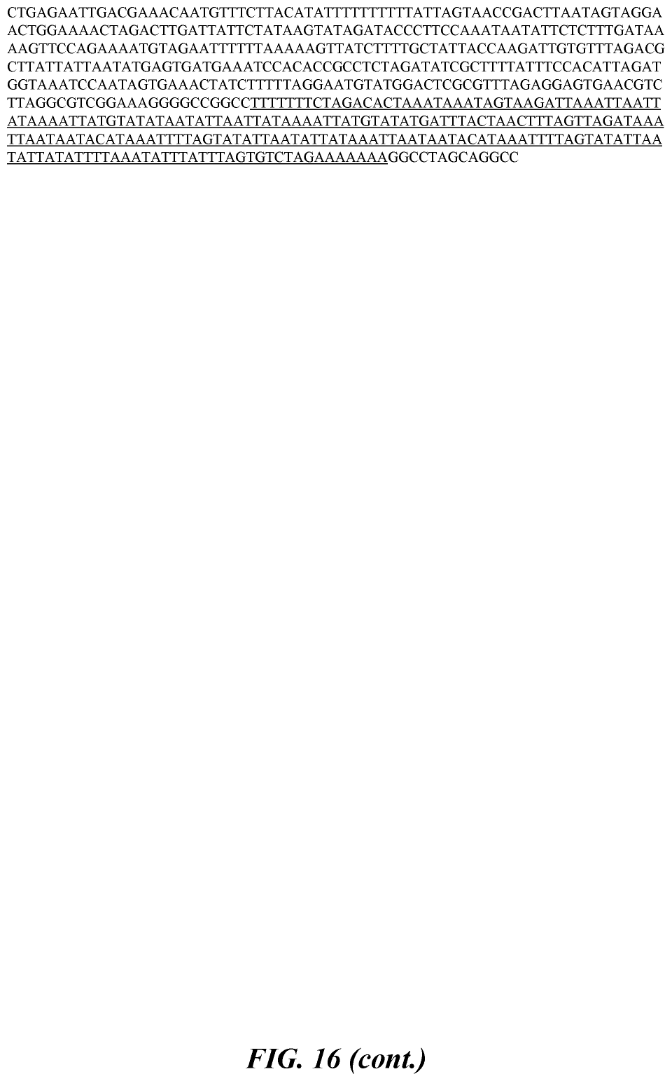

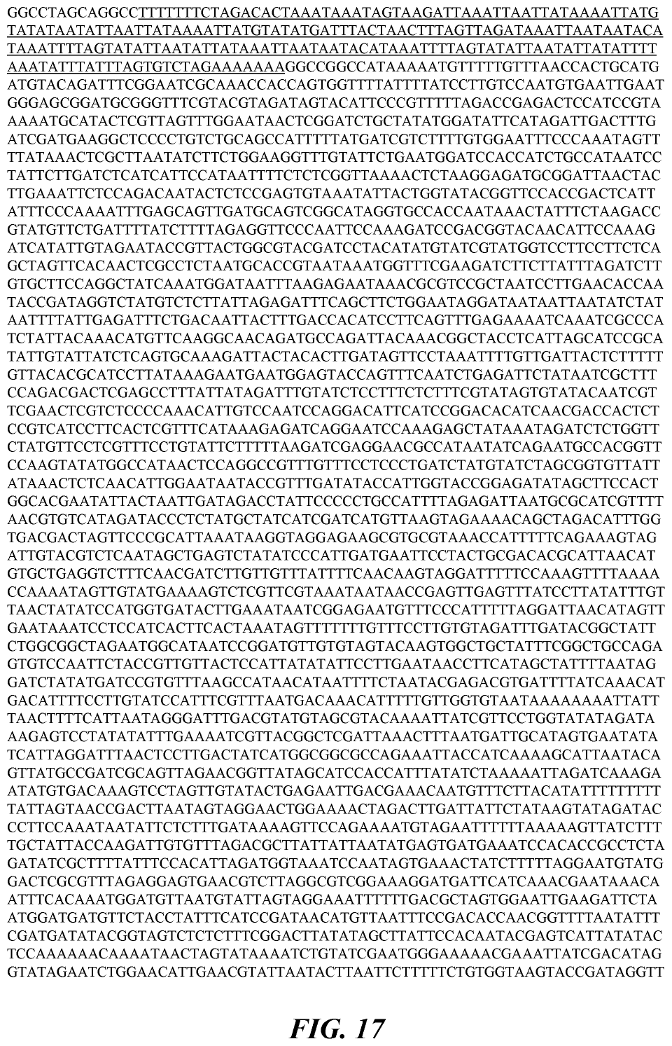

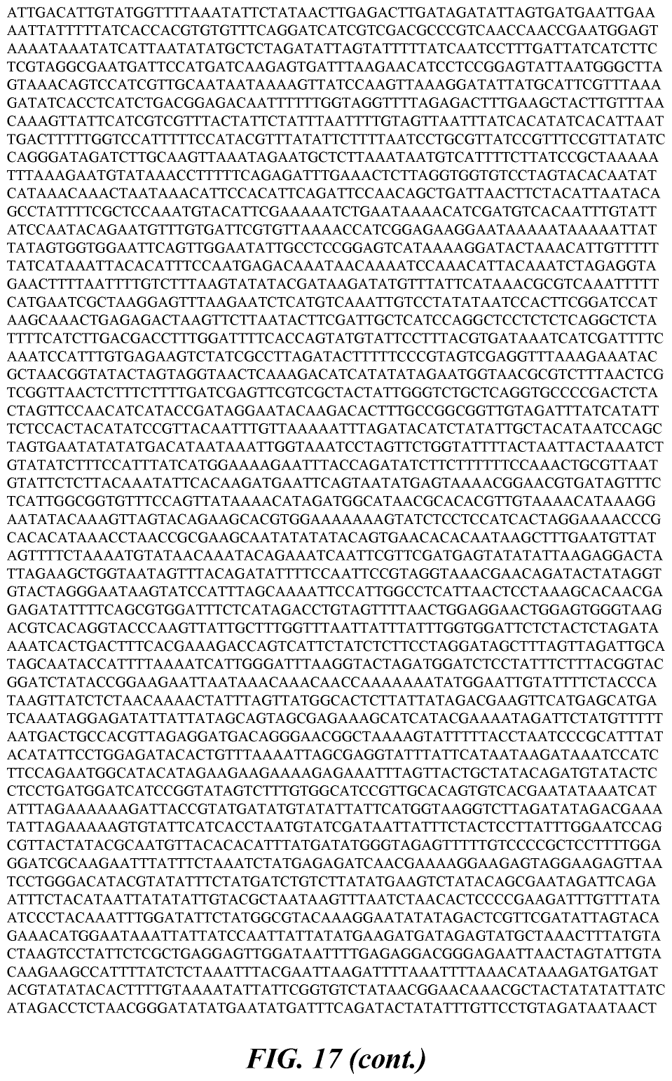

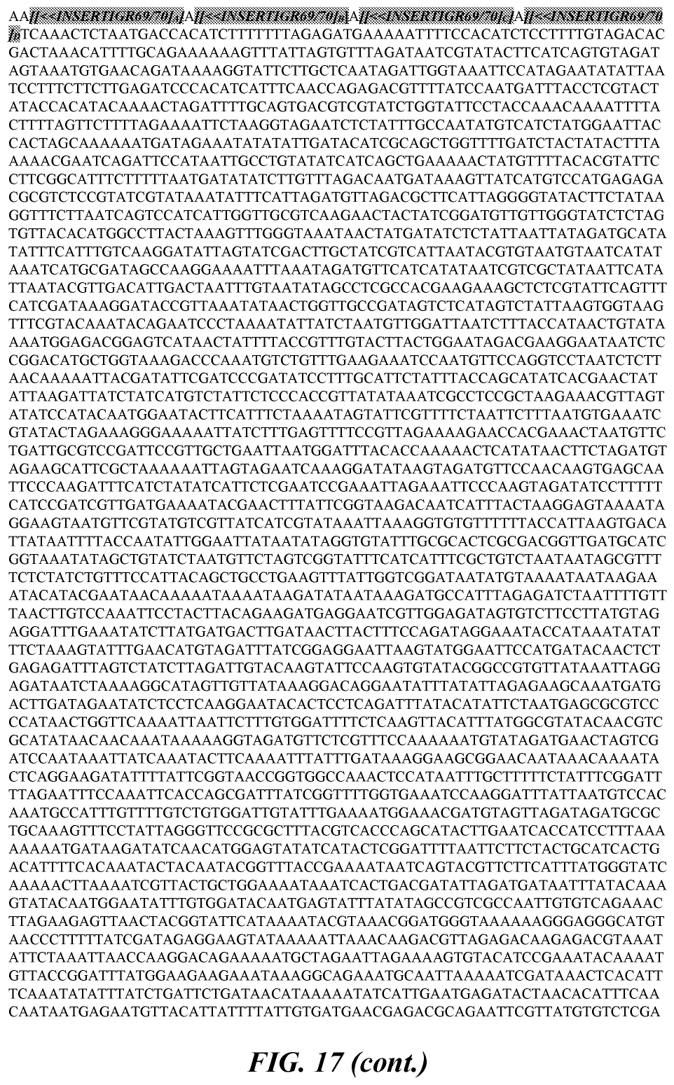

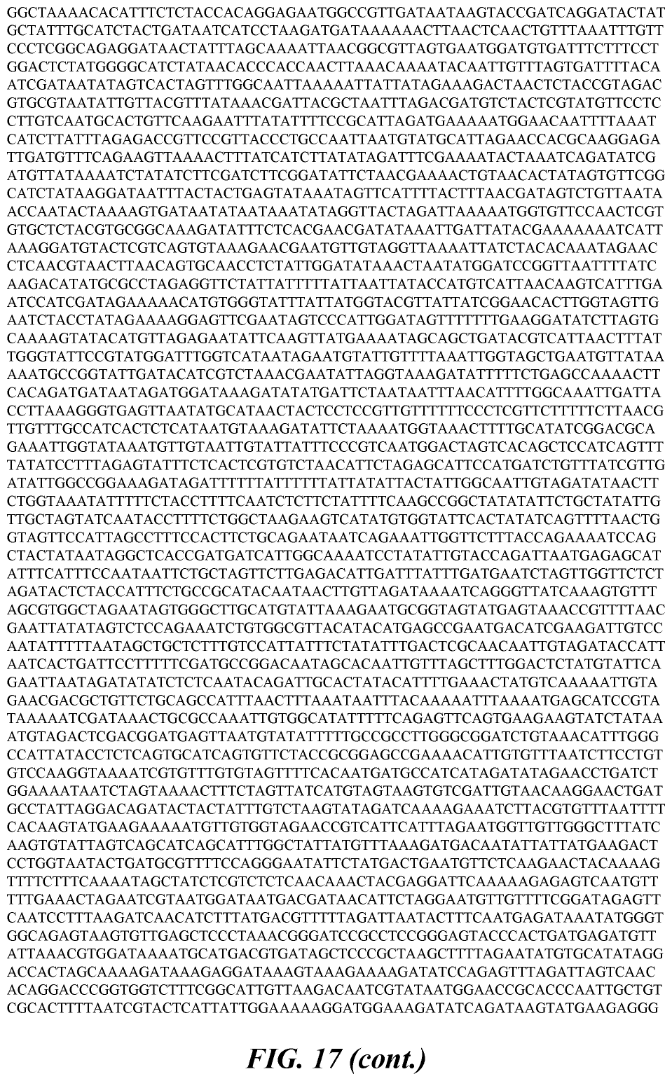

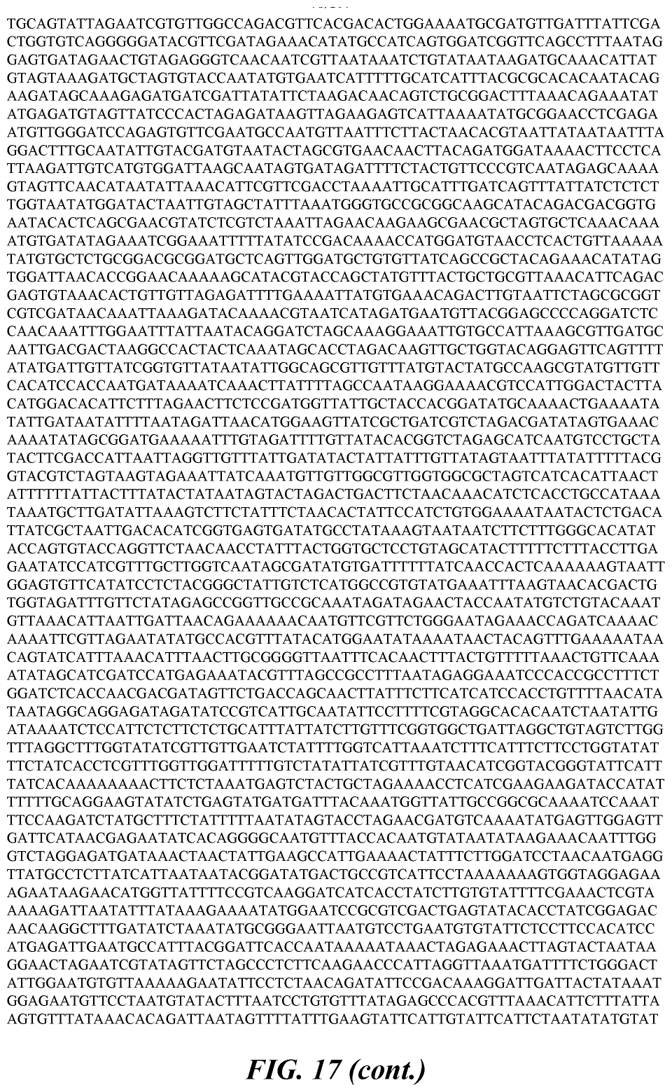

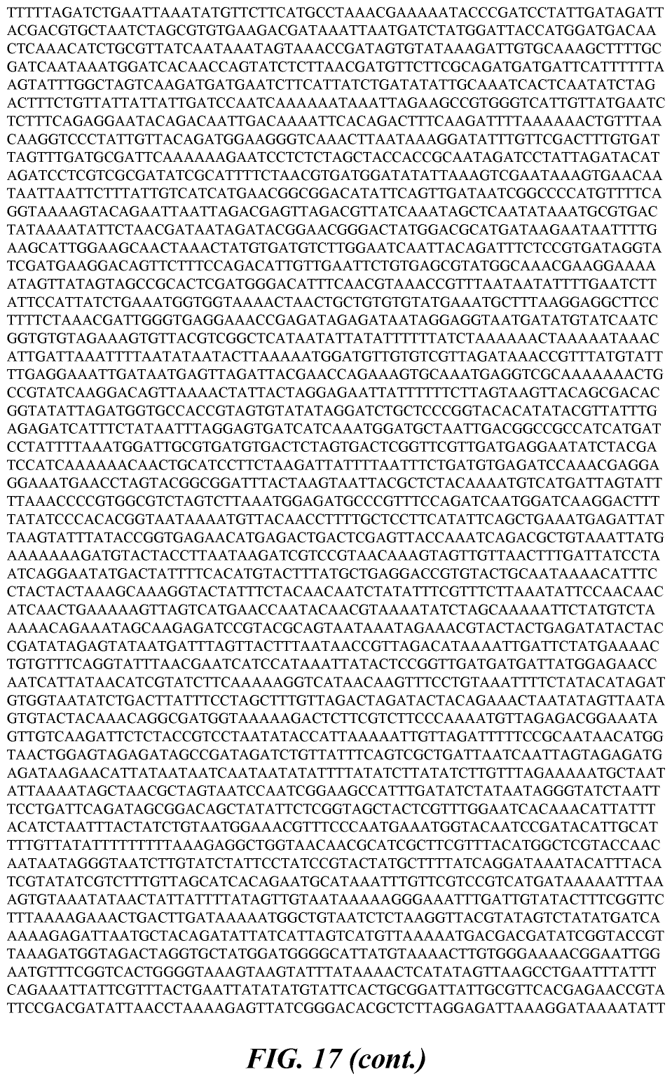

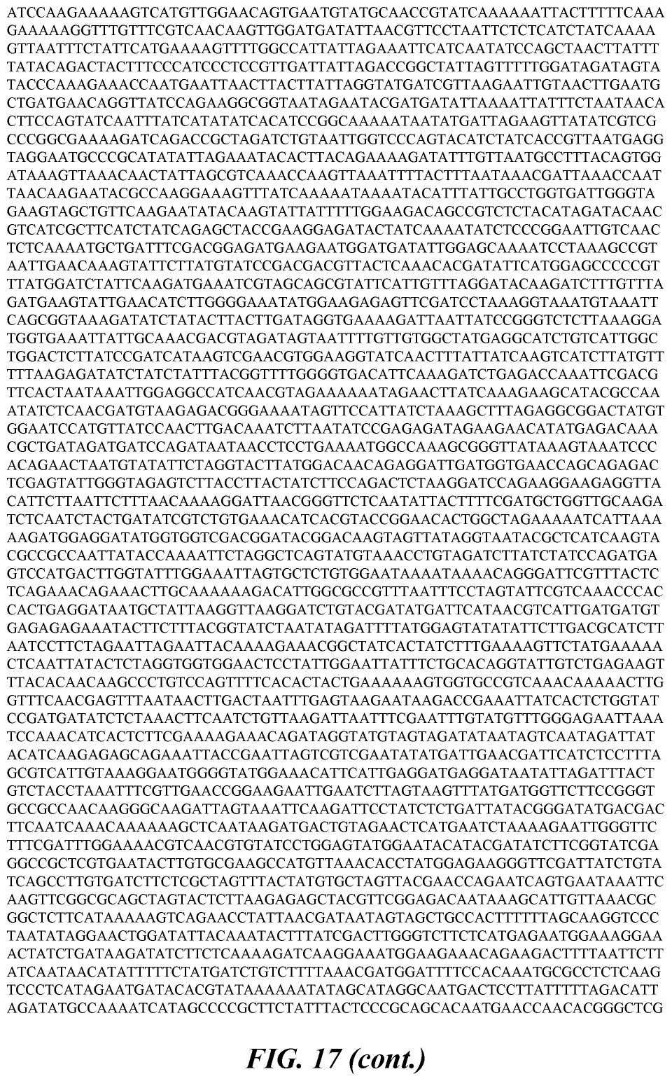

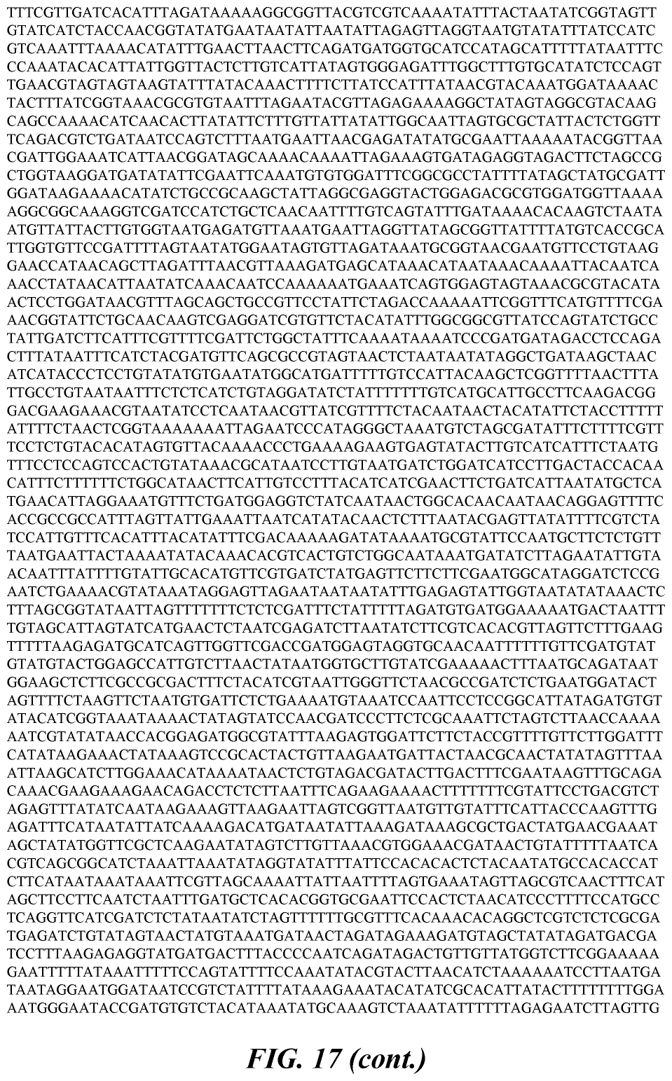

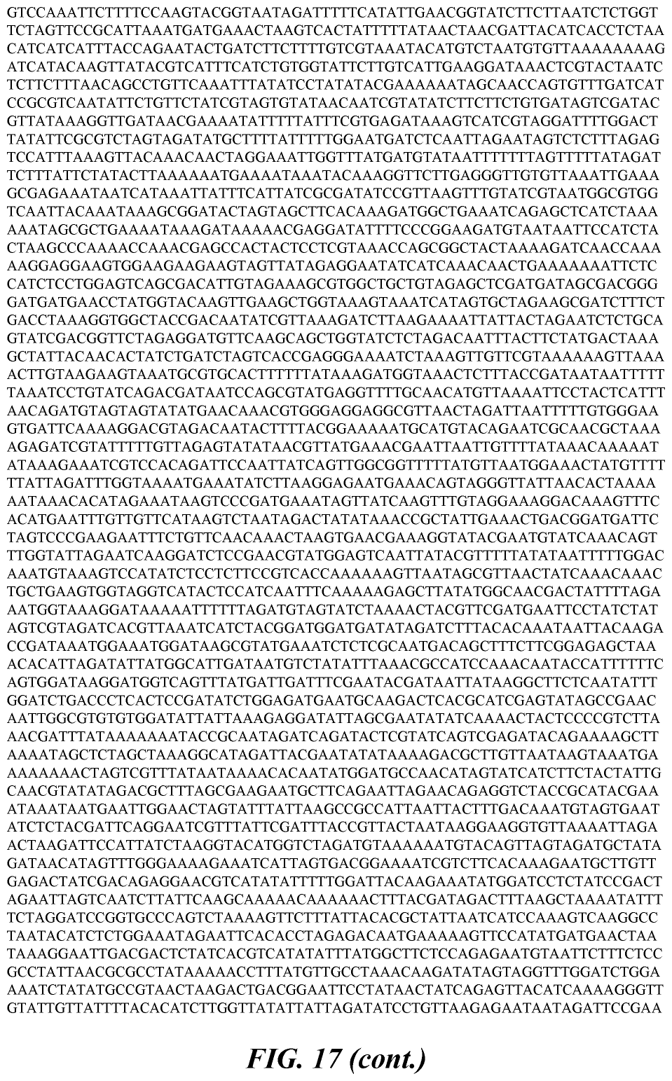

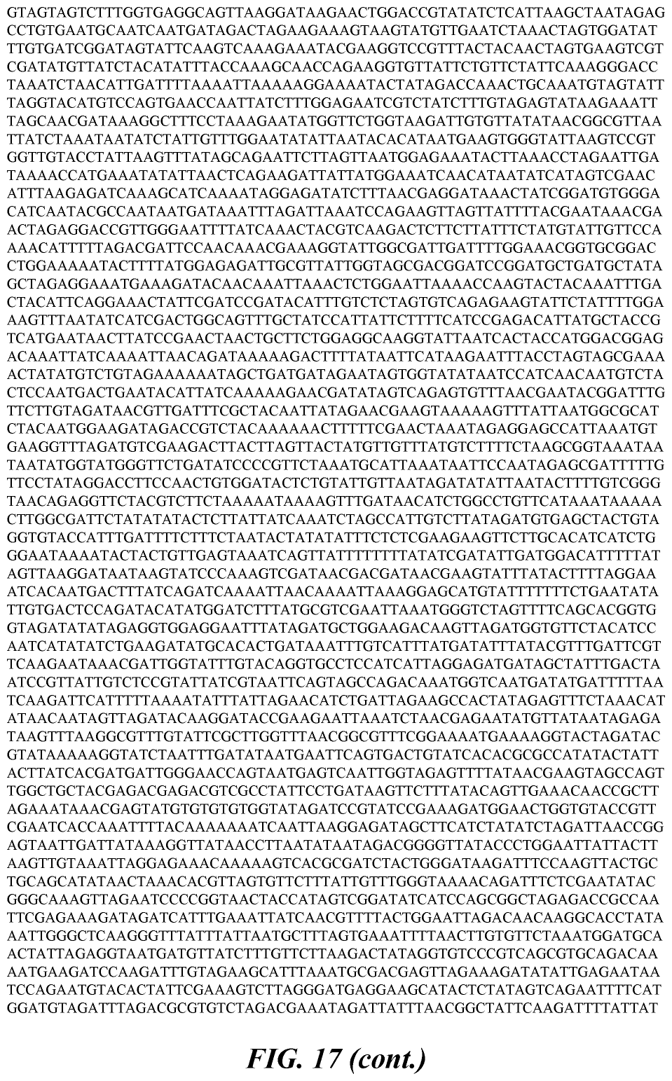

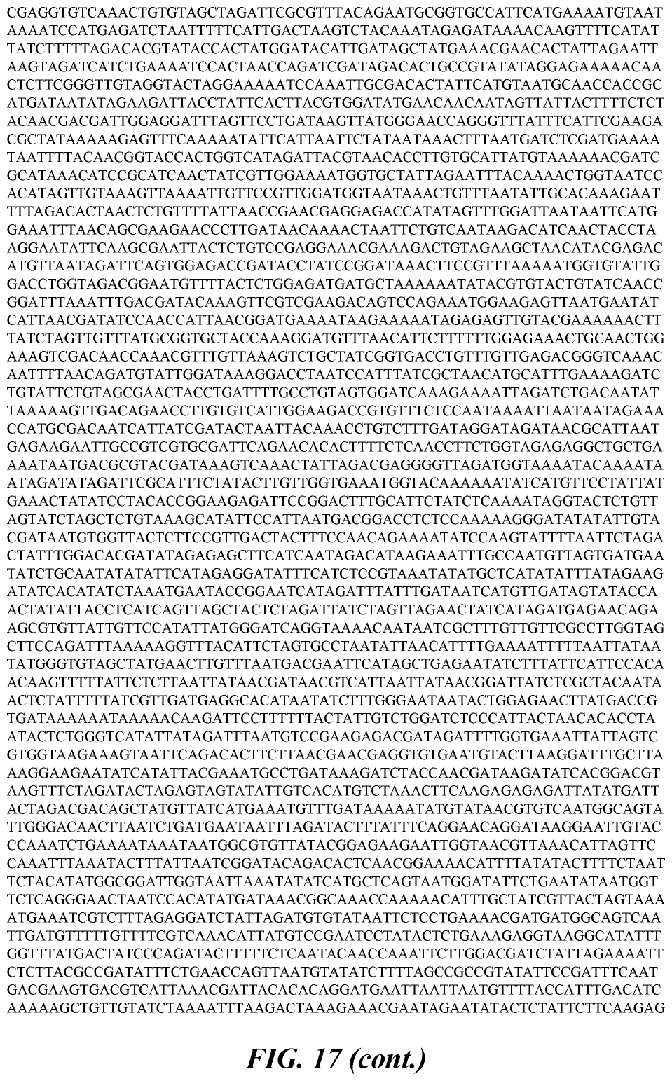

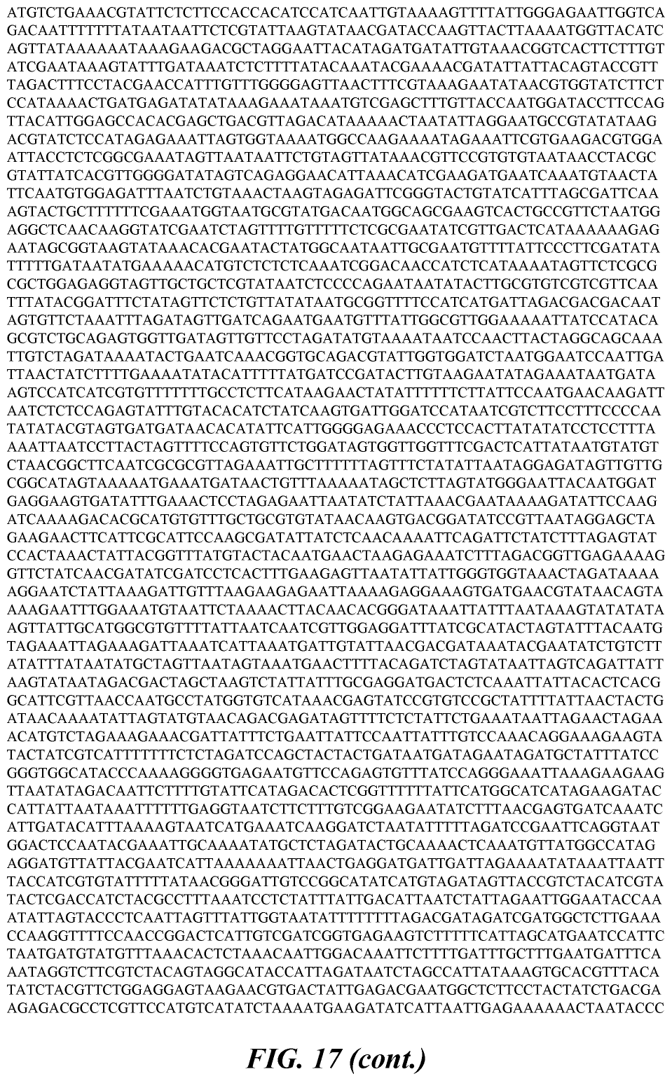

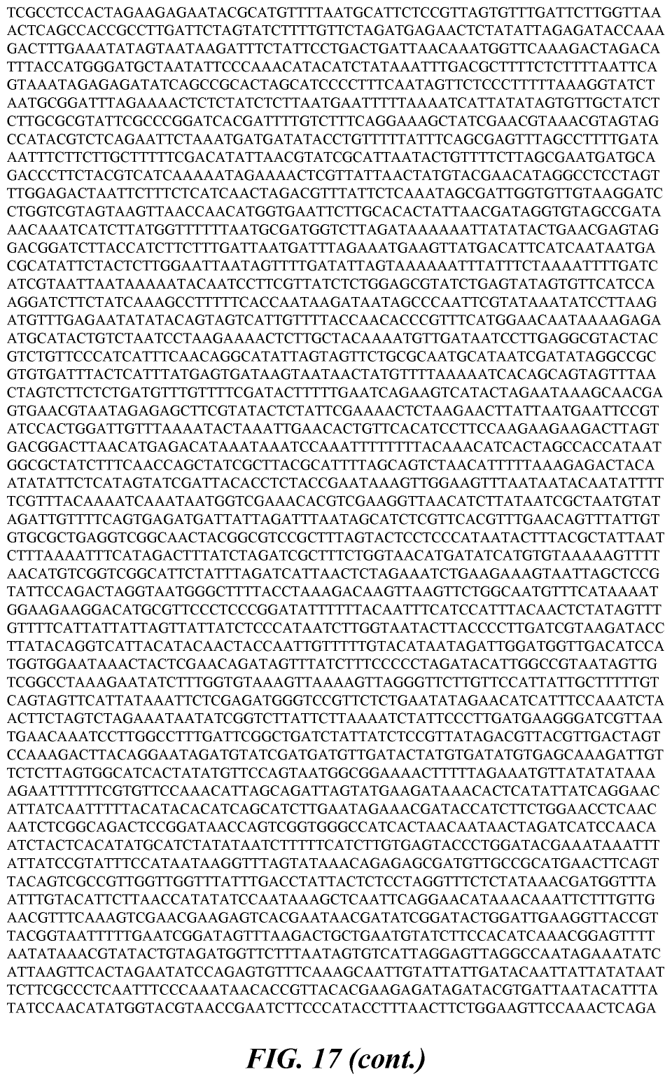

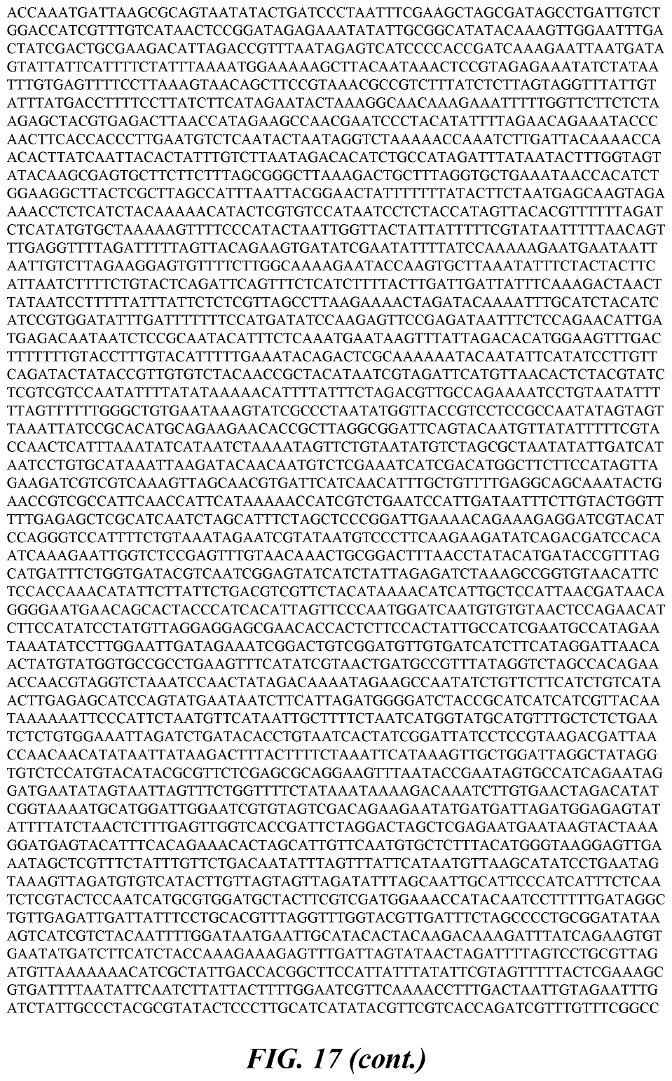

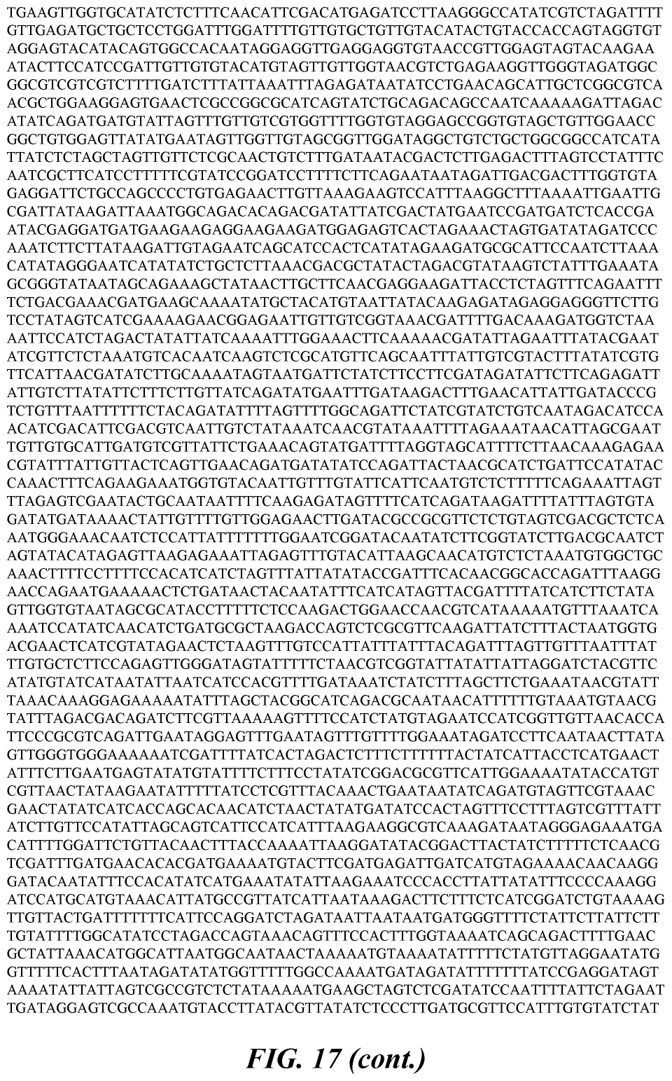

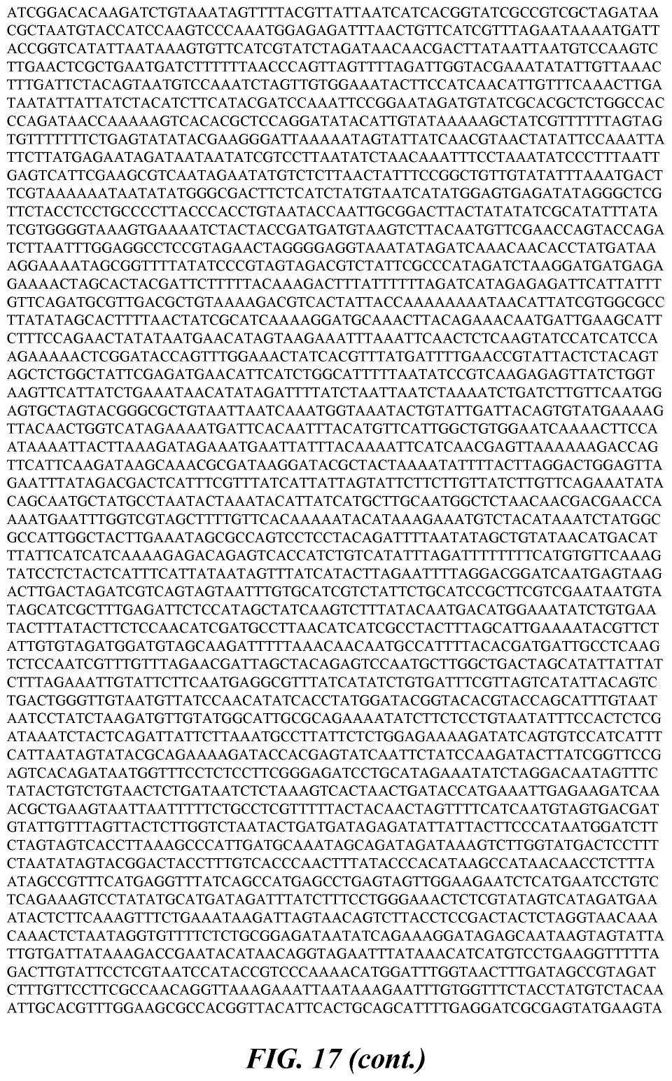

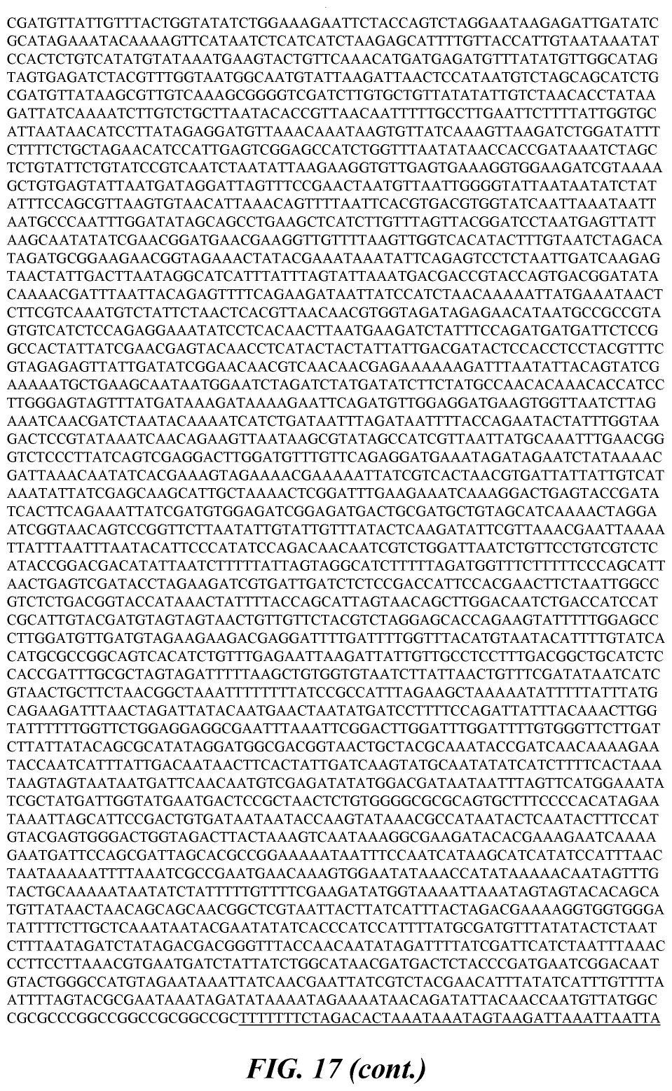

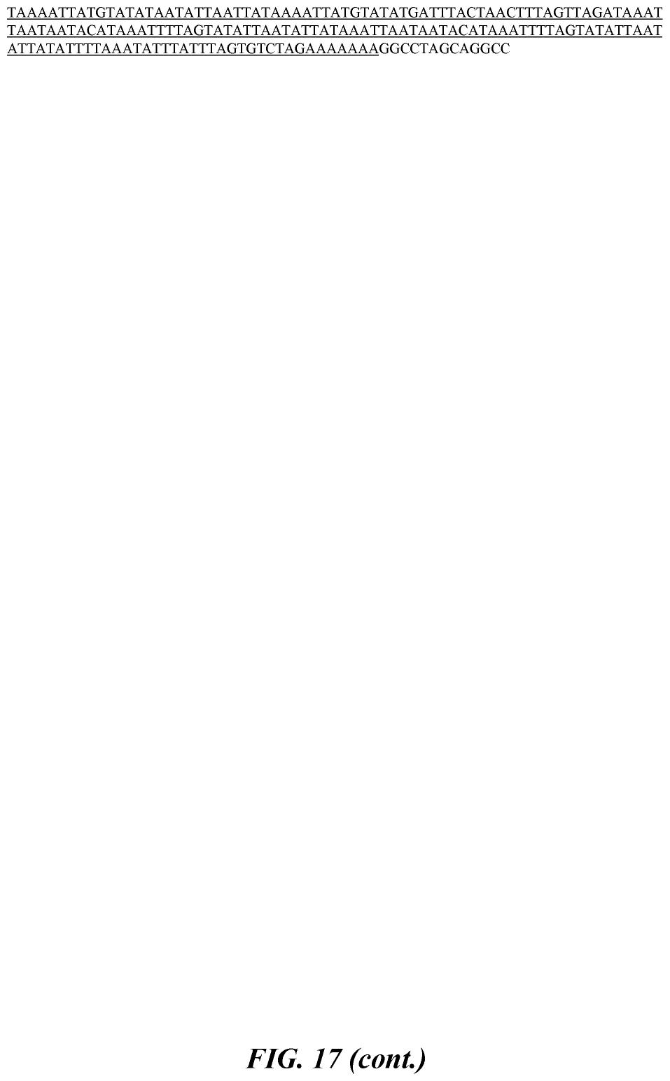

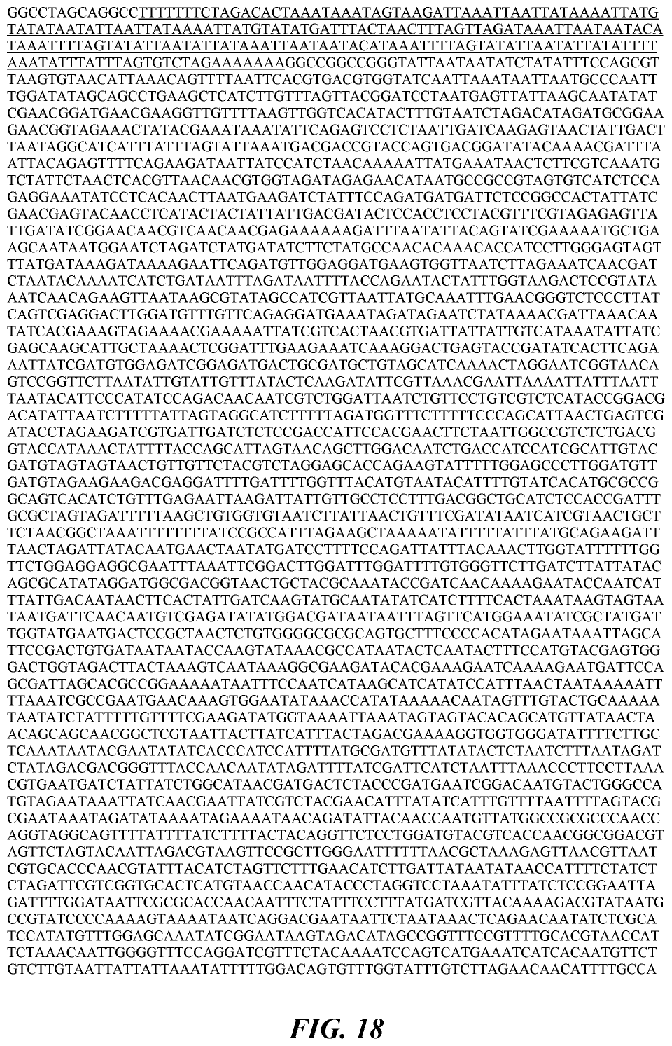

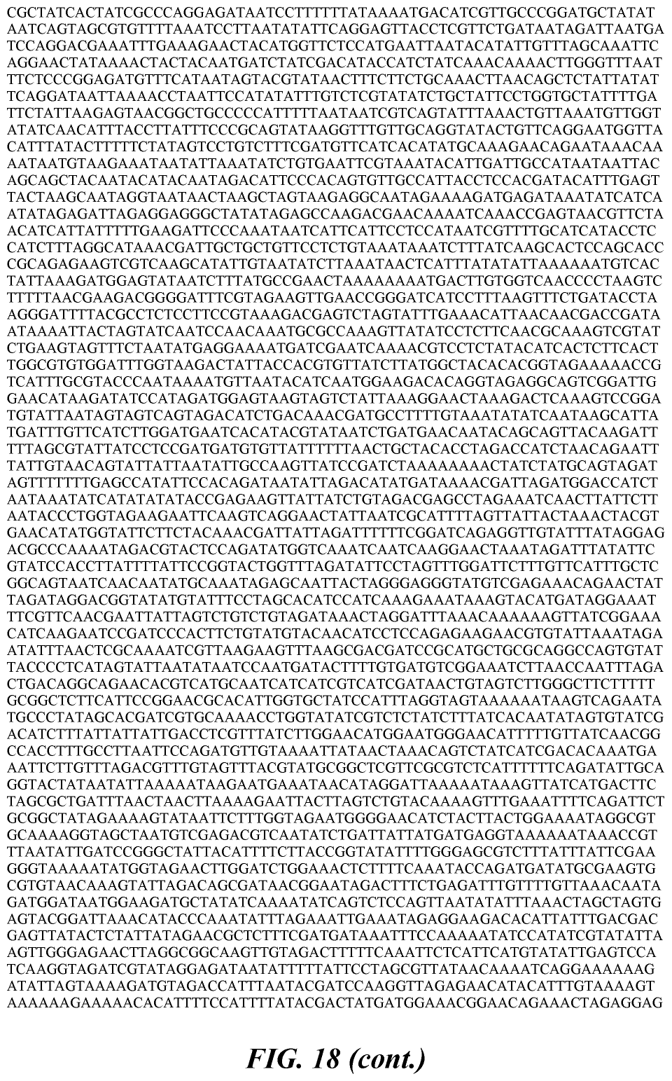

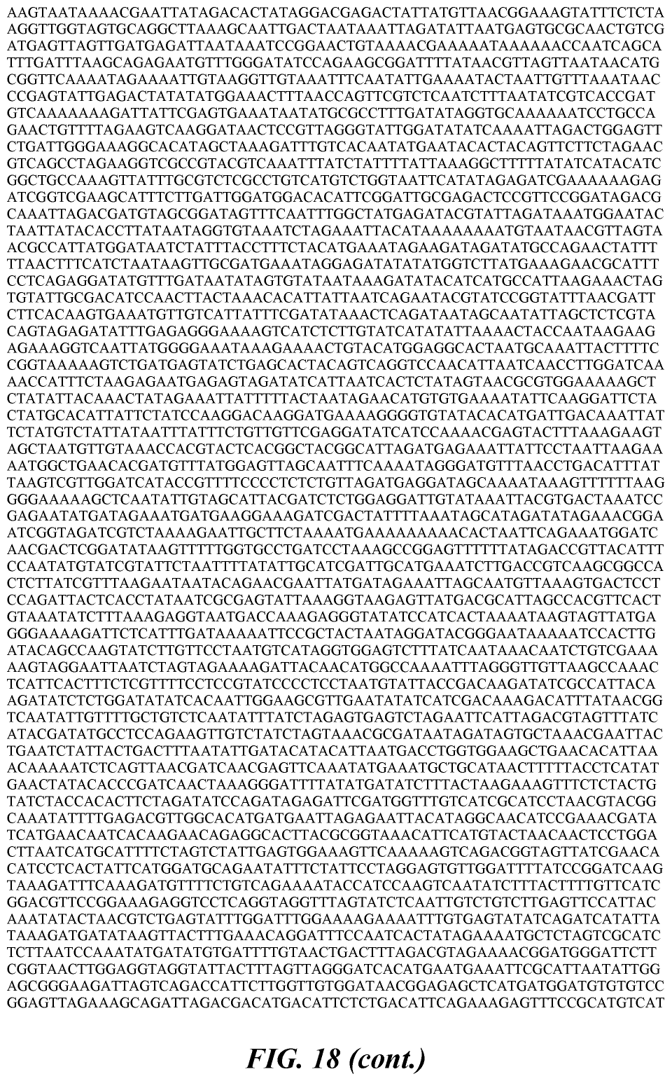

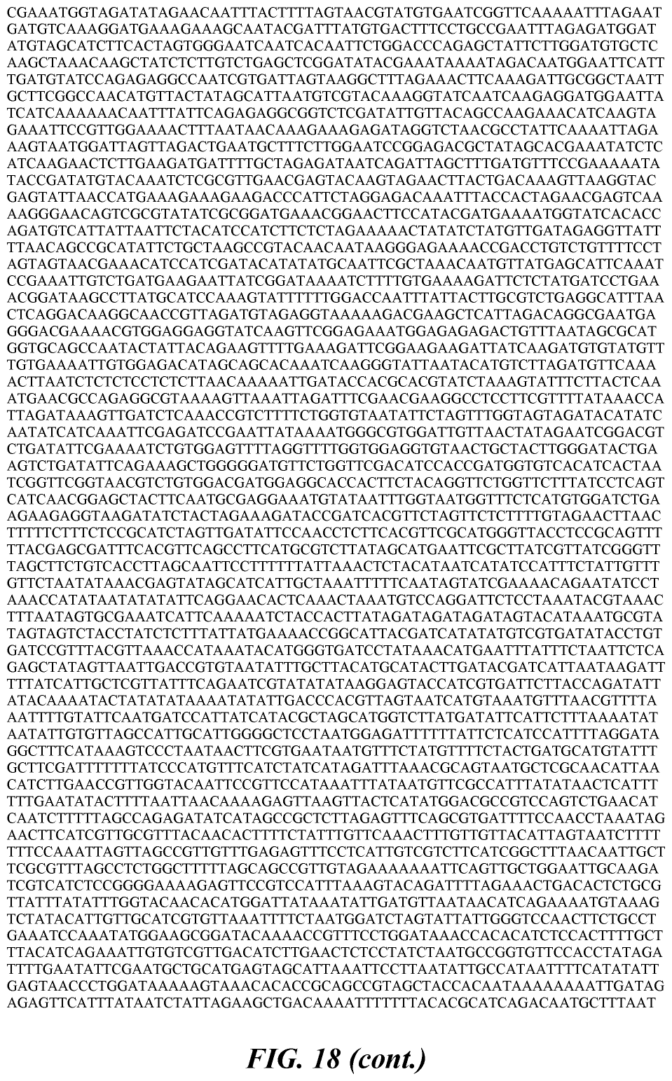

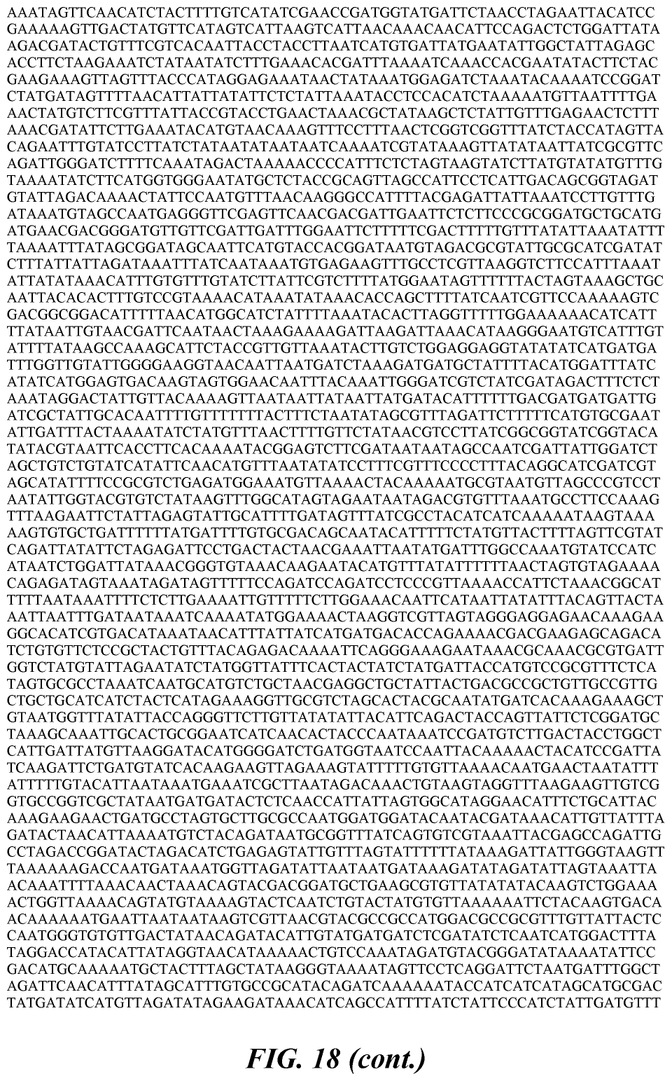

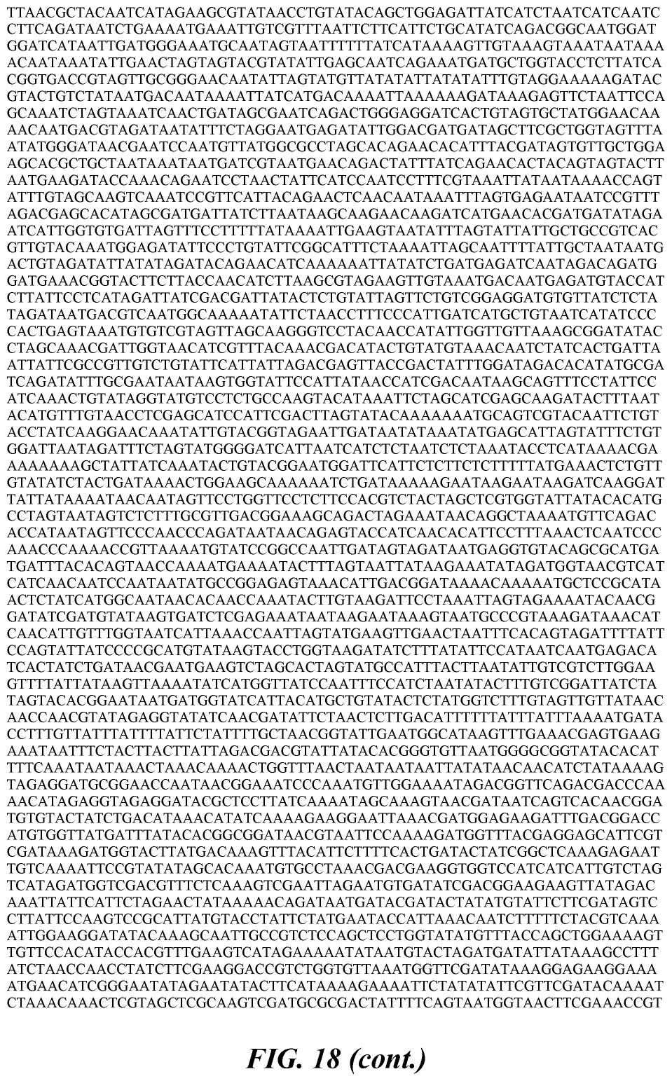

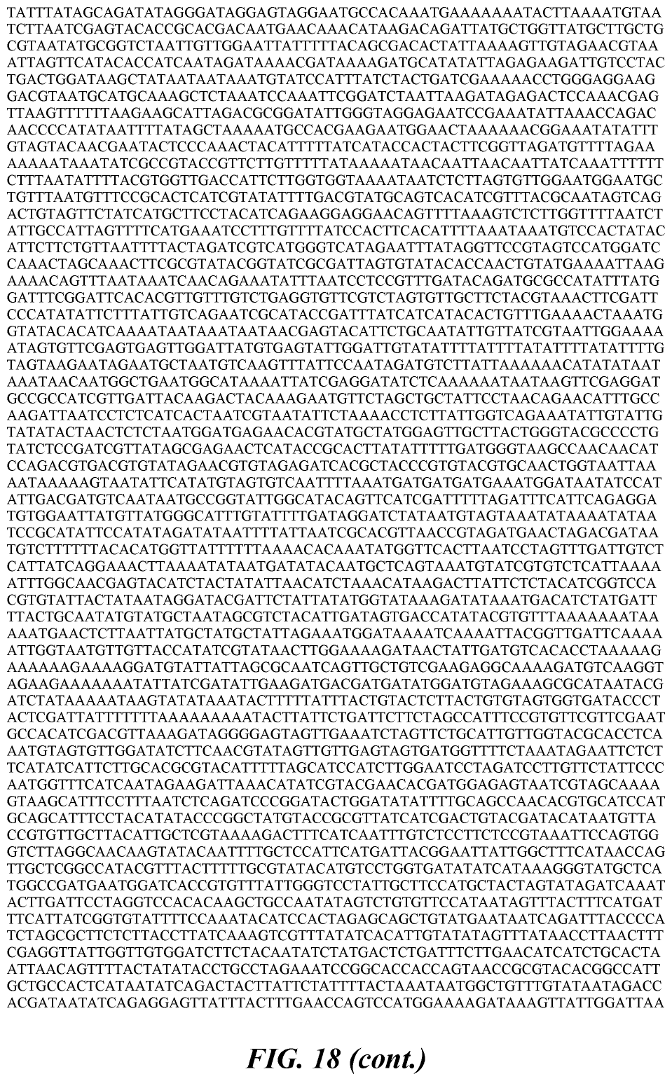

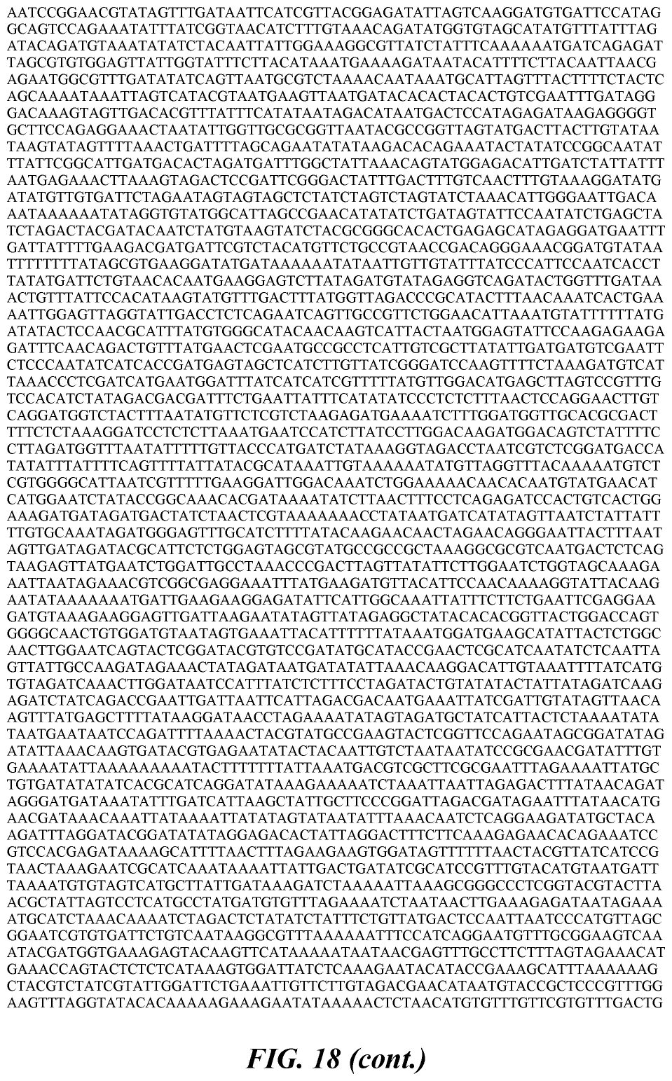

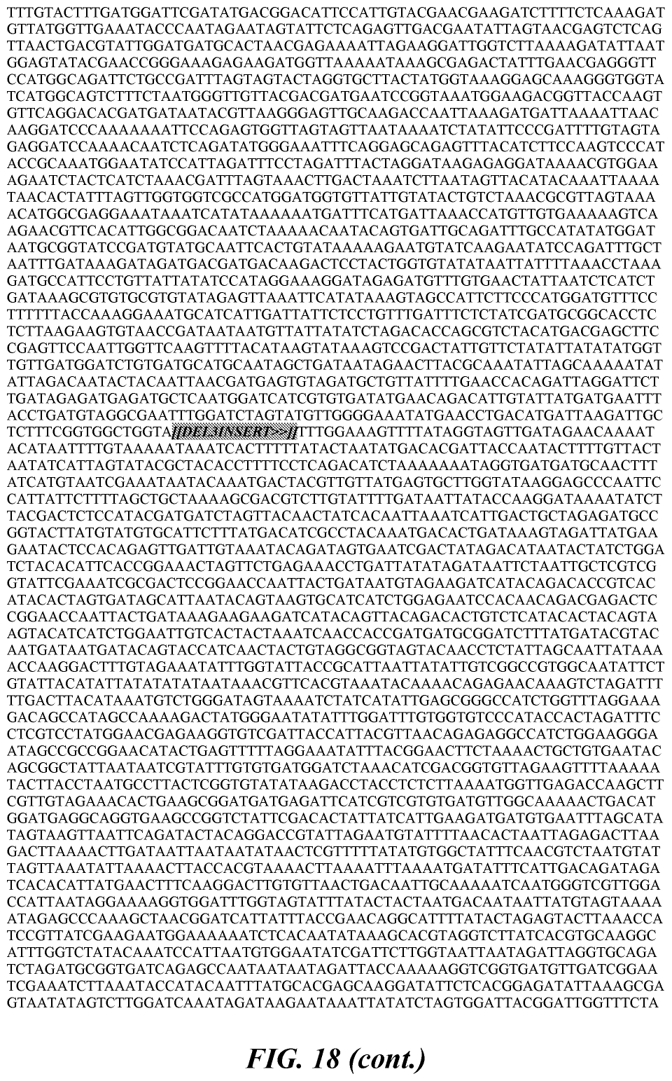

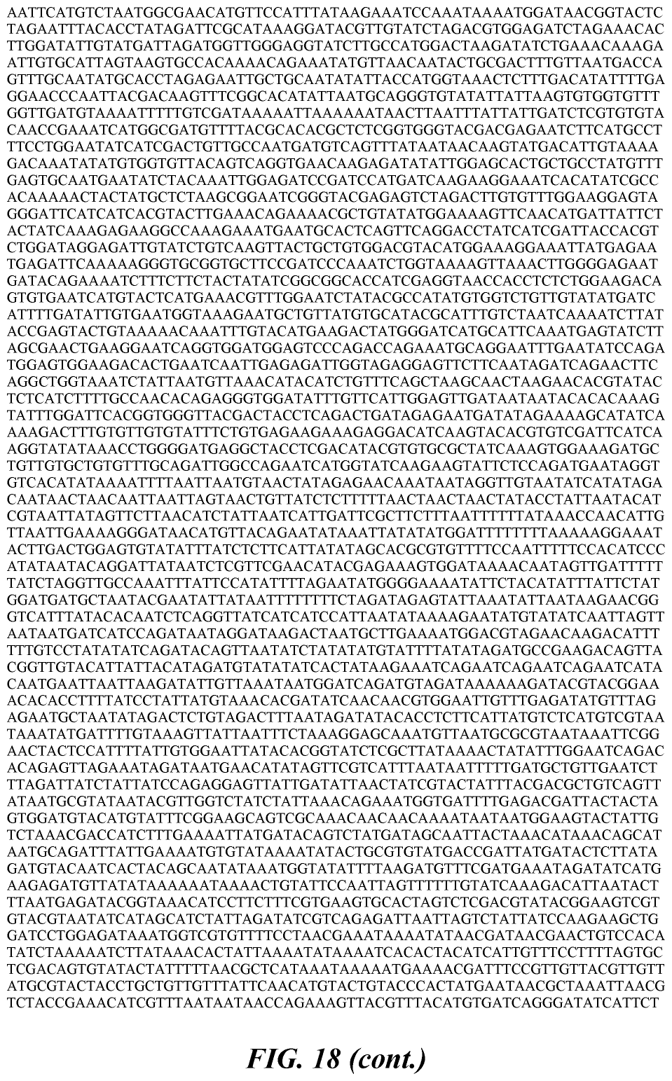

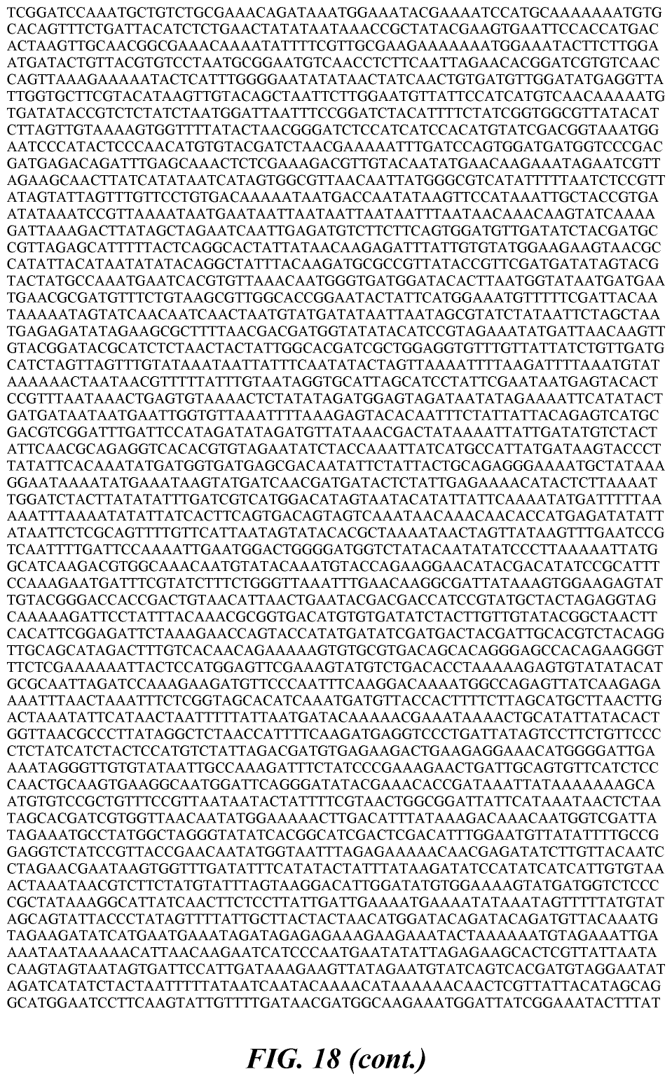

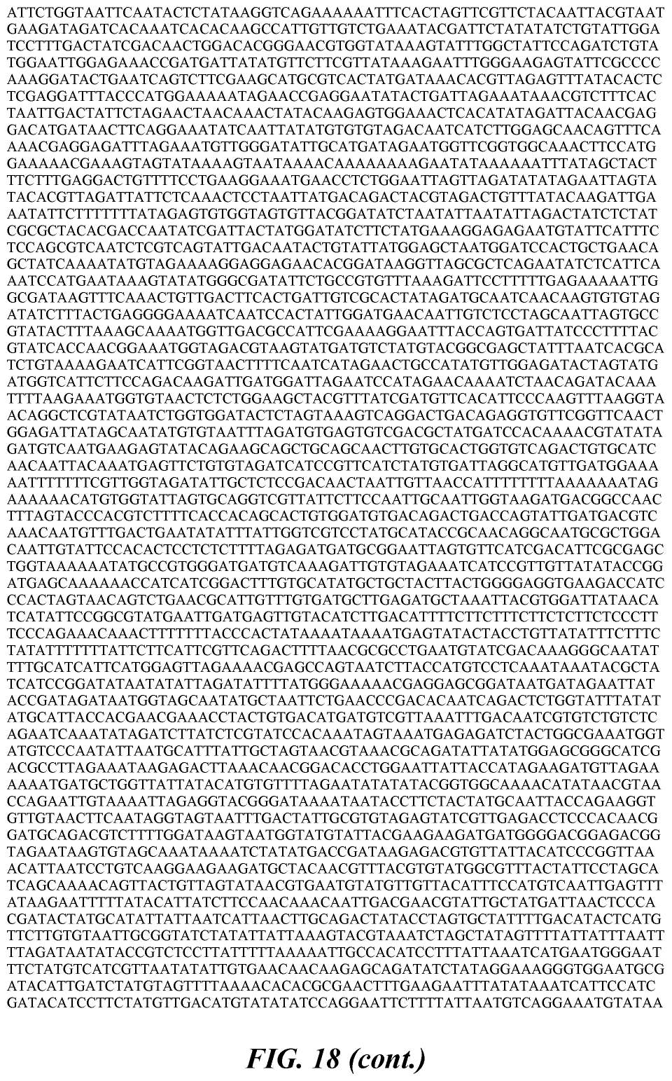

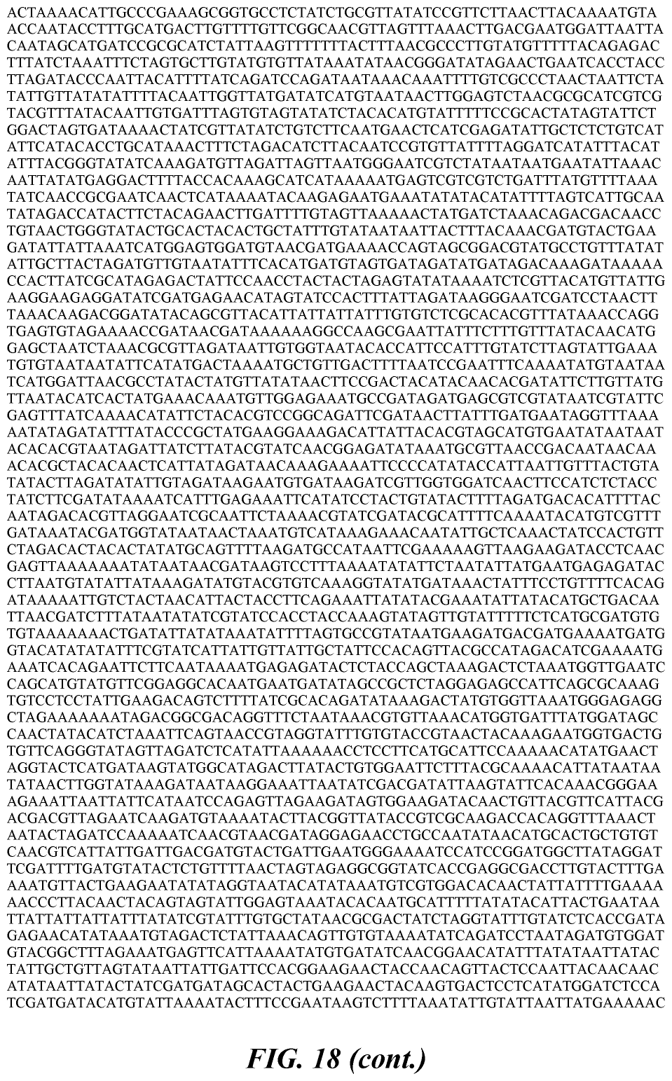

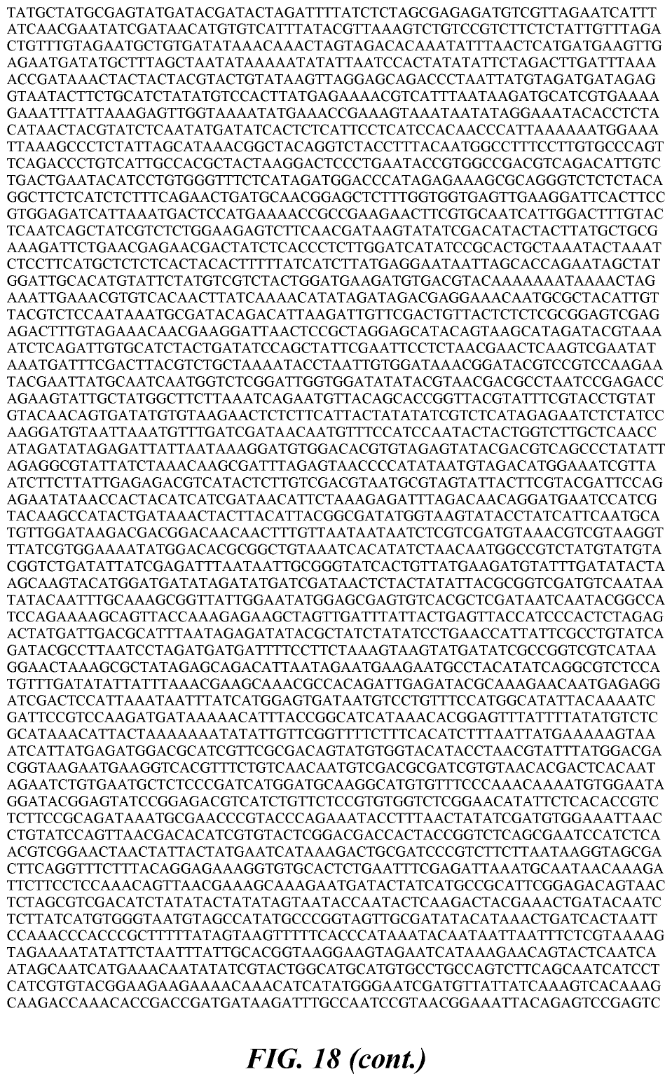

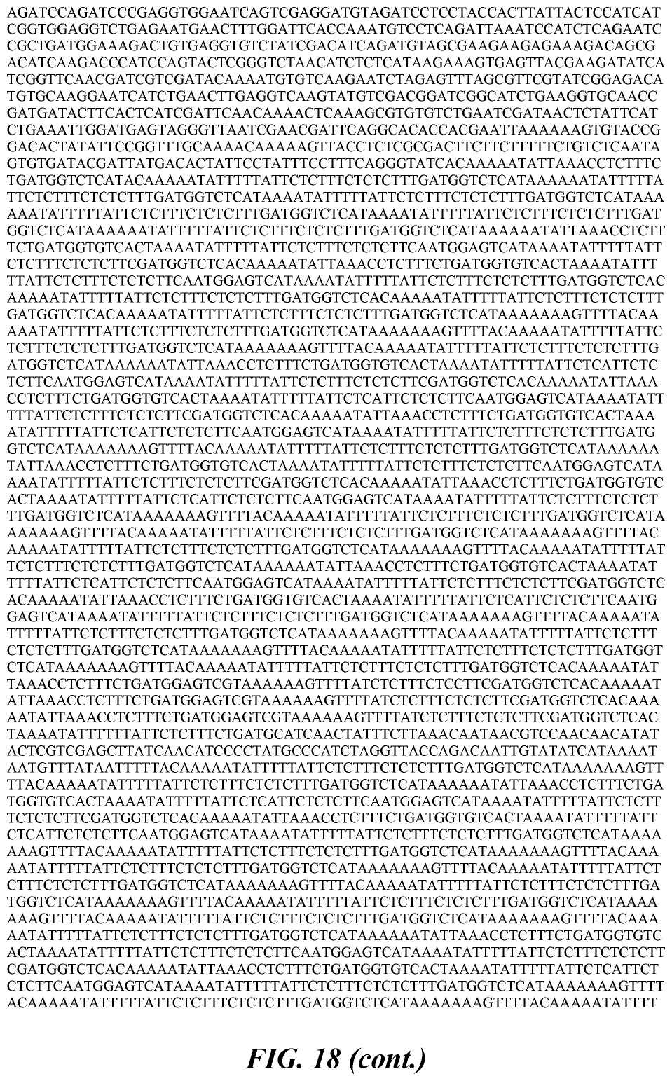

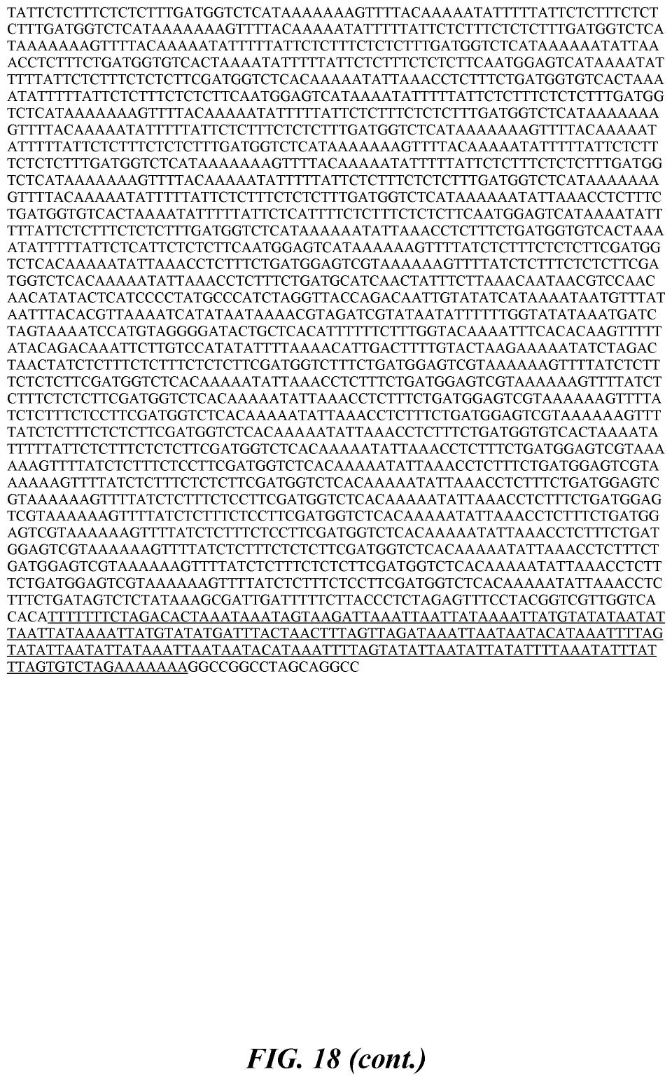

According to some embodiments, the rsMVA viral vector that is used in the compositions disclosed herein is generated according to the methods disclosed in International Application Publication No. WO 2021/236550, which is hereby incorporated by reference as if fully disclosed herein and as indicated in the working examples below. In certain embodiments, three nucleotide fragments, F1, F2, and F3 are synthesized, each fragment including a portion of the full MVA backbone sequence flanked by an MVA concatemer resolution-hairpin loop-concatemer resolution (CR/HL/CR) sequence (FIGS. 14B-14C). F1 encompasses the left ITR and ˜50 kbp of the left end of the internal UR of the MVA genome; F2 contains ˜60 kbp of the middle part of the internal UR of the MVA genome; and F3 encompasses ˜50 kbp of the right end of the internal UR and the right ITR of the MVA genome. sMVA F1 and F2 as well as sMVA F2 and F3 are designed to share ˜3 kbp overlapping sequences to allow the reconstitution of the complete MVA genome by homologous recombination (FIG. 14B). A duplex copy of the 165-nucleotide-long MVA terminal hairpin loop (HL) flanked by MVA CR sequences is added to both ends of each of the three fragments to promote MVA genome resolution and packaging (FIG. 14C). The three sMVA fragments are cloned and maintained in E. coli (DH10B, EP1300, GS1783) by a yeast-bacterial shuttle vector, termed pCCI-Brick (GeneScript), which contains a bacterial mini-F replicon element that can be used as a bacterial artificial chromosome (BAC) vector to stably propagate the three fragments at a low copy number in bacteria. Next-generation sequencing analysis confirmed the integrity of the MVA genomic sequences of the fragments, with the notable exception of an unknown single-point mutation within sMVA fragment F1 located in a non-coding determining region at 3 bp downstream of 021L. FIGS. 16-18 (SEQ ID NOs: 2-4) show sequences of F1, F2, F3, respectively, according to some embodiments. The CR/HL/CR sequences are underlined. The Del2, IGR69/70, and Del3 insertion sites are also indicated in FIGS. 16-18. As such, in some embodiments, each fragment may serve to carry a separate SARS-CoV-2 protein sequence (or variant, mutant, and/or immunogenic fragment thereof). F1, F2, and F3 are then cotransfected into host cells with a helper virus (e.g., fowl pox virus) wherein the full rsMVA virus is reconstituted and capable of expressing the SARS-CoV-2 protein sequence (or variant, mutant, and/or immunogenic fragment thereof) or sequences inserted therein. Each fragment, F1, F2, and F3, includes an overlapping sequence with the adjacent sequence such that when reconstituted, the MVA genome is sequentially reconstituted in the order F1□F2□F3 via homologous recombination between fragments according to some embodiments.

In some embodiments, a nucleotide sequence that encodes a SARS-CoV-2 S protein (or variant, mutant, and/or immunogenic fragment thereof) is inserted into the Del2 site within F1, the IGR69/70 site within F2, or the Del3 site within F3.

In some embodiments, a nucleotide sequence that encodes a SARS-CoV-2 N protein (or variant, mutant, and/or immunogenic fragment thereof) is inserted into the Del2 site within F1 and a nucleotide sequence that encodes a SARS-CoV-2 S protein (or variant, mutant, and/or immunogenic fragment thereof) is inserted into the Del3 site within F3.

In some embodiments, a nucleotide sequence that encodes a SARS-CoV-2 S protein (or variant, mutant, and/or immunogenic fragment thereof) is inserted into the Del2 site within F1 and a nucleotide sequence that encodes a SARS-CoV-2 N protein (or variant, mutant, and/or immunogenic fragment thereof) is inserted into the Del3 site within F3.

In some embodiments, a nucleotide sequence that encodes a SARS-CoV-2 S protein (or variant, mutant, and/or immunogenic fragment thereof) is inserted into the IGR69/70 site within F2 and a nucleotide sequence that encodes a SARS-CoV-2 N protein (or variant, mutant, and/or immunogenic fragment thereof) is inserted into the Del3 site within F3.

In some embodiments, a nucleotide sequence that encodes a SARS-CoV-2 N protein (or variant, mutant, and/or immunogenic fragment thereof) is inserted into the IGR69/70 site within F2 and a nucleotide sequence that encodes a SARS-CoV-2 S protein (or variant, mutant, and/or immunogenic fragment thereof) is inserted into the Del3 site within F3.

In some embodiments, a nucleotide sequence that encodes a SARS-CoV-2 S protein (or variant, mutant, and/or immunogenic fragment thereof) is inserted into the Del2 site within F1 and a nucleotide sequence that encodes a SARS-CoV-2 N protein (or variant, mutant, and/or immunogenic fragment thereof) is inserted into the IGR69/70 site within F2.

In some embodiments, a nucleotide sequence that encodes a SARS-CoV-2 N protein (or variant, mutant, and/or immunogenic fragment thereof) is inserted into the Del2 site within F1 and a nucleotide sequence that encodes a SARS-CoV-2 S protein (or variant, mutant, and/or immunogenic fragment thereof) is inserted into the IGR69/70 site within F2.

The SARS-CoV-2 S protein (or variant, mutant, and/or immunogenic fragment thereof) and/or the SARS-CoV-2 N protein (or variant, mutant, and/or immunogenic fragment thereof) that are inserted into the F1, F2, and/or F3 fragments as discussed herein are encoded by any SARS-CoV2 S or N protein, including a reference sequence or any variant or mutants thereof. Exemplary SARS-CoV2 S or N proteins that are encoded by the heterologous sequences that can be inserted into the F1, F2, and/or F3 fragments as discussed herein (and thus incorporated into the rsMVA vector and reconstituted rsMVA virus) are found in International Application Publication No. WO 2021/236550, as well as additional sequences and mutations discussed below.

In some embodiments, the rsMVA vector comprises one or more heterologous DNA sequences encoding the S protein and/or N protein of the SARS-CoV-2 Wuhan-Hu-1 reference stain. In some embodiments, the rsMVA vector comprises one or more heterologous DNA sequences encoding a mutant S protein and/or N protein such as mutant S protein and/or N protein based on a variant of concern, including B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron). Exemplary sequences of the variants of the S proteins and N proteins are illustrated in Table 1 below. According to some embodiments, the rsMVA vectors are reconstituted rsMVA viruses that express or are capable of expressing the one or more S proteins and/or N proteins (or mutants thereof) disclosed herein.

TABLE 1
Exemplary Sequences of SARS-COV-2 S and N Proteins

| SEQ ID | | |
|---|---|---|
| NO: | Sequences | Descriptions |
| 5 | atgtttgtctttcttgtcttattgccactagtctctagtcagtgtgttaatcttaca | Nucleotide |
| | accagaactcaattaccacctgcatacactaattctttcacacgtggtgttt | sequence/ORF (5′ to |
| | attaccctgacaaagtattcagatcctcagtattacattcaactcaggactt | 3′ end) of the SARS- |
| | gttcttacctttcttctccaatgttacttggttccatgctatacatgtctctggga | CoV-2 S antigen |
| | ccaatggtactaagaggtttgataaccctgtcctaccatttaatgatggtgt | sequence based on |
| | ttatttcgcttccactgagaagtctaacataataagaggctggatatttggt | the Wuhan-Hu-1 |
| | actactttagattcgaagacccagtccctacttattgttaataacgctacta | reference strain |
| | atgttgttattaaagtctgtgaatttcaattctgtaatgatccattcttgggtgttt | (#NC_045512) and |
| | attaccacaagaacaacaagagttggatggaaagtgagttcagagttt | optimized for stability |
| | attctagtgcgaataattgcactttcgaatatgtctctcagcctttccttatgg | in vaccinia by silent |
| | accttgaaggtaaacagggtaatttcaagaatcttagggaatttgtgttta | codon alteration to |
| | agaatattgatggttatttcaagatatattctaagcacacgcctattaattta | avoid 4 or more of the |
| | gtgcgtgatctccctcagggtttctcggctttagaaccattggtagatttgcc | same nucleotides in |
| | aataggtattaacatcactaggtttcaaactttacttgctttacatagaagtt | consecutive order |
| | atttgactcctggtgattcttcttcaggttggacagctggtgctgcagcttatt | |
| | atgtgggttatcttcaacctaggactttcctattgaaatataatgagaatgg | |
| | aaccattacagatgctgtagactgtgcacttgaccctctctcagaaacaa | |
| | agtgtacgttgaaatccttcactgtagagaaaggaatctatcaaacttcta | |
| | actttagagtccaaccaacagaatctattgttagatttcctaatattacaaa | |
| | cttgtgccctttcggtgaagtatttaacgccaccagatttgcatctgtttatgc | |
| | ttggaacaggaagagaatcagcaactgtgttgctgattattctgtcctatat | |
| | aattccgcatcattctccacatttaagtgttatggagtgtctcctactaaatta | |
| | aatgatctctgctttactaatgtctatgcagattcatttgtaattagaggtgat | |
| | gaagtcagacaaatcgctccagggcaaactggaaagattgctgattat | |
| | aattataaattaccagatgactttacaggctgcgttatagcttggaattcta | |
| | acaatcttgattctaaggttggtggtaattataattacctgtatagattgttta | |
| | ggaagtctaatctcaaacctttcgagagagatatttcaactgaaatctatc | |
| | aggccggtagcacaccttgtaatggtgttgaaggatttaattgttactttcct | |
| | ttacaatcatatggtttccaacccactaatggtgttggttaccaaccataca | |
| | gagtagtagtactttcatttgaacttctacatgcaccagcaactgtttgtgg | |
| | acctaagaagtctactaatttggttaagaacaaatgtgtcaatttcaacttc | |
| | aatggtttaacaggcacaggtgttcttactgagtctaacaagaagtttctg | |
| | cctttccaacaatttggcagagacattgctgacactactgatgctgtccgt | |
| | gatccacagacacttgagattcttgacattacaccatgttcatttggtggtgt | |
| | cagtgttataacaccaggaacaaatacttctaaccaggttgctgttctttat | |
| | caggatgttaactgcacagaagtccctgttgctattcatgcagatcaactt | |
| | actcctacttggcgtgtttattctacaggttctaatgtatttcaaacacgtgca | |
| | ggctgtttaataggagctgaacatgtcaacaactcatatgagtgtgacat | |
| | acccattggtgcaggtatatgcgctagttatcagactcagactaattctcct | |
| | cggcgggcacgtagtgtagctagtcaatccatcattgcctacactatgtc | |
| | acttggtgcagagaattcagttgcttactctaataactctattgccataccc | |
| | acaaactttactattagtgttaccacagaaattctaccagtgtctatgacca | |
| | agacatcagtagattgtacaatgtacatttgtggtgattcaactgaatgca | |
| | gcaatctattgttgcaatatggcagtttctgtacacaattaaaccgtgcttta | |
| | actggaatagctgttgaacaagacaagaacacccaagaagtatttgca | |
| | caagtcaaacaaatttacaagacaccaccaattaaagatttcggtggat | |
| | ttaatttctcacaaatattaccagatccatctaaaccaagcaagaggtcat | |
| | ttattgaagatctactattcaacaaagtgacacttgcagatgctggcttcat | |
| | caaacaatatggtgattgccttggtgatattgctgctagagacctcatttgt | |
| | gcacagaagtttaacggccttactgtattgccacctttgctcacagatgaa | |
| | atgattgctcaatacacttctgcactgttagcgggtacaatcacttctggttg | |
| | gacctttggtgcaggtgctgcattacaaataccatttgctatgcaaatggct | |
| | tataggtttaatggtattggagttacacagaatgttctctatgagaaccag | |
| | aaattgattgccaaccaatttaatagtgctattggcaagattcaagactca | |
| | ctttcttccacagcaagtgcacttggtaaacttcaagatgtggtcaaccag | |
| | aatgcacaagctttaaacacgcttgttaaacaacttagctccaactttggt | |
| | gcaatttcaagtgtattaaatgatatcctttcacgtcttgacaaagttgagg | |
| | ctgaagtgcaaattgataggttgatcacaggcagacttcaaagtttgcag | |
| | acatatgtgactcaacaattaattagagctgcagaaatcagagcttctgc | |
| | taatcttgctgctactaagatgtcagagtgtgtacttggacaatctaagag | |
| | agttgatttctgtggaaagggctatcatcttatgtccttccctcagtcagcac | |
| | ctcatggtgtagtcttcttgcatgtgacttatgtccctgcacaagagaagaa | |
| | cttcacaactgctcctgccatttgtcatgatggtaaagcacactttcctcgtg | |
| | aaggtgtctttgtttcaaatggcacacactggtttgtaacacaaaggaattt | |
| | ctatgaaccacaaatcattactacagacaacacatttgtgtctggtaactg | |
| | tgatgttgtaataggaattgtcaacaacacagtttatgatcctttgcaacct | |
| | gaattagactcattcaaggaggagttagataaatatttcaagaatcatac | |
| | atcaccagatgttgatttaggtgacatctctggcattaatgcttcagttgtaa | |
| | acattcagaaagaaattgaccgcctcaatgaggttgccaagaatttaaa | |
| | tgaatctctcatcgatctccaagaacttggaaagtatgagcagtatatca | |
| | aatggccatggtacatttggctaggtttcatagctggcttgattgccatagt | |
| | aatggtgacaattatgctttgctgtatgaccagttgctgtagttgtctcaagg | |
| | gctgttgttcttgtggatcctgctgcaaatttgatgaagacgactctgagcc | |
| | agtgctcaaaggagtcaaattacattacacataa | |
| 6 | MFVFLVLLPLVSSQCVNLTTRTQLPPAYTNSFTRGV | Amino acid sequence |
| | YYPDKVFRSSVLHSTQDLFLPFFSNVTWFHAIHVSG | (N- to C-terminus) of |
| | TNGTKRFDNPVLPFNDGVYFASTEKSNIIRGWIFGT | the SARS-COV-2 S |
| | TLDSKTQSLLIVNNATNVVIKVCEFQFCNDPFLGVY | antigen sequence |
| | YHKNNKSWMESEFRVYSSANNCTFEYVSQPFLMD | based on the Wuhan- |
| | LEGKQGNFKNLREFVFKNIDGYFKIYSKHTPINLVRD | Hu-1 reference strain |
| | LPQGFSALEPLVDLPIGINITRFQTLLALHRSYLTPG | (#NC_045512) |
| | DSSSGWTAGAAAYYVGYLQPRTFLLKYNENGTITD | |
| | AVDCALDPLSETKCTLKSFTVEKGIYQTSNFRVQPT | |
| | ESIVRFPNITNLCPFGEVFNATRFASVYAWNRKRIS | |
| | NCVADYSVLYNSASFSTFKCYGVSPTKLNDLCFTN | |
| | VYADSFVIRGDEVRQIAPGQTGKIADYNYKLPDDFT | |
| | GCVIAWNSNNLDSKVGGNYNYLYRLFRKSNLKPFE | |
| | RDISTEIYQAGSTPCNGVEGFNCYFPLQSYGFQPT | |
| | NGVGYQPYRVVVLSFELLHAPATVCGPKKSTNLVK | |
| | NKCVNFNFNGLTGTGVLTESNKKFLPFQQFGRDIA | |
| | DTTDAVRDPQTLEILDITPCSFGGVSVITPGTNTSNQ | |
| | VAVLYQDVNCTEVPVAIHADQLTPTWRVYSTGSNV | |
| | FQTRAGCLIGAEHVNNSYECDIPIGAGICASYQTQT | |
| | NSPRRARSVASQSIIAYTMSLGAENSVAYSNNSIAIP | |
| | TNFTISVTTEILPVSMTKTSVDCTMYICGDSTECSNL | |
| | LLQYGSFCTQLNRALTGIAVEQDKNTQEVFAQVKQI | |
| | YKTPPIKDFGGFNFSQILPDPSKPSKRSFIEDLLFNK | |
| | VTLADAGFIKQYGDCLGDIAARDLICAQKFNGLTVLP | |
| | PLLTDEMIAQYTSALLAGTITSGWTFGAGAALQIPFA | |
| | MQMAYRFNGIGVTQNVLYENQKLIANQFNSAIGKIQ | |
| | DSLSSTASALGKLQDVVNQNAQALNTLVKQLSSNF | |
| | GAISSVLNDILSRLDKVEAEVQIDRLITGRLQSLQTY | |
| | VTQQLIRAAEIRASANLAATKMSECVLGQSKRVDFC | |
| | GKGYHLMSFPQSAPHGVVFLHVTYVPAQEKNFTTA | |
| | PAICHDGKAHFPREGVFVSNGTHWFVTQRNFYEP | |
| | QIITTDNTFVSGNCDVVIGIVNNTVYDPLQPELDSFK | |
| | EELDKYFKNHTSPDVDLGDISGINASVVNIQKEIDRL | |
| | NEVAKNLNESLIDLQELGKYEQYIKWPWYIWLGFIA | |
| | GLIAIVMVTIMLCCMTSCCSCLKGCCSCGSCCKFDE | |
| | DDSEPVLKGVKLHYT | |
| 7 | atgtctgataacggaccacagaatcagcgaaacgcaccacgcattac | Nucleotide sequence/ |
| | gtttggtggaccctcagattcaactggcagtaaccagaatggagaacg | ORF (5′ to 3′ end) of |
| | cagtggagcgcgatctaaacaacgtcggcctcaaggtttacccaataat | the SARS-CoV-2 N |
| | actgcgtcttggttcaccgctctcactcaacatggcaaggaagaccttaa | antigen sequence |
| | attccctcgaggacaaggcgttccaattaacaccaatagcagtccagat | based on the Wuhan- |
| | gaccaaattggctactaccgaagagctaccagacgaattcgtggtggt | Hu-1 reference strain |
| | gacggtaagatgaaagatctcagtccaagatggtatttctactacctagg | (#NC_045512) and |
| | aactgggccagaagctggacttccctatggtgctaacaaagacggcat | optimized for stability |
| | catatgggttgcaactgagggagccttgaatacacctaaagatcacatt | in vaccinia by silent |
| | ggcacccgcaatcctgctaacaatgctgcaatcgtgctacaacttcctca | codon alteration to |
| | aggaacaacattgcctaaaggcttctacgcagaagggagcagaggc | avoid 4 or more of the |
| | ggcagtcaagcctcttctcgttcctcatcacgtagtcgcaacagttcaaga | same nucleotides in |
| | aattcaactccaggcagcagtagaggaacttctcctgctagaatggctg | consecutive order |
| | gcaatggcggtgatgctgctcttgctttgctgctgcttgacagattgaacca | |
| | gcttgagagcaagatgtctggtaaaggccaacaacaacaaggccaa | |
| | actgtcactaagaaatctgctgctgaggcttctaagaagcctcggcaga | |
| | aacgtactgccactaaagcatacaatgtaacacaagctttcggcagac | |
| | gtggtccagaacaaacccaaggaaactttggagaccaggaactaatc | |
| | agacaaggaactgattacaaacattggccgcaaattgcacaatttgctc | |
| | ccagcgcttcagcgttcttcggaatgtcgcgcattggcatggaagtcaca | |
| | ccttcgggaacgtggttgacctacacaggtgccatcaaattggatgaca | |
| | aagatccaaatttcaaagatcaagtcatattgctgaataagcatattgac | |
| | gcatacaagacattcccaccaacagagcctaagaaggacaagaaga | |
| | agaaggctgatgaaactcaagccttaccgcagagacagaagaaaca | |
| | gcaaactgtgactcttcttcctgctgcagatttggatgatttctccaaacaat | |
| | tgcaacaatccatgagcagtgctgactcaactcaggcctaa | |
| 8 | MSDNGPQNQRNAPRITFGGPSDSTGSNQNGERSG | Amino acid sequence |
| | ARSKQRRPQGLPNNTASWFTALTQHGKEDLKFPR | (N- to C-terminus) of |
| | GQGVPINTNSSPDDQIGYYRRATRRIRGGDGKMKD | the SARS-CoV-2 N |
| | LSPRWYFYYLGTGPEAGLPYGANKDGIIWVATEGA | antigen sequence |
| | LNTPKDHIGTRNPANNAAIVLQLPQGTTLPKGFYAE | based on the Wuhan- |
| | GSRGGSQASSRSSSRSRNSSRNSTPGSSRGTSPA | Hu-1 reference strain |
| | RMAGNGGDAALALLLLDRLNQLESKMSGKGQQQQ | (#NC_045512) |
| | GQTVTKKSAAEASKKPRQKRTATKAYNVTQAFGRR | |
| | GPEQTQGNFGDQELIRQGTDYKHWPQIAQFAPSA | |
| | SAFFGMSRIGMEVTPSGTWLTYTGAIKLDDKDPNF | |
| | KDQVILLNKHIDAYKTFPPTEPKKDKKKKADETQAL | |
| | PQRQKKQQTVTLLPAADLDDFSKQLQQSMSSADS | |
| | TQA | |
| 9 | atgtttgtctttcttgtcttattgccactagtctctagtcagtgtgttaatcttaga | Nucleotide |
| | accagaactcaattaccacctgcatacactaattctttcacacgtggtgttt | sequence/ORF (5′ to |
| | attaccctgacaaagtattcagatcctcagtattacattcaactcaggactt | 3′ end) of a codon- |
| | gttcttacctttcttctccaatgttacttggttccatgctatacatgtctctggga | optimized SARS-CoV- |
| | ccaatggtactaagaggtttgataaccctgtcctaccatttaatgatggtgt | 2 S antigen sequence |
| | ttatttcgcttccactgagaagtctaacataataagaggctggatatttggt | based on the Wuhan- |
| | actactttagattcgaagacccagtccctacttattgttaataacgctacta | Hu-1 reference strain |
| | atgttgttattaaagtctgtgaatttcaattctgtaatgatccattcttggatgttt | (#NC_045512) and |
| | attaccacaagaacaacaagagttggatggaaagtggagtttattctag | optimized for vaccinia, |
| | tgcgaataattgcactttcgaatatgtctctcagcctttccttatggaccttga | which is further |
| | aggtaaacagggtaatttcaagaatcttagggaatttgtgtttaagaatatt | modified to encode for |
| | gatggttatttcaagatatattctaagcacacgcctattaatttagtgcgtga | an S antigen that |
| | tctccctcagggtttctcggctttagaaccattggtagatttgccaataggta | includes mutations of |
| | ttaacatcactaggtttcaaactttacttgctttacatagaagttatttgactcc | the B.1.617.2 variant |
| | tggtgattcttcttcaggttggacagctggtgctgcagcttattatgtgggtta | lineage identified in |
| | tcttcaacctaggactttcctattgaaatataatgagaatggaaccattac | India, including T19R, |
| | agatgctgtagactgtgcacttgaccctctctcagaaacaaagtgtacgtt | G142D, Del156/157, |
| | gaaatccttcactgtagagaaaggaatctatcaaacttctaactttagagt | R158G, L452R, |
| | ccaaccaacagaatctattgttagatttcctaatattacaaacttgtgccctt | T478K, D614G, |
| | tcggtgaagtatttaacgccaccagatttgcatctgtttatgcttggaacag | P681R, and D950N |
| | gaagagaatcagcaactgtgttgctgattattctgtcctatataattccgca | |
| | tcattctccacatttaagtgttatggagtgtctcctactaaattaaatgatctct | |
| | gctttactaatgtctatgcagattcatttgtaattagaggtgatgaagtcag | |
| | acaaatcgctccagggcaaactggaaagattgctgattataattataaat | |
| | taccagatgactttacaggctgcgttatagcttggaattctaacaatcttga | |
| | ttctaaggttggtggtaattataattaccggtatagattgtttaggaagtcta | |
| | atctcaaacctttcgagagagatatttcaactgaaatctatcaggccggta | |
| | gcaaaccttgtaatggtgttgaaggatttaattgttactttcctttacaatcat | |
| | atggtttccaacccactaatggtgttggttaccaaccatacagagtagtag | |
| | tactttcatttgaacttctacatgcaccagcaactgtttgtggacctaagaa | |
| | gtctactaatttggttaagaacaaatgtgtcaatttcaacttcaatggtttaa | |
| | caggcacaggtgttcttactgagtctaacaagaagtttctgcctttccaac | |
| | aatttggcagagacattgctgacactactgatgctgtccgtgatccacag | |
| | acacttgagattcttgacattacaccatgttcatttggtggtgtcagtgttata | |
| | acaccaggaacaaatacttctaaccaggttgctgttctttatcagggtgtta | |
| | actgcacagaagtccctgttgctattcatgcagatcaacttactcctacttg | |
| | gcgtgtttattctacaggttctaatgtatttcaaacacgtgcaggctgtttaat | |
| | aggagctgaacatgtcaacaactcatatgagtgtgacatacccattggt | |
| | gcaggtatatgcgctagttatcagactcagactaattctcgtcggcgggc | |
| | acgtagtgtagctagtcaatccatcattgcctacactatgtcacttggtgca | |
| | gagaattcagttgcttactctaataactctattgccatacccacaaacttta | |
| | ctattagtgttaccacagaaattctaccagtgtctatgaccaagacatcag | |
| | tagattgtacaatgtacatttgtggtgattcaactgaatgcagcaatctattg | |
| | ttgcaatatggcagtttctgtacacaattaaaccgtgctttaactggaatag | |
| | ctgttgaacaagacaagaacacccaagaagtatttgcacaagtcaaa | |
| | caaatttacaagacaccaccaattaaagatttcggtggatttaatttctcac | |
| | aaatattaccagatccatctaaaccaagcaagaggtcatttattgaagat | |
| | ctactattcaacaaagtgacacttgcagatgctggcttcatcaaacaatat | |
| | ggtgattgccttggtgatattgctgctagagacctcatttgtgcacagaagt | |
| | ttaacggccttactgtattgccacctttgctcacagatgaaatgattgctca | |
| | atacacttctgcactgttagcgggtacaatcacttctggttggacctttggtg | |
| | caggtgctgcattacaaataccatttgctatgcaaatggcttataggtttaa | |
| | tggtattggagttacacagaatgttctctatgagaaccagaaattgattgc | |
| | caaccaatttaatagtgctattggcaagattcaagactcactttcttccaca | |
| | gcaagtgcacttggtaaacttcagaatgtggtcaaccagaatgcacaa | |
| | gctttaaacacgcttgttaaacaacttagctccaactttggtgcaatttcaa | |
| | gtgtattaaatgatatcctttcacgtcttgacaaagttgaggctgaagtgca | |
| | aattgataggttgatcacaggcagacttcaaagtttgcagacatatgtga | |
| | ctcaacaattaattagagctgcagaaatcagagcttctgctaatcttgctg | |
| | ctactaagatgtcagagtgtgtacttggacaatctaagagagttgatttctg | |
| | tggaaagggctatcatcttatgtccttccctcagtcagcacctcatggtgta | |
| | gtcttcttgcatgtgacttatgtccctgcacaagagaagaacttcacaact | |
| | gctcctgccatttgtcatgatggtaaagcacactttcctcgtgaaggtgtctt | |
| | tgtttcaaatggcacacactggtttgtaacacaaaggaatttctatgaacc | |
| | acaaatcattactacagacaacacatttgtgtctggtaactgtgatgttgta | |
| | ataggaattgtcaacaacacagtttatgatcctttgcaacctgaattagac | |
| | tcattcaaggaggagttagataaatatttcaagaatcatacatcaccaga | |
| | tgttgatttaggtgacatctctggcattaatgcttcagttgtaaacattcaga | |
| | aagaaattgaccgcctcaatgaggttgccaagaatttaaatgaatctctc | |
| | atcgatctccaagaacttggaaagtatgagcagtatatcaaatggccat | |
| | ggtacatttggctaggtttcatagctggcttgattgccatagtaatggtgac | |
| | aattatgctttgctgtatgaccagttgctgtagttgtctcaagggctgttgttct | |
| | tgtggatcctgctgcaaatttgatgaagacgactctgagccagtgctcaa | |
| | aggagtcaaattacattacacataa | |
| 10 | MFVFLVLLPLVSSQCVNLRTRTQLPPAYTNSFTRGV | Amino acid sequence |
| | YYPDKVFRSSVLHSTQDLFLPFFSNVTWFHAIHVSG | (N- to C-terminus) of |
| | TNGTKRFDNPVLPFNDGVYFASTEKSNIIRGWIFGT | a codon-optimized |
| | TLDSKTQSLLIVNNATNVVIKVCEFQFCNDPFLDVYY | SARS-CoV-2 S |
| | HKNNKSWMESGVYSSANNCTFEYVSQPFLMDLEG | antigen sequence |
| | KQGNFKNLREFVFKNIDGYFKIYSKHTPINLVRDLPQ | based on the Wuhan- |
| | GFSALEPLVDLPIGINITRFQTLLALHRSYLTPGDSS | Hu-1 reference strain |
| | SGWTAGAAAYYVGYLQPRTFLLKYNENGTITDAVD | (#NC_045512) and |
| | CALDPLSETKCTLKSFTVEKGIYQTSNFRVQPTESIV | optimized for vaccinia, |
| | RFPNITNLCPFGEVFNATRFASVYAWNRKRISNCVA | which is further |
| | DYSVLYNSASFSTFKCYGVSPTKLNDLCFTNVYAD | modified to encode for |
| | SFVIRGDEVRQIAPGQTGKIADYNYKLPDDFTGCVI | an S antigen that |
| | AWNSNNLDSKVGGNYNYRYRLFRKSNLKPFERDIS | includes mutations of |
| | TEIYQAGSKPCNGVEGFNCYFPLQSYGFQPTNGVG | the B.1.617.2 variant |
| | YQPYRVVVLSFELLHAPATVCGPKKSTNLVKNKCV | lineage identified in |
| | NFNFNGLTGTGVLTESNKKFLPFQQFGRDIADTTDA | India, including T19R, |
| | VRDPQTLEILDITPCSFGGVSVITPGTNTSNQVAVLY | G142D, Del156/157, |
| | QGVNCTEVPVAIHADQLTPTWRVYSTGSNVFQTRA | R158G, L452R, |
| | GCLIGAEHVNNSYECDIPIGAGICASYQTQTNSRRR | T478K, D614G, |
| | ARSVASQSIIAYTMSLGAENSVAYSNNSIAIPTNFTIS | P681R, and D950N |
| | VTTEILPVSMTKTSVDCTMYICGDSTECSNLLLQYG | |
| | SFCTQLNRALTGIAVEQDKNTQEVFAQVKQIYKTPP | |
| | IKDFGGFNFSQILPDPSKPSKRSFIEDLLFNKVTLAD | |
| | AGFIKQYGDCLGDIAARDLICAQKFNGLTVLPPLLTD | |
| | EMIAQYTSALLAGTITSGWTFGAGAALQIPFAMQMA | |
| | YRFNGIGVTQNVLYENQKLIANQFNSAIGKIQDSLSS | |
| | TASALGKLQNVVNQNAQALNTLVKQLSSNFGAISSV | |
| | LNDILSRLDKVEAEVQIDRLITGRLQSLQTYVTQQLI | |
| | RAAEIRASANLAATKMSECVLGQSKRVDFCGKGYH | |
| | LMSFPQSAPHGVVFLHVTYVPAQEKNFTTAPAICH | |
| | DGKAHFPREGVFVSNGTHWFVTQRNFYEPQIITTD | |
| | NTFVSGNCDVVIGIVNNTVYDPLQPELDSFKEELDK | |
| | YFKNHTSPDVDLGDISGINASVVNIQKEIDRLNEVAK | |
| | NLNESLIDLQELGKYEQYIKWPWYIWLGFIAGLIAIV | |
| | MVTIMLCCMTSCCSCLKGCCSCGSCCKFDEDDSE | |
| | PVLKGVKLHYT | |
| 11 | atgtttgtctttcttgtcttattgccactagtctctagtcagtgtgttaatcttaga | Nucleotide |
| | accagaactcaattaccacctgcatacactaattctttcacacgtggtgttt | sequence/ORF (5′ to |
| | attaccctgacaaagtattcagatcctcagtattacattcaactcaggactt | 3′ end) of a codon- |
| | gttcttacctttcttctccaatgttacttggttccatgctatacatgtctctggga | optimized SARS-CoV- |
| | ccaatggtactaagaggtttgataaccctgtcctaccatttaatgatggtgt | 2 S antigen sequence |
| | ttatttcgcttccactgagaagtctaacataataagaggctggatatttggt | based on the Wuhan- |
| | actactttagattcgaagacccagtccctacttattgttaataacgctacta | Hu-1 reference strain |
| | atgttgttattaaagtctgtgaatttcaattctgtaatgatccattcttgggtgttt | (#NC_045512) and |
| | attaccacaagaacaacaagagttggatggaaagtggtgtttattctagt | optimized for vaccinia, |
| | gcgaataattgcactttcgaatatgtctctcagcctttccttatggaccttga | which is further |
| | aggtaaacagggtaatttcaagaatcttagggaatttgtgtttaagaatatt | modified to encode for |
| | gatggttatttcaagatatattctaagcacacgcctattaatttagtgcgtga | an S antigen that |
| | tctccctcagggtttctcggctttagaaccattggtagatttgccaataggta | includes mutations of |
| | ttaacatcactaggtttcaaactttacttgctttacatagaagttatttgactcc | the B.1.617.2 variant |
| | tggtgattctttctcaggttggacagctggtgctgcagcttattatgtgggtta | lineage identified in |
| | tcttcaacctaggactttcctattgaaatataatgagaatggaaccattac | India, including T19R, |
| | agatgctgtagactgtgcacttgaccctctctcagaaacaaagtgtacgtt | E156G, Del157/158, |
| | gaaatccttcactgtagagaaaggaatctatcaaacttctaactttagagt | S255F, L452R, |
| | ccaaccaacagaatctattgttagatttcctaatattacaaacttgtgccctt | T478K, D614G, |
| | tcggtgaagtatttaacgccaccagatttgcatctgtttatgcttggaacag | P681R, and D950N |
| | gaagagaatcagcaactgtgttgctgattattctgtcctatataattccgca | |
| | tcattctccacatttaagtgttatggagtgtctcctactaaattaaatgatctct | |
| | gctttactaatgtctatgcagattcatttgtaattagaggtgatgaagtcag | |
| | acaaatcgctccagggcaaactggaaagattgctgattataattataaat | |
| | taccagatgactttacaggctgcgttatagcttggaattctaacaatcttga | |
| | ttctaaggttggtggtaattataattaccggtatagattgtttaggaagtcta | |
| | atctcaaacctttcgagagagatatttcaactgaaatctatcaggccggta | |
| | gcaaaccttgtaatggtgttgaaggatttaattgttactttcctttacaatcat | |
| | atggtttccaacccactaatggtgttggttaccaaccatacagagtagtag | |
| | tactttcatttgaacttctacatgcaccagcaactgtttgtggacctaagaa | |
| | gtctactaatttggttaagaacaaatgtgtcaatttcaacttcaatggtttaa | |
| | caggcacaggtgttcttactgagtctaacaagaagtttctgcctttccaac | |
| | aatttggcagagacattgctgacactactgatgctgtccgtgatccacag | |
| | acacttgagattcttgacattacaccatgttcatttggtggtgtcagtgttata | |
| | acaccaggaacaaatacttctaaccaggttgctgttctttatcagggtgtta | |
| | actgcacagaagtccctgttgctattcatgcagatcaacttactcctacttg | |
| | gcgtgtttattctacaggttctaatgtatttcaaacacgtgcaggctgtttaat | |
| | aggagctgaacatgtcaacaactcatatgagtgtgacatacccattggt | |
| | gcaggtatatgcgctagttatcagactcagactaattctcgtcgggggc | |
| | acgtagtgtagctagtcaatccatcattgcctacactatgtcacttggtgca | |
| | gagaattcagttgcttactctaataactctattgccatacccacaaacttta | |
| | ctattagtgttaccacagaaattctaccagtgtctatgaccaagacatcag | |
| | tagattgtacaatgtacatttgtggtgattcaactgaatgcagcaatctattg | |
| | ttgcaatatggcagtttctgtacacaattaaaccgtgctttaactggaatag | |
| | ctgttgaacaagacaagaacacccaagaagtatttgcacaagtcaaa | |
| | caaatttacaagacaccaccaattaaagatttcggtggatttaatttctcac | |
| | aaatattaccagatccatctaaaccaagcaagaggtcatttattgaagat | |
| | ctactattcaacaaagtgacacttgcagatgctggcttcatcaaacaatat | |
| | ggtgattgccttggtgatattgctgctagagacctcatttgtgcacagaagt | |
| | ttaacggccttactgtattgccacctttgctcacagatgaaatgattgctca | |
| | atacacttctgcactgttagcgggtacaatcacttctggttggacctttggtg | |
| | caggtgctgcattacaaataccatttgctatgcaaatggcttataggtttaa | |
| | tggtattggagttacacagaatgttctctatgagaaccagaaattgattgc | |
| | caaccaatttaatagtgctattggcaagattcaagactcactttcttccaca | |
| | gcaagtgcacttggtaaacttcagaatgtggtcaaccagaatgcacaa | |
| | gctttaaacacgcttgttaaacaacttagctccaactttggtgcaatttcaa | |
| | gtgtattaaatgatatcctttcacgtcttgacaaagttgaggctgaagtgca | |
| | aattgataggttgatcacaggcagacttcaaagtttgcagacatatgtga | |
| | ctcaacaattaattagagctgcagaaatcagagcttctgctaatcttgctg | |
| | ctactaagatgtcagagtgtgtacttggacaatctaagagagttgatttctg | |
| | tggaaagggctatcatcttatgtccttccctcagtcagcacctcatggtgta | |
| | gtcttcttgcatgtgacttatgtccctgcacaagagaagaacttcacaact | |
| | gctcctgccatttgtcatgatggtaaagcacactttcctcgtgaaggtgtctt | |
| | tgtttcaaatggcacacactggtttgtaacacaaaggaatttctatgaacc | |
| | acaaatcattactacagacaacacatttgtgtctggtaactgtgatgttgta | |
| | ataggaattgtcaacaacacagtttatgatcctttgcaacctgaattagac | |
| | tcattcaaggaggagttagataaatatttcaagaatcatacatcaccaga | |
| | tgttgatttaggtgacatctctggcattaatgcttcagttgtaaacattcaga | |
| | aagaaattgaccgcctcaatgaggttgccaagaatttaaatgaatctctc | |
| | atcgatctccaagaacttggaaagtatgagcagtatatcaaatggccat | |
| | ggtacatttggctaggtttcatagctggcttgattgccatagtaatggtgac | |
| | aattatgctttgctgtatgaccagttgctgtagttgtctcaagggctgttgttct | |
| | tgtggatcctgctgcaaatttgatgaagacgactctgagccagtgctcaa | |
| | aggagtcaaattacattacacataa | |
| 12 | MFVFLVLLPLVSSQCVNLRTRTQLPPAYTNSFTRGV | Amino acid sequence |
| | YYPDKVFRSSVLHSTQDLFLPFFSNVTWFHAIHVSG | (N- to C-terminus) of a |
| | TNGTKRFDNPVLPFNDGVYFASTEKSNIIRGWIFGT | codon-optimized |
| | TLDSKTQSLLIVNNATNVVIKVCEFQFCNDPFLGVY | SARS-CoV-2 S |
| | YHKNNKSWMESGVYSSANNCTFEYVSQPFLMDLE | antigen sequence |
| | GKQGNFKNLREFVFKNIDGYFKIYSKHTPINLVRDLP | based on the Wuhan- |
| | QGFSALEPLVDLPIGINITRFQTLLALHRSYLTPGDS | Hu-1 reference strain |
| | FSGWTAGAAAYYVGYLQPRTFLLKYNENGTITDAV | (#NC_045512) and |
| | DCALDPLSETKCTLKSFTVEKGIYQTSNFRVQPTESI | optimized for vaccinia, |
| | VRFPNITNLCPFGEVFNATRFASVYAWNRKRISNCV | which is further |
| | ADYSVLYNSASFSTFKCYGVSPTKLNDLCFTNVYA | modified to encode for |
| | DSFVIRGDEVRQIAPGQTGKIADYNYKLPDDFTGCV | an S antigen that |
| | IAWNSNNLDSKVGGNYNYRYRLFRKSNLKPFERDI | includes mutations of |
| | STEIYQAGSKPCNGVEGFNCYFPLQSYGFQPTNGV | the B.1.617.2 variant |
| | GYQPYRVVVLSFELLHAPATVCGPKKSTNLVKNKC | lineage identified in |
| | VNFNFNGLTGTGVLTESNKKFLPFQQFGRDIADTTD | India, including T19R, |
| | AVRDPQTLEILDITPCSFGGVSVITPGTNTSNQVAVL | E156G, Del157/158, |
| | YQGVNCTEVPVAIHADQLTPTWRVYSTGSNVFQTR | S255F, L452R, |
| | AGCLIGAEHVNNSYECDIPIGAGICASYQTQTNSRR | T478K, D614G, |
| | RARSVASQSIIAYTMSLGAENSVAYSNNSIAIPTNFTI | P681R, and D950N |
| | SVTTEILPVSMTKTSVDCTMYICGDSTECSNLLLQY | |
| | GSFCTQLNRALTGIAVEQDKNTQEVFAQVKQIYKTP | |
| | PIKDFGGFNFSQILPDPSKPSKRSFIEDLLFNKVTLA | |
| | DAGFIKQYGDCLGDIAARDLICAQKFNGLTVLPPLLT | |
| | DEMIAQYTSALLAGTITSGWTFGAGAALQIPFAMQM | |
| | AYRFNGIGVTQNVLYENQKLIANQFNSAIGKIQDSLS | |
| | STASALGKLQNVVNQNAQALNTLVKQLSSNFGAISS | |
| | VLNDILSRLDKVEAEVQIDRLITGRLQSLQTYVTQQL | |
| | IRAAEIRASANLAATKMSECVLGQSKRVDFCGKGY | |
| | HLMSFPQSAPHGVVFLHVTYVPAQEKNFTTAPAIC | |
| | HDGKAHFPREGVFVSNGTHWFVTQRNFYEPQIITT | |
| | DNTFVSGNCDVVIGIVNNTVYDPLQPELDSFKEELD | |
| | KYFKNHTSPDVDLGDISGINASVVNIQKEIDRLNEVA | |
| | KNLNESLIDLQELGKYEQYIKWPWYIWLGFIAGLIAIV | |
| | MVTIMLCCMTSCCSCLKGCCSCGSCCKFDEDDSE | |
| | PVLKGVKLHYT | |
| 13 | atgtttgtctttcttgtcttattgccactagtctctagtcagtgtgttaatcttaca | Nucleotide |
| | accagaactcaattaccacctgcatacactaattctttcacacgtggtgttt | sequence/ORF (5′ to |
| | attaccctgacaaagtattcagatcctcagtattacattcaactcaggactt | 3′ end) of a codon- |
| | gttcttacctttcttctccaatgttacttggttccatgctatacatgtctctggga | optimized SARS-CoV- |
| | ccaatggtactaagaggtttgataaccctgtcctaccatttaatgatggtgt | 2 S antigen sequence |
| | ttatttcgcttccattgagaagtctaacataataagaggctggatatttggta | based on the Wuhan- |
| | ctactttagattcgaagacccagtccctacttattgttaataacgctactaat | Hu-1 reference strain |
| | gttgttattaaagtctgtgaatttcaattctgtaatgatccattcttgggtgtttct | (#NC_045512) and |
| | aaccacaagaacaacaagagttggatggaaagtgagttcagagtttat | optimized for vaccinia, |
| | tctagtgcgaataattgcactttcgaatatgtctctcagcctttccttatggac | which is further |
| | cttgaaggtaaacagggtaatttcaagaatcttagggaatttgtgtttaag | modified to encode for |
| | aatattgatggttatttcaagatatattctaagcacacgcctattaatttagtg | an S antigen that |
| | cgtgatctccctcagggtttctcggctttagaaccattggtagatttgccaat | includes mutations of |
| | aggtattaacatcactaggtttcaaactttacttgctttacatagaagttattt | the B.1.621 variant |
| | gactcctggtgattcttcttcaggttggacagctggtgctgcagcttattatgt | lineage, including |
| | gggttatcttcaacctaggactttcctattgaaatataatgagaatggaac | T95N, Y144S, Y145N, |
| | cattacagatgctgtagactgtgcacttgaccctctctcagaaacaaagt | R346K, E484K, |
| | gtacgttgaaatccttcactgtagagaaaggaatctatcaaacttctaact | N501Y, D614G, |
| | ttagagtccaaccaacagaatctattgttagatttcctaatattacaaactt | P681H, and D950N |
| | gtgccctttcggtgaagtatttaacgccaccaaatttgcatctgtttatgcttg | |
| | gaacaggaagagaatcagcaactgtgttgctgattattctgtcctatataa | |
| | ttccgcatcattctccacatttaagtgttatggagtgtctcctactaaattaaa | |
| | tgatctctgctttactaatgtctatgcagattcatttgtaattagaggtgatga | |
| | agtcagacaaatcgctccagggcaaactggaaagattgctgattataat | |
| | tataaattaccagatgactttacaggctgcgttatagcttggaattctaaca | |
| | atcttgattctaaggttggtggtaattataattacctgtatagattgtttagga | |
| | agtctaatctcaaacctttcgagagagatatttcaactgaaatctatcagg | |
| | ccggtagcacaccttgtaatggtgttaaaggatttaattgttactttcctttac | |
| | aatcatatggtttccaacccacttatggtgttggttaccaaccatacagagt | |
| | agtagtactttcatttgaacttctacatgcaccagcaactgtttgtggaccta | |
| | agaagtctactaatttggttaagaacaaatgtgtcaatttcaacttcaatgg | |
| | tttaacaggcacaggtgttcttactgagtctaacaagaagtttctgcctttcc | |
| | aacaatttggcagagacattgctgacactactgatgctgtccgtgatcca | |
| | cagacacttgagattcttgacattacaccatgttcatttggtggtgtcagtgt | |
| | tataacaccaggaacaaatacttctaaccaggttgctgttctttatcagggt | |
| | gttaactgcacagaagtccctgttgctattcatgcagatcaacttactccta | |
| | cttggcgtgtttattctacaggttctaatgtatttcaaacacgtgcaggctgtt | |
| | taataggagctgaacatgtcaacaactcatatgagtgtgacatacccatt | |
| | ggtgcaggtatatgcgctagttatcagactcagactaattctcatcggcgg | |
| | gcacgtagtgtagctagtcaatccatcattgcctacactatgtcacttggtg | |
| | cagagaattcagttgcttactctaataactctattgccatacccacaaactt | |
| | tactattagtgttaccacagaaattctaccagtgtctatgaccaagacatc | |
| | agtagattgtacaatgtacatttgtggtgattcaactgaatgcagcaatcta | |
| | ttgttgcaatatggcagtttctgtacacaattaaaccgtgctttaactggaat | |
| | agctgttgaacaagacaagaacacccaagaagtatttgcacaagtca | |
| | aacaaatttacaagacaccaccaattaaagatttcggtggatttaatttct | |
| | cacaaatattaccagatccatctaaaccaagcaagaggtcatttattga | |
| | agatctactattcaacaaagtgacacttgcagatgctggcttcatcaaac | |
| | aatatggtgattgccttggtgatattgctgctagagacctcatttgtgcaca | |
| | gaagtttaacggccttactgtattgccacctttgctcacagatgaaatgatt | |
| | gctcaatacacttctgcactgttagcgggtacaatcacttctggttggacct | |
| | ttggtgcaggtgctgcattacaaataccatttgctatgcaaatggcttatag | |
| | gtttaatggtattggagttacacagaatgttctctatgagaaccagaaatt | |
| | gattgccaaccaatttaatagtgctattggcaagattcaagactcactttctt | |
| | ccacagcaagtgcacttggtaaacttcagaatgtggtcaaccagaatgc | |
| | acaagctttaaacacgcttgttaaacaacttagctccaactttggtgcaatt | |
| | tcaagtgtattaaatgatatcctttcacgtcttgacaaagttgaggctgaag | |
| | tgcaaattgataggttgatcacaggcagacttcaaagtttgcagacatat | |
| | gtgactcaacaattaattagagctgcagaaatcagagcttctgctaatctt | |
| | gctgctactaagatgtcagagtgtgtacttggacaatctaagagagttgat | |
| | ttctgtggaaagggctatcatcttatgtccttccctcagtcagcacctcatg | |
| | gtgtagtcttcttgcatgtgacttatgtccctgcacaagagaagaacttcac | |
| | aactgctcctgccatttgtcatgatggtaaagcacactttcctcgtgaaggt | |
| | gtctttgtttcaaatggcacacactggtttgtaacacaaaggaatttctatg | |
| | aaccacaaatcattactacagacaacacatttgtgtctggtaactgtgatg | |
| | ttgtaataggaattgtcaacaacacagtttatgatcctttgcaacctgaatt | |
| | agactcattcaaggaggagttagataaatatttcaagaatcatacatcac | |
| | cagatgttgatttaggtgacatctctggcattaatgcttcagttgtaaacatt | |
| | cagaaagaaattgaccgcctcaatgaggttgccaagaatttaaatgaat | |
| | ctctcatcgatctccaagaacttggaaagtatgagcagtatatcaaatgg | |
| | ccatggtacatttggctaggtttcatagctggcttgattgccatagtaatggt | |
| | gacaattatgctttgctgtatgaccagttgctgtagttgtctcaagggctgtt | |
| | gttcttgtggatcctgctgcaaatttgatgaagacgactctgagccagtgct | |
| | caaaggagtcaaattacattacacataa | |
| 14 | MFVFLVLLPLVSSQCVNLTTRTQLPPAYTNSFTRGV | Amino acid sequence |
| | YYPDKVFRSSVLHSTQDLFLPFFSNVTWFHAIHVSG | (N- to C-terminus) of a |
| | TNGTKRFDNPVLPFNDGVYFASIEKSNIIRGWIFGTT | codon-optimized |
| | LDSKTQSLLIVNNATNVVIKVCEFQFCNDPFLGVSN | SARS-CoV-2 S |
| | HKNNKSWMESEFRVYSSANNCTFEYVSQPFLMDL | antigen sequence |
| | EGKQGNFKNLREFVFKNIDGYFKIYSKHTPINLVRDL | based on the Wuhan- |
| | PQGFSALEPLVDLPIGINITRFQTLLALHRSYLTPGD | Hu-1 reference strain |
| | SSSGWTAGAAAYYVGYLQPRTFLLKYNENGTITDA | (#NC_045512) and |
| | VDCALDPLSETKCTLKSFTVEKGIYQTSNFRVQPTE | optimized for vaccinia, |
| | SIVRFPNITNLCPFGEVFNATKFASVYAWNRKRISN | which is further |
| | CVADYSVLYNSASFSTFKCYGVSPTKLNDLCFTNV | modified to encode for |
| | YADSFVIRGDEVRQIAPGQTGKIADYNYKLPDDFTG | an S antigen that |
| | CVIAWNSNNLDSKVGGNYNYLYRLFRKSNLKPFER | includes mutations of |
| | DISTEIYQAGSTPCNGVKGFNCYFPLQSYGFQPTY | the B.1.621 variant |
| | GVGYQPYRVVVLSFELLHAPATVCGPKKSTNLVKN | lineage, including |
| | KCVNFNFNGLTGTGVLTESNKKFLPFQQFGRDIAD | T95N, Y144S, Y145N, |
| | TTDAVRDPQTLEILDITPCSFGGVSVITPGTNTSNQV | R346K, E484K, |
| | AVLYQGVNCTEVPVAIHADQLTPTWRVYSTGSNVF | N501Y, D614G, |
| | QTRAGCLIGAEHVNNSYECDIPIGAGICASYQTQTN | P681H, and D950N |
| | SHRRARSVASQSIIAYTMSLGAENSVAYSNNSIAIPT | |
| | NFTISVTTEILPVSMTKTSVDCTMYICGDSTECSNLL | |
| | LQYGSFCTQLNRALTGIAVEQDKNTQEVFAQVKQIY | |
| | KTPPIKDFGGFNFSQILPDPSKPSKRSFIEDLLFNKV | |
| | TLADAGFIKQYGDCLGDIAARDLICAQKFNGLTVLPP | |
| | LLTDEMIAQYTSALLAGTITSGWTFGAGAALQIPFA | |
| | MQMAYRFNGIGVTQNVLYENQKLIANQFNSAIGKIQ | |
| | DSLSSTASALGKLQNVVNQNAQALNTLVKQLSSNF | |
| | GAISSVLNDILSRLDKVEAEVQIDRLITGRLQSLQTY | |
| | VTQQLIRAAEIRASANLAATKMSECVLGQSKRVDFC | |
| | GKGYHLMSFPQSAPHGVVFLHVTYVPAQEKNFTTA | |
| | PAICHDGKAHFPREGVFVSNGTHWFVTQRNFYEP | |
| | QIITTDNTFVSGNCDVVIGIVNNTVYDPLQPELDSFK | |
| | EELDKYFKNHTSPDVDLGDISGINASVVNIQKEIDRL | |
| | NEVAKNLNESLIDLQELGKYEQYIKWPWYIWLGFIA | |
| | GLIAIVMVTIMLCCMTSCCSCLKGCCSCGSCCKFDE | |
| | DDSEPVLKGVKLHYT | |
| 15 | atgtttgtctttcttgtcttattgctactagtctctagtcagtgtgttaatcttaca | Nucleotide |
| | accagaactcaattaccacctgcatacactaattctttcacacgtggtgttt | sequence/ORF (5′ to |
| | attaccctgacaaagtattcagatcctcagtattacattcaactcaggactt | 3′ end) of a codon- |
| | gttcttacctttcttctccaatgttacttggttccatgctatacatgtctctggga | optimized SARS-CoV- |
| | ccaatggtactaagaggtttgataaccctgtcctaccatttaatgatggtgt | 2 S antigen sequence |
| | ttatttcgcttccactgagaagtctaacataataagaggctggatatttggt | based on the Wuhan- |
| | actactttagattcgaagacccagtccctacttattgttaataacgctacta | Hu-1 reference strain |
| | atgttgttattaaagtctgtgaatttcaattctttaatgatccattcttgggtgttt | (#NC_045512) and |
| | accacaagaacaacaagagttggatggaaagtgagttcagagtttattc | optimized for vaccinia, |
| | tagtgcgaataattgcactttcgaatatgtctctcagcctttccttatggacct | which is further |
| | tgaaggtaaacagggtaatttcaagaatcttagcgaatttgtgtttaagaa | modified to encode for |
| | tattgatggttatttcaagatatattctaagcacacgcctattaatttagtgcg | an S antigen that |
| | tggtctccctcagggtttctcggctttagaaccattggtagatttgccaatag | includes mutations of |
| | gtattaacatcactaggtttcaaactttattacatagaagttatttgactcctg | the C.1.2 variant |
| | gtgattcttcttcaggttggacagctggtgctgcagcttattatgtgggttatc | lineage, including |
| | ttcaacctaggactttcctattgaaatataatgagaatggaaccattacag | P9L, C136F, R190S, |
| | atgctgtagactgtgcacttgaccctctctcagaaacaaagtgtacgttga | D215G, Del242/243, |
| | aatccttcactgtagagaaaggaatctatcaaacttctaactttagagtcc | Y449Y, E484K, |
| | aaccaacagaatctattgttagatttcctaatattacaaacttgtgccctttc | N501Y, D614G, |
| | ggtgaagtatttaacgccaccagatttgcatctgtttatgcttggaacagg | H655Y, N679K, |
| | aagagaatcagcaactgtgttgctgattattctgtcctatataattccgcatc | T716I, and T859N |
| | attctccacatttaagtgttatggagtgtctcctactaaattaaatgatctctg | |
| | ctttactaatgtctatgcagattcatttgtaattagaggtgatgaagtcagac | |
| | aaatcgctccagggcaaactggaaagattgctgattataattataaatta | |
| | ccagatgactttacaggctgcgttatagcttggaattctaacaatcttgattc | |
| | taaggttggtggtaatcataattacctgtatagattgtttaggaagtctaatc | |
| | tcaaacctttcgagagagatatttcaactgaaatctatcaggccggtagc | |
| | acaccttgtaatggtgttaaaggatttaattgttactttcctttacaatcatatg | |
| | gtttccaacccacttatggtgttggttaccaaccatacagagtagtagtact | |
| | ttcatttgaacttctacatgcaccagcaactgtttgtggacctaagaagtct | |
| | actaatttggttaagaacaaatgtgtcaatttcaacttcaatggtttaacag | |
| | gcacaggtgttcttactgagtctaacaagaagtttctgcctttccaacaattt | |
| | ggcagagacattgctgacactactgatgctgtccgtgatccacagacac | |
| | ttgagattcttgacattacaccatgttcatttggtggtgtcagtgttataacac | |
| | caggaacaaatacttctaaccaggttgctgttctttatcagggtgttaactg | |
| | cacagaagtccctgttgctattcatgcagatcaacttactcctacttggcgt | |
| | gtttattctacaggttctaatgtatttcaaacacgtgcaggctgtttaatagg | |
| | agctgaatatgtcaacaactcatatgagtgtgacatacccattggtgcag | |
| | gtatatgcgctagttatcagactcagactaagtctcctcgggggcacgt | |
| | agtgtagctagtcaatccatcattgcctacactatgtcacttggtgcagag | |
| | aattcagttgcttactctaataactctattgccatacccataaactttactatt | |
| | agtgttaccacagaaattctaccagtgtctatgaccaagacatcagtag | |
| | attgtacaatgtacatttgtggtgattcaactgaatgcagcaatctattgttg | |
| | caatatggcagtttctgtacacaattaaaccgtgctttaactggaatagct | |
| | gttgaacaagacaagaacacccaagaagtatttgcacaagtcaaaca | |
| | aatttacaagacaccaccaattaaagatttcggtggatttaatttctcaca | |
| | aatattaccagatccatctaaaccaagcaagaggtcatttattgaagatc | |
| | tactattcaacaaagtgacacttgcagatgctggcttcatcaaacaatatg | |
| | gtgattgccttggtgatattgctgctagagacctcatttgtgcacagaagttt | |
| | aacggccttaatgtattgccacctttgctcacagatgaaatgattgctcaat | |
| | acacttctgcactgttagcgggtacaatcacttctggttggacctttggtgc | |
| | aggtgctgcattacaaataccatttgctatgcaaatggcttataggtttaat | |
| | ggtattggagttacacagaatgttctctatgagaaccagaaattgattgcc | |
| | aaccaatttaatagtgctattggcaagattcaagactcactttcttccacag | |
| | caagtgcacttggtaaacttcaagatgtggtcaaccagaatgcacaag | |
| | ctttaaacacgcttgttaaacaacttagctccaactttggtgcaatttcaagt | |
| | gtattaaatgatatcctttcacgtcttgacaaagttgaggctgaagtgcaa | |
| | attgataggttgatcacaggcagacttcaaagtttgcagacatatgtgact | |
| | caacaattaattagagctgcagaaatcagagcttctgctaatcttgctgct | |
| | actaagatgtcagagtgtgtacttggacaatctaagagagttgatttctgt | |
| | ggaaagggctatcatcttatgtccttccctcagtcagcacctcatggtgta | |
| | gtcttcttgcatgtgacttatgtccctgcacaagagaagaacttcacaact | |
| | gctcctgccatttgtcatgatggtaaagcacactttcctcgtgaaggtgtctt | |
| | tgtttcaaatggcacacactggtttgtaacacaaaggaatttctatgaacc | |
| | acaaatcattactacagacaacacatttgtgtctggtaactgtgatgttgta | |
| | ataggaattgtcaacaacacagtttatgatcctttgcaacctgaattagac | |
| | tcattcaaggaggagttagataaatatttcaagaatcatacatcaccaga | |
| | tgttgatttaggtgacatctctggcattaatgcttcagttgtaaacattcaga | |
| | aagaaattgaccgcctcaatgaggttgccaagaatttaaatgaatctctc | |
| | atcgatctccaagaacttggaaagtatgagcagtatatcaaatggccat | |
| | ggtacatttggctaggtttcatagctggcttgattgccatagtaatggtgac | |
| | aattatgctttgctgtatgaccagttgctgtagttgtctcaagggctgttgttct | |
| | tgtggatcctgctgcaaatttgatgaagacgactctgagccagtgctcaa | |
| | aggagtcaaattacattacacataa | |
| 16 | MFVFLVLLLLVSSQCVNLTTRTQLPPAYTNSFTRGV | Amino acid sequence |
| | YYPDKVFRSSVLHSTQDLFLPFFSNVTWFHAIHVSG | (N- to C-terminus) of a |
| | TNGTKRFDNPVLPFNDGVYFASTEKSNIIRGWIFGT | codon-optimized |
| | TLDSKTQSLLIVNNATNVVIKVCEFQFFNDPFLGVYH | SARS-COV-2 S |
| | KNNKSWMESEFRVYSSANNCTFEYVSQPFLMDLE | antigen sequence |
| | GKQGNFKNLSEFVFKNIDGYFKIYSKHTPINLVRGLP | based on the Wuhan- |
| | QGFSALEPLVDLPIGINITRFQTLLHRSYLTPGDSSS | Hu-1 reference strain |
| | GWTAGAAAYYVGYLQPRTFLLKYNENGTITDAVDC | (#NC_045512) and |
| | ALDPLSETKCTLKSFTVEKGIYQTSNFRVQPTESIVR | optimized for vaccinia, |
| | FPNITNLCPFGEVFNATRFASVYAWNRKRISNCVAD | which is further |
| | YSVLYNSASFSTFKCYGVSPTKLNDLCFTNVYADSF | modified to encode for |
| | VIRGDEVRQIAPGQTGKIADYNYKLPDDFTGCVIAW | an S antigen that |
| | NSNNLDSKVGGNHNYLYRLFRKSNLKPFERDISTEI | includes mutations of |
| | YQAGSTPCNGVKGFNCYFPLQSYGFQPTYGVGYQ | the C.1.2 variant |
| | PYRVVVLSFELLHAPATVCGPKKSTNLVKNKCVNF | lineage, including |
| | NFNGLTGTGVLTESNKKFLPFQQFGRDIADTTDAV | P9L, C136F, R190S, |
| | RDPQTLEILDITPCSFGGVSVITPGTNTSNQVAVLYQ | D215G, Del242/243, |
| | GVNCTEVPVAIHADQLTPTWRVYSTGSNVFQTRAG | Y449Y, E484K, |
| | CLIGAEYVNNSYECDIPIGAGICASYQTQTKSPRRA | N501Y, D614G, |
| | RSVASQSIIAYTMSLGAENSVAYSNNSIAIPINFTISV | H655Y, N679K, |
| | TTEILPVSMTKTSVDCTMYICGDSTECSNLLLQYGS | T716I, and T859N |
| | FCTQLNRALTGIAVEQDKNTQEVFAQVKQIYKTPPI | |
| | KDFGGFNFSQILPDPSKPSKRSFIEDLLFNKVTLADA | |
| | GFIKQYGDCLGDIAARDLICAQKFNGLNVLPPLLTDE | |
| | MIAQYTSALLAGTITSGWTFGAGAALQIPFAMQMAY | |
| | RFNGIGVTQNVLYENQKLIANQFNSAIGKIQDSLSST | |
| | ASALGKLQDVVNQNAQALNTLVKQLSSNFGAISSVL | |
| | NDILSRLDKVEAEVQIDRLITGRLQSLQTYVTQQLIR | |
| | AAEIRASANLAATKMSECVLGQSKRVDFCGKGYHL | |
| | MSFPQSAPHGVVFLHVTYVPAQEKNFTTAPAICHD | |
| | GKAHFPREGVFVSNGTHWFVTQRNFYEPQIITTDN | |
| | TFVSGNCDVVIGIVNNTVYDPLQPELDSFKEELDKY | |
| | FKNHTSPDVDLGDISGINASVVNIQKEIDRLNEVAKN | |
| | LNESLIDLQELGKYEQYIKWPWYIWLGFIAGLIAIVM | |
| | VTIMLCCMTSCCSCLKGCCSCGSCCKFDEDDSEPV | |
| | LKGVKLHYT | |
| 17 | atgtctgataacggaccacagaatcagcgaaacgcaccacgcattac | Nucleotide |
| | gtttggtggaccctcagattcaactggcagtaaccagaatggagaacg | sequence/ORF (5′ to |
| | cagtggagcgcgatctaaacaacgtcggcctcaaggtttacccaataat | 3′ end) of a codon- |
| | actgcgtcttggttcaccgctctcactcaacatggcaaggaaggccttaa | optimized SARS-COV- |
| | attccctcgaggacaaggcgttccaattaacaccaatagcagtccagat | 2 N antigen sequence |
| | gaccaaattggctactaccgaagagctaccagacgaattcgtggtggt | based on the Wuhan- |
| | gacggtaagatgaaagatctcagtccaagatggtatttctactacctagg | Hu-1 reference strain |
| | aactgggccagaagctggacttccctatggtgctaacaaagacggcat | (#NC_045512) and |
| | catatgggttgcaactgagggagccttgaatacacctaaagatcacatt | optimized for vaccinia, |
| | ggcacccgcaatcctgctaacaatgctgcaatcgtgctacaacttcctca | which is further |
| | aggaacaacattgcctaaaggcttctacgcagaagggagcagaggc | modified to encode for |
| | ggcagtcaagcctcttctcgttcctcatcacgtagtcgcaacagttcaaga | an N antigen that |
| | aattcaactccaggcagcagtatgggaacttctcctgctagaatggctgg | includes mutations of |
| | caatggctgtgatgctgctcttgctttgctgctgcttgacagattgaaccag | the B. 1.617.2 variant |
| | cttgagagcaagatgtctggtaaaggccaacaacaacaaggccaaa | lineage identified in |
| | ctgtcactaagaaatctgctgctgaggcttctaagaagcctcggcagaa | India, including D63G, |
| | acgtactgccactaaagcatacaatgtaacacaagctttcggcagacgt | R203M, G215C, and |
| | ggtccagaacaaacccaaggaaactttggagaccaggaactaatca | D377Y |
| | gacaaggaactgattacaaacattggccgcaaattgcacaatttgctcc | |
| | cagcgcttcagcgttcttcggaatgtcgcgcattggcatggaagtcacac | |
| | cttcgggaacgtggttgacctacacaggtgccatcaaattggatgacaa | |
| | agatccaaatttcaaagatcaagtcatattgctgaataagcatattgacg | |
| | catacaagacattcccaccaacagagcctaagaaggacaagaagaa | |
| | gaaggcttatgaaactcaagccttaccgcagagacagaagaaacag | |
| | caaactgtgactcttcttcctgctgcagatttggatgatttctccaaacaatt | |
| | gcaacaatccatgagcagtgctgactcaactcaggcctaa | |
| 18 | MSDNGPQNQRNAPRITFGGPSDSTGSNQNGERSG | Amino acid sequence |
| | ARSKQRRPQGLPNNTASWFTALTQHGKEGLKFPR | (N- to C-terminus) of a |
| | GQGVPINTNSSPDDQIGYYRRATRRIRGGDGKMKD | codon-optimized |
| | LSPRWYFYYLGTGPEAGLPYGANKDGIIWVATEGA | SARS-COV-2 N |
| | LNTPKDHIGTRNPANNAAIVLQLPQGTTLPKGFYAE | antigen sequence |
| | GSRGGSQASSRSSSRSRNSSRNSTPGSSMGTSPA | based on the Wuhan- |
| | RMAGNGCDAALALLLLDRLNQLESKMSGKGQQQQ | Hu-1 reference strain |
| | GQTVTKKSAAEASKKPRQKRTATKAYNVTQAFGRR | (#NC_045512) and |
| | GPEQTQGNFGDQELIRQGTDYKHWPQIAQFAPSA | optimized for vaccinia, |
| | SAFFGMSRIGMEVTPSGTWLTYTGAIKLDDKDPNF | which is further |
| | KDQVILLNKHIDAYKTFPPTEPKKDKKKKAYETQALP | modified to encode for |
| | QRQKKQQTVTLLPAADLDDFSKQLQQSMSSADST | an N antigen that |
| | QA | includes mutations of |
| | | the B.1.617.2 variant |
| | | lineage identified in |
| | | India, including D63G, |
| | | R203M, G215C, and |
| | | D377Y |
| 19 | atgtctgataacggaccacagaatcagcgaaacgcactacgcattacg | Nucleotide |
| | tttggtggaccctcagattcaactggcagtaaccagaatggagaacgc | sequence/ORF (5′ to |
| | agtggagcgcgatctaaacaacgtcggcctcaaggtttacccaataata | 3′ end) of a codon- |
| | ctgcgtcttggttcaccgctctcactcaacatggcaaggaagaccttaaa | optimized SARS-CoV- |
| | ttccctcgaggacaaggcgttccaattaacaccaatagcagtccagatg | 2 N antigen sequence |
| | accaaattggctactaccgaagagctaccagacgaattcgtggtggtg | based on the Wuhan- |
| | acggtaagatgaaagatctcagtccaagatggtatttctactacctagga | Hu-1 reference strain |
| | actgggccagaagctggacttccctatggtgctaacaaagacggcatc | (#NC_045512) and |
| | atatgggttgcaactgagggagccttgaatacacctaaagatcacattg | optimized for vaccinia, |
| | gcacccgcaatcctgctaacaatgctgcaatcgtgctacaacttcctcaa | which is further |
| | ggaacaacattgcctaaaggcttctacgcagaagggagcagaggcg | modified to encode for |
| | gcagtcaagcctcttctcgttcctcatcacgtagtcgcaacagttcaagaa | an N antigen that |
| | attcaactccaggcagcagtaaacgaacttctcctgctagaatggctgg | includes mutations of |
| | caatggcggtgatgctgctcttgctttgctgctgcttgacagattgaaccag | the C.1.2 variant |
| | cttgagagcaagatgtctggtaaaggccaacaacaacaaggccaaa | lineage, including |
| | ctgtcactaagaaatctgctgctgaggcttctaagaagcctcggcagaa | P13L, R203K, G204R, |
| | acgtactgccactaaagcatacaatgtaacacaagctttcggcagacgt | and Q384H |
| | ggtccagaacaaacccaaggaaactttggagaccaggaactaatca | |
| | gacaaggaactgattacaaacattggccgcaaattgcacaatttgctcc | |
| | cagcgcttcagcgttcttcggaatgtcgcgcattggcatggaagtcacac | |
| | cttcgggaacgtggttgacctacacaggtgccatcaaattggatgacaa | |
| | agatccaaatttcaaagatcaagtcatattgctgaataagcatattgacg | |
| | catacaagacattcccaccaacagagcctaagaaggacaagaagaa | |
| | gaaggctgatgaaactcaagccttaccgcacagacagaagaaacag | |
| | caaactgtgactcttcttcctgctgcagatttggatgatttctccaaacaatt | |
| | gcaacaatccatgagcagtgctgactcaactcaggcctaa | |
| 20 | MSDNGPQNQRNALRITFGGPSDSTGSNQNGERSG | Amino acid sequence |
| | ARSKQRRPQGLPNNTASWFTALTQHGKEDLKFPR | (N- to C-terminus) of a |
| | GQGVPINTNSSPDDQIGYYRRATRRIRGGDGKMKD | codon-optimized |
| | LSPRWYFYYLGTGPEAGLPYGANKDGIIWVATEGA | SARS-CoV-2 N |
| | LNTPKDHIGTRNPANNAAIVLQLPQGTTLPKGFYAE | antigen sequence |
| | GSRGGSQASSRSSSRSRNSSRNSTPGSSKRTSPA | based on the Wuhan- |
| | RMAGNGGDAALALLLLDRLNQLESKMSGKGQQQQ | Hu-1 reference strain |
| | GQTVTKKSAAEASKKPRQKRTATKAYNVTQAFGRR | (#NC_045512) and |
| | GPEQTQGNFGDQELIRQGTDYKHWPQIAQFAPSA | optimized for vaccinia, |
| | SAFFGMSRIGMEVTPSGTWLTYTGAIKLDDKDPNF | which is further |
| | KDQVILLNKHIDAYKTFPPTEPKKDKKKKADETQAL | modified to encode for |
| | PHRQKKQQTVTLLPAADLDDFSKQLQQSMSSADST | an N antigen that |
| | QA | includes mutations of |
| | | the C.1.2 variant |
| | | lineage, including |
| | | P13L, R203K, G204R, |
| | | and Q384H |
| 21 | atgtttgtctttcttgtcttattgccactagtctctagtcagtgtgttaactttaca | Nucleotide |
| | accagaactcaattaccacctgcatacactaattctttcacacgtggtgttt | sequence/ORF (5′ to |
| | attaccctgacaaagtattcagatcctcagtattacattcaactcaggactt | 3′ end) of a codon- |
| | gttcttacctttcttctccaatgttacttggttccatgctatacatgtctctggga | optimized SARS-CoV- |
| | ccaatggtactaagaggtttgctaaccctgtcctaccatttaatgatggtgtt | 2 S antigen sequence |
| | tatttcgcttccactgagaagtctaacataataagaggctggatatttggta | based on the Wuhan- |
| | ctactttagattcgaagacccagtccctacttattgttaataacgctactaat | Hu-1 reference strain |
| | gttgttattaaagtctgtgaatttcaattctgtaatgatccattcttgggtgtttat | (#NC_045512) and |
| | taccacaagaacaacaagagttggatggaaagtgagttcagagtttatt | optimized for vaccinia, |
| | ctagtgcgaataattgcactttcgaatatgtctctcagcctttccttatggac | which is further |
| | cttgaaggtaaacagggtaatttcaagaatcttagggaatttgtgtttaag | modified to encode for |
| | aatattgatggttatttcaagatatattctaagcacacgcctattaatttagtg | an S antigen that |
| | cgtggtctccctcagggtttctcggctttagaaccattggtagatttgccaat | includes mutations of |
| | aggtattaacatcactaggtttcaaactttacatataagttatttgactcctg | the B.1.351 variant |
| | gtgattcttcttcaggttggacagctggtgctgcagcttattatgtgggttatc | lineage, including |
| | ttcaacctaggactttcctattgaaatataatgagaatggaaccattacag | L18F, D80A, D215G, |
| | atgctgtagactgtgcacttgaccctctctcagaaacaaagtgtacgttga | Del242-244, R246I, |
| | aatccttcactgtagagaaaggaatctatcaaacttctaactttagagtcc | N501Y, E484K, |
| | aaccaacagaatctattgttagatttcctaatattacaaacttgtgccctttc | K417N, D614G, and |
| | ggtgaagtatttaacgccaccagatttgcatctgtttatgcttggaacagg | A701V |
| | aagagaatcagcaactgtgttgctgattattctgtcctatataattccgcatc | |
| | attctccacatttaagtgttatggagtgtctcctactaaattaaatgatctctg | |
| | ctttactaatgtctatgcagattcatttgtaattagaggtgatgaagtcagac | |
| | aaatcgctccagggcaaactggaaatattgctgattataattataaattac | |
| | cagatgactttacaggctgcgttatagcttggaattctaacaatcttgattct | |
| | aaggttggtggtaattataattacctgtatagattgtttaggaagtctaatct | |
| | caaacctttcgagagagatatttcaactgaaatctatcaggccggtagc | |
| | acaccttgtaatggtgttaaaggatttaattgttactttcctttacaatcatatg | |
| | gtttccaacccacttatggtgttggttaccaaccatacagagtagtagtact | |
| | ttcatttgaacttctacatgcaccagcaactgtttgtggacctaagaagtct | |
| | actaatttggttaagaacaaatgtgtcaatttcaacttcaatggtttaacag | |
| | gcacaggtgttcttactgagtctaacaagaagtttctgcctttccaacaattt | |
| | ggcagagacattgctgacactactgatgctgtccgtgatccacagacac | |
| | ttgagattcttgacattacaccatgttcatttggtggtgtcagtgttataacac | |
| | caggaacaaatacttctaaccaggttgctgttctttatcagggtgttaactg | |
| | cacagaagtccctgttgctattcatgcagatcaacttactcctacttggcgt | |
| | gtttattctacaggttctaatgtatttcaaacacgtgcaggctgtttaatagg | |
| | agctgaacatgtcaacaactcatatgagtgtgacatacccattggtgca | |
| | ggtatatgcgctagttatcagactcagactaattctcctcggcgggcacgt | |
| | agtgtagctagtcaatccatcattgcctacactatgtcacttggtgtagaga | |
| | attcagttgcttactctaataactctattgccatacccacaaactttactatta | |
| | gtgttaccacagaaattctaccagtgtctatgaccaagacatcagtagatt | |
| | gtacaatgtacatttgtggtgattcaactgaatgcagcaatctattgttgca | |
| | atatggcagtttctgtacacaattaaaccgtgctttaactggaatagctgtt | |
| | gaacaagacaagaacacccaagaagtatttgcacaagtcaaacaaa | |
| | tttacaagacaccaccaattaaagatttcggtggatttaatttctcacaaat | |
| | attaccagatccatctaaaccaagcaagaggtcatttattgaagatctact | |
| | attcaacaaagtgacacttgcagatgctggcttcatcaaacaatatggtg | |
| | attgccttggtgatattgctgctagagacctcatttgtgcacagaagtttaa | |
| | cggccttactgtattgccacctttgctcacagatgaaatgattgctcaatac | |
| | acttctgcactgttagcgggtacaatcacttctggttggacctttggtgcag | |
| | gtgctgcattacaaataccatttgctatgcaaatggcttataggtttaatggt | |
| | attggagttacacagaatgttctctatgagaaccagaaattgattgccaa | |
| | ccaatttaatagtgctattggcaagattcaagactcactttcttccacagca | |
| | agtgcacttggtaaacttcaagatgtggtcaaccagaatgcacaagcttt | |
| | aaacacgcttgttaaacaacttagctccaactttggtgcaatttcaagtgta | |
| | ttaaatgatatcctttcacgtcttgacaaagttgaggctgaagtgcaaattg | |
| | ataggttgatcacaggcagacttcaaagtttgcagacatatgtgactcaa | |
| | caattaattagagctgcagaaatcagagcttctgctaatcttgctgctacta | |
| | agatgtcagagtgtgtacttggacaatctaagagagttgatttctgtggaa | |
| | agggctatcatcttatgtccttccctcagtcagcacctcatggtgtagtcttct | |
| | tgcatgtgacttatgtccctgcacaagagaagaacttcacaactgctcct | |
| | gccatttgtcatgatggtaaagcacactttcctcgtgaaggtgtctttgtttca | |
| | aatggcacacactggtttgtaacacaaaggaatttctatgaaccacaaa | |
| | tcattactacagacaacacatttgtgtctggtaactgtgatgttgtaatagg | |
| | aattgtcaacaacacagtttatgatcctttgcaacctgaattagactcattc | |
| | aaggaggagttagataaatatttcaagaatcatacatcaccagatgttg | |
| | atttaggtgacatctctggcattaatgcttcagttgtaaacattcagaaaga | |
| | aattgaccgcctcaatgaggttgccaagaatttaaatgaatctctcatcga | |
| | tctccaagaacttggaaagtatgagcagtatatcaaatggccatggtac | |
| | atttggctaggtttcatagctggcttgattgccatagtaatggtgacaattat | |
| | gctttgctgtatgaccagttgctgtagttgtctcaagggctgttgttcttgtgg | |
| | atcctgctgcaaatttgatgaagacgactctgagccagtgctcaaagga | |
| | gtcaaattacattacacataa | |
| 22 | MFVFLVLLPLVSSQCVNFTTRTQLPPAYTNSFTRGV | Amino acid sequence |
| | YYPDKVFRSSVLHSTQDLFLPFFSNVTWFHAIHVSG | (N- to C-terminus) of a |
| | TNGTKRFANPVLPFNDGVYFASTEKSNIIRGWIFGT | codon-optimized |
| | TLDSKTQSLLIVNNATNVVIKVCEFQFCNDPFLGVY | SARS-CoV-2 S |
| | YHKNNKSWMESEFRVYSSANNCTFEYVSQPFLMD | antigen sequence |
| | LEGKQGNFKNLREFVFKNIDGYFKIYSKHTPINLVR | based on the Wuhan- |
| | GLPQGFSALEPLVDLPIGINITRFQTLHISYLTPGDSS | Hu-1 reference strain |
| | SGWTAGAAAYYVGYLQPRTFLLKYNENGTITDAVD | (#NC_045512) and |
| | CALDPLSETKCTLKSFTVEKGIYQTSNFRVQPTESIV | optimized for vaccinia, |
| | RFPNITNLCPFGEVFNATRFASVYAWNRKRISNCVA | which is further |
| | DYSVLYNSASFSTFKCYGVSPTKLNDLCFTNVYAD | modified to encode for |
| | SFVIRGDEVRQIAPGQTGNIADYNYKLPDDFTGCVI | an S antigen that |
| | AWNSNNLDSKVGGNYNYLYRLFRKSNLKPFERDIS | includes mutations of |
| | TEIYQAGSTPCNGVKGFNCYFPLQSYGFQPTYGVG | the B.1.351 variant |
| | YQPYRVVVLSFELLHAPATVCGPKKSTNLVKNKCV | lineage, including |
| | NFNFNGLTGTGVLTESNKKFLPFQQFGRDIADTTDA | L18F, D80A, D215G, |
| | VRDPQTLEILDITPCSFGGVSVITPGTNTSNQVAVLY | Del242-244, R246I, |
| | QGVNCTEVPVAIHADQLTPTWRVYSTGSNVFQTRA | N501Y, E484K, |
| | GCLIGAEHVNNSYECDIPIGAGICASYQTQTNSPRR | K417N, D614G, and |
| | ARSVASQSIIAYTMSLGVENSVAYSNNSIAIPTNFTIS | A701V |
| | VTTEILPVSMTKTSVDCTMYICGDSTECSNLLLQYG | |
| | SFCTQLNRALTGIAVEQDKNTQEVFAQVKQIYKTPP | |
| | IKDFGGFNFSQILPDPSKPSKRSFIEDLLFNKVTLAD | |
| | AGFIKQYGDCLGDIAARDLICAQKFNGLTVLPPLLTD | |
| | EMIAQYTSALLAGTITSGWTFGAGAALQIPFAMQMA | |
| | YRFNGIGVTQNVLYENQKLIANQFNSAIGKIQDSLSS | |
| | TASALGKLQDVVNQNAQALNTLVKQLSSNFGAISSV | |
| | LNDILSRLDKVEAEVQIDRLITGRLQSLQTYVTQQLI | |
| | RAAEIRASANLAATKMSECVLGQSKRVDFCGKGYH | |
| | LMSFPQSAPHGVVFLHVTYVPAQEKNFTTAPAICH | |
| | DGKAHFPREGVFVSNGTHWFVTQRNFYEPQIITTD | |
| | NTFVSGNCDVVIGIVNNTVYDPLQPELDSFKEELDK | |
| | YFKNHTSPDVDLGDISGINASVVNIQKEIDRLNEVAK | |
| | NLNESLIDLQELGKYEQYIKWPWYIWLGFIAGLIAIV | |
| | MVTIMLCCMTSCCSCLKGCCSCGSCCKFDEDDSE | |
| | PVLKGVKLHYT | |
| 23 | atgtctgataacggaccacagaatcagcgaaacgcaccacgcattac | Nucleotide |
| | gtttggtggaccctcagattcaactggcagtaaccagaatggagaacg | sequence/ORF (5′ to |
| | cagtggagcgcgatctaaacaacgtcggcctcaaggtttacccaataat | 3′ end) of a codon- |
| | actgcgtcttggttcaccgctctcactcaacatggcaaggaagaccttaa | optimized SARS-CoV- |
| | attccctcgaggacaaggcgttccaattaacaccaatagcagtccagat | 2 N antigen sequence |
| | gaccaaattggctactaccgaagagctaccagacgaattcgtggtggt | based on the Wuhan- |
| | gacggtaagatgaaagatctcagtccaagatggtatttctactacctagg | Hu-1 reference strain |
| | aactgggccagaagctggacttccctatggtgctaacaaagacggcat | (#NC_045512) and |
| | catatgggttgcaactgagggagccttgaatacacctaaagatcacatt | optimized for vaccinia, |
| | ggcacccgcaatcctgctaacaatgctgcaatcgtgctacaacttcctca | which is further |
| | aggaacaacattgcctaaaggcttctacgcagaagggagcagaggc | modified to encode for |
| | ggcagtcaagcctcttctcgttcctcatcacgtagtcgcaacagttcaaga | an N antigen that |
| | aattcaactccaggcagcagtagaggaatctctcctgctagaatggctg | includes mutations of |
| | gcaatggcggtgatgctgctcttgctttgctgctgcttgacagattgaacca | the B.1.351 variant |
| | gcttgagagcaagatgtctggtaaaggccaacaacaacaaggccaa | lineage, including |
| | actgtcactaagaaatctgctgctgaggcttctaagaagcctcggcaga | T2051 |
| | aacgtactgccactaaagcatacaatgtaacacaagctttcggcagac | |
| | gtggtccagaacaaacccaaggaaactttggagaccaggaactaatc | |
| | agacaaggaactgattacaaacattggccgcaaattgcacaatttgctc | |
| | ccagcgcttcagcgttcttcggaatgtcgcgcattggcatggaagtcaca | |
| | ccttcgggaacgtggttgacctacacaggtgccatcaaattggatgaca | |
| | aagatccaaatttcaaagatcaagtcatattgctgaataagcatattgac | |
| | gcatacaagacattcccaccaacagagcctaagaaggacaagaaga | |
| | agaaggctgatgaaactcaagccttaccgcagagacagaagaaaca | |
| | gcaaactgtgactcttcttcctgctgcagatttggatgatttctccaaacaat | |
| | tgcaacaatccatgagcagtgctgactcaactcaggcctaa | |
| 24 | MSDNGPQNQRNAPRITFGGPSDSTGSNQNGERSG | Amino acid sequence |
| | ARSKQRRPQGLPNNTASWFTALTQHGKEDLKFPR | (N- to C-terminus) of a |
| | GQGVPINTNSSPDDQIGYYRRATRRIRGGDGKMKD | codon-optimized |
| | LSPRWYFYYLGTGPEAGLPYGANKDGIIWVATEGA | SARS-CoV-2 N |
| | LNTPKDHIGTRNPANNAAIVLQLPQGTTLPKGFYAE | antigen sequence |
| | GSRGGSQASSRSSSRSRNSSRNSTPGSSRGISPA | based on the Wuhan- |
| | RMAGNGGDAALALLLLDRLNQLESKMSGKGQQQQ | Hu-1 reference strain |
| | GQTVTKKSAAEASKKPRQKRTATKAYNVTQAFGRR | (#NC_045512) and |
| | GPEQTQGNFGDQELIRQGTDYKHWPQIAQFAPSA | optimized for vaccinia, |
| | SAFFGMSRIGMEVTPSGTWLTYTGAIKLDDKDPNF | which is further |
| | KDQVILLNKHIDAYKTFPPTEPKKDKKKKADETQAL | modified to encode for |
| | PQRQKKQQTVTLLPAADLDDFSKQLQQSMSSADS | an N antigen that |
| | TQA | includes mutations of |
| | | the B.1.351 variant |
| | | lineage, including |
| | | T205I |
| 25 | atgtttgtctttcttgtcttattgccactagtctctagtcagtgtgttaatcttaca | Nucleotide |
| | accagaactcaattaccacctgcatacactaattctttcacacgtggtgttt | sequence/ORF (5′ to |
| | attaccctgacaaagtattcagatcctcagtattacattcaactcaggactt | 3′ end) of a codon- |
| | gttcttacctttcttctccaatgttacttggttccatgttatatctgggaccaatg | optimized SARS-CoV- |
| | gtactaagaggtttgataaccctgtcctaccatttaatgatggtgtttatttcg | 2 S antigen sequence |
| | cttccattgagaagtctaacataataagaggctggatatttggtactacttt | based on the Wuhan- |
| | agattcgaagacccagtccctacttattgttaataacgctactaatgttgtta | Hu-1 reference strain |
| | ttaaagtctgtgaatttcaattctgtaatgatccattcttggatcacaagaac | (#NC_045512) and |
| | aacaagagttggatggaaagtgagttcagagtttattctagtgcgaataa | optimized for vaccinia, |
| | ttgcactttcgaatatgtctctcagcctttccttatggaccttgaaggtaaac | which is further |
| | agggtaatttcaagaatcttagggaatttgtgtttaagaatattgatggttatt | modified to encode for |
| | tcaagatatattctaagcacacgcctattatagtgcgtgaacctgaagatc | an S antigen that |
| | tccctcagggtttctcggctttagaaccattggtagatttgccaataggtatt | includes mutations of |
| | aacatcactaggtttcaaactttacttgctttacatagaagttatttgactcct | the B.1.1.529/BA.1 |
| | ggtgattcttcttcaggttggacagctggtgctgcagcttattatgtgggttat | variant lineage, |
| | cttcaacctaggactttcctattgaaatataatgagaatggaaccattaca | including A67V, |
| | gatgctgtagactgtgcacttgaccctctctcagaaacaaagtgtacgttg | Del69-70 (HV), T95I, |
| | aaatccttcactgtagagaaaggaatctatcaaacttctaactttagagtc | G142D, Del143-145 |
| | caaccaacagaatctattgttagatttcctaatattacaaacttgtgcccttt | (VYY), Del211 (N), |
| | cgacgaagtatttaacgccaccagatttgcatctgtttatgcttggaacag | L212I, Ins214 (EPE), |
| | gaagagaatcagcaactgtgttgctgattattctgtcctatataatctcgca | G339D, S371L, |
| | ccattcttcacatttaagtgttatggagtgtctcctactaaattaaatgatctct | S373P, S375F, |
| | gctttactaatgtctatgcagattcatttgtaattagaggtgatgaagtcag | K417N, N440K, |
| | acaaatcgctccagggcaaactggaaatattgctgattataattataaatt | G446S, S477N, |
| | accagatgactttacaggctgcgttatagcttggaattctaacaaacttga | T478K, E484A, |
| | ttctaaggttagtggtaattataattacctgtatagattgtttaggaagtctaa | Q493R, G496S, |
| | tctcaaacctttcgagagagatatttcaactgaaatctatcaggccggtaa | Q498R, N501Y, |
| | caaaccttgtaatggtgttgcaggatttaattgttactttcctttacgatcatat | Y505H, T547K, |
| | agtttccgacccacttatggtgttggtcaccaaccatacagagtagtagta | D614G, H655Y, |
| | ctttcatttgaacttctacatgcaccagcaactgtttgtggacctaagaagt | N679K, P681H, |
| | ctactaatttggttaagaacaaatgtgtcaatttcaacttcaatggtttgaaa | N764K, D796Y, |
| | ggcacaggtgttcttactgagtctaacaagaagtttctgcctttccaacaat | N856K, Q954H, |
| | ttggcagagacattgctgacactactgatgctgtccgtgatccacagaca | N969K, and L981F |
| | cttgagattcttgacattacaccatgttcatttggtggtgtcagtgttataaca | |
| | ccaggaacaaatacttctaaccaggttgctgttctttatcagggtgttaact | |
| | gcacagaagtccctgttgctattcatgcagatcaacttactcctacttggc | |
| | gtgtttattctacaggttctaatgtatttcaaacacgtgcaggctgtttaatag | |
| | gagctgaatatgtcaacaactcatatgagtgtgacatacccattggtgca | |
| | ggtatatgcgctagttatcagactcagactaagtctcatcggcgggcacg | |
| | tagtgtagctagtcaatccatcattgcctacactatgtcacttggtgcagag | |
| | aattcagttgcttactctaataactctattgccatacccacaaactttactatt | |
| | agtgttaccacagaaattctaccagtgtctatgaccaagacatcagtag | |
| | attgtacaatgtacatttgtggtgattcaactgaatgcagcaatctattgttg | |
| | caatatggcagtttctgtacacaattaaagcgtgctttaactggaatagct | |
| | gttgaacaagacaagaacacccaagaagtatttgcacaagtcaaaca | |
| | aatttacaagacaccaccaattaagtatttcggtggatttaatttctcacaa | |
| | atattaccagatccatctaaaccaagcaagaggtcatttattgaagatct | |
| | actattcaacaaagtgacacttgcagatgctggcttcatcaaacaatatg | |
| | gtgattgccttggtgatattgctgctagagacctcatttgtgcacagaagttt | |
| | aagggccttactgtattgccacctttgctcacagatgaaatgattgctcaat | |
| | acacttctgcactgttagcgggtacaatcacttctggttggacctttggtgc | |
| | aggtgctgcattacaaataccatttgctatgcaaatggcttataggtttaat | |
| | ggtattggagttacacagaatgttctctatgagaaccagaaattgattgcc | |
| | aaccaatttaatagtgctattggcaagattcaagactcactttcttccacag | |
| | caagtgcacttggtaaacttcaagatgtggtcaaccacaatgcacaagc | |
| | tttaaacacgcttgttaaacaacttagctccaagtttggtgcaatttcaagt | |
| | gtattaaatgatatcttctcacgtcttgacaaagttgaggctgaagtgcaa | |
| | attgataggttgatcacaggcagacttcaaagtttgcagacatatgtgact | |
| | caacaattaattagagctgcagaaatcagagcttctgctaatcttgctgct | |
| | actaagatgtcagagtgtgtacttggacaatctaagagagttgatttctgt | |
| | ggaaagggctatcatcttatgtccttccctcagtcagcacctcatggtgta | |
| | gtcttcttgcatgtgacttatgtccctgcacaagagaagaacttcacaact | |
| | gctcctgccatttgtcatgatggtaaagcacactttcctcgtgaaggtgtctt | |
| | tgtttcaaatggcacacactggtttgtaacacaaaggaatttctatgaacc | |
| | acaaatcattactacagacaacacatttgtgtctggtaactgtgatgttgta | |
| | ataggaattgtcaacaacacagtttatgatcctttgcaacctgaattagac | |
| | tcattcaaggaggagttagataaatatttcaagaatcatacatcaccaga | |
| | tgttgatttaggtgacatctctggcattaatgcttcagttgtaaacattcaga | |
| | aagaaattgaccgcctcaatgaggttgccaagaatttaaatgaatctctc | |
| | atcgatctccaagaacttggaaagtatgagcagtatatcaaatggccat | |
| | ggtacatttggctaggtttcatagctggcttgattgccatagtaatggtgac | |
| | aattatgctttgctgtatgaccagttgctgtagttgtctcaagggctgttgttct | |
| | tgtggatcctgctgcaaatttgatgaagacgactctgagccagtgctcaa | |
| | aggagtcaaattacattacacataa | |
| 26 | MFVFLVLLPLVSSQCVNLTTRTQLPPAYTNSFTRGV | Amino acid sequence |
| | YYPDKVFRSSVLHSTQDLFLPFFSNVTWFHVISGTN | (N- to C-terminus) of a |
| | GTKRFDNPVLPFNDGVYFASIEKSNIIRGWIFGTTLD | codon-optimized |
| | SKTQSLLIVNNATNVVIKVCEFQFCNDPFLDHKNNK | SARS-CoV-2 S |
| | SWMESEFRVYSSANNCTFEYVSQPFLMDLEGKQG | antigen sequence |
| | NFKNLREFVFKNIDGYFKIYSKHTPIIVREPEDLPQG | based on the Wuhan- |
| | FSALEPLVDLPIGINITRFQTLLALHRSYLTPGDSSS | Hu-1 reference strain |
| | GWTAGAAAYYVGYLQPRTFLLKYNENGTITDAVDC | (#NC_045512) and |
| | ALDPLSETKCTLKSFTVEKGIYQTSNFRVQPTESIVR | optimized for vaccinia, |
| | FPNITNLCPFDEVFNATRFASVYAWNRKRISNCVAD | which is further |
| | YSVLYNLAPFFTFKCYGVSPTKLNDLCFTNVYADSF | modified to encode for |
| | VIRGDEVRQIAPGQTGNIADYNYKLPDDFTGCVIAW | an S antigen that |
| | NSNKLDSKVSGNYNYLYRLFRKSNLKPFERDISTEI | includes mutations of |
| | YQAGNKPCNGVAGFNCYFPLRSYSFRPTYGVGHQ | the B.1.1.529/BA.1 |
| | PYRVVVLSFELLHAPATVCGPKKSTNLVKNKCVNF | variant lineage, |
| | NFNGLKGTGVLTESNKKFLPFQQFGRDIADTTDAV | including A67V, |
| | RDPQTLEILDITPCSFGGVSVITPGTNTSNQVAVLYQ | Del69-70 (HV), T951, |
| | GVNCTEVPVAIHADQLTPTWRVYSTGSNVFQTRAG | G142D, Del143-145 |
| | CLIGAEYVNNSYECDIPIGAGICASYQTQTKSHRRA | (VYY), Del211 (N), |
| | RSVASQSIIAYTMSLGAENSVAYSNNSIAIPTNFTISV | L212I, Ins214 (EPE), |
| | TTEILPVSMTKTSVDCTMYICGDSTECSNLLLQYGS | G339D, S371L, |
| | FCTQLKRALTGIAVEQDKNTQEVFAQVKQIYKTPPIK | S373P, S375F, |
| | YFGGFNFSQILPDPSKPSKRSFIEDLLFNKVTLADA | K417N, N440K, |
| | GFIKQYGDCLGDIAARDLICAQKFKGLTVLPPLLTDE | G446S, S477N, |
| | MIAQYTSALLAGTITSGWTFGAGAALQIPFAMQMAY | T478K, E484A, |
| | RFNGIGVTQNVLYENQKLIANQFNSAIGKIQDSLSST | Q493R, G496S, |
| | ASALGKLQDVVNHNAQALNTLVKQLSSKFGAISSVL | Q498R, N501Y, |
| | NDIFSRLDKVEAEVQIDRLITGRLQSLQTYVTQQLIR | Y505H, T547K, |
| | AAEIRASANLAATKMSECVLGQSKRVDFCGKGYHL | D614G, H655Y, |
| | MSFPQSAPHGVVFLHVTYVPAQEKNFTTAPAICHD | N679K, P681H, |
| | GKAHFPREGVFVSNGTHWFVTQRNFYEPQIITTDN | N764K, D796Y, |
| | TFVSGNCDVVIGIVNNTVYDPLQPELDSFKEELDKY | N856K, Q954H, |
| | FKNHTSPDVDLGDISGINASVVNIQKEIDRLNEVAKN | N969K, and L981F |
| | LNESLIDLQELGKYEQYIKWPWYIWLGFIAGLIAIVM | |
| | VTIMLCCMTSCCSCLKGCCSCGSCCKFDEDDSEPV | |
| | LKGVKLHYT | |
| 27 | atgtctgataacggaccacagaatcagcgaaacgcactacgcattacg | Nucleotide |
| | tttggtggaccctcagattcaactggcagtaaccagaatggaggagcg | sequence/ORF (5′ to |
| | cgatctaaacaacgtcggcctcaaggtttacccaataatactgcgtcttg | 3′ end) of a codon- |
| | gttcaccgctctcactcaacatggcaaggaagaccttaaattccctcgag | optimized SARS-CoV- |
| | gacaaggcgttccaattaacaccaatagcagtccagatgaccaaattg | 2 N antigen sequence |
| | gctactaccgaagagctaccagacgaattcgtggtggtgacggtaaga | based on the Wuhan- |
| | tgaaagatctcagtccaagatggtatttctactacctaggaactgggcca | Hu-1 reference strain |
| | gaagctggacttccctatggtgctaacaaagacggcatcatatgggttg | (#NC_045512) and |
| | caactgagggagccttgaatacacctaaagatcacattggcacccgca | optimized for vaccinia, |
| | atcctgctaacaatgctgcaatcgtgctacaacttcctcaaggaacaac | which is further |
| | attgcctaaaggcttctacgcagaagggagcagaggcggcagtcaag | modified to encode for |
| | cctcttctcgttcctcatcacgtagtcgcaacagttcaagaaattcaactcc | an N antigen that |
| | aggcagcagtaaacgaacttctcctgctagaatggctggcaatggcgg | includes mutations of |
| | tgatgctgctcttgctttgctgctgcttgacagattgaaccagcttgagagc | the B.1.1.529/BA. 1 |
| | aagatgtctggtaaaggccaacaacaacaaggccaaactgtcactaa | variant lineage, |
| | gaaatctgctgctgaggcttctaagaagcctcggcagaaacgtactgcc | including P13L, |
| | actaaagcatacaatgtaacacaagctttcggcagacgtggtccagaa | Del31-33 (ERS), |
| | caaacccaaggaaactttggagaccaggaactaatcagacaaggaa | R203K, G204R |
| | ctgattacaaacattggccgcaaattgcacaatttgctcccagcgcttca | |
| | gcgttcttcggaatgtcgcgcattggcatggaagtcacaccttcgggaac | |
| | gtggttgacctacacaggtgccatcaaattggatgacaaagatccaaat | |
| | ttcaaagatcaagtcatattgctgaataagcatattgacgcatacaagac | |
| | attcccaccaacagagcctaagaaggacaagaagaagaaggctgat | |
| | gaaactcaagccttaccgcagagacagaagaaacagcaaactgtga | |
| | ctcttcttcctgctgcagatttggatgatttctccaaacaattgcaacaatcc | |
| | atgagcagtgctgactcaactcaggcctaa | |
| 28 | MSDNGPQNQRNALRITFGGPSDSTGSNQNGGARS | Amino acid sequence |
| | KQRRPQGLPNNTASWFTALTQHGKEDLKFPRGQG | (N- to C-terminus) of a |
| | VPINTNSSPDDQIGYYRRATRRIRGGDGKMKDLSP | codon-optimized |
| | RWYFYYLGTGPEAGLPYGANKDGIIWVATEGALNT | SARS-CoV-2 N |
| | PKDHIGTRNPANNAAIVLQLPQGTTLPKGFYAEGSR | antigen sequence |
| | GGSQASSRSSSRSRNSSRNSTPGSSKRTSPARMA | based on the Wuhan- |
| | GNGGDAALALLLLDRLNQLESKMSGKGQQQQGQT | Hu-1 reference strain |
| | VTKKSAAEASKKPRQKRTATKAYNVTQAFGRRGPE | (#NC_045512) and |
| | QTQGNFGDQELIRQGTDYKHWPQIAQFAPSASAFF | optimized for vaccinia, |
| | GMSRIGMEVTPSGTWLTYTGAIKLDDKDPNFKDQVI | which is further |
| | LLNKHIDAYKTFPPTEPKKDKKKKADETQALPQRQK | modified to encode for |
| | KQQTVTLLPAADLDDFSKQLQQSMSSADSTQA | an N antigen that |
| | | includes mutations of |
| | | the B.1.1.529/BA.1 |
| | | variant lineage, |
| | | including P13L, |
| | | Del31-33 (ERS), |
| | | R203K, G204R |
| 29 | atgtttgtctttcttgtcttattgccactagtctctagtcagtgtgttaactttaca | Nucleotide |
| | aacagaactcaattaccatctgcatacactaattctttcacacgtggtgttt | sequence/ORF (5′ to |
| | attaccctgacaaagtattcagatcctcagtattacattcaactcaggactt | 3′ end) of a codon- |
| | gttcttacctttcttctccaatgttacttggttccatgctatacatgtctctggga | optimized SARS-CoV- |
| | ccaatggtactaagaggtttgataaccctgtcctaccatttaatgatggtgt | 2 S antigen sequence |
| | ttatttcgcttccactgagaagtctaacataataagaggctggatatttggt | based on the Wuhan- |
| | actactttagattcgaagacccagtccctacttattgttaataacgctacta | Hu-1 reference strain |
| | atgttgttattaaagtctgtgaatttcaattctgtaattatccattcttgggtgttt | (#NC_045512) and |
| | attaccacaagaacaacaagagttggatggaaagtgagttcagagttt | optimized for vaccinia, |
| | attctagtgcgaataattgcactttcgaatatgtctctcagcctttccttatgg | which is further |
| | accttgaaggtaaacagggtaatttcaagaatcttagcgaatttgtgttta | modified to encode for |
| | agaatattgatggttatttcaagatatattctaagcacacgcctattaattta | an S antigen that |
| | gtgcgtgatctccctcagggtttctcggctttagaaccattggtagatttgcc | includes mutations of |
| | aataggtattaacatcactaggtttcaaactttacttgctttacatagaagtt | the P.1 variant |
| | atttgactcctggtgattcttcttcaggttggacagctggtgctgcagcttatt | lineage, including |
| | atgtgggttatcttcaacctaggactttcctattgaaatataatgagaatgg | L18F, T20N, P26S, |
| | aaccattacagatgctgtagactgtgcacttgaccctctctcagaaacaa | D138Y, R190S, |
| | agtgtacgttgaaatccttcactgtagagaaaggaatctatcaaacttcta | K417T, E484K, |
| | actttagagtccaaccaacagaatctattgttagatttcctaatattacaaa | N501Y, D614G, |
| | cttgtgccctttcggtgaagtatttaacgccaccagatttgcatctgtttatgc | H655Y, T1027I, and |
| | ttggaacaggaagagaatcagcaactgtgttgctgattattctgtcctatat | V1167F |
| | aattccgcatcattctccacatttaagtgttatggagtgtctcctactaaatta | |
| | aatgatctctgctttactaatgtctatgcagattcatttgtaattagaggtgat | |
| | gaagtcagacaaatcgctccagggcaaactggaacgattgctgattat | |
| | aattataaattaccagatgactttacaggctgcgttatagcttggaattcta | |
| | acaatcttgattctaaggttggtggtaattataattacctgtatagattgttta | |
| | ggaagtctaatctcaaacctttcgagagagatatttcaactgaaatctatc | |
| | aggccggtagcacaccttgtaatggtgttaaaggatttaattgttactttcct | |
| | ttacaatcatatggtttccaacccacttatggtgttggttaccaaccataca | |
| | gagtagtagtactttcatttgaacttctacatgcaccagcaactgtttgtgg | |
| | acctaagaagtctactaatttggttaagaacaaatgtgtcaatttcaacttc | |
| | aatggtttaacaggcacaggtgttcttactgagtctaacaagaagtttctg | |
| | cctttccaacaatttggcagagacattgctgacactactgatgctgtccgt | |
| | gatccacagacacttgagattcttgacattacaccatgttcatttggtggtgt | |
| | cagtgttataacaccaggaacaaatacttctaaccaggttgctgttctttat | |
| | cagggtgttaactgcacagaagtccctgttgctattcatgcagatcaactt | |
| | actcctacttggcgtgtttattctacaggttctaatgtatttcaaacacgtgca | |
| | ggctgtttaataggagctgaatatgtcaacaactcatatgagtgtgacata | |
| | cccattggtgcaggtatatgcgctagttatcagactcagactaattctcctc | |
| | ggcgggcacgtagtgtagctagtcaatccatcattgcctacactatgtca | |
| | cttggtgcagagaattcagttgcttactctaataactctattgccataccca | |
| | caaactttactattagtgttaccacagaaattctaccagtgtctatgaccaa | |
| | gacatcagtagattgtacaatgtacatttgtggtgattcaactgaatgcag | |
| | caatctattgttgcaatatggcagtttctgtacacaattaaaccgtgctttaa | |
| | ctggaatagctgttgaacaagacaagaacacccaagaagtatttgcac | |
| | aagtcaaacaaatttacaagacaccaccaattaaagatttcggtggattt | |
| | aatttctcacaaatattaccagatccatctaaaccaagcaagaggtcattt | |
| | attgaagatctactattcaacaaagtgacacttgcagatgctggcttcatc | |
| | aaacaatatggtgattgccttggtgatattgctgctagagacctcatttgtg | |
| | cacagaagtttaacggccttactgtattgccacctttgctcacagatgaaa | |
| | tgattgctcaatacacttctgcactgttagcgggtacaatcacttctggttgg | |
| | acctttggtgcaggtgctgcattacaaataccatttgctatgcaaatggctt | |
| | ataggtttaatggtattggagttacacagaatgttctctatgagaaccaga | |
| | aattgattgccaaccaatttaatagtgctattggcaagattcaagactcac | |
| | tttcttccacagcaagtgcacttggtaaacttcaagatgtggtcaaccaga | |
| | atgcacaagctttaaacacgcttgttaaacaacttagctccaactttggtg | |
| | caatttcaagtgtattaaatgatatcctttcacgtcttgacaaagttgaggct | |
| | gaagtgcaaattgataggttgatcacaggcagacttcaaagtttgcaga | |
| | catatgtgactcaacaattaattagagctgcagaaatcagagcttctgct | |
| | aatcttgctgctattaagatgtcagagtgtgtacttggacaatctaagaga | |
| | gttgatttctgtggaaagggctatcatcttatgtccttccctcagtcagcacc | |
| | tcatggtgtagtcttcttgcatgtgacttatgtccctgcacaagagaagaac | |
| | ttcacaactgctcctgccatttgtcatgatggtaaagcacactttcctcgtg | |
| | aaggtgtctttgtttcaaatggcacacactggtttgtaacacaaaggaattt | |
| | ctatgaaccacaaatcattactacagacaacacatttgtgtctggtaactg | |
| | tgatgttgtaataggaattgtcaacaacacagtttatgatcctttgcaacct | |
| | gaattagactcattcaaggaggagttagataaatatttcaagaatcatac | |
| | atcaccagatgttgatttaggtgacatctctggcattaatgcttcatttgtaa | |
| | acattcagaaagaaattgaccgcctcaatgaggttgccaagaatttaaa | |
| | tgaatctctcatcgatctccaagaacttggaaagtatgagcagtatatca | |
| | aatggccatggtacatttggctaggtttcatagctggcttgattgccatagt | |
| | aatggtgacaattatgctttgctgtatgaccagttgctgtagttgtctcaagg | |
| | gctgttgttcttgtggatcctgctgcaaatttgatgaagacgactctgagcc | |
| | agtgctcaaaggagtcaaattacattacacataa | |
| 30 | MFVFLVLLPLVSSQCVNFTNRTQLPSAYTNSFTRGV | Amino acid sequence |
| | YYPDKVFRSSVLHSTQDLFLPFFSNVTWFHAIHVSG | (N- to C-terminus) of a |
| | TNGTKRFDNPVLPFNDGVYFASTEKSNIIRGWIFGT | codon-optimized |
| | TLDSKTQSLLIVNNATNVVIKVCEFQFCNYPFLGVYY | SARS-CoV-2 S |
| | HKNNKSWMESEFRVYSSANNCTFEYVSQPFLMDL | antigen sequence |
| | EGKQGNFKNLSEFVFKNIDGYFKIYSKHTPINLVRDL | based on the Wuhan- |
| | PQGFSALEPLVDLPIGINITRFQTLLALHRSYLTPGD | Hu-1 reference strain |
| | SSSGWTAGAAAYYVGYLQPRTFLLKYNENGTITDA | (#NC_045512) and |
| | VDCALDPLSETKCTLKSFTVEKGIYQTSNFRVQPTE | optimized for vaccinia, |
| | SIVRFPNITNLCPFGEVFNATRFASVYAWNRKRISN | which is further |
| | CVADYSVLYNSASFSTFKCYGVSPTKLNDLCFTNV | modified to encode for |
| | YADSFVIRGDEVRQIAPGQTGTIADYNYKLPDDFTG | an S antigen that |
| | CVIAWNSNNLDSKVGGNYNYLYRLFRKSNLKPFER | includes mutations of |
| | DISTEIYQAGSTPCNGVKGFNCYFPLQSYGFQPTY | the P.1 variant |
| | GVGYQPYRVVVLSFELLHAPATVCGPKKSTNLVKN | lineage, including |
| | KCVNFNFNGLTGTGVLTESNKKFLPFQQFGRDIAD | L18F, T20N, P26S, |
| | TTDAVRDPQTLEILDITPCSFGGVSVITPGTNTSNQV | D138Y, R190S, |
| | AVLYQGVNCTEVPVAIHADQLTPTWRVYSTGSNVF | K417T, E484K, |
| | QTRAGCLIGAEYVNNSYECDIPIGAGICASYQTQTN | N501Y, D614G, |
| | SPRRARSVASQSIIAYTMSLGAENSVAYSNNSIAIPT | H655Y, T1027I and |
| | NFTISVTTEILPVSMTKTSVDCTMYICGDSTECSNLL | V1167F |
| | LQYGSFCTQLNRALTGIAVEQDKNTQEVFAQVKQIY | |
| | KTPPIKDFGGFNFSQILPDPSKPSKRSFIEDLLFNKV | |
| | TLADAGFIKQYGDCLGDIAARDLICAQKFNGLTVLPP | |
| | LLTDEMIAQYTSALLAGTITSGWTFGAGAALQIPFA | |
| | MQMAYRFNGIGVTQNVLYENQKLIANQFNSAIGKIQ | |
| | DSLSSTASALGKLQDVVNQNAQALNTLVKQLSSNF | |
| | GAISSVLNDILSRLDKVEAEVQIDRLITGRLQSLQTY | |
| | VTQQLIRAAEIRASANLAAIKMSECVLGQSKRVDFC | |
| | GKGYHLMSFPQSAPHGVVFLHVTYVPAQEKNFTTA | |
| | PAICHDGKAHFPREGVFVSNGTHWFVTQRNFYEP | |
| | QIITTDNTFVSGNCDVVIGIVNNTVYDPLQPELDSFK | |
| | EELDKYFKNHTSPDVDLGDISGINASFVNIQKEIDRL | |
| | NEVAKNLNESLIDLQELGKYEQYIKWPWYIWLGFIA | |
| | GLIAIVMVTIMLCCMTSCCSCLKGCCSCGSCCKFDE | |
| | DDSEPVLKGVKLHYT | |
| 31 | atgtctgataacggaccacagaatcagcgaaacgcaccacgcattac | Nucleotide |
| | gtttggtggaccctcagattcaactggcagtaaccagaatggagaacg | sequence/ORF (5′ to |
| | cagtggagcgcgatctaaacaacgtcggcctcaaggtttacccaataat | 3′ end) of a codon- |
| | actgcgtcttggttcaccgctctcactcaacatggcaaggaagaccttaa | optimized SARS-CoV- |
| | attccctcgaggacaaggcgttccaattaacaccaatagcagtcgagat | 2 N antigen sequence |
| | gaccaaattggctactaccgaagagctaccagacgaattcgtggtggt | based on the Wuhan- |
| | gacggtaagatgaaagatctcagtccaagatggtatttctactacctagg | Hu-1 reference strain |
| | aactgggccagaagctggacttccctatggtgctaacaaagacggcat | (#NC_045512) and |
| | catatgggttgcaactgagggagccttgaatacacctaaagatcacatt | optimized for vaccinia, |
| | ggcacccgcaatcctgctaacaatgctgcaatcgtgctacaacttcctca | which is further |
| | aggaacaacattgcctaaaggcttctacgcagaagggagcagaggc | modified to encode for |
| | ggcagtcaagcctcttctcgttcctcatcacgtagtcgcaacagttcaaga | an N antigen that |
| | aattcaactccaggcagcagtaaacgaacttctcctgctagaatggctg | includes mutations of |
| | gcaatggcggtgatgctgctcttgctttgctgctgcttgacagattgaacca | the P.1 variant |
| | gcttgagagcaagatgtctggtaaaggccaacaacaacaaggccaa | lineage, including |
| | actgtcactaagaaatctgctgctgaggcttctaagaagcctcggcaga | P80R, R203K, and |
| | aacgtactgccactaaagcatacaatgtaacacaagctttcggcagac | G204K |
| | gtggtccagaacaaacccaaggaaactttggagaccaggaactaatc | |
| | agacaaggaactgattacaaacattggccgcaaattgcacaatttgctc | |
| | ccagcgcttcagcgttcttcggaatgtcgcgcattggcatggaagtcaca | |
| | ccttcgggaacgtggttgacctacacaggtgccatcaaattggatgaca | |
| | aagatccaaatttcaaagatcaagtcatattgctgaataagcatattgac | |
| | gcatacaagacattcccaccaacagagcctaagaaggacaagaaga | |
| | agaaggctgatgaaactcaagccttaccgcagagacagaagaaaca | |
| | gcaaactgtgactcttcttcctgctgcagatttggatgatttctccaaacaat | |
| | tgcaacaatccatgagcagtgctgactcaactcaggcctaa | |
| 32 | MSDNGPQNQRNAPRITFGGPSDSTGSNQNGERSG | Amino acid sequence |
| | ARSKQRRPQGLPNNTASWFTALTQHGKEDLKFPR | (N- to C-terminus) of a |
| | GQGVPINTNSSRDDQIGYYRRATRRIRGGDGKMKD | codon-optimized |
| | LSPRWYFYYLGTGPEAGLPYGANKDGIIWVATEGA | SARS-CoV-2 N |
| | LNTPKDHIGTRNPANNAAIVLQLPQGTTLPKGFYAE | antigen sequence |
| | GSRGGSQASSRSSSRSRNSSRNSTPGSSKRTSPA | based on the Wuhan- |
| | RMAGNGGDAALALLLLDRLNQLESKMSGKGQQQQ | Hu-1 reference strain |
| | GQTVTKKSAAEASKKPRQKRTATKAYNVTQAFGRR | (#NC_045512) and |
| | GPEQTQGNFGDQELIRQGTDYKHWPQIAQFAPSA | optimized for vaccinia, |
| | SAFFGMSRIGMEVTPSGTWLTYTGAIKLDDKDPNF | which is further |
| | KDQVILLNKHIDAYKTFPPTEPKKDKKKKADETQAL | modified to encode for |
| | PQRQKKQQTVTLLPAADLDDFSKQLQQSMSSADS | an N antigen that |
| | TQA | includes mutations of |
| | | the P.1 variant |
| | | lineage, including |
| | | P80R, R203K, and |
| | | G204K |

In some embodiments, the DNA sequence encoding the S protein is based on the Wuhan-Hu-1 reference strain or is derived from a variant of concern (VOC). In some embodiments, the DNA sequence encoding the N protein is based on the Wuhan-Hu-1 reference strain or is derived from a VOC. In some embodiments, the VOC is selected from the group consisting of B.1.1.7 (Alpha), B.1.351 (Beta), P.1 (Gamma), B.1.617.2 (Delta), B.1.621 (Mu), 0.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments, the DNA sequence encoding the S protein comprises a nucleotide sequence that is at least 80% identical to any one of SEQ ID NOs: 5, 9, 11, 13, 15, 21, 25, and 29. In some embodiments, the DNA sequence encoding the N protein comprises a nucleotide sequence that is at least 80% identical to any one of SEQ ID NOs: 7, 17, 19, 23, 27, and 31.

In some embodiments, the DNA sequences encode an S protein and an N protein based on the ancestral Wuhan-Hu-1 reference strain. In certain of these embodiments, the DNA sequences comprise or consist of nucleotide sequences set forth in SEQ ID NOs: 5 and 7, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 5 and 7, respectively. The corresponding S protein and N protein encoded by the]DNA sequences comprise or consist of amino acid sequences set forth in SEQ ID NOs: 6 and 8, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 6 and 8, respectively.

In some embodiments, the DNA sequences encode an S protein and an N protein based on the B.1.351 (Beta) strain. In certain of these embodiments, the DNA sequences comprise or consist of nucleotide sequences set forth in SEQ ID NOs: 21 and 23, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 21 and 23, respectively. The corresponding S protein and N protein encoded by the DNA sequences comprise or consist of amino acid sequences set forth in SEQ ID NOs: 22 and 24, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 22 and 24, respectively.

In some embodiments, the DNA sequences encode an S protein and an N protein based on the P.1 (Gamma) strain. In certain of these embodiments, the DNA sequences comprise or consist of nucleotide sequences set forth in SEQ ID NOs: 29 and 3′, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 29 and 31, respectively. The corresponding S protein and N protein encoded by the DNA sequences comprise or consist of amino acid sequences set forth in SEQ ID NOs: 30 and 32, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 30 and 32, respectively.

In some embodiments, the DNA sequences encode an S protein and an N protein based on the B.1.617.2 (Delta) strain. In certain of these embodiments, the DNA sequences comprise or consist of nucleotide sequences set forth in SEQ ID NOs: 9 and 17, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 9 and 17, respectively. The corresponding S protein and N protein encoded by the DNA sequences comprise or consist of amino acid sequences set forth in SEQ ID NOs: 10 and 18, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 10 and 18, respectively.

In some embodiments, the DNA sequences encode an S protein and an N protein based on the B.1.617.2 (Delta) strain. In certain of these embodiments, the DNA sequences comprise or consist of nucleotide sequences set forth in SEQ ID NOs: 11 and 17, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 11 and 17, respectively. The corresponding S protein and N protein encoded by the hDNA sequences comprise or consist of amino acid sequences set forth in SEQ ID NOs: 12 and 18, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 12 and 18, respectively.

In some embodiments, the DNA sequences encode an S protein and an N protein based on the C.1.2 strain. In certain of these embodiments, the DNA sequences comprise or consist of nucleotide sequences set forth in SEQ ID NOs: 15 and 19, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 15 and 19, respectively. The corresponding S protein and N protein encoded by the DNA sequences comprise or consist of amino acid sequences set forth in SEQ ID NOs: 16 and 20, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 16 and 20, respectively.

In some embodiments, the DNA sequences encode an S protein and an N protein based on the B.1.1.529/BA.1 (Omicron) strain. In certain of these embodiments, the DNA sequences comprise or consist of nucleotide sequences set forth in SEQ ID NOs: 25 and 27, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 25 and 27, respectively. The corresponding S protein and N protein encoded by the DNA sequences comprise or consist of amino acid sequences set forth in SEQ ID NOs: 26 and 28, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 26 and 28, respectively.

In some embodiments, the SARS-CoV-2 comprises the Wuhan-Hu-1 reference strain or a VOC. In some embodiments, the VOC is selected from the group consisting of B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments, the vaccines and/or immunogenic compositions comprise one or more sMVA vectors, wherein the one or more sMVA vectors comprise one or more heterologous DNA sequences encoding the S protein and/or N protein of the SARS-CoV-2 Wuhan-Hu-1 reference stain. In some embodiments, the one or more sMVA vectors comprise one or more heterologous DNA sequences encoding a mutant S protein and/or N protein such as mutant S protein and/or N protein based on a variant of concern (VOC), including B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments, the vaccines and/or immunogenic compositions are used for preventing or treating a coronavirus infection, for example, SARS-CoV-2 infection, caused by the ancestral Wuhan-Hu-1 reference strain or a VOC, including, but not limited to, B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments, the encoded mutant S protein based on the B.1.617.2 lineage comprises one or more of the following mutations: T19R, E156G, Del157/158, S255F, L452R, T478K, D614G, P681R, D950N, G142D, Del156/157, R158G, A222, L5F, R21T, T51I, H66Y, K77T, D80Y, T951, G181V, R214H, P251L, D253A, V289I, V308L, A411S, G446V, T547I, A570S, T572I, Q613H, S640F, E661D, Q675H, T7191, P809S, A845S, I850L, A879S, D979E, A1078S, H1101Y, D1127G, L1141W, G1167V, K1191N, G1291V, and V1264L. Other mutations such as K417T may also be included.

In some embodiments, the encoded mutant S protein based on VOC lineage B.1.351 (Beta) comprises one or more of the following mutations: L18F, D80A, D215G, Del242-244, R2461, N501Y, E484K, K417N, D614G, and A701V.

In some embodiments, the encoded mutant S protein based on VOC lineage P.1 (Gamma) comprises one or more of the following mutations: L18F, T20N, P26S, Di 38Y, R190S, K417T, E484K, N501Y, D614G, H655Y, T10271, and V1167F.

In some embodiments, the encoded mutant S protein based on VOC lineage B.1.617.2 (Delta) comprises one or more of the following mutations: T19R, G142D, Del156-157, R158G, L452R, T478K, D614G, P681R, and D950N.

In some embodiments, the encoded mutant S protein based on VOC lineage B.1.617.2 (Delta) comprises one or more of the following mutations: T19R, K77T, Del157-158, L452R, T478K, D614G, P681R, and D950N.

In some embodiments, the encoded mutant S protein based on VOC lineage B.1.617.2 (Delta) comprises one or more of the following mutations: T19R, E156G, Del157-158, S255F, L452R, T478K, D614G, P681R, and D950N.

In some embodiments, the encoded mutant S protein based on VOC lineage B.1.617.2 (Delta) comprises one or more of the following mutations: T19R, L452R, T478K, D614G, P681R, and D950N and additionally one or more mutations comprising G142D, R158G, A222V, S255F, E156G, T951, K77T or deletions Del156-157 or Del157-158.

In some embodiments, the encoded mutant S protein based on VOC lineage B.1.1.529/BA.1 (Omicron) comprises one or more of the following mutations: A67V, Del69-70 (HV), T951, G142D, Del143-145 (VYY), Del211 (N), L212I, Ins214 (EPE), G339D, S371 L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F.

In some embodiments, the encoded mutant S protein based on VOC lineage BQ.1 (Omicron) comprises one or more of the following mutations: T191, Del24-26 (LPS), A27S, Del69-70 (HV), G142D, V213G, G339D, S371F, S373P, S375F, T376A, D405N, R408S, K417N, N440K, K444T, L452R, N460K, S477N, T478K, E484A, F486V, Q493Q, Q498R, N501Y, Y505H, D614G, H655Y, N679K, P681H, N764K, D796Y, Q954H, and N969K.

In some embodiments, the encoded mutant S protein based on VOC lineage BQ.1.1 (Omicron) comprises one or more of the following mutations: T191, Del24-26 (LPS), A27S, Del69-70 (HV), G142D, V213G, G339D, R346T, S371F, S373P, S375F, T376A, D405N, R408S, K417N, N440K, K444T, L452R, N460K, S477N, T478K, E484A, F486V, Q493Q, Q498R, N501Y, Y505H, D614G, H655Y, N679K, P681H, N764K, D796Y, Q954H, and N969K.

In some embodiments, the encoded mutant S protein based on VOC lineage XBB (Omicron) comprises one or more of the following mutations: T191, Del24-26 (LPS), A27S, V83A, G142D, Del144 (Y), H146Q, Q183E, V213E, G339H, R346T, L3681, S371F, S373P, S375F, T376A, D405N, R408S, K417N, N440K, V445P, G446S, N460K, S477N, T478K, E484A, F486S, F490S, R493Q, Q498R, N501Y, Y505H, D614G, H655Y, N679K, P681H, N764K, D796Y, Q954H, and N969K.

In some embodiments, the encoded mutant N protein based on VOC lineage B.1.351 (Beta) comprises a T2051 mutation.

In some embodiments, the encoded mutant N protein based on VOC lineage P.1 (Gamma) comprises one or more of the following mutations: P80R, R203K, and G204R.

In some embodiments, the encoded mutant N protein based on VOC lineage P.1 (Gamma) comprises one or more of the following mutations: P80R, R203K, and G204K.

In some embodiments, the encoded mutant N protein based on VOC lineage B.1.617.2 (Delta) comprises one or more of the following mutations: D63G, R203M, G215C, and D377Y.

In some embodiments, the encoded mutant N protein based on VOC lineage B.1.617.2 (Delta) comprises one or more of the following mutations: D63G, R203M, and D377Y.

In some embodiments, the encoded mutant N protein based on VOC lineage B.1.617.2 (Delta) comprises one or more of the following mutations: D63G, R203M, D377Y, and R385K.

In some embodiments, the encoded mutant N protein based on VOC lineage B.1.1.529/BA.1 (Omicron) comprises one or more of the following mutations: P13L, Del31-33 (ERS), R203K, G204R.

In some embodiments, the encoded mutant N protein based on VOC lineage BQ.1 (Omicron) comprises one or more of the following mutations: P13L, Del31-33 (ERS), E136D, R203K, G204R, and S413R.

In some embodiments, the encoded mutant N protein based on VOC lineage BQ.1.1 (Omicron) comprises one or more of the following mutations: P13L, Del31-33 (ERS), E136D, R203K, G204R, and S413R.

In some embodiments, the encoded mutant N protein based on VOC lineage XBB (Omicron) comprises one or more of the following mutations: P13L, Del31-33 (ERS), R203K, G204R, and S413R.

In some embodiments, the rsMVA vector used in the methods and compositions disclosed herein is used in a candidate vaccine composition referred to herein as sMVA-N/S (or COH04S1). COH04S1 is based on an rsMVA vector capable of expressing S and N antigens of SARS-CoV-2. MVA vectors have a robust safety record and are known for inducing humoral and cellular immune responses that provide long-term protection against several infectious diseases, including smallpox and cytomegalovirus. In a mouse model, robust immunogenicity of COH04S1 was demonstrated, and pre-clinical data in hamsters and non-human primates demonstrating protection from upper and lower respiratory tract infections following SARS-CoV-2 challenge.

A fully synthetic Modified Vaccinia Ankara (sMVA)-based vaccine platform is used to develop COH04S1, a multi-antigenic poxvirus-vectored SARS-CoV-2 vaccine that co-expresses full-length spike (S) and nucleocapsid (N) antigens. SEQ ID NO: 33 shows the sequence of COH04S1. The DNA and protein sequences for the S antigen of COH04S1 are represented by SEQ ID NOs: 5 and 6 in the Sequence Listing, and the DNA and protein sequences for the N antigen of COH04S1 are represented by SEQ ID NOs: 7 and 8 in the Sequence Listing. Additional constructs (e.g., sMVA-S/N, sMVA-S, sMVA-N) are also disclosed in International Application Publication No. WO 2021/236550, which is hereby incorporated by reference as if fully disclosed herein. In some embodiments, the sMVA-based vaccine comprises or consists of a nucleotide sequence set forth in SEQ ID NO: 33, or a nucleotide sequence that is at least about 80% identical (e.g., at least about 80%, at least about 85%, at least about 90%, at least about 95%, at least about 96%, at least about 97%, at least about 98%, at least about 99%, or about 100% identical) to the nucleotide sequence set forth in SEQ ID NO: 33.

In patients who have received HCT and B cell directed therapies (such as CD19, BCMA, or CD22-directed CAR-T cell therapies), inactivated vaccines have generally shown low incremental risks and have not caused or worsened graft-versus-host disease (GVHD); thus, inactivated vaccines are generally started after 3-6 months. Most experts recommend vaccination as long as the vaccine is safe for use, even if the expected protection rate is lower than the general population. At the end January 2021, the National Comprehensive Cancer Network® (NCCN®) released preliminary guidelines on COVID-19 vaccination in cancer patients. NCCN® indicated to prioritize vaccinating patients with active cancer on treatment (including hematopoietic and cellular therapy), those planned to start treatment and those immediately post treatment. There is no data on timing of vaccine administration, nonetheless NCCN® preliminarily recommends starting at least 3 months post HCT and cellular therapy.

The candidate vaccine is based on a synthetic attenuated modified vaccinia Ankara (MVA) vector expressing spike (S) and nucleocapsid (N) antigens of SARS-CoV-2. MVA vectors are used because they are known for inducing humoral and cellular immune responses that provide long-term protection against a number of infectious diseases, including smallpox and cytomegalovirus (CMV).

Although non-pathogenic and highly attenuated, MVA-based vaccines maintain high immunogenicity as demonstrated in various animal models and clinically in humans (6). In the late phase of the smallpox eradication campaign, MVA was used as a priming vector for the replication competent vaccinia-based vaccine in over 120,000 individuals in Germany, and no AE were reported. Since then, MVA has been used to develop a smallpox vaccine that is stored in the US Strategic National Stockpile in case of a smallpox outbreak.

Triplex, a MVA vectored CMV vaccine was specifically developed at City of Hope (COH) for HCT recipients at high risk for CMV sequalae. MVA was known to be highly tolerable and immunogenic when used in HCT recipients. Triplex safely induced robust and long-lasting T cell responses when tested first in healthy adults, and subsequently in immunosuppressed CMV seropositive HCT recipients, in whom significantly reduced clinically relevant CMV viremia. HCT recipients received two injections of Triplex early post-HCT on day 28 and 56 post-HCT, at a dose of 5.1×10⁸ pfu/mL, in a multicenter efficacy phase 2 trial. Overall, few adverse events (AE) have been observed in trials with adult and pediatric transplant recipients (NCT03354728, NCT03560752, and NCT04060277 studies performed at COH and NCT03383055 in Minnesota), and this demonstrates safety and tolerability of the MVA-based vaccine for hematology patients.

Based on safety records, durability and immunogenicity of MVA vectored vaccines in immunocompromised HCT recipients even early post-HCT when immune reconstitution is still incomplete, COH04S1 may be a valid SARS-CoV-2 candidate vaccine for patients with hematology malignancies who have received cellular therapy at least about 28 days or about 1 month previously, when immunocompetence is increased.

COH04S1, a poxvirus vectored SARS-CoV-2 vaccine that expresses SARS-CoV-2 spike (S) and nucleocapsid (N) proteins, uses the same MVA platform and is tested for the prevention of COVID-19 in healthy adults and immunosuppressed hematology patients.

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Additional Embodiments

In embodiments, disclosed herein are methods of vaccinating or protecting a subject against coronavirus disease 2019 (COVID-19) caused by a coronavirus infection in a subject comprising administering to the subject a composition comprising a recombinant synthetic MVA (rsMVA) vector comprising or capable of expressing one or more DNA sequences encoding a spike (S) protein and a nucleocapsid (N) protein or variants or mutants thereof, wherein the subject is a blood cancer patient that has been treated for a hematological malignancy with a cellular therapy.

In embodiments, disclosed herein are methods of preventing coronavirus infection in a subject comprising administering to the subject a composition comprising a recombinant synthetic MVA (rsMVA) vector comprising or capable of expressing one or more DNA sequences encoding a spike (S) protein and a nucleocapsid (N) protein or variants or mutants thereof, wherein the subject is a blood cancer patient that has been treated for a hematological malignancy with a cellular therapy.

In embodiments, disclosed herein are methods of preventing or reducing the severity of COVID-19 caused by coronavirus infection in a subject comprising administering to the subject a composition comprising a recombinant synthetic MVA (rsMVA) vector comprising or capable of expressing one or more DNA sequences encoding a spike (S) protein and a nucleocapsid (N) protein or variants or mutants thereof, wherein the subject is a blood cancer patient that has been treated for a hematological malignancy with a cellular therapy.

In embodiments, disclosed herein are methods of treating COVID-19 caused by coronavirus infection in a subject comprising administering to the subject a composition comprising a recombinant synthetic MVA (rsMVA) vector comprising or capable of expressing one or more DNA sequences encoding a spike (S) protein and a nucleocapsid (N) protein or variants or mutants thereof, wherein the subject is a blood cancer patient that has been treated for a hematological malignancy with a cellular therapy.

In embodiments, disclosed herein are uses of a composition for preventing or reducing the severity of COVID-19 caused by a coronavirus infection in a subject, wherein the composition comprises an rsMVA vector comprising or capable of expressing one or more DNA sequences encoding the S protein and the N protein or variants or mutants thereof, and wherein the subject is a blood cancer patient that has been treated for a hematological malignancy with a cellular therapy.

In embodiments, disclosed herein are uses of a composition for the manufacture of a medicament for the treatment of COVID-19 caused by coronavirus infection in a subject, wherein the composition comprises an rsMVA vector comprising or capable of expressing one or more DNA sequences encoding the S protein and the N protein or variants or mutants thereof, and wherein the subject is a blood cancer patient that has previously been treated for a hematological malignancy with a cellular therapy.

In embodiments, the composition is administered to the subject.

In embodiments, disclosed herein are compositions for use as a medicament, wherein the composition comprises an rsMVA vector comprising or capable of expressing one or more DNA sequences encoding the S protein and the N protein or variants or mutants thereof.

In embodiments, the compositions are for use in the treatment of COVID-19 caused by coronavirus infection.

In embodiments, disclosed herein are compositions for use in the treatment of COVID-19 caused by coronavirus infection, wherein the composition comprises an rsMVA vector comprising or capable of expressing one or more DNA sequences encoding the S protein and the N protein or variants or mutants thereof.

In embodiments, the composition is administered to a subject, wherein the subject is a blood cancer patient that has been treated for a hematological malignancy with a cellular therapy.

In embodiments, disclosed herein are methods of vaccinating or protecting a subject against coronavirus disease 2019 (COVID-19) caused by a coronavirus infection in a subject comprising administering to the subject a composition comprising a recombinant synthetic MVA (rsMVA) vector comprising or capable of expressing one or more DNA sequences encoding a spike (S) protein and a nucleocapsid (N) protein or variants or mutants thereof, wherein the subject is a blood cancer patient who has had or is likely to have a poor immune response to a different COVID-19 vaccination.

In embodiments, disclosed herein are methods of preventing coronavirus infection in a subject comprising administering to the subject a composition comprising a recombinant synthetic MVA (rsMVA) vector comprising or capable of expressing one or more DNA sequences encoding a spike (S) protein and a nucleocapsid (N) protein or variants or mutants thereof, wherein the subject is a blood cancer patient who has had or is likely to have a poor immune response to a different COVID-19 vaccination.

In embodiments, disclosed herein are methods of preventing or reducing the severity of COVID-19 caused by coronavirus infection in a subject comprising administering to the subject a composition comprising a recombinant synthetic MVA (rsMVA) vector comprising or capable of expressing one or more DNA sequences encoding the Spike (S) protein and the Nucleocapsid (N) protein or variants or mutants thereof, wherein the subject is a blood cancer patient who has had or is likely to have a poor immune response to a different COVID-19 vaccination.

In embodiments, disclosed herein are methods of treating COVID-19 caused by a coronavirus infection in a subject comprising administering to the subject a composition comprising a recombinant synthetic MVA (rsMVA) vector comprising or capable of expressing one or more DNA sequences encoding the Spike (S) protein and the Nucleocapsid (N) protein or variants or mutants thereof, wherein the subject is a blood cancer patient who has had or is likely to have a poor immune response to a different COVID-19 vaccination.

In embodiments, disclosed herein are uses of a composition for preventing or reducing the severity of COVID-19 caused by a coronavirus infection in a subject, wherein the composition comprises an rsMVA vector comprising or capable of expressing one or more DNA sequences encoding the S protein and the N protein or variants or mutants thereof, and wherein the subject is a blood cancer patient who has had or is likely to have a poor immune response to a different COVID-19 vaccination.

In embodiments, disclosed herein are uses of a composition for the manufacture of a medicament for the treatment of COVID-19 caused by a coronavirus infection in a subject, wherein the composition comprises an rsMVA vector comprising or capable of expressing one or more DNA sequences encoding the S protein and the N protein or variants or mutants thereof, and wherein the subject is a blood cancer patient who has had or is likely to have a poor immune response to a different COVID-19 vaccination.

In embodiments, the composition is administered to the subject.

In embodiments, disclosed herein are compositions for use as a medicament, wherein the composition comprises an rsMVA vector comprising or capable of expressing one or more DNA sequences encoding the S protein and the N protein or variants or mutants thereof.

In embodiments, the compositions are for use in the treatment of COVID-19 caused by a coronavirus infection.

In embodiments, disclosed herein are compositions for use in the treatment of COVID-19 caused by a coronavirus infection, wherein the composition comprises an rsMVA vector comprising or capable of expressing one or more DNA sequences encoding the S protein and the N protein or variants or mutants thereof.

In embodiments, the composition is administered to a subject, wherein the subject is a blood cancer patient who has had or is likely to have a poor immune response to a different COVID-19 vaccination.

In embodiments, the cellular therapy is selected from the group consisting of an autologous hematopoietic cell transplant, an allogeneic hematopoietic cell transplant, a chimeric antigen receptor (CAR) T cell therapy, and a combination thereof. In embodiments, the cellular therapy is an autologous hematopoietic cell transplant. In embodiments, the cellular therapy is an allogeneic hematopoietic cell transplant. In embodiments, the cellular therapy is a CAR T cell therapy.

In embodiments, the subject received the cellular therapy at least 1 week, at least 2 weeks, at least 3 weeks, at least 4 weeks, at least 1 month, at least 2 months, at least 3 months, at least 4 months, at least 4 months, at least 5 months, at least 6 months, at least 7 months, at least 8 months, at least 9 months, at least 10 months, at least 11 months, or at least 12 months prior to administration of the composition. In embodiments, the subject received the cellular therapy at least 1 week prior to administration of the composition. In embodiments, the subject received the cellular therapy at least 2 weeks prior to administration of the composition. In embodiments, the subject received the cellular therapy at least 3 weeks prior to administration of the composition. In embodiments, the subject received the cellular therapy at least 4 weeks prior to administration of the composition. In embodiments, the subject received the cellular therapy at least 1 month prior to administration of the composition. In embodiments, the subject received the cellular therapy at least 2 months prior to administration of the composition. In embodiments, the subject received the cellular therapy at least 3 months prior to administration of the composition. In embodiments, the subject received the cellular therapy at least 4 months prior to administration of the composition. In embodiments, the subject received the cellular therapy at least 5 months prior to administration of the composition. In embodiments, the subject received the cellular therapy at least 6 months prior to administration of the composition. In embodiments, the subject received the cellular therapy at least 7 months prior to administration of the composition. In embodiments, the subject received the cellular therapy at least 8 months prior to administration of the composition. In embodiments, the subject received the cellular therapy at least 9 months prior to administration of the composition. In embodiments, the subject received the cellular therapy at least 10 months prior to administration of the composition. In embodiments, the subject received the cellular therapy at least 11 months prior to administration of the composition. In embodiments, the subject received the cellular therapy at least 12 months prior to administration of the composition.

In embodiments, the subject received the cellular therapy between 1 week and 6 months, between 6 months and 12 months, or more than 12 months prior to administration of the composition. In embodiments, the subject received the cellular therapy between 1 week and 6 months prior to administration of the composition. In embodiments, the subject received the cellular therapy between 6 months and 12 months prior to administration of the composition. In embodiments, the subject received the cellular therapy more than 12 months prior to administration of the composition.

In embodiments, the coronavirus infection is caused by a variant of concern (VOC) including B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In embodiments, the composition is administered to the subject by intramuscular injection or intranasal administration (e.g., instillation). In embodiments, the composition is administered to the subject by intramuscular injection. In embodiments, the composition is administered to the subject by intranasal administration. In some embodiments, the composition is administered to the subject by intradermal injection. In some embodiments, the composition is administered to the subject by scarification.

In embodiments, the composition is administered to the subject in a single dose, two doses, three doses, four doses, or more than four doses. In embodiments, the composition is administered to the subject in a single dose. In embodiments, the composition is administered to the subject in two doses. In embodiments, the composition is administered to the subject in three doses. In embodiments, the composition is administered to the subject in four doses. In embodiments, the composition is administered to the subject in more than four doses.

In embodiments, the composition is administered to the subject in a prime dose followed by one or more booster doses.

In embodiments, the interval between the prime dose and the first booster dose is about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, or about 16 weeks. In embodiments, the interval is about 3 weeks. In embodiments, the interval is about 4 weeks. In embodiments, the interval is about 5 weeks. In embodiments, the interval is about 6 weeks. In embodiments, the interval is about 7 weeks. In embodiments, the interval is about 8 weeks. In embodiments, the interval is about 9 weeks. In embodiments, the interval is about 10 weeks. In embodiments, the interval is about 11 weeks. In embodiments, the interval is about 12 weeks. In embodiments, the interval is about 13 weeks. In embodiments, the interval is about 14 weeks. In embodiments, the interval is about 15 weeks. In embodiments, the interval is about 16 weeks.

In embodiments, the prime dose is between 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose. In embodiments, the prime dose is about 1.0×10⁷ PFU/dose. In embodiments, the prime dose is about 1.5×10⁷ PFU/dose. In embodiments, the prime dose is about 2.0×10⁷ PFU/dose. In embodiments, the prime dose is about 2.5×10⁷ PFU/dose. In embodiments, the prime dose is about 3.0×10⁷ PFU/dose. In embodiments, the prime dose is about 3.5×10⁷ PFU/dose. In embodiments, the prime dose is about 4.0×10⁷ PFU/dose. In embodiments, the prime dose is about 4.5×10⁷ PFU/dose. In embodiments, the prime dose is about 5×10⁷ PFU/dose. In embodiments, the prime dose is about 5.5×10⁷ PFU/dose. In embodiments, the prime dose is about 6×10⁷ PFU/dose. In embodiments, the prime dose is about 6.5×10⁷ PFU/dose. In embodiments, the prime dose is about 7×10⁷ PFU/dose. In embodiments, the prime dose is about 7.5×10⁷ PFU/dose. In embodiments, the prime dose is about 8×10⁷ PFU/dose. In embodiments, the prime dose is about 8.5×10⁷ PFU/dose. In embodiments, the prime dose is about 9×10⁷ PFU/dose. In embodiments, the prime dose is about 9.5×10⁷ PFU/dose. In embodiments, the prime dose is about 1×10⁸ PFU/dose. In embodiments, the prime dose is about 1.5×10⁸ PFU/dose. In embodiments, the prime dose is about 2×10⁸ PFU/dose. In embodiments, the prime dose is about 2.5×10⁸ PFU/dose. In embodiments, the prime dose is about 3×10⁸ PFU/dose. In embodiments, the prime dose is about 3.5×10⁸ PFU/dose. In embodiments, the prime dose is about 4×10⁸ PFU/dose. In embodiments, the prime dose is about 4.5×10⁸ PFU/dose. In embodiments, the prime dose is about 5×10⁸ PFU/dose.

In embodiments, the booster dose is between 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose. In embodiments, the booster dose is about 1.0×10⁷ PFU/dose. In embodiments, the booster dose is about 1.5×10⁷ PFU/dose. In embodiments, the booster dose is about 2.0×10⁷ PFU/dose. In embodiments, the booster dose is about 2.5×10⁷ PFU/dose. In embodiments, the booster dose is about 3.0×10⁷ PFU/dose. In embodiments, the booster dose is about 3.5×10⁷ PFU/dose. In embodiments, the booster dose is about 4.0×10⁷ PFU/dose. In embodiments, the booster dose is about 4.5×10⁷ PFU/dose. In embodiments, the booster dose is about 5×10⁷ PFU/dose. In embodiments, the booster dose is about 5.5×10⁷ PFU/dose. In embodiments, the booster dose is about 6×10⁷ PFU/dose. In embodiments, the booster dose is about 6.5×10⁷ PFU/dose. In embodiments, the booster dose is about 7×10⁷ PFU/dose. In embodiments, the booster dose is about 7.5×10⁷ PFU/dose. In embodiments, the booster dose is about 8×10⁷ PFU/dose. In embodiments, the booster dose is about 8.5×10⁷ PFU/dose. In embodiments, the booster dose is about 9×10⁷ PFU/dose. In embodiments, the booster dose is about 9.5×10⁷ PFU/dose. In embodiments, the booster dose is about 1×10⁸ PFU/dose. In embodiments, the booster dose is about 1.5×10⁸ PFU/dose. In embodiments, the booster dose is about 2×10⁸ PFU/dose. In embodiments, the booster dose is about 2.5×10⁸ PFU/dose. In embodiments, the booster dose is about 3×10⁸ PFU/dose. In embodiments, the booster dose is about 3.5×10⁸ PFU/dose. In embodiments, the booster dose is about 4×10⁸ PFU/dose. In embodiments, the booster dose is about 4.5×10⁸ PFU/dose. In embodiments, the booster dose is about 5×10⁸ PFU/dose.

In embodiments, the booster dose is in a dosage the same as the prime dose.

In embodiments, the booster dose is in a dosage lower than the prime dose.

In embodiments, the subject suffers from or previously suffered from a hematological malignancy. In embodiments, the hematological malignancy is a B-cell hematological malignancy.

In embodiments, the subject suffers from or previously suffered from a B-cell lymphoid malignancy selected from the group consisting of chronic lymphocytic leukemia (CLL), B-cell non-Hodgkin lymphoma (B-NHL), Hodgkin lymphoma, B-cell acute lymphoblastic leukemia (ALL), and multiple myeloma. In embodiments, the B-cell lymphoid malignancy is CLL. In embodiments, the B-cell lymphoid malignancy is B-NHL. In embodiments, the B-cell lymphoid malignancy is Hodgkin lymphoma. In embodiments, the B-cell lymphoid malignancy is B-cell ALL. In embodiments, the B-cell lymphoid malignancy is multiple myeloma.

In embodiments, the subject has previously received one or more SARS-CoV-2 vaccines. In some embodiments, the previously received SARS-CoV-2 vaccine is an mRNA-, adenovirus-, or protein-based vaccine. In some embodiments, the previously received SARS-CoV-2 vaccine is Pfizer-BioNTech COVID-19 vaccine (BNT162b2), Moderna COVID-19 vaccine (mRNA-1273), Janssen COVID-19 vaccine (Ad26.COV2.S), Novavax COVID-19 vaccine adjuvanted, or Oxford-AstraZeneca ChAdOx1 nCoV-19 vaccine (AZD1222). In some embodiments, the previously received SARS-CoV-2 vaccine comprises an S antigen or a coding sequence for an S antigen only.

In embodiments, the subject developed poor immune response to the previous vaccination.

In embodiments, the subject received the previous vaccination at least 1 month, at least 2 months, at least 3 months, at least 4 months, at least 4 months, at least 5 months, at least 6 months, at least 7 months, at least 8 months, at least 9 months, at least 10 months, at least 11 months, or at least 12 months prior to administration of the rsMVA composition. In embodiments, the subject received the previous vaccination at least 1 month prior to administration of the rsMVA composition. In embodiments, the subject received the previous vaccination at least 2 months prior to administration of the rsMVA composition. In embodiments, the subject received the previous vaccination at least 3 months prior to administration of the rsMVA composition. In embodiments, the subject received the previous vaccination at least 4 months prior to administration of the rsMVA composition. In embodiments, the subject received the previous vaccination at least 5 months prior to administration of the rsMVA composition. In embodiments, the subject received the previous vaccination at least 6 months prior to administration of the rsMVA composition. In embodiments, the subject received the previous vaccination at least 7 months prior to administration of the rsMVA composition. In embodiments, the subject received the previous vaccination at least 8 months prior to administration of the rsMVA composition. In embodiments, the subject received the previous vaccination at least 9 months prior to administration of the rsMVA composition. In embodiments, the subject received the previous vaccination at least 10 months prior to administration of the rsMVA composition. In embodiments, the subject received the previous vaccination at least 11 months prior to administration of the rsMVA composition. In embodiments, the subject received the previous vaccination at least 12 months prior to administration of the rsMVA composition.

### EXAMPLES

### Example 1. Materials and Methods for Studies of COH04S1 Vaccination in Immunocompromised Individuals

Examples 2 and 3, describing studies of COH04S1 vaccinations in immunocompromised individuals, employed materials and methods as described below.

COH04S1 generation: Three unique synthetic sub-genomic sMVA fragments were designed based on the MVA genome sequence published previously (9). The entire sMVA was cloned as three fragments in Escherichia coli as bacterial artificial chromosome (BAC) clones using highly efficient BAC recombination techniques. The full-length SARS-CoV-2 S and N antigen sequences were inserted into commonly used MVA insertion sites located at different positions within the three sMVA fragments. The sMVA SARS-CoV-2 virus was reconstituted with fowl pox virus (FPV) as a helper virus upon co-transfection of the DNA plasmids into BHK-21 cells, which are non-permissive for FPV (10). The virus stocks were propagated on chicken embryo fibroblast (CEF) cells, which are commonly used for MVA vaccine production. The infected CEF cells were grown further, and the infected cells were harvested, freeze-thawed and stored at −80° C., then titrated on CEF cells to grow expanded virus stocks. To transition vaccine candidates into clinical production, viruses were plaque purified and clones expanded. Clone COH04S1 was selected for clinical vaccine production and the clinical stock used in this trial was produced on CEF at the COH Center for Biomedicine and Genetics (CBG).

SARS-CoV-2-specific IgA, IgG, and IgM measured in serum by ELISA: To evaluate humoral immunity with the COH04S1 vaccine, SARS-CoV-2 specific antibodies, including IgA, IgG, and IgM, in serum were measured by ELISA at various timepoints. The ELISA test was developed by and conducted in the Diamond Laboratory at COH, Dept. of Hematology & Hematopoietic Cell Transplantation. The assay identifies SARS-CoV-2 antibodies specific for the S receptor-binding domain (RBD) that interacts with ACE2 on the surface of the cells; the N protein that is one of the first B cell targets, during the initial phase of the SARS-CoV-2 infection; and the open reading frame (ORF)3b and 8 that are accurate serological markers of early and late SARS-CoV-2 infection (23, 25). The qualitative assays, based on previously established protocols (26), were developed to investigate Spike subunit 1 (S1)-, N-ORF3b- and 8-specific antibodies of the IgG, IgM and IgA subclasses in serum. Pools of SARS-CoV-2 convalescent serum or SARS-CoV-2 negative serum were used as a positive- and negative-controls (University of California at San Diego), respectively. End-point binding antibody titers were expressed as the reciprocal of the last sample dilution to give an OD value above the cut-off (26). Antibody levels in recipients were graphed on a time plot and compared to baseline level in donors.

SARS-CoV-2-specific neutralizing antibodies: Evaluation of SARS-CoV-2 neutralizing antibody titers in serum samples of COH04S1 vaccinated volunteers were performed at various timepoints. SARS-CoV-2 lentiviral-pseudovirus expressing the Spike antigen and infecting 293T cell lines engineered to express ACE2 is used (22). Spike incorporation into the pseudovirus was verified and quantified by Western blot using Spike-specific antibodies and by ELISA (18).

Th1 vs Th2 polarization: To evaluate the Th1 vs Th2 polarization of immune responses, dual fluorescence ELISPOT assay was performed to detect and quantify cells secreting IFNγ and IL-4. Briefly, isolated PBMCs are stimulated with Spike and Nucleocapsid peptide libraries (15-mers with 11 aa overlap) using fluorospot plates coated with IFNγ and IL-4 capture antibodies. Following 48 h co-incubation, the plates were washed, and IFNγ and IL-4 detection antibodies followed by fluorophore conjugates were added. The plates were read and analyzed with a fluorescent ELISPOT reader and number of spots after stimulation expressed following subtraction of background from unstimulated samples. As an exploratory endpoint, in selected samples, a cytokine-based cytofluorimetric analysis (ICS) was performed to analyzed multiple Th1 and Th2 cytokines. PBMCs (1-2×10⁶) were stimulated for 16 hours with SARS-CoV-2-S or SARS-CoV-2-N overlapping peptide libraries (15-mers with 11 aa overlap). Lymphocytes were stained with viability dye and surface stained with antibodies to CD3, CD8 and CD4. After fixing and permeabilization, the cells were stained intracellularly with antibodies against IFNγ, TNF-alpha, IL-2, IL-4, IL6, IL-13. After washing, the cells were acquired using BD FACS Celesta Cell Analyzer and analyzed with FlowJo software.

SARS-CoV-2-specific T-cell responses and evolution of activated/cycling and memory phenotype markers on the surface of antigens-specific T cells: Cellular immunity to SARS-CoV-2-S and —N, major domains of antiviral T cell immunity was investigated in PBMCs of COH04S1 vaccinated subjects, using multiparameter flow cytometry as previously disclosed (17). Frequencies of T lymphocyte precursors responsive to SARS-CoV-2-S or SARS-CoV-2-N overlapping peptide libraries were longitudinally monitored. In vaccine responders, SARS-CoV-2 specific T cells are further evaluated by measuring levels (17) of CD137 surface marker expressed on CD3+CD8+ and CD3+CD4+T cells stimulated for 24 hours with either SARS-CoV-2-S or SARS-CoV-2-N overlapping peptide libraries. CD137 is expressed only on recently activated T cells, and its expression correlates with functional activation of T cells (27). Measurements of CD137 levels were combined with immunophenotyping studies, by using antibodies to CD28 and CD45RA cell surface markers to assess and identify memory phenotype profiles percentage of effector memory (TEM and TEMRA), central memory (TCM) and naïve SARS-CoV-2-S or SARS-CoV-2-N specific T cells (7). The activated/cycling phenotype was assessed by using the CD38, HLA-DR, Ki67 and PD1 surface markers (24). Approximately 300,000 events per sample were acquired on a Gallios flow cytometer and analyzed by Kaluza software.

Pseudovirus production: SARS-CoV-2 pseudovirus was produced using a plasmid lentiviral system based on pALD-gag-pol, pALD-rev, and pALD-GFP (Aldevron). Plasmid pALD-GFP was modified to express Firefly luciferase (pALD-Fluc). Plasmid pCMV3-S(Sino Biological VG40589-UT) was utilized and modified to express SARS-CoV-2 Wuhan-Hu-1 S with D614G modification. Customized gene sequences cloned into pTwist-CMV-BetaGlobin (Twist Biosciences) were used to express SARS-CoV-2 VOC-specific S variants. All S antigens were expressed with C-terminal 19aa deletion. A transfection mixture was prepared 1 ml OptiMEM that contained 30 μl of TransIT-LT1 transfection reagent (Mirus MIR2300) and 6 μg pALD-Fluc, 6 μg pALD-gag-pol, 2.4 μg pALD-rev, and 6.6 μg S expression plasmid. The transfection mix was added to 5×10⁶ HEK293T/17 cells (ATCC CRL11268) seeded the day before in 10 cm dishes and the cells were incubated for 72 h at 37° C. Supernatant containing pseudovirus was harvested and frozen in aliquots at −80° C. Lentivirus was titrated using the Lenti-XTM p24 Rapid Titer Kit (Takara) according to the manufacturer's instructions.

### Example 2. A Phase 2 Randomized, Multi-Center, Observer-Blind Study of COH04S1 Versus EUA SARS-COV-2 Vaccine in Patients Post Cellular Therapy for Hematological Malignancies

### Study Design

The study was designed to evaluate the biological activity of COH04S1 compared to Pfizer vaccine using observer blinded randomization. Since the volumes and handling of the Pfizer and COH04S1 vaccinations were noticeably different, a double-blind study was not feasible; however, patients, physicians, and laboratory personnel analyzing the data were blinded to the participant study arm. The optimal timing post-transplant or CAR T cell therapies was assessed for safely eliciting an effective SARS-CoV-2 humoral and cellular immune response in HCT and CAR T cell recipients vaccinated with COH04S1 vs Pfizer vaccine. Detailed demographic characteristics of the participants are listed in Table 2.

TABLE 2
Demographic Characteristics

| | Allo | | Auto | | CART | | Total | |
|---|---|---|---|---|---|---|---|---|
| | (N = 7) | | (N = 5) | | (N = 1) | | (N = 13) | |
| Age at consent, years | | | | | | | | |
| Median | 55 | | 52 | | 73 | | 55 | |
| Interquartile range | 44, 65 | | 46, 67 | | 73, 73 | | 46, 66 | |
| Range | (38-66) | | (41-69) | | (73-73) | | (38-73) | |
| Age at consent, years | | | | | | | | |
| 18-64 | 5 | (71.4%) | 3 | (60%) | 0 | (0%) | 8 | (61.5%) |
| 65+ | 2 | (28.6%) | 2 | (40%) | 1 | (100%) | 5 | (38.5%) |
| Gender | | | | | | | | |
| Female | 4 | (57.1%) | 4 | (80%) | 0 | (0%) | 8 | (61.5%) |
| Male | 3 | (42.9%) | 1 | (20%) | 1 | (100%) | 5 | (38.5%) |
| Ethnicity | | | | | | | | |
| Hispanic or Latino | 1 | (14.3%) | 1 | (20%) | 0 | (0%) | 2 | (15.4%) |
| Non-Hispanic or Non- | 5 | (71.4%) | 4 | (80%) | 0 | (0%) | 9 | (69.2%) |
| Latino | | | | | | | | |
| Non-disclosed | 1 | (14.3%) | 0 | (0%) | 1 | (100%) | 2 | (15.4%) |
| Caucasian | 5 | (71.4%) | 4 | (80%) | 1 | (100%) | 10 | (76.9%) |
| Black | 0 | (0%) | 1 | (20%) | 0 | (0%) | 1 | (7.7%) |
| Asian | 1 | (14.3%) | 0 | (0%) | 0 | (0%) | 1 | (7.7%) |
| NonDisclosed | 1 | (14.3%) | 0 | (0%) | 0 | (0%) | 1 | (7.7%) |
| TimeCohort | | | | | | | | |
| 3 −< 6 months | 4 | (57.1%) | 4 | (80%) | 0 | (0%) | 8 | (61.5%) |
| 6 −< 12 months | 3 | (42.9%) | 1 | (20%) | 1 | (100%) | 5 | (38.5%) |

(report generated on 15 Nov. 2022)

A further objective of the study was to test safety and immunogenicity of COH04S1 given as two doses vaccination course to patients post hematopoietic cell transplant (HCT) or chimeric antigen receptor (CAR)-T cell therapy for hematologic malignancies at least 3 months prior. Two injections (or four per the amended protocol which applied to 5 patients) of COH04S1 vaccine will be administered at 2.5×10⁸ PFU/dose by intramuscular (IM) injection in the upper arm, 4 weeks apart compared to standard of care (SOC) vaccine (Comirnaty or similar). Primary objectives are safety and at least a 3-fold increase in neutralizing antibodies and/or SAR-CoV-2 S specific IFNγ levels 28 days after the second injection. Accrual is set to 240 subjects. A safety lead in segment (single arm) with 3 parallel treatment groups in 3+3 design and open label COH04S1 vaccination will precede the randomized blinded phase 2 segment. The safety lead in segment will enroll 6 autologous transplant (AUTO), allogeneic transplant (ALLO), and CAR-T cell therapy patients. If no safety concerns arise, the randomized portion of each treatment group will be started and retrospectively stratified by time from transplant into 3<6, 6<12, and ≥12 months.

COH04S1, a multiantigen synthetic modified vaccinia Ankara (sMVA) vector that co-expresses Wuhan-Hu-1-based S and nucleocapsid (N) antigens, was developed. The N antigen was included in COH04S1 primarily based on the rationale to broaden the stimulation of T cells, which are known to be less susceptible to antigen variation than NAb and therefore considered a critical second line of defense to provide long-term protective immunity against SARS-CoV-2. COH04S1 afforded protection against SARS-CoV-2 ancestral virus and Beta and Delta variants in Syrian hamsters and non-human primates and was safe and immunogenic in a Phase 1 clinical trial in healthy adults. Importantly, T cell responses to both the S and N antigens elicited in COH04S1-vaccinated individuals maintained potent cross-reactivity to SARS-CoV-2 Delta and Omicron BA.1 variants for up to six months post-vaccination, whereas NAb responses elicited by COH04S1, as shown for other COVID-19 vaccines, decreased and conferred reduced neutralizing activity against Delta and Omicron BA.1 variants. COH04S1 is currently being tested in multiple Phase 2 clinical trials in healthy volunteers and in cancer patients.

Critical materials and reagents used are listed in Table 3.

TABLE 3
Critical Materials and Reagents

| Item | Manufacturer | Product ID/Lot # |
|---|---|---|
| Spike peptide library | Genscript | Custom order |
| Spike MegaPool (MP) | LJII | A gift from A. Grifoni/A. |
| | | Sette (LJII) |
| Nucleocapsid peptide library | Genscript | Custom made |
| Membrane peptide library | COH peptide core | Custom made |
| S1 + S2 | SinoBiological | 40589-V08B1 |
| N | SinoBiological | 40588-V08B |
| RBD | SinoBiological | 40592-V08H |
| Goat anti-human IgG-HRP | BioRad | 204005 |
| Natural Goat Serum NGS | Vector Labs | S-1000-500-1 |
| WHO reference panel | NIBSC | 20/268 |
| Hammerstein Casein | Thermo Scientific | J12840-22 |
| TMB-ELISA | Thermo Scientific | 34029 |
| Spike-D614G expression vector | Twist Bioscience | Custom order |
| Spike-Beta expression vector | Twist Bioscience | Custom order |
| Spike-Delta expression vector | Twist Bioscience | Custom order |
| Spike-Omicron BA.1 expression | Twist Bioscience | Custom order |
| vector | | |
| pALD-Lenti system | Aldevron | pALD-LentiEGFP-K |
| | | (modified to express |
| | | Luciferase) |
| | | pALD-GagPol-K |
| | | pALD-Rev-K |
| HEK293T/17 | ATCC | CRL11268 |
| TransIT-Lenti transfection reagent | Mirus | MIR6600 |
| Lenti-XTM p24 Rapid Titer Kit | Takara | |
| HEK293T-ACE2 cells | | A gift from J. Bloom |
| | | (Fred Hutchinson |
| | | Cancer Research |
| | | Center) |
| Viability Dye L/D Near IR | Invitrogen | L34975 |
| Anti-CD3 | Biolegend | 300440 |
| Anti-CD4 | BD | 566703 |
| Anti-CD8 | BD | 301040 |
| Anti-CD137 | Biolegend | 309810 |
| Anti-OX40 | Biolegend | 350012 |
| Anti-CD69 | BD | 555531 |
| Anti-CD14/CD19 | Biolegend | 561391/561121 |
| Anti-CD45RA | Biolegend | 304156 |
| Anti-CCR7 | Biolegend | 353236 |
| Human Serum | BioIVT | HP1022HI |
| PHA | Roche | 11082132001 |
| Human IFN-g/IL-4 FluoroSpot Flex Kit | Mabtech | X-01A16B-10 |
| Benzonase Nuclease | Santa Cruz | SC-202391A |
| | Biotech | |

Clinical-grade COH04S1 produced by Applicant's GMP manufacturing facility using CEF cells was used for this Phase 2 clinical trial. Before each injection, COH04S1 was thawed and diluted with sterile diluent (phosphate-buffered saline with 7-5% lactose) to the appropriate dose of 2.5×10⁸ pfu.

COH04S1 was injected in two doses (or four in the amended protocol covering few patients before the original protocol was reinstated). After the safety lead in portion, the trial was randomized and blinded to the study participants, the data investigator(s) or data collector(s) and the data analyzer(s) to the vaccine administered (COH04S1 or SOC). Blood collection for serum and PBMC evaluation was carried out at screening and at days 28, 56, 120, 180, 270, and 365 post-vaccination.

A total of 258 cell therapy recipients comprised of 3 cohorts were enrolled: auto HCT, allo HCT, and CAR-T cell therapy recipients and were retrospectively stratified into 3 time interval cohorts: those who received cellular therapy in the last 3-<6 months, those who received cellular therapy in the last 6-<12 months, and those who received cellular therapy 12 months or greater.

Eligible patients were retrospectively stratified by time interval cohort and prospectively by type of cellular therapies (9 total strata) and 1:1 randomized into either COH04S1 or Pfizer vaccine. Each patient received 2 injections of 2.5×10⁸ PFU/dose of COH04S1 or Pfizer vaccine on days 0 (prime) and 28 (boost) and was followed for 1 year. The dose of COH04S1 was chosen based on experiences with other MVA-based vaccines (19-21). All adverse events were evaluated from first vaccination to 28 days after the second injection (expected to be day 35), and serious or unexpected adverse events at any time through one year. Rates of non-relapse mortality (NRM), severe GVHD, severe COVID-19 infection and UT/MOD were assessed at the End of Treatment (EOT) visit ˜28 days post last vaccination. Long-term assessment evaluations continued through 1 year after vaccination. At ˜ 28 days after the second injection, all fully vaccinated HCT and CAR T cell therapy recipients received the primary immunological assessment (PIA).

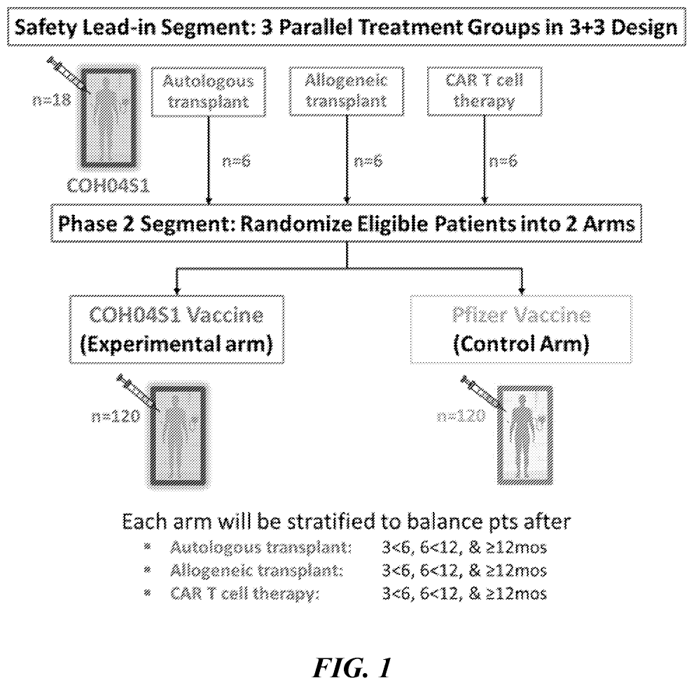

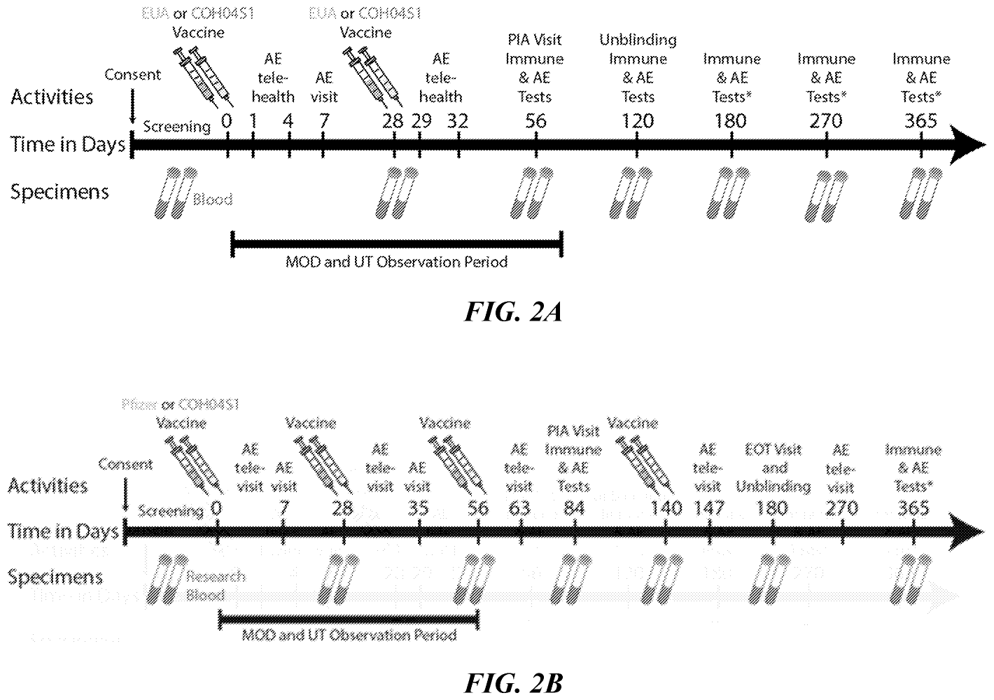

FIG. 1 shows the subject flow. Long-term assessment on evaluations continued through 365-days after vaccination. FIGS. 2A-2B show the treatment schema.

The immunologic activity and safety of the COH04S1 vaccine were evaluated in former recipients of cellular therapy for hematological malignancies. Participants received two IM injections (2.5×10⁸ PFU/dose) of either COH04S1 or a 2-dose Pfizer COVID-19 vaccine in the upper arm in the outpatient setting, 28 days apart, on Days 0 and 28 of the study (Table 4).

TABLE 4
Vaccine Type and Administration

| Vaccine | Injection 1 (day 0) | Injection 2 (day 28 + 7*) |
|---|---|---|
| COH04S1 | 2.5 × 10⁸ PFU/dose (IM) | 2.5 × 10⁸ PFU/dose (IM) |
| Pfizer | SOC dose | SOC dose |

*There was a 7-day window to allow for scheduling issues. Delay of up to 6 weeks was allowed.

All AEs were evaluated from the first vaccination to 28 days after the last injection (˜day 56). Long-term assessment with limited safety data collection and immunological response sampling continued through 365-days post-vaccination (first injection), with follow up at days 7, 90, 120, 180, and 365. Toxicity was graded according to the Common Terminology Criteria for Adverse Events (CTCAE) version 5.0.

A comprehensive and innovative panel of immune assays combined with safe and versatile virological tools was designed to characterize the COH04S1 vaccine induced SARS-CoV-2-specific adaptive immunity in this clinical trial. The integrated platform of immune- and pseudovirus-based methods included analytical multiparameter flow cytometry; qualitative in house developed ELISA, and neutralization assays based on a SARS-CoV-2 lentiviral-pseudovirus system, expressing the Spike antigen and infecting cell lines engineered to express ACE2 (18). The assay system with Spike antigen “pseudotyped” onto non-replicative lentiviral particles alleviates the biosafety-level-3 (BSL3) hazard associated with working directly with SARS-CoV-2 and allows a safer approach to assess sera neutralizing activity to SARS-CoV-2.

Humoral immunity (IgA, IgG, and IgM) in serum was assessed by ELISA. The neutralizing capability of the antibodies to prevent infection of a susceptible cell line was evaluated using a pseudo-type of the SARS-CoV-2 virus. To evaluate the Th1 vs Th2 polarization of immune responses, which has been observed in convalescing COVID-19 cases, a SARS-CoV-2-specific ELISPOT was performed to measure IFNγ and IL-4 cytokine levels, by using overlapping peptide libraries specific for SARS-CoV-2. Additionally, the following were evaluated: a) levels of antigen-specific T cell responses using overlapping peptide library specific for SARS-CoV-2; and b) functional activated/cycling and memory phenotype marker evolution on the surface of antigen specific T cells elicited as a result of the vaccination.

All subjects with intercurrent infections were tested for SARS-CoV-2 PCR assay, as a record any incidental COVID-19 infection during the study follow-up period, and the biological correlatives of infected subjects were compared with those uninfected, along with recording the severity of disease to evaluate for the potential of vaccine-induced disease enhancement.

The immune response measured at the PIA visit (28 days+7 post second injection) was the primary endpoint and was counted as positive if meeting any of the following criteria:
1. ≥3-fold increase in SARS-CoV-2-neutralizing antibodies over baseline or lower limit of quantification (LLOQ), whichever is higher;
2. ≥3-fold increase in SARS-CoV-2-IFNγ levels over baseline or LLOQ, whichever is higher.

The immune response was categorized as negative if none of the above defined criteria were met.

The primary statistical analysis compared the immune response at day 28 post the second injection between COH04S1 and Pfizer using a one-sided stratified CMH test. The point estimate and 95% CI were calculated per arm for immune response at day 28 post the second injection. Bar charts were generated to show the immune response rate by arm overall, and by arm and strata. All randomized participants were included in the primary analytic set. The comparison of the primary endpoint was based on intent-to-treat analysis.

Secondary analyses: Per protocol analysis was performed (1) comparison of the primary endpoint by the first vaccine participants actually received, (2) two injections were completed, (3) the primary endpoint was successfully measured. Continuous immune response markers were summarized by means or geometric means and standard deviations if the assumption of normal distribution was not violated. Repeated immune response measurements at the multiple time points were analyzed using generalized estimating equations (GEE) or mixed regression models. Scatterplots of immune response markers across time points by arm were generated to visualize the differences.

The primary analytic set of the safety data in both the safety lead-in and randomized phase 2 segments included subjects who received at least one injection of a vaccine. Descriptive statistics was used to summarize the safety profile. Tables or graphs were constructed to summarize solicited local and systemic adverse events, MOD, UT, NRM, and other safety endpoints by type of cellular therapy in the safety lead-in segment and by arm and each stratum in the randomized phase 2 segment.

### Study Analysis

SARS-CoV-2 binding antibodies. Binding antibody titers were evaluated by ELISA. ELISA plates were coated overnight with 1 μg/ml of S (S1+S2, 40589-V08B1, SinoBiological), RBD (40592-V08H, SinoBiological), or N (40588-V08B, SinoBiological) in coating buffer (1×PBS pH 7.4). Plates were washed 5 times with 250 μl/well PBST (PBS pH 7.4+0.1% Tween-20). Plates were blocked with 250 μl/well of assay diluent (154 mM NaCl/0.5% Casein/10 mM Tris-HCl/0.1% Tween-20 pH 7.6/8% NGS) for 2 hours at 37° C. Sample dilutions and WHO standards were prepared in assay diluent. Serum samples were diluted 1:150, 1:900, 1:4500, and 1:13500). WHO standards (High, Medium, Low S, Low) were initially diluted 1:9 and then further diluted 1:6. Sample dilutions and WHO standards were added to the plate (100 μl/well) after washing 5× and incubated wrapped in foil at 37° C. and 5% CO2. After washing 5×, 1:3,000 dilution of HRP-conjugated anti-human IgG secondary antibody was added and incubated for one additional hour at room temperature. After 7× washing, plates were developed using 1-Step Ultra TMB-ELISA for 3 (S), 4 (N), and 5 (RBD) minutes after which the reaction was stopped with 1M H2SO4. Plates were read at 450 nm wavelengths using FilterMax F3 microplate reader. Sample concentration expressed in BAU/ml was extrapolated from the standard curve obtained with the assigned WHO standard values transformed based on the assay dilutions (Table 5).

TABLE 5
WHO standards assigned values and values used
to create standard curve in FilterMax F3

| Antigen | Standard | Cat# | WHO BAU/ul | FilterMax BAU/ml |
|---|---|---|---|---|
| S | High | 20/150 | 832 | 616.296 |
| | Mid | 20/148 | 241 | 178 |
| | Low-S/High-N | 20/144 | 86 | 63.704 |
| | Low | 20/140 | 53 | 39.259 |
| RBD | High | 20/150 | 817 | 605.185 |
| | Mid | 20/148 | 205 | 151.852 |
| | Low-S/High-N | 20/144 | 66 | 48.889 |
| | Low | 20/140 | 45 | 33.33 |
| N | High | 20/150 | 713 | 528.148 |
| | Mid | 20/148 | 295 | 218.519 |
| | Low-S/High-N | 20/144 | 146 | 108.148 |
| | Low | 20/140 | 12 | 8.889 |

SARS-CoV-2 pseudovirus neutralization assay. Serum samples were heat inactivated, diluted using 2-fold serial dilutions in complete DMEM. Diluted serum samples were co-incubated overnight at 4° C. with pseudotyped luciferase lentiviral vector expressing SARS-CoV-2 Spike glycoprotein on the envelope in a poly-L-lysine coated 96-well plate. The amount of pseudovirus was pre-determined based on the target relative luciferase units (RLU) of each variant and ranged between 5×10⁵ and 2×10⁶. Next day, the 96 well plates were allowed to equilibrate to room temperature. HEK293T cells overexpressing ACE-2 receptor were then seeded at a density of 1×10⁵ cells/ml in complete DMEM containing 10 μg/ml of polybrene. The cells were incubated for 48 hours at 37° C. and 5% CO₂ atmosphere. Following incubation, media was aspirated, and the cells lysed in a shaker at room temperature using 40 μl/well of Luciferase Cell Culture Lysis Reagent. Cell lysates were transferred to white 96-well plates and Luciferase activity were measured by sequential injection of 100 μl/well of Luciferase Assay Reagent substrate. RLU were quantified using a microplate reader with injector at a 570 nm wavelength.

IFNγ/IL-4 ELISpot. FluoroSpot plates were prepared by adding 15 μl/well of 35% EtOH for less than a minute. Plates were washed 5× with 200 μl/well of sterile H2O. IFNγ and IL-4 capture antibodies were diluted to 15 μg/ml in sterile PBS and 100 μl/well of antibody were added in each well and incubated overnight at 4° C. PBMCs were thawed, and 1 ml RPMI with benzonase (50 U/ml) was added to the tube. Cells were transferred to a 15 ml conical pre-filled with 12 ml RPMI with benzonase (50 U/ml). Conicals were centrifuged at 300×G for 10 minutes. Media was aspirated and cells resuspended in 12 ml of fresh, warm media without benzonase. Conicals were centrifuged at 300×G for 10 minutes. Cells were resuspended in 2 ml RPMI medium and rested for 2 hours at 37° C./5% CO2. Coated plates were washed 5× with sterile PBS and 200 μl/well of CTL test medium added to each well and the plate incubated at 37° C./5% CO2 for at least 30 minutes. Conicals were centrifuged at 300×G for 10 minutes and resuspended in 1 ml CTL test medium. Cells were counted and resuspended to 3×10⁶ cells/ml in CTL test medium. Genscript Spike peptide library consisting of 316 peptides was divided into four sub-libraries: 1S1 (peptides 1-86), 2S1 (87-168), 1S2 (169-242, excluding peptide 173), 2S2 (243-316, excluding peptides 304-309). Peptide dilutions were prepared in CTL test media added with anti-CD28 0.2 μg/ml as shown in Table 6. 50 μl/well of peptide mix were added to the corresponding rows in the FluoroSpot plate. 50 μl/well of cell suspension (1.5×10⁵ cells) were added to the corresponding columns in the FluoroSpot plate. 5×10⁴ cells/wells were added to the PHA controls. Plates were wrapped in foil and incubated 37° C./5% CO2. After 40-42 hours, plates were washed 5× with PBS. IFNγ and IL-4 detection antibodies were diluted 200× with 0.1% BSA/PBS and sterile filtered (0.22 μm). 100 μl/well of detection antibody in CTL test medium were added to each well and incubated 2 hours at room temperature. Plates were washed 5× with PBS. Fluorophore-conjugated antibodies were diluted 200× in 0.1% BSA/PBS and sterile filtered (0.22 μm). 100 μl/well of detection antibody in CTL test medium were added to each well and incubated 1 hour at room temperature. Plates were washed 5× with PBS. 50 μl/well of fluorescence enhancer were added to each well and incubated 10 minutes at room temperature away from light. Fluorescence enhancer was removed by flicking the plate and plates were dried away from light under the airflow of a biological cabinet. Plates were scanned (490 nm and 550 nm wavelength) and analyzed using ImmunoSpot plate reader.

TABLE 6
Antibody Mix Composition

| # | Ab | Fluorochrome | Dilution |
|---|---|---|---|
| 1 | CD3 | FITC | 1:40 |
| 2 | CD45RA | PerCp | 1:20 |
| 3 | CD137 | APC | 1:40 |
| 4 | LIVE/DEAD | NearIR | 1:1000 |
| 5 | CD4 | BV421 | 1:50 |
| Dump channel | CD14 | V500 | 1:100 |
| Dump channel | CD19 | V500 | 1:100 |
| 7 | CD8 | BV605 | 1:100 |
| 8 | CD69 | PE | 1:10 |
| 9 | CCR7 | PE/Dazzle | 1:20 |
| 10 | OX40 | PE-Cy7 | 1:20 |

AIM and T cell memory markers. PBMCs were thawed and counted, and concentration adjusted to 10×10⁶ cells/ml using HR-5 media. 1 million cells (100 μl) were plated in 96-well plates and stimuli were added at a concentration of 2 μg/ml (2×) in 100 μl HR-5 media. Plates were incubated for 24 hours at 37° C./5% CO₂. After the stimulation, plates were spun at 2000 rpm at 4° C. for 5 min. In each well, 50 μl of antibody mix was added (Table 7) and incubated 15 minutes at room temperature in the dark. After incubation, 150 μl PBS were added in each well and plates were spun at 2000 rpm at 4° C. for 3 min. Plates were further washed with 150 μl PBS and spun at 2000 rpm at 4° C. for 3 min. Cells were resuspended in 250 μl of PBS and maintained at 4° C. until acquisition using Attune NxT cytofluorimeter.

TABLE 7
Stimuli Dilution

| | | | | Assay | |
|---|---|---|---|---|---|
| | Stock | Dilution | Dilution | Conc. | Volume/ |
| Stimulation | (mg/ml) | (ug/ml) | Factor | (ug/ml) | well (ul) |
| DMSO | | | 2 | | 50 |
| 1S1 | 1 | 2 | 2 | 1 | 50 |
| 2S1 | 1 | 2 | 2 | 1 | 50 |
| 1S2 | 1 | 2 | 2 | 1 | 50 |
| 2S2 | 1 | 2 | 2 | 1 | 50 |
| N | 1 | 2 | 2 | 1 | 50 |
| M | 1 | 2 | 2 | 1 | 50 |
| PHA | 5 | 10 | 2 | 5 | 50 |

Statistics. Statistical evaluation was pursued using GraphPad Prism (v8.3.0). Wilcoxon matched-pairs signed rank test was used to compare baseline values to post-vaccination values.

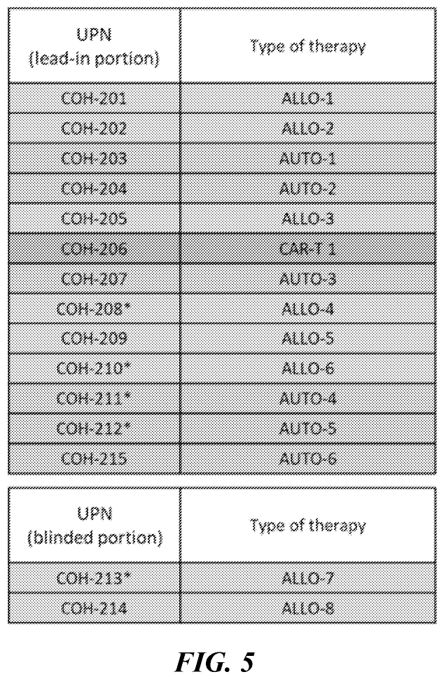

Immunogenicity Results. As of Nov. 7, 2022, 13 volunteers in the safety lead-in portion (6 ALLO, 6 AUTO, and 1 CAR-T) had received at least one dose of COH04S1. Of these, 8 had reached day 180 timepoint. Analysis is provided for a subgroup of samples. In the randomized portion, 2 ALLO patients had received at least one dose of vaccine (COH04S1 or Pfizer, FIG. 5).

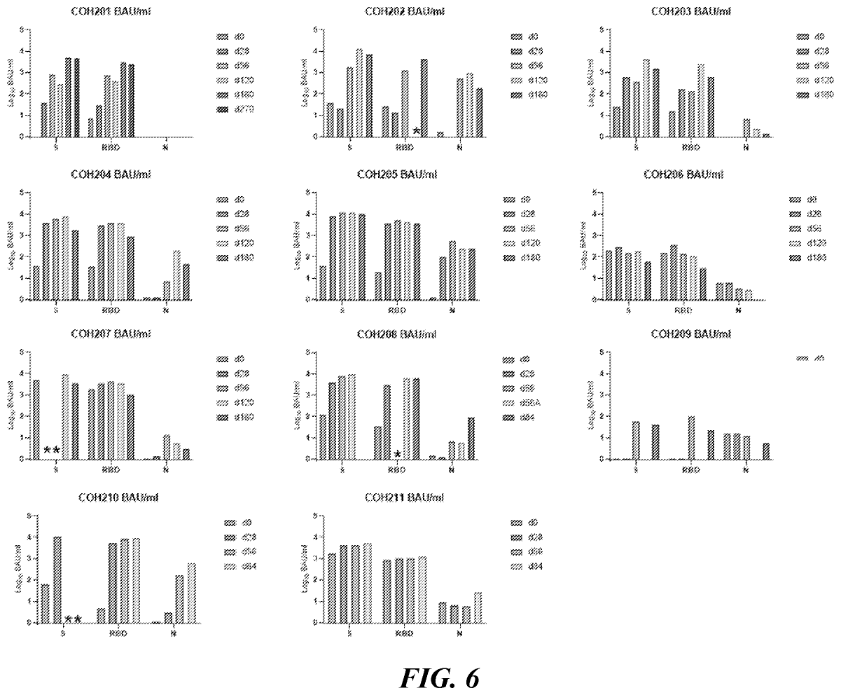

SARS-CoV-2 binding antibodies. Most patients showed a robust increase in S- and RBD-specific IgG titers after one COH04S1 dose and a booster effect after the second dose was evident (FIG. 6). However, CAR-T cell therapy patient COH206 did not show an increase in SARS-CoV-2 specific-IgG post-COH04S1 vaccination. An increase in N-specific IgG was observed in more than half of the patients.

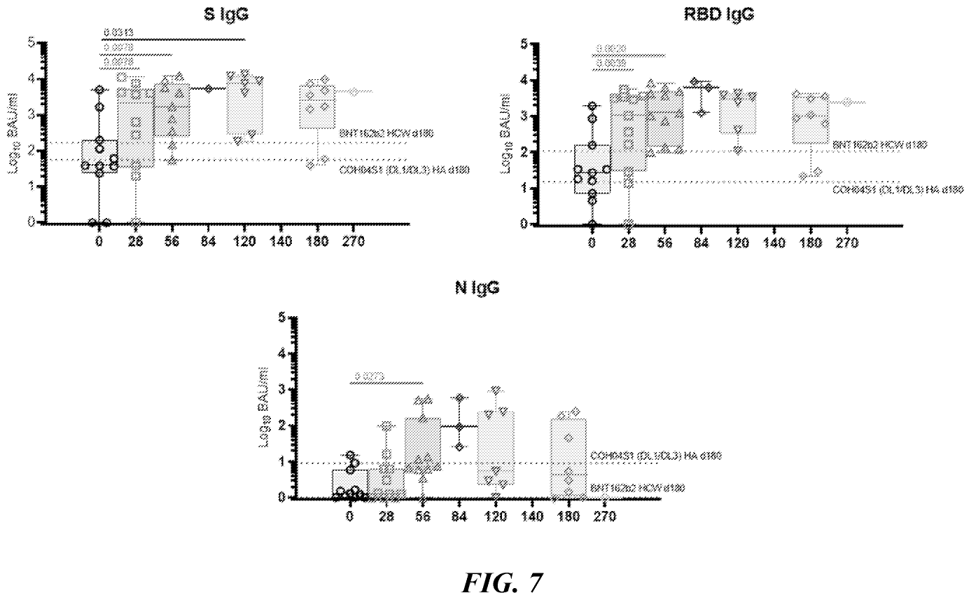

Statistical evaluation indicated a significant increase in S-specific IgG titers at day 28, 56 and 120 post-vaccination. RBD-specific IgG titers were significantly elevated compared to baseline at days 28 and 56 post-vaccination. N-specific IgG titers were significantly elevated compared to baseline one month after the second COH04S1 vaccination (FIG. 7). Of note, median S and RBD IgG titers at day 180 were more elevated that median titers measured in both healthcare workers (n=30) vaccinated with BNT162b2 (Comirnaty, Pfizer) at the beginning of the pandemic, and median IgG titers measured in healthy adults (n=30) vaccinated with COH04S1 (mostly at 1×10⁷ pfu, but also at 1×10⁸ pfu and 2.5×10⁸ pfu).

SARS-CoV-2 neutralizing antibodies. Titers of neutralizing antibodies against ancestral SARS-CoV-2 and SARS-CoV-2 Beta, Delta, and Omicron BA.1 VOC were measured in serum of patients at baseline and at 28, 56, 120 and 180 days after COH04S1 vaccination. In most cases, COH04S1 booster vaccination resulted in a >3-fold increase in NAb titers against SARS-CoV-2 and its VOC compared to baseline (FIG. 7). Fold increase in NAb titers ranged from 6× to 1,970× with the exception of CAR-T cell recipient COH206 who did not respond to COH04S1 with an increase in SARS-CoV-2 specific NAb. Magnitude of NAb response to SARS-CoV-2 variants was comparable to ancestral SARS-CoV-2 (D614G) but with slightly reduced titers especially when evaluated against Omicron.

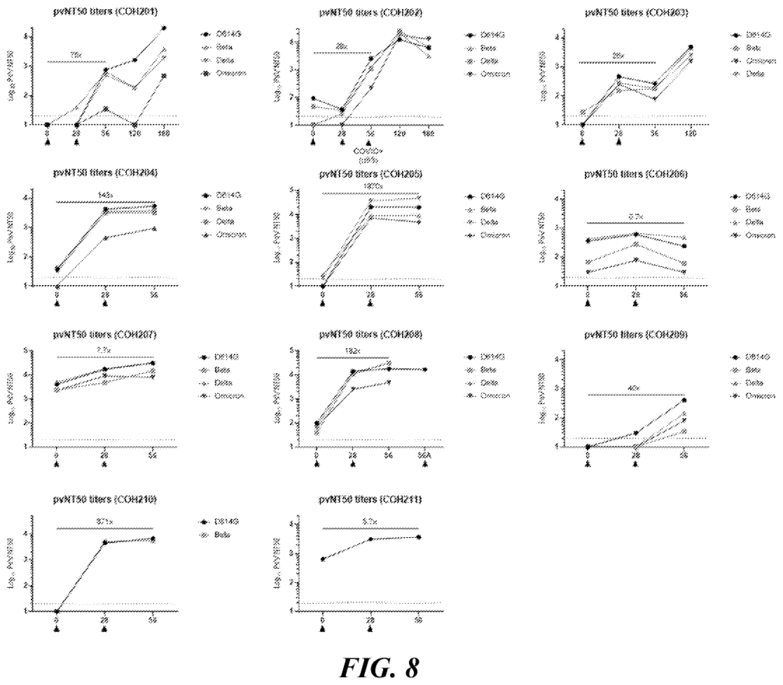

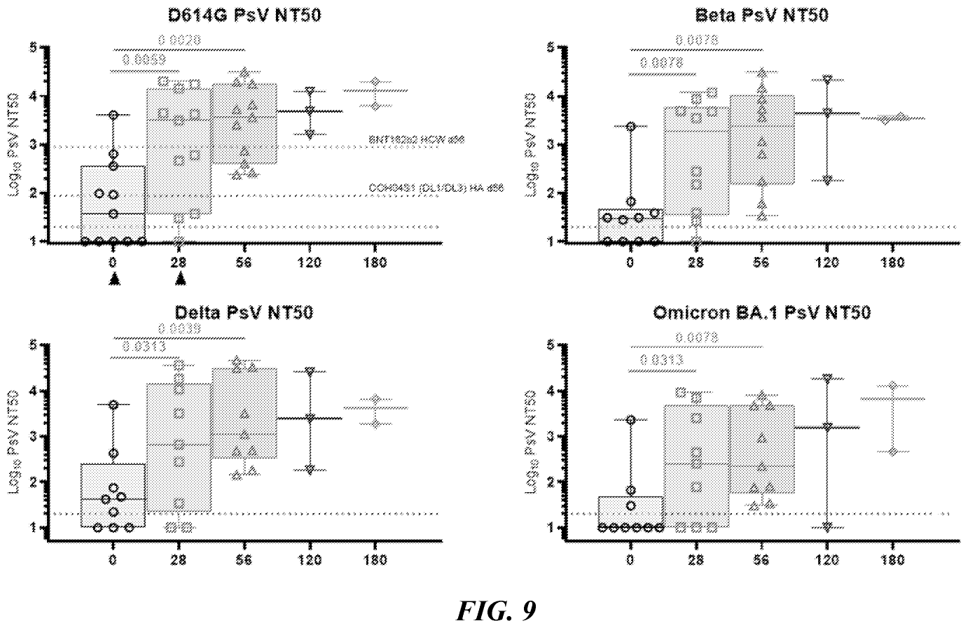

Statistical evaluation revealed a significant increase in SARS-CoV-2 specific NAb titers at 28- and 56-days post-vaccination for all the strains evaluated (FIG. 8). Remarkably, median NT50 titers approaching 103 were measured in boosted volunteers against Omicron BA.1 which is known to escape NAb resulting in lower titers. Of note, median ancestral-specific NAb titers at day 56 were more elevated that median titers measured in healthcare workers (n=30) vaccinated with BNT162b2 (Comirnaty, Pfizer) at the beginning of the pandemic, and compared to median titers measured in healthy adults (n=30) vaccinated with COH04S1 (mostly at 1×10⁷ pfu, but also at 1×10⁸ pfu and 2.5×10⁸ pfu).

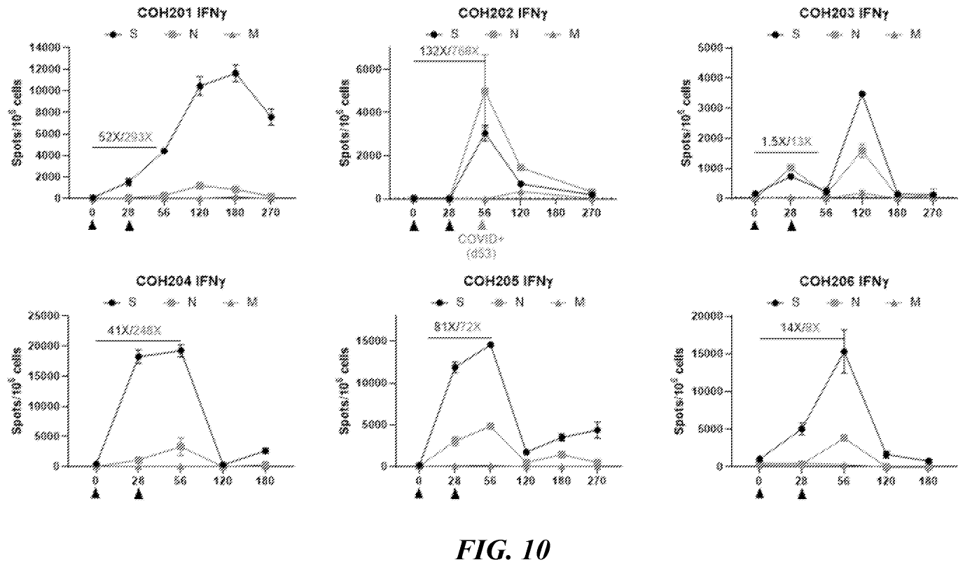

IFNγ/IL-4 T cell responses. T cells secreting IFNγ and/or IL-4 cytokines upon stimulation with SARS-CoV-2 S-, N-, and M-specific peptide libraries were measured in PBMCs from patients at 28-, 56-, 120-, 180- and 270-days post-vaccination with COH04S1 using FluoroSpot assay. An increase in S- and/or N-specific T cells secreting IFNγ was observed in most patients after COH04S1 vaccination (FIG. 10). The majority of patients had a more robust response to S than N, possibly due to the presence in the graft of vaccine-induced S-specific T cells acquired pre-transplant. The only exception was COH202, who was COVID-19-positive few days before day 56 sampling and showed balanced T cell response to N and S, probably because SARS-CoV-2 infection induced a robust recall response to both vaccine antigens. Interestingly, CAR-T cell therapy patient COH206, despite the absence of vaccine-induced humoral responses, developed robust T cell responses to both S and N antigens with S-specific T cells peaking at about 15,000 spots/10⁶ cells. This result was unexpected, as the immunity was mainly T cell-based as opposed to other EUA and FDA-approved vaccines in these patients.

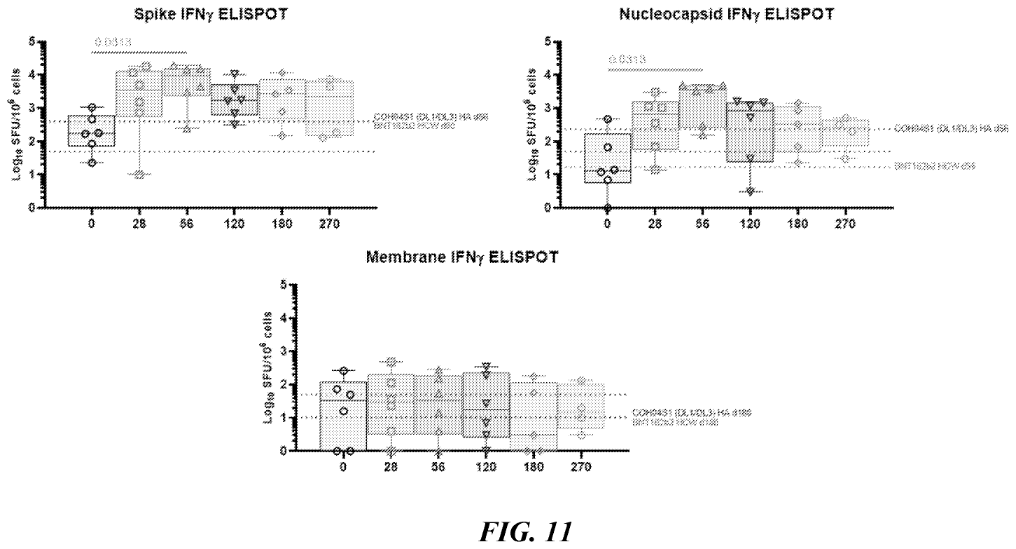

Statistical evaluation indicated that both S- and N-specific IFNγ T cell responses were significantly elevated after the second dose of COH04S1 compared to baseline (FIG. 11). Of note, median S- and N-specific IFNγ T cell responses at day 56 were more elevated that median T cell levels measured in healthcare workers (n=30) vaccinated with BNT162b2 (Comirnaty, Pfizer) at the beginning of the pandemic, and compared to median S- and N-specific T cell responses measured in healthy adults (n=30) vaccinated with COH04S1.

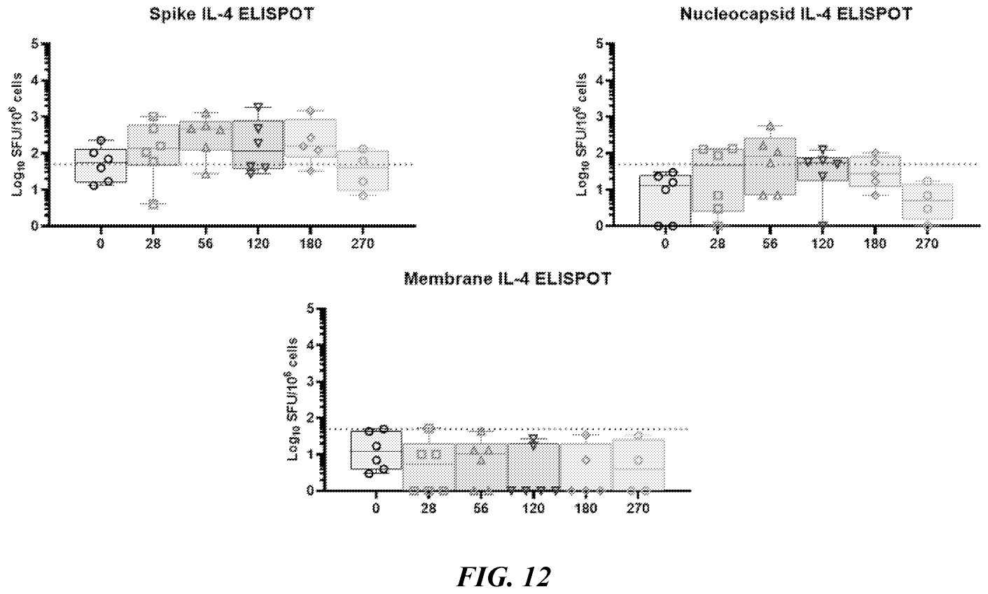

IL-4 responses against all antigens were low throughout the study. Both S- and N-specific T cells secreting IL-4 did not increase significantly following vaccination with COH04S1 (FIG. 12).

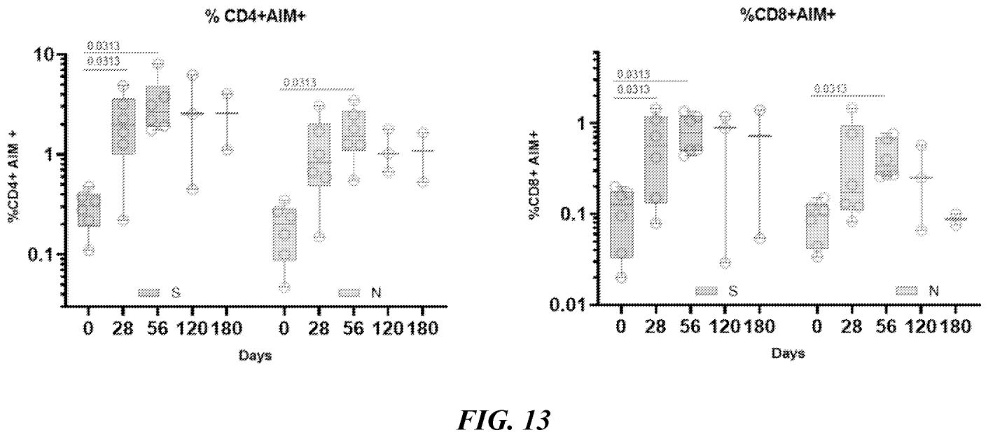

Activation-induced marker positive T cells. T cells expressing activation induced markers (AIM+) upon stimulation with SARS-CoV-2 S and N peptides were evaluated in samples of COH04S1 vaccinated patients at baseline and at days 28, 56, 120 and 180 post-vaccination. As shown in FIG. 13, a significant increase in S-specific AIM+CD4+ and CD8+ T cells was measured after the prime dose, whereas a significant increase in both S- and N-specific AIM+CD4+ and CD8+ T cells was measured after two vaccine doses.

Vaccination of autologous and allogeneic transplant patients with COH04S1 at 2.5×10⁸ pfu resulted in a significant increase in S- and N-specific IgG and T cells and SARS-CoV-2 specific NAb. The only CAR-T cell patient enrolled at this moment, despite the absence of SARS-CoV-2 specific antibodies, developed a robust T cell response post-vaccination. These results indicate a better response in this patient population compared to healthy adults naïve for SARS-CoV-2 possibly due to SARS-CoV-2 immunity acquired pre-transplant and present in the graft.

### Example 3. Trial of COH04S1 in Patients Having Poor Immune Response to Previous COVID-19 Vaccination

This study is designed as a single-center, phase 2, multiple-patient-type trial following an open label safety lead-in phase to evaluate immune response at day 56 post COVID-19 vaccine boost using a synthetic MVA-based SARS-CoV-2 vaccine boost (COH04S1). Patients will receive a single intramuscular booster injection of COH04S1 at 2.5×10⁸ PFU/dose or a Pfizer COVID-19 vaccine SOC. The various patient types tested consist of groups of patients with related malignant disease or therapy. The first type is patients with B-cell hematologic malignancies.

Toxicity is the primary endpoint in the safety lead-in phase. A standard 3+3 design is used as ‘go’ or ‘not to go’ vaccinate additional subjects. A cohort of 3 subjects is vaccinated with COH04S1 first. Moderate toxicity (MOD): grade 2 AEs (based on CTCAE version 5.0) probably or definitely attributable to COH04S1 vaccines, and lasting 7 days or longer are monitored from vaccination of COH04S1 to the first occurrence of MOD or 28 days post injection, whichever comes first. If at most one subject experiences MOD, vaccinate another cohort of 3 subjects will be vaccinated. Vaccination will be suspended as soon as two or more subjects experiencing MOD are observed during the observation period. Vaccination will be paused if any subjects having UT or NRM events is observed during the observation period: from the vaccination to 28 days post injection. If at most 1 out of 6 evaluable subjects has MOD and the safety profile is acceptable (no UT and NRM are observed), the phase 2 segment will be started.

In the parallel phase 2 segment, a Simon two-stage design is used to determine the sample size, boundaries of immune responses, and whether accrual is suspended in a therapy type at interim analysis. Up to 37 subjects are treated in each therapy type in the phase 2 segment. Six subjects vaccinated in the safety-lead in segment will be counted as part of the first stage of the phase 2 design. A total of ˜40 patients in each therapy type will be accrued to account for an unevaluable rate of 8%. Immune responses are evaluated at interim analysis and final analysis in each arm.

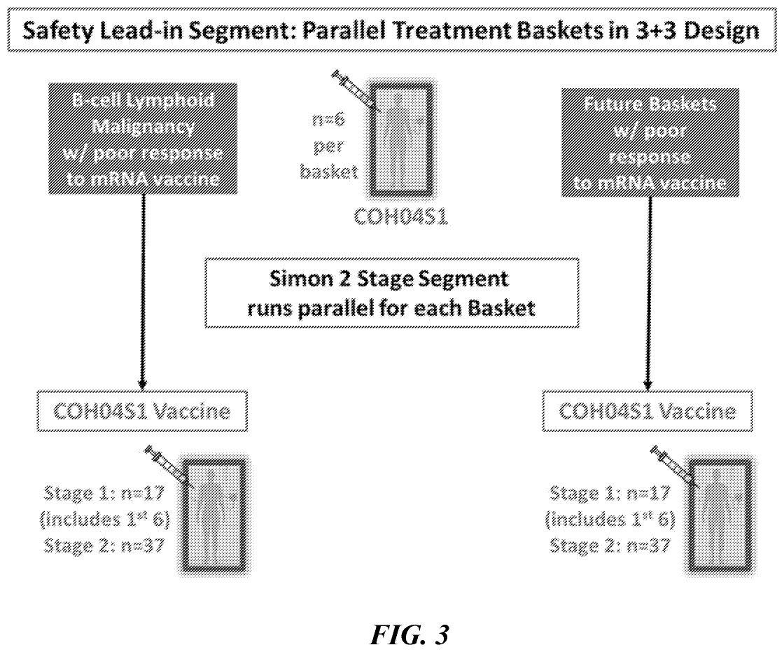

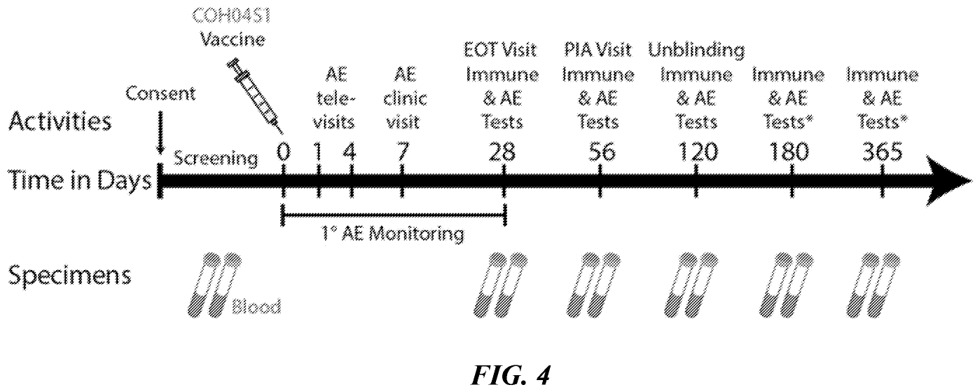

FIG. 3 shows the design schema and FIG. 4 shows the treatment schema.

A comprehensive and innovative panel of immune assays combined with safe and versatile virological tools is designed to characterize the COH04S1 vaccine induced SARS-CoV-2-specific adaptive immunity in this clinical trial. The integrated platform of immune- and pseudovirus-based methods includes analytical multiparameter flow cytometry; qualitative in house developed ELISA, and neutralization assays based on a SARS-CoV-2 lentiviral-pseudovirus system, expressing the Spike antigen and infecting cell lines engineered to express ACE2. The assay system with Spike antigen “pseudotyped” onto non-replicative lentiviral particles alleviates the biosafety-level-3 (BSL3) hazard associated with working directly with SARS-CoV-2 and allows a safer approach to assess sera neutralizing activity to SARS-CoV-2.

Humoral immunity (IgA, IgG, and IgM) in serum is assessed by ELISA. The neutralizing capability of the antibodies to prevent infection of a susceptible cell line is evaluated using a pseudo-type of the SARS-CoV-2 virus. To evaluate the Th1 vs Th2 polarization of immune responses, which has been observed in convalescing COVID-19 cases, a SARS-CoV-2-specific ELISPOT is performed to measure IFNγ and IL-4 cytokine levels, by using overlapping peptide libraries specific for SARS-CoV-2. Additionally, the following are evaluated: a) levels of antigen-specific T cell responses using overlapping peptide library specific for SARS-CoV-2; and b) functional activated/cycling and memory phenotype marker evolution on the surface of antigen specific T cells elicited as a result of the vaccination.

### REFERENCES

The references, patents and published patent applications listed below, and all references cited in the specification above are hereby incorporated by reference in their entirety, as if fully set forth herein.
1. Krause P R, Fleming T R, Longini I M, et al. SARS-CoV-2 Variants and Vaccines. N Engl J Med 2021; 385(2): 179-86.
2. Edara V V, Pinsky B A, Suthar M S, et al. Infection and Vaccine-Induced Neutralizing-Antibody Responses to the SARS-CoV-2 B.1.617 Variants. N Engl J Med 2021; 385(7): 664-6.
3. Liu Y, Liu J, Xia H, et al. BNT162b2-Elicited Neutralization against New SARS-CoV-2 Spike Variants. N Engl J Med 2021; 385(5): 472-4.
4. Lopez Bernal J, Andrews N, Gower C, et al. Effectiveness of Covid-19 Vaccines against the B.1.617.2 (Delta) Variant. N Engl J Med 2021; 385(7): 585-94.
5. Chiuppesi F, Salazar M D, Contreras H, et al. Development of a multi-antigenic SARS-CoV-2 vaccine candidate using a synthetic poxvirus platform. Nat Commun 2020; 11(1): 6121.
6. Volz A, Sutter G. Modified Vaccinia Virus Ankara: History, Value in Basic Research, and Current Perspectives for Vaccine Development. Adv Virus Res 2017; 97: 187-243.
7. Aldoss I, La Rosa C, Baden L R, et al. Poxvirus Vectored Cytomegalovirus Vaccine to Prevent Cytomegalovirus Viremia in Transplant Recipients: A Phase 2, Randomized Clinical Trial. Ann Intern Med 2020; 172(5): 306-16.
8. Chiuppesi F, Nguyen V H, Park Y, et al. Synthetic Multiantigen MVA Vaccine COH04S1 Protects Against SARS-CoV-2 in Syrian Hamsters and Non-Human Primates. bioRxiv 2021.
9. Antoine G, Scheiflinger F, Dorner F, Falkner F G. The Complete Genomic Sequence of the Modified Vaccinia Ankara Strain: Comparison With Other Orthopoxviruses. Virology. 10 May 1998; 244(2):365-96.
10. Scheiflinger F, Dorner F, Falkner F G. Construction of Chimeric Vaccinia Viruses by Molecular Cloning and Packaging. Proceedings of the National Academy of Sciences of the United States of America. 1 Nov. 1992; 89(21):9977-81.
11. Liang, W., W. Guan, R. Chen, W. Wang, J. Li, K. Xu, C. Li, Q. Ai, W. Lu, H. Liang, S. Li, and J. He. 2020. Cancer patients in SARS-CoV-2 infection: a nationwide analysis in China. Lancet Oncol 21: 335-337.
12. Sanders, J. M., M. L. Monogue, T. Z. Jodlowski, and J. B. Cutrell. 2020. Pharmacologic Treatments for Coronavirus Disease 2019 (COVID-19): A Review. JAMA.
13. Dagnew, A. F., O. Ilhan, W. S. Lee, D. Woszczyk, J. Y. Kwak, S. Bowcock, S. K. Sohn, G. Rodriguez Macias, T. J. Chiou, D. Quiel, M. Aoun, M. B. Navarro Matilla, J. de la Serna, S. Milliken, J. Murphy, S. A. McNeil, B. Salaun, E. Di Paolo, L. Campora, M. Lopez-Fauqued, M. El Idrissi, A. Schuind, T. C. Heineman, P. Van den Steen, L. Oostvogels, and g. Zoster-039 study. 2019. Immunogenicity and safety of the adjuvanted recombinant zoster vaccine in adults with haematological malignancies: a phase 3, randomised, clinical trial and post-hoc efficacy analysis. The Lancet. Infectious diseases 19: 988-1000.
14. Rubin, L. G., M. J. Levin, P. Ljungman, E. G. Davies, R. Avery, M. Tomblyn, A. Bousvaros, S. Dhanireddy, L. Sung, H. Keyserling, I. Kang, and A. Infectious Diseases Society of. 2014. 2013 IDSA clinical practice guideline for vaccination of the immunocompromised host. Clinical infectious diseases: an official publication of the Infectious Diseases Society of America 58: 309-318.
15. Alharbi, N. K., E. Padron-Regalado, C. P. Thompson, A. Kupke, D. Wells, M. A. Sloan, K. Grehan, N. Temperton, T. Lambe, G. Warimwe, S. Becker, A. V. S. Hill, and S. C. Gilbert. 2017. ChAdOx1 and MVA based vaccine candidates against MERS-CoV elicit neutralising antibodies and cellular immune responses in mice. Vaccine 35: 3780-3788.
16. Walsh, S. R., M. B. Wilck, D. J. Dominguez, E. Zablowsky, S. Bajimaya, L. S. Gagne, K. A. Verrill, J. A. Kleinjan, A. Patel, Y. Zhang, H. Hill, A. Acharyya, D. C. Fisher, J. H. Antin, M. S. Seaman, R. Dolin, and L. R. Baden. 2013. Safety and immunogenicity of modified vaccinia Ankara in hematopoietic stem cell transplant recipients: a randomized, controlled trial. The Journal of infectious diseases 207: 1888-1897.
17. La Rosa, C., J. Longmate, J. Martinez, Q. Zhou, T. I. Kaltcheva, W. Tsai, J. Drake, M. Carroll, F. Wussow, F. Chiuppesi, N. Hardwick, S. Dadwal, I. Aldoss, R. Nakamura, J. A. Zaia, and D. J. Diamond. 2017. MVA vaccine encoding CMV antigens safely induces durable expansion of CMV-specific T cells in healthy adults. Blood 129: 114-125.
18. Crawford, K. H. D., R. Eguia, A. S. Dingens, A. N. Loes, K. D. Malone, C. R. Wolf, H. Y. Chu, M. A. Tortorici, D. Veesler, M. Murphy, D. Pettie, N. P. King, A. B. Balazs, and J. D. Bloom. 2020. Protocol and Reagents for Pseudotyping Lentiviral Particles with SARS-CoV-2 Spike Protein for Neutralization Assays. Viruses 12.
19. Goonetilleke, N., S. Moore, L. Dally, N. Winstone, I. Cebere, A. Mahmoud, S. Pinheiro, G. Gillespie, D. Brown, V. Loach, J. Roberts, A. Guimaraes-Walker, P. Hayes, K. Loughran, C. Smith, J. De Bont, C. Verlinde, D. Vooijs, C. Schmidt, M. Boaz, J. Gilmour, P. Fast, L. Dorrell, T. Hanke, and A. J. McMichael. 2006. Induction of multifunctional human immunodeficiency virus type 1 (HIV-1)-specific T cells capable of proliferation in healthy subjects by using a prime-boost regimen of DNA- and modified vaccinia virus Ankara-vectored vaccines expressing HIV-1 Gag coupled to CD8+ T-cell epitopes. J Virol 80: 4717-4728.
20. Robinson, H. L., D. C. Montefiori, F. Villinger, J. E. Robinson, S. Sharma, L. S. Wyatt, P. L. Earl, H. M. McClure, B. Moss, and R. R. Amara. 2006. Studies on G M-CSF DNA as an adjuvant for neutralizing Ab elicited by a DNA/MVA immunodeficiency virus vaccine. Virology 352: 285-294.
21. Santra, S., Y. Sun, J. G. Parvani, V. Philippon, M. S. Wyand, K. Manson, A. Gomez-Yafal, G. Mazzara, D. Panicali, P. D. Markham, D. C. Montefiori, and N. L. Letvin. 2007. Heterologous prime/boost immunization of rhesus monkeys by using diverse poxvirus vectors. J Virol 81: 8563-8570.
22. Ou, X., Y. Liu, X. Lei, P. Li, D. Mi, L. Ren, L. Guo, R. Guo, T. Chen, J. Hu, Z. Xiang, Z. Mu, X. Chen, J. Chen, K. Hu, Q. Jin, J. Wang, and Z. Qian. 27 Mar. 2020. Characterization of Spike Glycoprotein of SARS-CoV-2 on Virus Entry and Its Immune Cross-Reactivity With SARS-CoV. Nature communications 11: 1620.
23. Tay, M. Z., C. M. Poh, L. Renia, P. A. MacAry, and L. F. P. Ng. 2020. The trinity of COVID-19: immunity, inflammation and intervention. Nat Rev Immunol 20: 363-374.
24. Sekine, T., A. Perez-Potti, S. Nguyen, J. B. Gorin, V. H. Wu, E. Gostick, S. Llewellyn-Lacey, Q. Hammer, S. Falck-Jones, S. Vangeti, M. Yu, A. Smed-Sorensen, A. Gaballa, M. Uhlin, J. K. Sandberg, C. Brander, P. Nowak, P. A. Goepfert, D. A. Price, M. R. Betts, and M. Buggert. 2020. TOX is expressed by exhausted and polyfunctional human effector memory CD8(+) T cells. Sci Immunol 5.
25. Epker, J. L., A. J. Valkenburg, and E. J. O. Kompanje. 2021. Double data and dubious conclusions, ‘Houston do we have a problem?’. Intensive Care Med.
26. Bagarazzi, M. L., J. Yan, M. P. Morrow, X. Shen, R. L. Parker, J. C. Lee, M. Giffear, P. Pankhong, A. S. Khan, K. E. Broderick, C. Knott, F. Lin, J. D. Boyer, R. Draghia-Akli, C. J. White, J. J. Kim, D. B. Weiner, and N. Y. Sardesai. 2012. Immunotherapy Against HPV16/18 Generates Potent TH1 and Cytotoxic Cellular Immune Responses. Science translational medicine 4.
27. Wolfl, M., M. Eyrich, P. Schlegel, and P. Greenberg. 2007. T-cells in stem cell transplants: dissecting the good, the bad and the ugly. Journal of stem cells & regenerative medicine 2: 26.

1. A method of vaccinating or protecting a subject against coronavirus disease 2019 (COVID-19); preventing a coronavirus infection; preventing, treating, or reducing the severity of COVID-19; or treating COVID-19 caused by a coronavirus infection, comprising administering to the subject a composition comprising a synthetic MVA (sMVA) vector comprising or capable of expressing one or more DNA sequences encoding a spike (S) protein and a nucleocapsid (N) protein or variants or mutants thereof, wherein the subject is a blood cancer patient that (1) has been treated for a hematological malignancy with a cellular therapy or (2) has had or is likely to have a poor immune response to a different COVID-19 vaccination.

2-16. (canceled)

17. The method of claim 1, wherein the cellular therapy is selected from the group consisting of an autologous hematopoietic cell transplant, an allogeneic hematopoietic cell transplant, a chimeric antigen receptor (CAR)-T cell therapy, and a combination thereof.

18. The method of claim 1, wherein the subject received the cellular therapy at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, or at least 12 months prior to administration of the composition.

19. The method of claim 1, wherein the coronavirus infection is caused by the Wuhan-Hu-1 reference strain or a variant of concern (VOC) selected from the group consisting of B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

20. The method of claim 1, wherein the composition is administered by intramuscular injection, intranasal instillation, intradermal injection, and/or scarification.

21. The method of claim 1, wherein the composition is administered to the subject in a single dose, two doses, three doses, four doses, or more than four doses.

22. The method of claim 1, wherein the composition is administered to the subject in a prime dose followed by one or more booster doses.

23. The method or of claim 22, wherein an interval between the prime dose and the first booster dose is about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, or about 16 weeks.

24. The method of claim 22, wherein the prime dose is between 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose.

25. The method of claim 22, wherein the booster dose is between 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose.

26. The method of claim 22, wherein the booster dose is administered in a dosage the same as the prime dose.

27. The method or of claim 22, wherein the booster dose is administered in a dosage lower than the prime dose.

28. The method of claim 1, wherein the subject suffers from or previously suffered from a hematological malignancy.

29. The method of claim 28, wherein the hematological malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

30. The method of claim 1, wherein the subject has previously received one or more COVID-19 vaccines.

31. The method of claim 30, wherein the previously received COVID-19 vaccine is an mRNA-, adenovirus-, or protein-based vaccine.

32. The method of claim 30, wherein the previously received COVID-19 vaccine is Pfizer-BioNTech COVID-19 vaccine (BNT162b2), Moderna COVID-19 vaccine (mRNA-1273), Janssen COVID-19 vaccine (Ad26.COV2.S), Novavax COVID-19 vaccine adjuvanted, or Oxford-AstraZeneca ChAdOx1 nCoV-19 vaccine (AZD1222).

33. The method of claim 30, wherein the previously received COVID vaccine comprises an S antigen or a coding sequence for an S antigen only.

34. The method of claim 30, wherein the subject has received the previous vaccination at least 1 month, at least 2 months, at least 3 months, at least 4 months, at least 4 months, at least 5 months, at least 6 months, at least 7 months, at least 8 months, at least 9 months, at least 10 months, at least 11 months, or at least 12 months prior to administration of the composition.
